(19) United States
(12) Patent Application Publication
(10) Pub. No.: US 2016/0318866 A1
(43) Pub. Date: Nov. 3, 2016

(54) SUBSTITUTED BIPIPERIDINYL DERIVATIVES

(71) Applicant: Bayer Pharma Aktiengesellschaft, Berlin (DE)

(72) Inventors: Eva Maria BECKER-PELSTER, Wuppertal (DE); Philipp BUCHGRABER, Berlin (DE); Anja BUCHMÜLLER, Essen (DE); Karen ENGEL, Roßdorf (DE); Mark Jean GNOTH, Mettmann (DE); Herbert HIMMEL, Essen (DE); Raimund KAST, Wuppertal (DE); Joerg KELDENICH, Berlin (DE); Jürgen KLAR, Wuppertal (DE); Andreas KNORR, Erkrath (DE); Dieter LANG, Velbert (DE); Niels LINDNER, Wuppertal (DE); Carsten SCHMECK, Mülheim (DE); Rudolf SCHOHE-LOOP, Wuppertal (DE); Hanna TINEL, Wuppertal (DE); Hubert TRÜBEL, Wuppertal (DE); Frank WUNDER, Wuppertal (DE)

(73) Assignee: Bayer Pharma Aktiengesellschaft, Berlin (DE)

(21) Appl. No.: 15/106,285

(22) PCT Filed: Dec. 16, 2014
(86) PCT No.: PCT/EP2014/077863

§ 371 Date: Jun. 18, 2016

(30) Foreign Application Priority Data
Dec. 19, 2013 (EP) 13198389.2
Nov. 12, 2014 (EP) 14192876.2

(51) Int. Cl. C07D 211/10; C07D 211/42; C07D 211/22; A61K 45/06; C07D 401/14; C07D 413/14; C07D 405/14; C07D 211/60; A61K 31/4545

(52) U.S. Cl. CPC C07D 211/10; C07D 211/60; C07D 211/42; C07D 211/22; A61K 31/4545; C07D 401/14; C07D 413/14; C07D 405/14; A61K 45/06

(57) ABSTRACT

The invention relates to novel substituted bipiperidinyl derivatives, to processes for their preparation, to their use for the treatment and/or prevention of diseases and to their use for preparing medicaments for the treatment and/or prevention of diseases, in particular for the treatment and/or prevention of diabetic microangiopathies, diabetic ulcers on the extremities, in particular for promoting wound healing of diabetic foot ulcers, diabetic heart failure, diabetic coronary microvascular heart disorders, peripheral and cardiac vascular disorders, thromboembolic disorders and ischaemias, peripheral circulatory disturbances, Raynaud's phenomenon, CREST syndrome, microcirculatory disturbances, intermittent claudication, and peripheral and autonomous neuropathies.

The invention relates to novel substituted bipiperidinyl derivatives, to processes for their preparation, to their use in a method for the treatment and/or prophylaxis of diseases and to their use for preparing medicaments for the treatment and/or prophylaxis of diseases, in particular of cardiovascular disorders, diabetic microangiopathies, diabetic ulcers on the extremities, in particular for promoting wound healing of diabetic foot ulcers, diabetic heart failure, diabetic coronary microvascular heart disorders, peripheral and cardiac vascular disorders, thromboembolic disorders and ischaemias, peripheral circulatory disturbances, Raynaud's phenomenon, CREST syndrome, microcirculatory disturbances, intermittent claudication, and peripheral and autonomous neuropathies.

Adrenoreceptor α₂ receptors (α₂-ARs) belong to the family of the G-protein-coupled receptors. They bind to the pertussis toxin-sensitive inhibitory G protein Gᵢ and G₀ and reduce adenylate cyclase activity. They are involved in the mediation of diverse physiological effects in various tissues following stimulation by endogenous catecholamines (adrenalinee, noradrenalinee) which are either released by synapses or reach the site of action via the blood. α₂-AR play an important physiological role, mainly for the cardiovascular system, but also in the central nervous system. Biochemical, physiological and pharmacological studies have shown that, in addition to various α₁-AR subtypes, there are three α₂-AR subtypes (α_{2A}, α_{2B} and α_{2C}) in many target cells and tissues of cardiovascular relevance, which makes them attractive target proteins for therapeutic interventions. However, the elucidation of the precise physiological task of the receptor subtypes remains difficult to date because of a lack of highly selective ligands and/or antagonists of the respective α₂-AR (Gyires et al., α₂-Adrenoceptor subtypes-mediated physiological, pharmacological actions, Neurochemistry International 55, 447-453, 2009; Tan and Limbird, The α₂₋Adrenergic Receptors: Adrenergic Receptors in the 21st Century/Receptors, 2005, 241-265).

Cardiovascular changes such as, for example, the regulation of the contractility of the heart are regulated, firstly, by the central modulation of the sympathetic efferent nerves. Furthermore, the sympathetic efferent system also regulates direct effects on smooth muscle cells and the endothelial cells of the vessels. Thus, the sympathetic system is involved in the regulation of the output performance of the heart, but also in the control of local perfusion of various vascular beds. This is also controlled via α₂-ARs involved in the regulation of the peripheral resistance. Thus, blood vessels are innervated by sympathetic nerve fibres which run in the adventitia and whose endings are provided with varicosities for the release of noradrenaline. Released noradrenaline modulates, via the α₂-AR in endothelial cells and smooth muscle cells, the respective local vascular tone.

In addition to the effects on the sympathetic efferent nerves, the peripheral cardiovascular function is also regulated by pre- and postsynaptic α₂-AR. Smooth muscle cells and endothelial cells express different α₂-AR subtypes. The activation of α_{2A}, α_{2B} and α_{2C} receptors on smooth muscle cells leads to contraction with resulting vasoconstriction (Kanagy, Clinical Science 109:431-437, 2005). However, the distribution of the respective receptor subtypes varies in the different vascular beds, between the species and between different vessel sizes. Thus, α_{2A}-AR appear to be expressed virtually exclusively in large arteries, whereas α_{2B}-AR contribute more to the vascular tone in small arteries and veins. ARα_{2B} appears to play a role in salt-induced hypertension (Gyires et al., α₂-Adrenoceptor subtypes-mediated physiological, pharmacological actions, Neurochemistry International 55, 447-453, 2009). The role of ARα_{2C} on haemodynamics is not yet completely understood; however, ARα_{2C} receptors appear to mediate venous vasoconstriction. They are also involved in cold-induced enhancement of adrenoceptor-induced vasoconstriction (Chotani et al., Silent α_{2C} adrenergic receptors enable cold-induced vasoconstriction in cutaneous arteries. Am J Physiol 278:H1075-H1083, 2000; Gyires et al., α₂-Adrenoceptor subtypes-mediated physiological, pharmacological actions, Neurochemistry International 55, 447-453, 2009). Cold and other factors (e.g. tissue proteins, oestrogen) regulate the functional coupling of ARα_{2C} to intracellular signal pathways (Chotani et al., Distinct cAMP signaling pathways differentially regulate α_{2C} adrenenoxceptor expression: role in serum induction in human arteriolar smooth muscle cells. Am J Physiol Heart Circ Physiol 288: H69-H76, 2005). For this reason, it makes sense to investigate selective inhibitors of AR-α₂ subtypes for their perfusion-modulating effect on different vascular beds under different pathophysiological conditions.

Under pathophysiological conditions, the adrenergic system may be activated, which can lead, for example, to hypertension, heart failure, increased platelet activation, endothelial dysfunction, atherosclerosis, angina pectoris, myocardial infarction, thromboses, peripheral circulatory disturbances, stroke and sexual dysfunction. Thus, for example, the pathophysiology of Raynaud's syndrome and scleroderma is substantially unclear, but is associated with a changed adrenergic activity. Thus, patients suffering from spastic Raynaud's syndrome show, for example, a significantly elevated expression of ARα₂ receptoren on their platelets. This may be connected with the vasospastic attacks observed in these patients (Keenan and Porter, α₂-Adrenergic receptors in platelets from patients with Raynaud's syndrome, Surgery, V94(2), 1983).

By virtue of the expected high efficiency and low level of side effects, a possible treatment for such disorders targeting the modulation of the activated adrenergic system in organisms is a promising approach. In particular in diabetics, who frequently have elevated catecholamine levels, peripheral circulatory disturbances (microangiopathies) such as diabetic retinopathy, nephropathy or else pronounced wound healing disorders (diabetic foot ulcers) play a large role. In peripheral occlusive disease, diabetes mellitus is one of the most important comorbidities and also plays a crucial role in the progression of the disease (micro- and macroangiopathy). Higher expression of the adrenoreceptor α_{2C} receptors associated with elevated catecholamine levels may be involved in these pathophysiological processes in diabetics. In 2011 there were 350 million diabetics world-wide (≈6.6% of the population), and this number is expected to double by 2028. Diabetic foot ulcers are the most frequent cause of hospitalizations of diabetics. The risk of a diabetic developing a diabetic foot ulcer in his or her lifetime is 15-25%, 15% of all diabetic foot ulcers lead to amputation. World-wide, 40-70% of all non-traumatic amputations are carried out on diabetics. Risk factors for diabetic foot ulcers are traumata, poor metabolic control, sensory, motoric and autonomous polyneuropathy, inappropriate footwear, infections and peripheral arterial disorders. The treatment of diabetic foot ulcers requires interdisciplinary teams and employs a multifactor approach: weight loss, revascularization (in the case of peripheral arterial occlusive disease, PAOD), improvements in metabolic control, wound excision, dressings, dalteparin, Regranex (PDGF) and amputation. The treatment costs per diabetic foot ulcer (without amputation) are 7000-10000 USD. 33% of all diabetic foot ulcers do not heal within 2 years, and there is a high relapse rate (34% within the first year, 61% over 3 years).

Accordingly, it is an object of the present invention to provide novel selective adrenoreceptor α_{2C} receptor antagonists for the treatment and/or prophylaxis of diseases such as, for example, cardiovascular disorders, in humans and animals.

It is another object of the present invention to provide novel selective adrenoreceptor α2C receptor antagonists for the treatment and/or prophylaxis of peripheral circulatory disturbances (microangiopathies) such as, for example, diabetic retinopathy, diabetic nephropathy and wound healing disorders (diabetic foot ulcers).

WO 2005/042517, WO 2003/020716, WO 2002/081449 and WO 2000/066559 describe structurally similar bipiperidinyl derivatives as inhibitors of the CCR5 receptor, inter alia for the treatment of HIV. WO 2005/077369 describes structurally similar bipiperidinyl derivatives as inhibitors of the CCR3 receptor, inter alia for the treatment of asthma. WO 94/22826 describes structurally similar piperidines as active compounds having peripheral vasodilating action. U.S. Pat. No. 6,444,681 B1 describes the general use of an α2C antagonist as peripheral vasodilator.

The invention provides compounds of the formula (I)

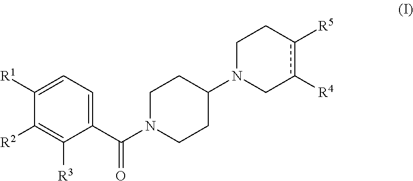

in which
[symbol] represents a single bond or a double bond,
R¹ is selected from the group consisting of C₃-C₆-alkyl, C₁-C₃-alkoxycarbonyl, oxetanyl, 5- or 6-membered heteroaryl, —(CR⁶R⁷)—R⁸ and —CONR⁹R¹⁰,
   where oxetanyl may be substituted by 1 or 2 substituents independently of one another selected from the group consisting of 3-hydroxy and 3-C₁-C₄-alkyl,
   and
   where
   R⁶ is selected from the group consisting of hydrogen, methyl and ethyl,
   R⁷ is selected from the group consisting of hydrogen, methyl and ethyl,
   or
   R⁶ and R⁷ together with the carbon atom to which they are attached form a cyclopropyl ring or cyclobutyl ring,
   R⁸ is selected from the group consisting of hydroxy, hydroxymethyl, C₁-C₄-alkyl, C₁-C₄-alkoxy, C₁-C₃-alkoxycarbonyl, C₁-C₄-alkylaminocarbonyl, phenoxy, oxetanyl, 5- or 6-membered heteroaryl and —CH₂NR¹³R¹⁴,
      where phenoxy and heteroaryl may be substituted by 1 to 3 substituents independently of one another selected from the group consisting of C₁-C₄-alkyl and C₁-C₄-alkoxy,
      where oxetanyl may be substituted by 1 or 2 substituents independently of one another selected from the group consisting of 3-C₁-C₄-alkyl and 3-OH,
      and
      where
      R¹³ is selected from the group consisting of hydrogen and C₁-C₄-alkyl,
      and
      R¹⁴ is selected from the group consisting of methyl, methylsulphonyl and formyl,
   R⁹ is selected from the group consisting of C₁-C₆-alkyl, C₃-C₆-cycloalkyl and 5- or 6-membered heteroaryl,
      where heteroaryl may be substituted by C₁-C₄-alkyl,
      where alkyl may be substituted by 1 to 3 substituents independently of one another selected from the group consisting of hydroxy, with the proviso that alkyl is C₂-C₆-alkyl, C₁-C₄-alkoxy, C₁-C₄-haloalkyl, C₃-C₆-cycloalkyl, phenyl, oxetanyl and 5- or 6-membered heteroaryl,
         in which this phenyl or heteroaryl for its part may be substituted by 1 to 3 substituents independently of one another selected from the group consisting of halogen, trifluoromethyl, difluoromethoxy, trifluoromethoxy and C₁-C₄-alkyl
         in which this oxetanyl for its part may be substituted by one or 2 substituents selected from the group consisting of 3-C₁-C₄-alkyl and 3-hydroxy;
   R¹⁰ is selected from the group consisting of hydrogen and C₁-C₄-alkyl,
   or
   R⁹ and R¹⁰ together with the nitrogen atom to which they are attached form a piperidinyl ring,
      where the piperidinyl ring may be substituted by 1 to 3 substituents independently of one another selected from the group consisting of C₁-C₄-alkyl,
R² is selected from the group consisting of hydrogen and halogen,
R³ is selected from the group consisting of hydrogen, halogen, hydroxy and C₁-C₄-alkoxy,
R⁴ is selected from the group consisting of C₁-C₃-alkyl, C₁-C₃-alkoxycarbonyl, C₃-C₆-cycloalkyl, C₃-C₆-cycloalkyl-C₁-C₃-alkoxy, C₃-C₆-cycloalkoxy, trifluoromethoxy-C₁-C₄-alkoxy, 5- or 6-membered heteroaryl and —OCONR¹¹R¹²,
   where alkyl may be substituted by a substituent selected from the group consisting of C₁-C₄-alkoxy, C₃-C₆-cycloalkoxy, trifluoromethoxy and phenoxy,
      in which this phenoxy for its part may be substituted by 1 to 3 substituents independently of one another selected from the group consisting of halogen,
   and
   where heteroaryl may be substituted by 1 to 3 substituents independently of one another selected from the group consisting of C₁-C₄-alkyl and C₃-C₆-cycloalkyl,
      in which this alkyl for its part may be substituted by a substituent selected from the group consisting of C₁-C₃-alkoxy and C₃-C₆-cycloalkyl,
   R¹¹ represents C₁-C₄-alkyl or C₃-C₆-cycloalkyl,
   R¹² is selected from the group consisting of hydrogen and C₁-C₄-alkyl,
   or
   R¹¹ and R¹² together with the nitrogen atom to which they are attached form a pyrrolidinyl ring,
R⁵ represents hydrogen or C₁-C₄-alkyl,
or one of the salts thereof, solvates thereof or solvates of the salts thereof.

Compounds of the invention are the compounds of the formula (I) and the salts, solvates and solvates of the salts thereof, the compounds that are encompassed by formula (I) and are of the formulae mentioned below and the salts, solvates and solvates of the salts thereof and the compounds that are encompassed by formula (I) and are mentioned below as embodiments and the salts, solvates and solvates of the salts thereof if the compounds that are encompassed by formula (I) and are mentioned below are not already salts, solvates and solvates of the salts.

In the context of the present invention, the term “x acid” in any formula does not mean a stoichiometrically defined ratio of acid to the respective substance. Depending, for example, on the basicity of the substance in question, the term “x acid” denotes various ratios of substance to acid, such as 10:1 to 1:10; 8:1 to 1:8; 7:1 to 1:7; 5:1 to 1:5; 4.5:1 to 1:4.5; 4:1 to 1:4; 3.5:1 to 1:3.5; 3:1 to 1:3; 2.5:1 to 1:2.5; 2:1 to 1:2; 1.5:1 to 1:1.5; and 1:1.

Preferred salts in the context of the present invention are physiologically acceptable salts of the compounds according to the invention. However, the invention also encompasses salts which themselves are unsuitable for pharmaceutical applications but which can be used, for example, for the isolation or purification of the compounds according to the invention.

Physiologically acceptable salts of the compounds according to the invention include acid addition salts of mineral acids, carboxylic acids and sulphonic acids, e.g. salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

According to one embodiment of the invention, salts of the compounds of the formula (I) are salts of trifluoroacetic acid, hydrochloric acid or formic acid.

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “sodium salt” or “x HCl”, “x CF3COOH”, “x Na+” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

In the context of the present invention, the term “x acid” in any formula does not mean a stoichiometrically defined ratio of acid to the respective substance. Depending, inter alia, on the basicity of the compound in question, the term “x acid” represents various ratios of substance to acid, such as 10:1 to 1:10; 8:1 to 1:8; 7:1 to 1:7; 5:1 to 1:5; 4.5:1 to 1:4.5; 4:1 to 1:4; 3.5:1 to 1:3.5; 3:1 to 1:3; 2.5:1 to 1:2.5; 2:1 to 1:2; 1.5:1 to 1:1.5; and 1:1.

Designated as solvates in the context of the invention are those forms of the compounds according to the invention which form a complex in the solid or liquid state by coordination with solvent molecules. Hydrates are a specific form of the solvates in which the coordination is with water.

The present invention additionally also encompasses prodrugs of the compounds of the invention. The term “prodrugs” encompasses compounds which for their part may be biologically active or inactive but are converted during their residence time in the body into compounds according to the invention (for example by metabolism or hydrolysis).

Depending on their structure, the compounds according to the invention may exist in stereoisomeric forms (enantiomers, diastereomers). The invention therefore encompasses the enantiomers or diastereomers and the respective mixtures thereof. It is possible to isolate the stereoisomerically homogeneous constituents from such mixtures of enantiomers and/or diastereomers in a known manner. Chromatographic methods, in particular HPLC chromatography using a chiral or achiral phase, are preferably used for this purpose.

If the compounds according to the invention can occur in tautomeric forms, the present invention encompasses all the tautomeric forms.

The present invention encompasses all possible stereoisomeric forms of the compounds of the formula (I) and of their starting materials, even if no stereoisomerism is stated.

The present invention also encompasses all suitable isotopic variants of the compounds of the invention. An isotopic variant of a compound of the invention is understood here to mean a compound in which at least one atom within the compound of the invention has been exchanged for another atom of the same atomic number, but with a different atomic mass from the atomic mass which usually or predominantly occurs in nature. Examples of isotopes which can be incorporated into a compound of the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound of the invention, especially those in which one or more radioactive isotopes have been incorporated, may be beneficial, for example, for the examination of the mechanism of action or of the active compound distribution in the body; due to comparatively easy preparability and detectability, especially compounds labelled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the compounds of the invention may therefore in some cases also constitute a preferred embodiment of the present invention. Isotopic variants of the compounds of the invention can be prepared by the processes known to those skilled in the art, for example by the methods described further down and the procedures described in the working examples, by using corresponding isotopic modifications of the respective reagents and/or starting materials.

In the context of the present invention, unless specified otherwise, the substituents are defined as follows:

Alkyl per se and “Alk” and “alkyl” in alkoxy, alkoxyalkyl, alkylamino and alkoxycarbonyl represent a straight-chain or branched alkyl radical having 1 to 6 carbon atoms, preferably 1 to 4 carbon atoms, by way of example and with preference methyl, ethyl, n-propyl, isopropyl, n-butyl, sec-butyl, tert-butyl, n-pentyl, sec-pentyl and n-hexyl.

Alkoxy, per se and “alkoxy” in cycloalkoxy, cycloalkylalkoxy, haloalkoxy, represents, by way of example and with preference, methoxy, ethoxy, n-propoxy, isopropoxy, n-butoxy, sec-butoxy and tert-butoxy.

Alkoxyalkyl, by way of example and with preference, represents methoxymethyl, ethoxymethyl, n-propoxymethyl, isopropoxymethyl, n-butoxymethyl, tert-butoxymethyl, methoxyethyl, ethoxyethyl, n-propoxyethyl, isopropoxyethyl, n-butoxyethyl and tert-butoxyethyl.

Haloalkoxy represents an alkoxy radical as defined above which is mono- or polyhalogenated up to the maximum possible number of substituents. In the case of polyhalogenation, the halogen atoms can be identical or different. In the context of the present invention, halogen is fluorine, chlorine, bromine or iodine, preferably fluorine or chlorine.

Alkylamino represents an alkylamino radical having one or two (independently selected) alkyl substituents, by way of example and with preference methylamino, ethylamino, n-propylamino, isopropylamino, tert-butylamino, N,N-dimethylamino, N,N-diethylamino, N-ethyl-N-methylamino, N-methyl-N-n-propylamino, N-isopropyl-N-n-propylamino and N-tert-butyl-N-methylamino. C₁-C₄-Alkylamino represents, for example, a monoalkylamino radical having 1 to 4 carbon atoms or a dialkylamino radical having in each case 1 to 4 carbon atoms per alkyl substituent.

By way of example and with preference, alkoxycarbonyl represents methoxycarbonyl, ethoxycarbonyl, n-propoxycarbonyl, isopropoxycarbonyl, n-butoxycarbonyl, sec-butoxy and tert-butoxycarbonyl.

Alkylaminocarbonyl represents an alkylaminocarbonyl radical having one or two (independently selected) alkyl substituents, by way of example and with preference methylaminocarbonyl, ethylaminocarbonyl, n-propylaminocarbonyl, isopropylaminocarbonyl, tert-butylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl and N-tert-butyl-N-methylaminocarbonyl. C₁-C₄-Alkylaminocarbonyl represents, for example, a monoalkylaminocarbonyl radical having 1 to 4 carbon atoms or a dialkylaminocarbonyl radical having in each case 1 to 4 carbon atoms per alkyl substituent.

Cycloalkyl represents a monocyclic cycloalkyl group having generally 3 to 6 carbon atoms; preferred examples of cycloalkyl include cyclopropyl, cyclobutyl, cyclopentyl and cyclohexyl.

Heteroaryl represents an aromatic monocyclic radical having generally 5 or 6 ring atoms and up to 4 heteroatoms from the group consisting of S, O and N, where a nitrogen atom may also form an N-oxide, by way of example and with preference thienyl, furyl, pyrrolyl, thiazolyl, oxazolyl, isoxazolyl, oxadiazolyl, pyrazolyl, imidazolyl, triazolyl, pyridyl, pyrimidyl, pyridazinyl, pyrazinyl. According to one embodiment, heteroaryl is selected from oxazolyl, isoxazolyl, oxadiazolyl, pyrazolyl, triazolyl and pyridyl.

Halogen represents fluorine, chlorine, bromine and iodine, preferably fluorine and chlorine.

Haloalkyl represents an alkyl radical as defined above which is mono- or polyhalogenated up to the maximum possible number of substituents. In the case of polyhalogenation, the halogen atoms can be identical or different. In the context of the present invention, halogen is fluorine, chlorine, bromine or iodine, preferably fluorine or chlorine.

When radicals in the compounds of the invention are substituted, the radicals may be mono- or polysubstituted, unless specified otherwise. In the context of the present invention, all radicals which occur more than once are defined independently of one another. Substitution by one, two or three identical or different substituents is preferred.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

Preference is given to compounds of the formula (I) in which
[symbol] represents a single bond or a double bond,
R¹ is selected from the group consisting of C₃-C₆-alkyl, C₁-C₃-alkoxycarbonyl, oxetanyl, 5- or 6-membered heteroaryl, —(CR⁶R⁷)—R⁸ and —CONR⁹R¹⁰
   where oxetanyl may be substituted by 1 or 2 substituents independently of one another selected from the group consisting of 3-hydroxy and 3-C₁-C₄-alkyl,
   and
   where
   R⁶ is selected from the group consisting of hydrogen, methyl and ethyl,
   R⁷ is selected from the group consisting of hydrogen, methyl and ethyl,
   or
   R⁶ and R⁷ together with the carbon atom to which they are attached form a cyclopropyl ring or cyclobutyl ring,
   R⁸ is selected from the group consisting of hydroxy, hydroxymethyl, C₁-C₄-alkyl, C₁-C₄-alkoxy, C₁-C₃-alkoxycarbonyl, C₁-C₄-alkylaminocarbonyl, phenoxy, oxetanyl, 5- or 6-membered heteroaryl and —CH₂NR¹³R¹⁴,
      where phenoxy and heteroaryl may be substituted by 1 to 3 substituents independently of one another selected from the group consisting of C₁-C₄-alkyl and C₁-C₄-alkoxy,
      where oxetanyl may be substituted by 1 or 2 substituents independently of one another selected from the group consisting of 3-C₁-C₄-alkyl and 3-OH,
      and
      where
      R¹³ is selected from the group consisting of hydrogen and C₁-C₄-alkyl,
      and
      R¹⁴ is selected from the group consisting of methyl, methylsulphonyl and formyl,
   R⁹ is selected from the group consisting of C₁-C₆-alkyl, C₃-C₆-cycloalkyl and 5- or 6-membered heteroaryl,
      where alkyl may be substituted by 1 to 3 substituents independently of one another selected from the group consisting of hydroxy, with the proviso that alkyl is C₂-C₆-alkyl, C₁-C₄-alkoxy, C₁-C₄-haloalkyl, C₃-C₆-cycloalkyl, phenyl, oxetanyl and 5- or 6-membered heteroaryl,
         in which this phenyl or heteroaryl for its part may be substituted by 1 to 3 substituents independently of one another selected from the group consisting of halogen, trifluoromethyl, difluoromethoxy, trifluoromethoxy and C₁-C₄-alkyl
         in which this oxetanyl for its part may be substituted by one or 2 substituents selected from the group consisting of 3-C₁-C₄-alkyl and 3-hydroxy;
   R¹⁰ is selected from the group consisting of hydrogen and C₁-C₄-alkyl,
   or
   R⁹ and R¹⁰ together with the nitrogen atom to which they are attached form a piperidinyl ring,
      where the piperidinyl ring may be substituted by 1 to 3 substituents independently of one another selected from the group consisting of C₁-C₄-alkyl,
R² is selected from the group consisting of hydrogen and halogen,
R³ is selected from the group consisting of hydrogen, halogen, hydroxy and C₁-C₄-alkoxy,
R⁴ is selected from the group consisting of C₁-C₃-alkyl, C₁-C₃-alkoxycarbonyl, C₃-C₆-cycloalkyl, C₃-C₆-cycloalkyl-C₁-C₃-alkoxy, C₃-C₆-cycloalkoxy, trifluoromethoxy-C₁-C₄-alkoxy, 5- or 6-membered heteroaryl and —OCONR¹¹R¹²,
   where alkyl may be substituted by a substituent selected from the group consisting of C₁-C₄-alkoxy, C₃-C₆-cycloalkoxy, trifluoromethoxy and phenoxy,
      in which this phenoxy for its part may be substituted by 1 to 3 substituents independently of one another selected from the group consisting of halogen,
   and
   where heteroaryl may be substituted by 1 to 3 substituents independently of one another selected from the group consisting of C₁-C₄-alkyl and C₃-C₆-cycloalkyl,
      in which this alkyl for its part may be substituted by a substituent selected from the group consisting of C₁-C₃-alkoxy and C₃-C₆-cycloalkyl,
   R¹¹ represents C₁-C₄-alkyl,
   R¹² is selected from the group consisting of hydrogen and C₁-C₄-alkyl,
   or
   R¹¹ and R¹² together with the nitrogen atom to which they are attached form a pyrrolidinyl ring,
R⁵ represents hydrogen or C₁-C₄-alkyl,
or one of the salts thereof, solvates thereof or solvates of the salts thereof.

Preference is given to compounds of the formula (I) in which
[symbol] represents a single bond,
R¹ represents C₃-C₄-alkyl, C₁-C₃-alkoxycarbonyl, oxetanyl, oxazolyl, —(CR⁶R⁷)—R⁸ or —CONR⁹R¹⁰,
   where oxetanyl may be substituted by 1 or 2 substituents independently of one another selected from the group consisting of 3-hydroxy and 3-C₁-C₃-alkyl,
   and
   where
   R⁶ is selected from the group consisting of hydrogen, methyl and ethyl,
   R⁷ is selected from the group consisting of hydrogen, methyl and ethyl,
   or
   R⁶ and R⁷ together with the carbon atom to which they are attached form a cyclopropyl ring or cyclobutyl ring,
   R⁸ is selected from the group consisting of hydroxy, hydroxymethyl, C₁-C₃-alkyl, C₁-C₃-alkoxy, C₁-C₃-alkoxycarbonyl, C₁-C₃-alkylaminocarbonyl, phenoxy, oxetanyl, pyrazolyl and —CH₂NR¹³R¹⁴,
      where phenoxy and pyrazolyl may be substituted by 1 to 3 substituents independently of one another selected from the group consisting of C₁-C₂-alkyl and C₁-C₂-alkoxy,
      where oxetanyl may be substituted by 1 or 2 substituents independently of one another selected from the group consisting of 3-C₁-C₂-alkyl,
      and
      where
      R¹³ is selected from the group consisting of hydrogen and C₁-C₂-alkyl,
      and
      R¹⁴ is selected from the group consisting of methyl, methylsulphonyl and formyl,
   R⁹ is selected from the group consisting of C₁-C₄-alkyl, C₃-C₆-cycloalkyl and oxazolyl,
      where alkyl may be substituted by 1 to 3 substituents independently of one another selected from the group consisting of hydroxy, with the proviso that alkyl is C₂-C₄-alkyl, C₁-C₂-alkoxy, C₁-C₂-haloalkyl, C₃-C₄-cycloalkyl, phenyl, oxetanyl, oxazolyl, pyrazolyl and pyridyl,
      in which this phenyl or pyridyl for its part may be substituted by 1 to 3 substituents independently of one another selected from the group consisting of halogen, trifluoromethyl, difluoromethoxy, trifluoromethoxy and methyl,
      in which this oxetanyl for its part may be substituted by 3-methyl
      and
      in which this oxazolyl for its part may be substituted by 1 to 3 methyl substituents,
   R¹⁰ is selected from the group consisting of hydrogen and C₁-C₃-alkyl,
R² is selected from the group consisting of hydrogen, fluorine and chlorine,
R³ is selected from the group consisting of hydrogen, fluorine, chlorine, hydroxy and C₁-C₂-alkoxy,
R⁴ is selected from the group consisting of C₁-C₂-alkyl, C₁-C₃-alkoxycarbonyl, C₃-C₄-cycloalkyl, C₃-C₄-cycloalkyl-C₁-C₃-alkoxy, C₃-C₄-cycloalkoxy, trifluoromethoxy-C₁-C₂-alkoxy, oxadiazole, triazole and pyrrolidine-1-carboxylate,
   where alkyl may be substituted by a substituent selected from the group consisting of C₁-C₄-alkoxy, C₃-C₄-cycloalkoxy, trifluoromethoxy and phenoxy,
      in which this phenoxy for its part may be substituted by 1 to 3 substituents independently of one another selected from the group consisting of fluorine and chlorine,
   and
   where oxadiazole or triazole may be substituted by 1 to 3 substituents independently of one another selected from the group consisting of C₁-C₂-alkyl and C₃-C₄-cycloalkyl,
   in which this alkyl for its part may be substituted by a substituent selected from the group consisting of C₁-C₃-alkoxy and C₃-C₄-cycloalkyl,
R⁵ represents hydrogen,
or one of the salts thereof, solvates thereof or solvates of the salts thereof.

Preference is also given to compounds of the formula (I) in which
[symbol] represents a single bond,
R¹ represents C₃-C₄-alkyl, oxetanyl, —(CR⁶R⁷)—R⁸ or —CONR⁹R¹⁰,
   where oxetanyl may be substituted by a substituent selected from the group consisting of 3-hydroxy and 3-methyl,
   and
   where
   R⁶ is selected from the group consisting of hydrogen, methyl and ethyl,
   R⁷ is selected from the group consisting of hydrogen, methyl and ethyl,
   or
   R⁶ and R⁷ together with the carbon atom to which they are attached form a cyclobutyl ring,
   R⁸ is selected from the group consisting of hydroxy, methyl, methoxy, oxetanyl, and —CH₂NR¹³R¹⁴,
      where oxetanyl may be substituted by a 3-methyl substituent,
      and
      where
      R¹³ is selected from the group consisting of hydrogen and methyl,
      and
      R¹⁴ is selected from the group consisting of methyl, methylsulphonyl and formyl,
   R⁹ is selected from the group consisting of C₁-C₄-alkyl and oxazolyl,
      where alkyl may be substituted by 1 to 3 substituents independently of one another selected from the group consisting of hydroxy, with the proviso that alkyl is C₂-C₆-alkyl, phenyl and pyridyl,
         in which this phenyl or pyridyl for its part may be substituted by 1 to 3 substituents independently of one another selected from the group consisting of chlorine, fluorine and trifluoromethyl,
      and
      where oxazolyl may be substituted by 1 to 3 methyl substituents,
   R¹⁰ is selected from the group consisting of hydrogen and methyl,
R² represents hydrogen,
R³ is selected from the group consisting of hydrogen and chlorine,
R⁴ is selected from the group consisting of methyl, ethyl, ethoxycarbonyl, cyclopropyl, C₃-C₄-cycloalkyl-C₁-C₂-alkoxy, oxadiazolyl and triazolyl,
   where methyl or ethyl may be substituted by a substituent selected from the group consisting of methoxy, ethoxy, tert-butoxy, C₃-C₄-cycloalkoxy and trifluoromethoxy,
   and
   where oxadiazolyl or triazolyl may be substituted by 1 to 3 methyl substituents,
      in which this methyl for its part may be substituted by C₃-C₄-cycloalkyl,
R⁵ represents hydrogen,
or one of the salts thereof, solvates thereof or solvates of the salts thereof.

Preference is also given to compounds of the formula (I) in which R¹ is selected from the group consisting of 1-hydroxy-1-methylethyl, 1-methoxy-1-methylethyl, tert-butylaminocarbonyl, tert-butyl and isobutyl.

Preference is also given to compounds of the formula (I) in which R¹ represents 1-hydroxy-1-methylethyl.

Preference is also given to compounds of the formula (I) in which R¹ represents tert-butylaminocarbonyl.

Preference is also given to compounds of the formula (I) in which R¹ represents tert-butyl.

Preference is also given to compounds of the formula (I) in which R¹ represents oxetanyl, where oxetanyl may be substituted by a substituent selected from the group consisting of 3-hydroxy and 3-methyl.

Preference is also given to compounds of the formula (I) in which R¹ represents —(CR⁶R⁷)—R⁸,
where
R⁶ is selected from the group consisting of hydrogen, methyl and ethyl,
R⁷ is selected from the group consisting of hydrogen, methyl and ethyl,
or
R⁶ and R⁷ together with the carbon atom to which they are attached form a cyclobutyl ring,
R⁸ is selected from the group consisting of hydroxy, methoxy, oxetanyl, and —CH₂NR¹³R¹⁴,
   where oxetanyl may be substituted by a 3-methyl substituent,
   and
   where
   R¹³ is selected from the group consisting of hydrogen and methyl,
   and
   R¹⁴ is selected from the group consisting of methyl, methylsulphonyl and formyl.

Preference is also given to compounds of the formula (I) in which R¹ represents —(CR⁶R⁷)—R⁸,
where
R⁶ and R⁷ are selected from the group consisting of methyl and ethyl,
R⁸ is selected from the group consisting of hydroxy and methoxy.

Preference is also given to compounds of the formula (I) in which R¹ represents —(CR⁶R⁷)—R⁸,

where
R⁶ and R⁷ are hydrogen,
R⁸ represents oxetanyl,
   where oxetanyl may be substituted by a 3-methyl substituent.

Preference is also given to compounds of the formula (I) in which R¹ represents —(CR⁶R⁷)—R⁸,
where
R⁶ and R⁷ together with the carbon atom to which they are attached form a cyclobutyl ring,
R⁸ represents —CH₂NR¹³R¹⁴,
   where
   R¹³ is selected from the group consisting of hydrogen and methyl,
   and
   R¹⁴ is selected from the group consisting of methyl and methylsulphonyl.

Preference is also given to compounds of the formula (I) in which R¹ represents —CONR⁹R¹⁰
where
R⁹ represents C₁-C₄-alkyl, or oxazolyl,
   where alkyl may be substituted by 1 to 3 substituents independently of one another selected from the group consisting ofhydroxy, with the proviso that alkyl is C₂-C₆-alkyl, trifluoromethyl, phenyl and pyridyl,
      in which this phenyl or pyridyl for its part may be substituted by 1 to 3 substituents independently of one another selected from the group consisting of chlorine, fluorine, trifluoromethyl and methyl,
   and
   where oxazolyl may be substituted by a 3-methyl substituent,
R¹⁰ is selected from the group consisting of hydrogen and methyl.

Preference is also given to compounds of the formula (I) in which R¹ represents —CONR⁹R¹⁰,
where
R⁹ represents C₁-C₄-alkyl,
   where alkyl may be substituted by 1 to 3 substituents independently of one another selected from the group consisting of hydroxy, with the proviso that alkyl is C₂-C₆-alkyl, trifluoromethyl, phenyl and pyridyl,
      in which this phenyl or pyridyl for its part may be substituted by 1 to 3 substituents independently of one another selected from the group consisting of chlorine, fluorine, trifluoromethyl and methyl,
R¹⁰ is selected from the group consisting of hydrogen and methyl.

Preference is also given to compounds of the formula (I) in which R¹ represents —CONR⁹R¹⁰,
where
R⁹ represents oxazolyl,
   where oxazolyl may be substituted by a 3-methyl substituent,
R¹⁰ is selected from the group consisting of hydrogen and methyl.

Preference is also given to a compound of the formula (I) in which
[symbol] represents a single bond,
R¹ represents —(CR⁶R⁷)—R⁸, where
R⁶ and R⁷ represent methyl,
R⁸ represents hydroxy,
R² and R³ represent hydrogen,
R⁴ represents methyl and
R⁵ represents hydrogen,
or one of the salts thereof, solvates thereof or solvates of the salts thereof.

Preference is also given to compounds of the formula (I) in which R² and R³ represent hydrogen.

Preference is also given to compounds of the formula (I) in which R⁴ is selected from the group consisting of methyl, ethyl, methoxymethyl, trifluoromethoxymethyl, ethoxycarbonyl, cyclopropylmethoxy, cyclobutylmethoxy, cyclopropoxymethyl, cyclobutoxymethyl, isopropoxy, methoxy, ethoxy, cyclopropyl and (cyclobutylmethyl)-4H-1,2,4-triazol-3-yl.

Preference is also given to compounds of the formula (I) in which R⁴ is selected from the group consisting of methyl and ethyl.

Preference is also given to compounds of the formula (I) in which R⁴ represents methoxymethyl.

Preference is also given to compounds of the formula (I) in which R⁴ represents trifluoromethoxymethyl.

Preference is also given to compounds of the formula (I) in which R⁴ represents ethoxycarbonyl.

Preference is also given to compounds of the formula (I) in which R⁴ is selected from the group consisting of cyclopropylmethoxy and cyclobutylmethoxy.

Preference is also given to compounds of the formula (I) in which R⁴ is selected from the group consisting of cyclopropoxymethyl and cyclobutoxymethyl.

Preference is also given to compounds of the formula (I) in which R⁴ is selected from the group consisting of isopropoxy, methoxy and ethoxy.

Preference is also given to compounds of the formula (I) in which R⁴ represents cyclopropyl.

Preference is also given to compounds of the formula (I) in which R⁴ represents triazolyl, where triazolyl may be substituted by 1 to 2 substituents independently of one another selected from the group consisting of C₁-C₄-alkyl and C₃-C₆-cycloalkyl, where alkyl may be substituted by a substituent selected from the group consisting of cyclopropyl and cyclobutyl.

Preference is also given to compounds of the formula (I) in which R⁴ represents (cyclobutylmethyl)-4H-1,2,4-triazol-3-yl.

Preference is also given to compounds of the formula (I) in which R⁵ represents hydrogen.

Irrespective of the particular combinations of the radicals specified, the individual radical definitions specified in the particular combinations or preferred combinations of radicals are also replaced as desired by radical definitions of other combinations.

Very particular preference is given to combinations of two or more of the abovementioned preferred ranges.

The invention further provides a process for preparing the compounds of the formula (I) and their starting materials and intermediates, or the salts thereof, the solvates thereof or the solvates of the salts thereof, where
[A] compounds of the formula (II)

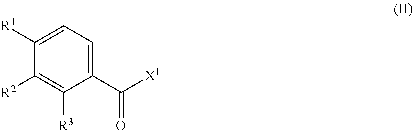

in which
R¹, R² and R³ have the meaning given above, and
X¹ is selected from the group consisting of halogen, preferably bromine or chlorine, and hydroxy, are reacted with compounds of the formula (III)

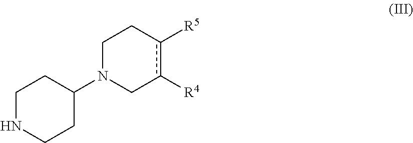

in which
[symbol] , R⁴ and R⁵ have the meaning given above,
if X¹ represents hydroxy in the presence of a dehydrating agent, if X¹ represents halogen in the presence of a base, to give compounds of the formula (I)
or
[B] compounds of the formula (II)

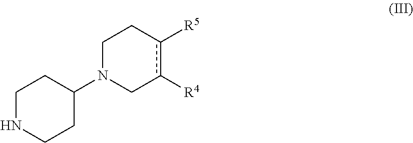

in which
R¹, R² and R³ have the meaning given above, and
X¹ represents hydroxy,
are reacted with 4-piperidinone in the presence of a dehydrating agent to give compounds of the formula (V)

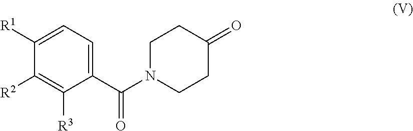

in which
R¹, R² and R³ have the meaning given above,
or
[C] compounds of the formula (V)

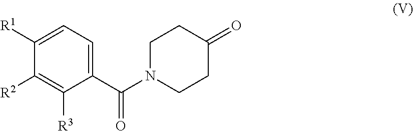

in which
R¹, R² and R³ have the meaning given above,
are reacted with compounds of the formula (VI)

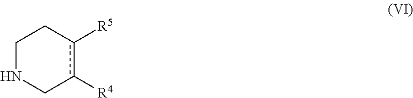

in which
[symbol] , R⁴ and R⁵ have the meaning given above,
in the presence of a reducing agent to give compounds of the formula (I)
or
[D] compounds of the formula (IV)

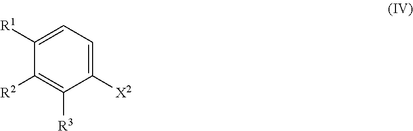

in which
R¹, R² and R³ have the meaning given above, and
X² is selected from the group consisting of halogen, preferably bromine, and trifluoromethanesulphonate,
are reacted with compounds of the formula (III)

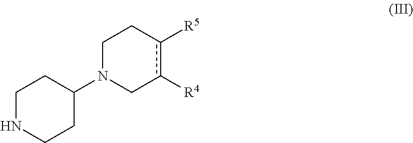

in which
[symbol] , R⁴ and R⁵ have the meaning given above,
in the presence of a carbon monoxide source and a catalyst to give compounds of the formula (I) or
[E] compounds of the formula (VII)

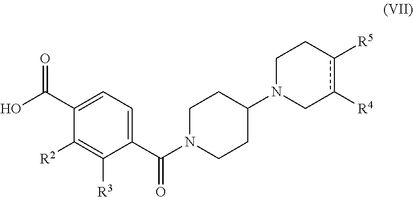

in which
[symbol] , R², R³, R⁴ and R⁵ have the meaning given above,
are reacted with compounds of the formula

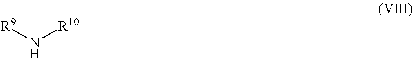

in which
R⁹ and R¹⁰ have the meaning given above,
in the presence of a dehydrating agent to give compounds of the formula

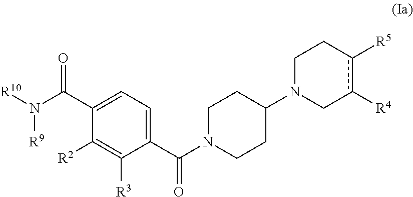

in which
[symbol] , R², R³, R⁴, R⁵, R⁹ and R¹⁰ have the meaning given above,
or
[F] compounds of the formula (VII)

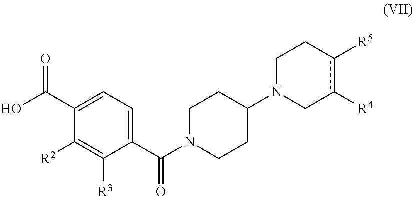

in which
[symbol] , R², R³, R⁴ and R⁵ have the meaning given above,
are, in a first step, reacted with oxalyl chloride or thionyl chloride and, in a second step, with compounds of the formula (VIII)

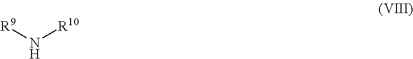

in which
R⁹ and R¹⁰ have the meaning given above,
to give compounds of the formula (Ia)
or
[G] compounds of the formula (IX)

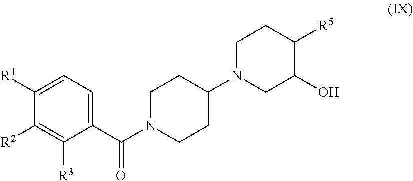

in which
R¹, R², R³ and R⁵ have the meaning given above,
are reacted with compounds of the formula (X)

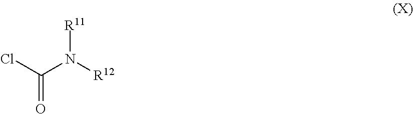

in which
R¹¹ and R¹² have the meaning given above,
to give compounds of the formula (Ib)

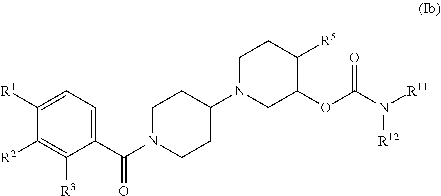

in which
R¹, R², R³, R⁵, R¹¹ and R¹² have the meaning given above,
or
[H] compounds of the formula (IX)

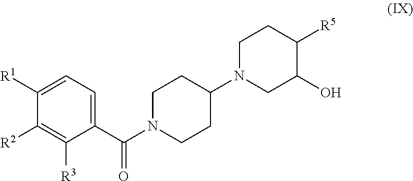

in which
R¹, R², R³ and R⁵ have the meaning given above,
are reacted with compounds of the formula (XI)

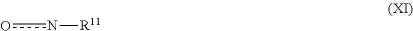

in which
R¹¹ has the meaning given above,
to give compounds of the formula (Ic)

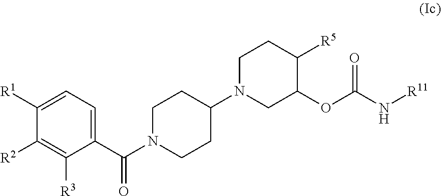

in which
R¹, R², R³, R⁵ and R¹¹ have the meaning given above,
or
[I] compounds of the formula (XII)

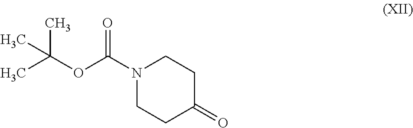

are reacted with compounds of the formula (XIII)

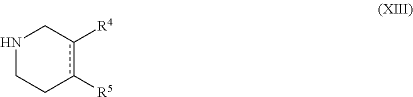

in which R⁴ and R⁵ have the meanings given above,
in the presence of a reducing agent to give compounds of the formula (XIV)

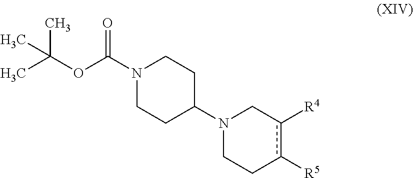

in which R⁴ and R⁵ have the meanings given above,
or
[J] compounds of the formula (XIV)

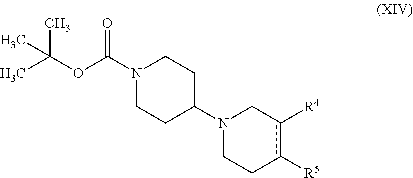

are reacted in the presence of an acid to give compounds of the formula (III)

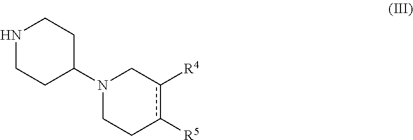

in which R⁴ and R⁵ have the meanings given above.

The compounds of the formula (Ia), the compounds of the formula (Ib) and the compounds of the formula (Ic) are a subset of the compounds of the formula (I).

One embodiment of the present invention is a process for preparing a compound of the formula (I), or one of the salts thereof, solvates thereof or solvates of the salts thereof as described above according to process [A].

The reaction according to process [A] is, if X¹ represents halogen, generally carried out in inert solvents, if appropriate in the presence of a base, preferably in a temperature range of from −30° C. to 50° C. at a pressure of from 1 to 20 bar.

Inert solvents are, for example, tetrahydrofuran, dichloromethane, dichloroethane, pyridine, acetonitrile, dimethoxyethane, N-methylpyrrolidione, dioxane, dimethylformamide, dimethyl sulphoxide, ethyl acetate or toluene. It is also possible to use mixtures of the solvents mentioned. Preference is given to tetrahydrofuran, dioxane or dichloromethane.

Bases are, for example, organic bases such as trialkylamines, for example triethylamine, diisopropylethylamine, 2,6-lutidine, N-methylmorpholine, pyridine, diazabicyclo[2.2.2]octane, 1,5-diazabicyclo[4.3.0]non-5-ene or 1,8-diazabicyclo[5.4.0]undec-7-ene; preference is given to triethylamine or diisopropylethylamine.

The reaction according to process [A] is, if X¹ represents hydroxy, generally carried out in inert solvents, in the presence of a dehydrating agent, if appropriate in the presence of a base, preferably in a temperature range of from −30° C. to 50° C. at a pressure of from 1 to 20 bar.

Inert solvents are, for example, halogenated hydrocarbons, such as dichloromethane or trichloromethane, hydrocarbon such as benzene, nitromethane, dioxane, dimethylformamide or acetonitrile. It is also possible to use mixtures of the solvents mentioned. Particular preference is given to acetonitrile.

Suitable dehydrating agents are, for example, carbodiimides such as, for example, N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulphate or 2-tert-butyl-5-methylisoxazolium perchlorate, or acylamino compounds such as 2-ethoxy-1-ethoxycarbonyl-1,2-dihydroquinoline, or propanephosphonic anhydride (T3P), or isobutyl chloroformate, or bis-(2-oxo-3-oxazolidinyl)phosphoryl chloride or benzotriazolyloxytri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or N-hydroxysuccinimide, or mixtures of these, with bases.

Bases are, for example, alkali metal carbonates such as sodium carbonate, potassium carbonate or caesium carbonate, or sodium bicarbonate, potassium bicarbonate or caesium bicarbonate, or organic bases such as trialkylamines, for example triethylamine, N-methylmorpholine, N-methylpiperidine, 4-dimethylaminopyridine or diisopropylethylamine, with diisopropylethylamine being preferred.

If X¹ represents hydroxy, the condensation is preferably carried out with HATU or with EDC in the presence of HOBt or with propanephosphonic anhydride (T3P).

The compounds of the formula (III) are known, can be synthesized by known processes from the appropriate starting materials or can be prepared according to the processes described under [I] and [J] from the appropriate starting materials.

The compounds of the formula (V) are known or can be prepared according to process [B]. The reaction according to process [B] is generally carried out in inert solvents, if appropriate in the presence of a base, preferably in a temperature range of from −30° C. to 50° C. at a pressure of from 1 to 20 bar.

Inert solvents are, for example, halogenated hydrocarbons, such as dichloromethane or trichloromethane, hydrocarbons, such as benzene, nitromethane, dioxane, dimethylformamide or acetonitrile, or alcohols, for example methanol, ethanol, isopropanol. It is also possible to use mixtures of the solvents mentioned. Particular preference is given to acetonitrile.

Suitable dehydrating agents are, for example, carbodiimides such as, for example, N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulphate or 2-tert-butyl-5-methylisoxazolium perchlorate, or acylamino compounds such as 2-ethoxy-1-ethoxycarbonyl-1,2-dihydroquinoline, or propanephosphonic anhydride (T3P), or isobutyl chloroformate, or bis-(2-oxo-3-oxazolidinyl)phosphoryl chloride or benzotriazolyloxytri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or N-hydroxysuccinimide, or mixtures of these, with bases.

Bases are, for example, alkali metal carbonates such as sodium carbonate, potassium carbonate or caesium carbonate, or sodium bicarbonate, potassium bicarbonate or caesium bicarbonate, or organic bases such as trialkylamines, for example triethylamine, N-methylmorpholine, N-methylpiperidine, 4-dimethylaminopyridine or diisopropylethylamine, with diisopropylethylamine being preferred.

The condensation is preferably carried out using propanephosphonic anhydride.

One embodiment of the present invention is a process for preparing a compound of the formula (I), or one of the salts thereof, solvates thereof or solvates of the salts thereof as described above according to process [C].

The reaction according to process [C] is generally carried out in inert solvents, preferably in a temperature range of from −20° C. to 60° C. at a pressure of from 1 to 20 bar.

Inert solvents are, for example, alcohols such as methanol, ethanol, n-propanol or isopropanol, or ethers such as diethyl ether, dioxane or tetrahydrofuran, or dimethylformamide, or acetic acid or glacial acetic acid. It is also possible to use mixtures of the solvents mentioned. Preference is given to a mixture of methanol and glacial acetic acid.

Reducing agents are, for example, sodium borohydride, lithium borohydride, sodium cyanoborohydride, lithium aluminium hydride, sodium bis-(2-methoxyethoxy)aluminium hydride or borane/tetrahydrofuran; preference is given to sodium cyanoborohydride.

One embodiment of the present invention is a process for preparing a compound of the formula (I), or one of the salts thereof, solvates thereof or solvates of the salts thereof as described above according to process [D].

The reaction according to process [D] is generally carried out in inert solvents, if appropriate in the presence of a base, if appropriate in the presence of a phosphonium salt or a phosphine, if appropriate in a microwave apparatus, preferably in a temperature range from 20° C. to 180° C., particularly preferably in a temperature range from 80° C. to 180° C., at a pressure of from 1 to 20 bar.

Inert solvents are, for example, dimethyl sulphoxide, dimethylformamide, dimethylacetamide, N-methylpyrrolidone, dioxane, tetrahydrofuran or water. It is also possible to use mixtures of the solvents mentioned. Particular preference is given to water or tetrahydrofuran.

Bases are, for example, alkali metal carbonates such as sodium carbonate, potassium carbonate or caesium carbonate, or sodium hydrogenphosphate or sodium bicarbonate or amines such as triethylamine, diisopropylethylamine, N-methylmorpholine or 1,8-diazabicyclo[5.4.0]undec-7-ene; preference is given to sodium carbonate.

Phosphonium salts are, for example, tri-tert-butylphosphonium tetrafluoroborate or triisoamylphosphonium tetrafluoroborate. Phosphines are, for example, tri-tert-butylphosphine or triisoamylphosphine.

Catalysts are, for example, palladium salts or nickel salts or palladium complexes or nickel complexes; preference is given to palladium complexes such as tetrakis(triphenylphosphine)palladium, 1,1′-bis(diphenylphosphino)ferrocenepalladium diacetate, trans-bis(acetato)bis[o-(di-o-tolylphosphine)benzyl]dipalladium(II) (Herrmann's palladacycle), bis(triphenylphosphine)palladium dichloride, 9,9-dimethyl-4,5-bis(diphenylphosphino)xanthenepalladium(II) acetate, bisbenzothiazolecarbenepalladium diiodide or 9,9-dimethyl-4,5-bis(diphenylphosphino)xanthenepalladium(II) acetate. Particular preference is given to trans-bis(acetato)bis[o-(di-o-tolylphosphine)benzyl]dipalladium(II) (Herrmann's palladacycle). Here, the catalyst is employed in a molar ratio of from 0.01 to 0.5 equivalents; preferably, it is employed in a range of from 0.03 to 0.15 equivalents.

Carbon monoxide sources are, for example, molybdenum hexacarbonyl or carbon monoxide gas; preference is given to molybdenum hexacarbonyl.

Preference is given to the reaction with molybdenum hexacarbonyl and trans-bis(acetato)bis[o-(di-o-tolylphosphine)benzyl]dipalladium(II) (Herrmann's palladacycle) in a molar ratio of from 0.03 to 0.08 equivalents, with aqueous sodium carbonate solution in water in a microwave apparatus or with trans-bis(acetato)bis[o-(di-o-tolylphosphine)benzyl]dipalladium(II) (Herrmann's palladacycle), 8-diazabicyclo[5.4.0]undec-7-ene and tri-tert-butylphosphonium tetrafluoroborate in tetrahydrofuran in a microwave apparatus.

The compounds of the formula (VI) are known or can be synthesized by known processes from the appropriate starting materials.

One embodiment of the present invention is a process for preparing a compound of the formula (I), in particular of the formula (Ia), or one of the salts thereof, solvates thereof or solvates of the salts thereof as described above according to process [E].

The reaction according to process [E] is generally carried out in inert solvents, if appropriate in the presence of a base, preferably in a temperature range of from −30° C. to 50° C. at atmospheric pressure.

Inert solvents are, for example, halogenated hydrocarbons, such as dichloromethane or trichloromethane, hydrocarbons, such as benzene, nitromethane, dioxane, dimethylformamide, dimethyl sulphoxide or acetonitrile. It is also possible to use mixtures of the solvents mentioned. Particular preference is given to dimethyl sulphoxide, dichloromethane or dimethylformamide.

Suitable dehydrating agents are, for example, carbodiimides such as, for example, N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulphate or 2-tert-butyl-5-methylisoxazolium perchlorate, or acylamino compounds such as 2-ethoxy-1-ethoxycarbonyl-1,2-dihydroquinoline, or propanephosphonic anhydride (T3P), or isobutyl chloroformate, or bis-(2-oxo-3-oxazolidinyl)phosphoryl chloride or benzotriazolyloxytri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), 2-(1H-benzotriazol-1-yl)-1,1,3,3-tetramethylaminium tetrafluoroborate (TBTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or N-hydroxysuccinimide, or mixtures of these, with bases.

Bases are, for example, alkali metal carbonates such as sodium carbonate, potassium carbonate or caesium carbonate, or sodium bicarbonate, potassium bicarbonate or caesium bicarbonate, or organic bases such as trialkylamines, for example triethylamine, N-methylmorpholine, N-methylpiperidine, 4-dimethylaminopyridine or diisopropylethylamine, with diisopropylethylamine being preferred.

Preferably, the condensation is carried out with HATU or with EDC in the presence of HOBt.

The compounds of the formula (VIII) are known or can be synthesized by known processes from the appropriate starting materials.

One embodiment of the present invention is a process for preparing a compound of the formula (I), in particular of the formula (Ia), or one of the salts thereof, solvates thereof or solvates of the salts thereof as described above according to process [F].

The reaction of the first step according to process [F] is generally carried out in inert solvents, preferably in a temperature range of from −30° C. to 50° C. at a pressure of from 1 to 20 bar.

Inert solvents are, for example, halogenated hydrocarbons such as dichloromethane or trichloromethane; preference is given to dichloromethane.

The reaction of the second step according to process [F] is generally carried out in inert solvents, if appropriate in the presence of a base, preferably in a temperature range of from −30° C. to 50° C. at atmospheric pressure.

Inert solvents are, for example, halogenated hydrocarbons such as dichloromethane or trichloromethane; preference is given to dichloromethane.

Bases are, for example, organic bases such as trialkylamines, for example triethylamine, N-methylmorpholine, N-methylpiperidine, 4-dimethylaminopyridine or diisopropylethylamine; preference is given to triethylamine.

One embodiment of the present invention is a process for preparing a compound of the formula (I), or one of the salts thereof, solvates thereof or solvates of the salts thereof as described above according to process [G].

The reaction according to process [G] is generally carried out in inert solvents, if appropriate in the presence of a base, preferably in a temperature range of from −30° C. to 50° C. at atmospheric pressure.

Inert solvents are, for example, halogenated hydrocarbons such as dichloromethane or trichloromethane; preference is given to dichloromethane.

Bases are, for example, organic bases such as trialkylamines, for example triethylamine, N-methylmorpholine, N-methylpiperidine, 4-dimethylaminopyridine or diisopropylethylamine; preference is given to triethylamine.

The compounds of the formula (IX) are known, can be synthesized by known processes from the appropriate starting materials or can be prepared according to processes [A] to [H].

The compounds of the formula (X) are known or can be synthesized by known processes from the appropriate starting materials.

One embodiment of the present invention is a process for preparing a compound of the formula (I), or one of the salts thereof, solvates thereof or solvates of the salts thereof as described above according to process [H].

The reaction according to process [H] is generally carried out in inert solvents, preferably in a temperature range from −30° C. to 50° C. at atmospheric pressure.

Inert solvents are, for example, halogenated hydrocarbons such as dichloromethane or trichloromethane, or tetrahydrofuran; preference is given to dichloromethane.

The compounds of the formula (XI) are known or can be synthesized by known processes from the appropriate starting materials.

The compounds of the formula (II) in which X¹ represents halogen are known or can be prepared by reacting compounds of the formula (II) in which X¹ represents hydroxy with oxalyl chloride, thionyl chloride or thionyl bromide.

The reaction is generally carried out in inert solvents or in the absence of a solvent, preferably in a temperature range from 0° C. to reflux of the solvent at atmospheric pressure.

Inert solvents are, for example, dichloromethane, trichloromethane, 1,2-dichloroethane, benzene, toluene, chlorobenzene, dioxane or tetrahydrofuran; preference is given to dichloromethane or a dichloromethane/tetrahydrofuran mixture. The reaction in the absence of a solvent has also been found to be advantageous.

The compounds of the formula (II) in which X¹ represents hydroxy are known or can be synthesized by known processes from the appropriate starting materials.

The compounds of the formula (IV) are known or can be synthesized by known processes from the appropriate starting materials.

The compounds of the formula (IV) in which R¹ contains an oxetanyl substituent are known, can be prepared by known processes from the appropriate starting materials or can be prepared as described under the starting materials under Example 5A to Example 12A.

The compounds of the formula (V) are known or can be prepared according to process [B] by reacting compounds of the formula (II) with piperidin-4-one. Piperidin-4-one can also be employed as piperidin-4-one hydrochloride hydrate or in the form of other salts and solvates.

The reaction is carried out as described for process [A].

The compounds of the formula (VII) are known or can be prepared by hydrolysing, in compounds of the formula

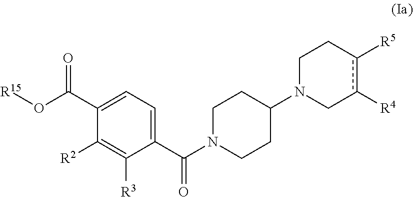

in which
[symbol] , R², R³, R⁴ and R⁵ have the meaning given above and
R¹⁵ represents C₁-C₃-alkyl,
the carboxylic ester.

The hydrolysis is generally carried out in inert solvents, in the presence of a base, preferably in a temperature range from 0° C. to 50° C. at atmospheric pressure.

Inert solvents are, for example, halogenated hydrocarbons such as dichloromethane, trichloromethane or 1,2-dichloroethane, or ethers such as diethyl ether, methyl tert-butyl ether, 1,2-dimethoxyethane, dioxane, tetrahydrofuran, or other solvents such as dimethylformamide, dimethylacetamide, dimethyl sulphoxide or acetonitrile. It is also possible to use mixtures of the solvents mentioned. Preference is given to dioxane or tetrahydrofuran.

Bases are, for example, alkali metal hydroxides such as sodium hydroxide, lithium hydroxide or potassium hydroxide, or alkali metal carbonates such as sodium carbonate, potassium carbonate or caesium carbonate; preference is given to sodium hydroxide or lithium hydroxide.

The compounds of the formula (Ia) are a subset of the compounds of the formula (I) and the preparation is as described for processes [A] to [I].

Starting materials for the preparation of the compounds of the formula (I) can be prepared, for example, as follows:
The present invention also provides a process for preparing 3-(cyclopropyloxy)piperidine, where in a first step 3-hydroxypyridine is reacted with cyclopropyl bromide in the presence of potassium iodide or tetrabutylammonium iodide and in the presence of an inorganic base in an inert solvent to give 3-(cyclopropyloxy)pyridine hydrochloride and the 3-(cyclopropyloxy)pyridine hydrochloride is reacted in a second step in the presence of hydrogen and a catalyst, preferably platinum(IV) oxide, to give 3-(cyclopropyloxy)piperidine hydrochloride. The reaction is generally carried out in an inert solvent and preferably at a pressure of 3 bar.

The first reaction step is optionally carried out in a microwave apparatus, preferably in a temperature range of from 20° C. to 180° C., particularly preferably in a temperature range of from 80° C. to 180° C., at a pressure of from 1 to 20 bar.

Inorganic bases for the first reaction step are, for example, alkali metal carbonates such as sodium carbonate, potassium carbonate or caesium carbonate, or sodium hydrogenphosphate or sodium bicarbonate; preference is given to caesium carbonate.

Inert solvents used in the first reaction step are, for example, dimethyl sulphoxide, dimethylformamide, dimethylacetamide, N-methylpyrrolidone, dioxane, tetrahydrofuran or water. It is also possible to use mixtures of the solvents mentioned. Particular preference is given to water or dimethylformamide.

Inert solvents used in the second reaction step are, for example, alcohols, for example methanol or ethanol. Preference is given to methanol.

The present invention also provides a process for preparing 3-[(trifluoromethoxy)methyl]piperidine which carries an amino protective group, where (piperidin-3-yl)methanol, carrying an amino protective group, is reacted in an inert solvent with carbon disulphide and iodomethane in the presence of sodium hydride in a first step to give S-methyl O-(piperidin-3-ylmethyl) carbonodithioate which carries an amino protective group and this is reacted in a second step with hydrogen fluoride/pyridine complex in an inert solvent to give 3-[(trifluoromethoxy)methyl]piperidine which carries an amino protective group.

Preferred amino protective groups are benzyloxycarbonyl (Boc) and benzyl.

Inert solvents for the first reaction step are, for example, dimethyl sulphoxide, dimethylformamide, dimethylacetamide, N-methylpyrrolidone, dioxane, tetrahydrofuran or water. It is also possible to use mixtures of the solvents mentioned. Particular preference is given to dimethylformamide.

Inert solvents used in the second reaction step are, for example, halogenated hydrocarbons such as dichloromethane or trichloromethane. Preference is given to dichloromethane.

The present invention also provides a process for preparing 3-[(cyclopropyloxy)methyl]piperidine which carries an amino protective group, where in a first reaction step hydroxymethylpiperidine, carrying an amino protective group, is reacted in the presence of a catalyst in an inert solvent with ethyl vinyl ether to give vinyloxymethylpiperidine, which carries an amino protective group, and this is reacted in a second step in an inert solvent with diethylzinc and diiodomethane to give 3-[(cyclopropyloxy)methyl]piperidine, which carries an amino protective group.

Suitable for use as catalysts in the first reaction step are, for example, chloro(triphenylphosphine)gold(I) and silver(I) acetate.

Preferred amino protective groups are benzyloxycarbonyl (Boc) and benzyl.

Suitable for use as inert solvents are, for example, ethers such as diethyl ether.

The present invention also provides 3-(cyclopropyloxy)piperidine.

The present invention also provides 3-[(trifluoromethoxy)methyl]piperidine.

The present invention also provides 3-[(cyclopropyloxy)methyl]piperidine.

The invention furthermore provides a process for preparing the compounds of the formula (I) or the salts thereof, the solvates thereof or the solvates of the salts thereof, where this process comprises reactions according to the processes described above, selected from a group comprising the combinations
[B] and [C], and
[I] and [J].

The preparation of the compounds of the formula (I) and the starting materials mentioned above can be illustrated by the synthesis schemes below.

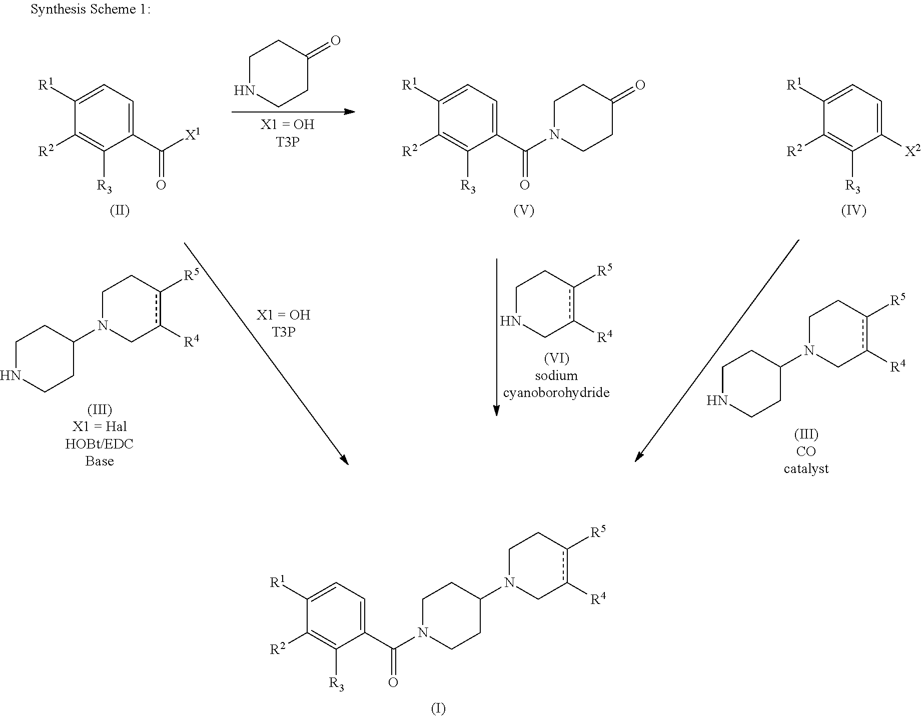

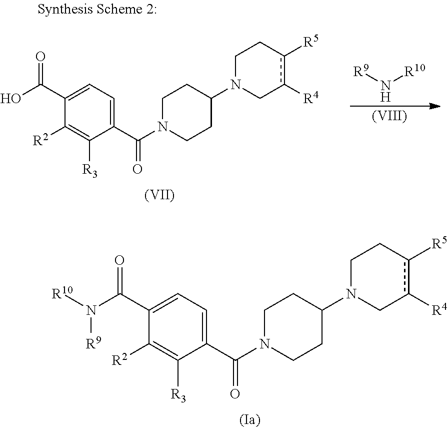

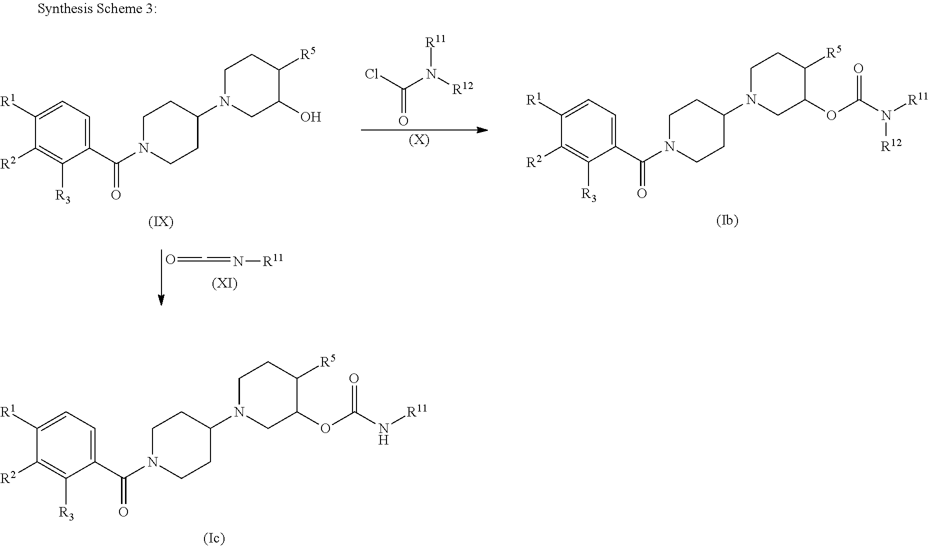

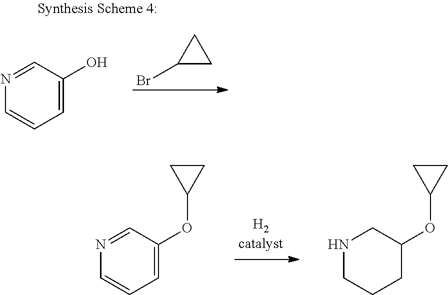

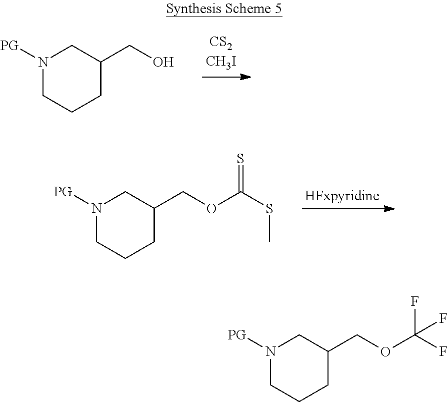

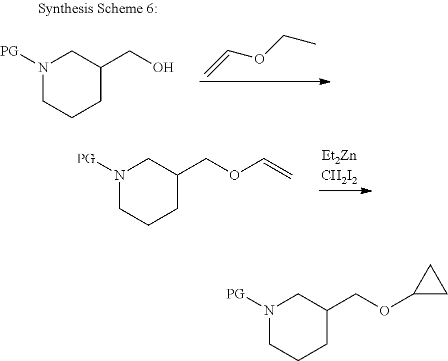

The compounds according to the invention have an unforeseeable useful spectrum of pharmacological activity, including useful pharmacokinetic properties. They are selective adrenoreceptor α_{2C} receptor antagonists which lead to vasorelaxation and/or inhibit platelet aggregation and/or lower blood pressure and/or increase coronary or peripheral blood flow. Accordingly, they are suitable for the treatment and/or prophylaxis of diseases, preferably cardiovascular disorders, diabetic microangiopathies, diabetic ulcers on the extremities, in particular for promoting wound healing of diabetic foot ulcers, diabetic heart failure, diabetic coronary microvascular heart disorders, peripheral and cardiac vascular disorders, thromboembolic disorders and ischaemias, peripheral circulatory disturbances, Raynaud's phenomenon, CREST syndrome, microcirculatory disturbances, intermittent claudication, and peripheral and autonomous neuropathies in humans and animals.

In particular, the compounds according to the invention show a disease-selective improvement of peripheral blood flow (micro- and macrocirculation) under pathophysiologically changed conditions, for example as a consequence of diabetes mellitus or atherosclerosis.

The compounds according to the invention are therefore suitable for use as medicaments for the treatment and/or prophylaxis of diseases in humans and animals.

Accordingly, the compounds according to the invention are suitable for the treatment of cardiovascular disorders such as, for example, for the treatment of high blood pressure, for primary and/or secondary prevention, and also for the treatment of heart failure, for the treatment of stable and unstable angina pectoris, pulmonary hypertension, peripheral and cardiac vascular disorders (e.g. peripheral occlusive disease), arrhythmias, for the treatment of thromboembolic disorders and ischaemias such as myocardial infarction, stroke, transitory and ischaemic attacks, peripheral circulatory disturbances, for the prevention of restenoses such as after thrombolysis therapies, percutaneous transluminal angioplasties (PTAs), percutaneous transluminal coronary angioplasties (PTCAs) and bypass, and also for the treatment of ischaemia syndrome, atherosclerosis, asthmatic disorders, diseases of the urogenital system such as, for example, prostate hypertrophy, erectile dysfunction, female sexual dysfunction and incontinence.

Moreover, the compounds according to the invention can be used for the treatment of primary and secondary Raynaud's phenomenon, of microcirculatory disturbances, intermittent claudication, peripheral and autonomous neuropathies, diabetic microangiopathies, diabetic nephropathy, diabetic retinopathy, diabetic ulcers on the extremities, diabetic erectile dysfunction, CREST syndrome, erythematosis, onychomycosis, tinnitus, dizzy spells, sudden deafness, Meniere's disease and of rheumatic disorders.

The compounds according to the invention are furthermore suitable for the treatment of respiratory distress syndromes and chronic-obstructive pulmonary disease (COPD), of acute and chronic kidney failure and for promoting wound healing and here in particular diabetic wound healing.

Moreover, the compounds of the formula (I) according to the invention are suitable for the treatment and/or prophylaxis of comorbidities and/or sequelae of diabetes mellitus. Examples of comorbidities and/or sequelae of diabetes mellitus are diabetic heart disorders such as, for example, diabetic coronary heart disorders, diabetic coronary microvascular heart disorders (coronary microvascular disease, MVD), diabetic heart failure, diabetic cardiomyopathy and myocardial infarction, hypertension, diabetic microangiopathies, diabetic retinopathy, diabetic neuropathy, stroke, diabetic nephropathy, diabetic erectile dysfunction, diabetic ulcers on the extremities and diabetic foot syndrome. Moreover, the compounds of the formula (I) according to the invention are suitable for promoting diabetic wound healing, in particular for promoting wound healing of diabetic foot ulcers. Promotion of wound healing of diabetic foot ulcers is defined, for example, as improved wound closure.

In addition, the compounds according to the invention are also suitable for controlling cerebral blood flow and are effective agents for controlling migraines. They are also suitable for the prophylaxis and control of sequelae of cerebral infarction (cerebral apoplexy) such as stroke, cerebral ischaemias and craniocerebral trauma. The compounds according to the invention can likewise be employed for controlling states of pain.

In addition, the compounds according to the invention can also be employed for the treatment and/or prevention of micro- and macrovascular damage (vasculitis), reperfusion damage, arterial and venous thromboses, oedemas, neoplastic disorders (skin cancer, liposarcomas, carcinomas of the gastrointestinal tract, of the liver, of the pancreas, of the lung, of the kidney, of the ureter, of the prostate and of the genital tract), of disorders of the central nervous system and neurodegenerative disorders (stroke, Alzheimer's disease, Parkinson's disease, dementia, epilepsy, depressions, multiple sclerosis, schizophrenia), of inflammatory disorders, autoimmune disorders (Crohn's disease, ulcerative colitis, lupus erythematosus, rheumatoid arthritis, asthma), kidney disorders (glomerulonephritis), thyroid disorders (hyperthyreosis), hyperhydrosis, disorders of the pancreas (pancreatitis), liver fibrosis, skin disorders (psoriasis, acne, eczema, neurodermitis, dermatitis, keratitis, formation of scars, formation of warts, chilblains), skin grafts, viral disorders (HPV, HCMV, HIV), cachexia, osteoporosis, avascular bone necrosis, gout, incontinence, for wound healing, for wound healing in patients having sickle cell anaemia, and for angiogenesis.

The present invention furthermore provides the use of the compounds according to the invention for the treatment and/or prophylaxis of disorders, preferably of thromboembolic disorders and/or thromboembolic complications. “Thromboembolic disorders” in the sense of the present invention include in particular disorders such as ST-segment elevation myocardial infarction (STEMI) and non-ST-segment elevation myocardial infarction (non-STEMI), stable angina pectoris, unstable angina pectoris, reocclusions and restenoses after coronary interventions such as angioplasty, stent implantation or aortocoronary bypass, peripheral arterial occlusion diseases, pulmonary embolisms, deep venous thromboses and renal vein thromboses, transitory ischaemic attacks and also thrombotic and thromboembolic stroke and pulmonary hypertension.

Accordingly, the substances are also suitable for the prevention and treatment of cardiogenic thromboembolisms, such as, for example, brain ischaemias, stroke and systemic thromboembolisms and ischaemias, in patients with acute, intermittent or persistent cardiac arrhythmias, such as, for example, atrial fibrillation, and those undergoing cardioversion, furthermore in patients with heart valve disorders or with intravasal objects, such as, for example, artificial heart valves, catheters, intraaortic balloon counterpulsation and pacemaker probes. In addition, the compounds according to the invention are suitable for the treatment of disseminated intravasal coagulation (DIC).

Thromboembolic complications are furthermore encountered in connection with microangiopathic haemolytic anaemias, extracorporeal circulation, such as, for example, haemodialysis, haemofiltration, ventricular assist device and artificial hearts, and also heart valve prostheses.

The compounds according to the invention are particularly suitable for the primary and/or secondary prevention and treatment of heart failure.

In the context of the present invention, the term heart failure also includes more specific or related types of disease, such as right heart failure, left heart failure, global failure, ischaemic cardiomyopathy, dilated cardiomyopathy, congenital heart defects, heart valve defects, heart failure associated with heart valve defects, mitral stenosis, mitral insufficiency, aortic stenosis, aortic insufficiency, tricuspid stenosis, tricuspid insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, and diastolic and systolic heart failure.

The compounds according to the invention are particularly suitable for the treatment and/or prophylaxis of cardiovascular disorders, in particular heart failure, and/or circulatory disturbances and microangiopathies associated with diabetes mellitus.

The compounds according to the invention are also suitable for the primary and/or secondary prevention and treatment of the abovementioned disorders in children.

The present invention further provides the compounds according to the invention for use in a method for the treatment and/or prophylaxis of disorders, especially the disorders mentioned above.

The present invention further provides for the use of the compounds according to the invention for the treatment and/or prophylaxis of disorders, especially the disorders mentioned above.

The present invention further provides for the use of the compounds according to the invention for preparing a medicament for the treatment and/or prophylaxis of disorders, especially the disorders mentioned above.

The present invention further provides a method for the treatment and/or prophylaxis of disorders, especially the disorders mentioned above, using a therapeutically effective amount of a compound according to the invention.

The present invention further provides adrenoreceptor α2C receptor antagonists for use in a method for the treatment and/or prophylaxis of comorbidities and/or sequelae of diabetes mellitus, diabetic heart disorders, diabetic coronary heart disorders, diabetic coronary microvascular heart disorders, diabetic heart failure, diabetic cardiomyopathy and myocardial infarction, diabetic microangiopathy, diabetic retinopathy, diabetic neuropathy, diabetic nephropathy, diabetic erectile dysfunction, diabetic ulcers on the extremities, diabetic foot ulcers, for promoting diabetic wound healing, and for promoting wound healing of diabetic foot ulcers.

The present invention further provides adrenoreceptor α2C receptor antagonists for use in a method for the treatment and/or prophylaxis of diabetic microangiopathies, diabetic retinopathy, diabetic neuropathy, diabetic nephropathy, diabetic erectile dysfunction, diabetic heart failure, diabetic coronary microvascular heart diseases, diabetic ulcers on the extremities, diabetic foot ulcers, for promoting diabetic wound healing and for promoting wound healing of diabetic foot ulcers.

The present invention further provides competitive adrenoreceptor α2C receptor antagonists for use in a method for the treatment and/or prophylaxis of comorbidities and/or sequelae of diabetes mellitus, diabetic heart disorders, diabetic coronary heart disorders, diabetic coronary microvascular heart disorders, diabetic heart failure, diabetic cardiomyopathy and myocardial infarction, diabetic microangiopathy, diabetic retinopathy, diabetic neuropathy, diabetic nephropathy, diabetic erectile dysfunction, diabetic ulcers on the extremities, diabetic foot ulcers, for promoting diabetic wound healing, and for promoting wound healing of diabetic foot ulcers.

The present invention further provides medicaments comprising at least one adrenoreceptor α2C receptor antagonist in combination with one or more inert non-toxic pharmaceutically suitable auxiliaries for the treatment and/or prophylaxis of comorbidities and/or sequelae of diabetes mellitus, diabetic heart disorders, diabetic coronary heart disorders, diabetic coronary microvascular heart disorders, diabetic heart failure, diabetic cardiomyopathy and myocardial infarction, diabetic microangiopathy, diabetic retinopathy, diabetic neuropathy, diabetic nephropathy, diabetic erectile dysfunction, diabetic ulcers on the extremities, diabetic foot ulcers, for promoting diabetic wound healing, and for promoting wound healing of diabetic foot ulcers.

The present invention further provides medicaments comprising at least one adrenoreceptor α2C receptor antagonist in combination with one or more inert non-toxic pharmaceutically suitable auxiliaries for the treatment and/or prophylaxis of diabetic microangiopathies, diabetic retinopathy, diabetic neuropathy, diabetic nephropathy, diabetic erectile dysfunction, diabetic heart failure, diabetic coronary microvascular heart diseases, diabetic ulcers on the extremities, diabetic foot ulcers, for promoting diabetic wound healing, and for promoting wound healing, of diabetic foot ulcers.

The present invention further provides medicaments comprising at least one competitive adrenoreceptor α2C receptor antagonist in combination with one or more inert non-toxic pharmaceutically suitable auxiliaries for the treatment and/or prophylaxis of comorbidities and/or sequelae of diabetes mellitus, diabetic heart disorders, diabetic coronary heart disorders, diabetic coronary microvascular heart disorders, diabetic heart failure, diabetic cardiomyopathy and myocardial infarction, diabetic microangiopathy, diabetic retinopathy, diabetic neuropathy, diabetic nephropathy, diabetic erectile dysfunction, diabetic ulcers on the extremities, diabetic foot ulcers, for promoting diabetic wound healing, and for promoting wound healing of diabetic foot ulcers.

The present invention further provides medicaments comprising at least one adrenoreceptor α2C receptor antagonist in combination with one or more further active compounds selected from the group consisting of lipid metabolism-modulating active compounds, antidiabetics, hypotensive agents, agents which lower the sympathetic tone, perfusion-enhancing and/or antithrombotic agents and also antioxidants, aldosterone and mineralocorticoid receptor antagonists, vasopressin receptor antagonists, organic nitrates and NO donors, IP receptor agonists, positive inotropic compounds, calcium sensitizers, ACE inhibitors, cGMP- and cAMP-modulating compounds, natriuretic peptides, NO-independent stimulators of guanylate cyclase, NO-independent activators of guanylate cyclase, inhibitors of human neutrophil elastase, compounds which inhibit the signal transduction cascade, compounds which modulate the energy metabolism of the heart, chemokine receptor antagonists, p38 kinase inhibitors, NPY agonists, orexin agonists, anorectics, PAF-AH inhibitors, antiphlogistics, analgesics, antidepressants and other psychopharmaceuticals.

The present invention further provides medicaments comprising at least one competitive adrenoreceptor α2C receptor antagonist in combination with one or more further active compounds selected from the group consisting of lipid metabolism-modulating active compounds, antidiabetics, hypotensive agents, agents which lower the sympathetic tone, perfusion-enhancing and/or antithrombotic agents and also antioxidants, aldosterone and mineralocorticoid receptor antagonists, vasopressin receptor antagonists, organic nitrates and NO donors, IP receptor agonists, positive inotropic compounds, calcium sensitizers, ACE inhibitors, cGMP- and cAMP-modulating compounds, natriuretic peptides, NO-independent stimulators of guanylate cyclase, NO-independent activators of guanylate cyclase, inhibitors of human neutrophil elastase, compounds which inhibit the signal transduction cascade, compounds which modulate the energy metabolism of the heart, chemokine receptor antagonists, p38 kinase inhibitors, NPY agonists, orexin agonists, anorectics, PAF-AH inhibitors, antiphlogistics, analgesics, antidepressants and other psychopharmaceuticals.

The present invention further provides a method for the treatment and/or prophylaxis of comorbidities and/or sequelae of diabetes mellitus, diabetic heart disorders, diabetic coronary heart disorders, diabetic coronary microvascular heart disorders, diabetic heart failure, diabetic cardiomyopathy and myocardial infarction, diabetic microangiopathy, diabetic retinopathy, diabetic neuropathy, diabetic nephropathy, diabetic erectile dysfunction, diabetic ulcers on the extremities, diabetic foot ulcers, for promoting diabetic wound healing, and for promoting wound healing of diabetic foot ulcers, in humans and animals by administration of an effective amount of at least one adrenoreceptor α2C receptor antagonist or of a medicament comprising at least one adrenoreceptor α2C receptor antagonist.

The present invention further provides a method for the treatment and/or prophylaxis of diabetic microangiopathies, diabetic retinopathy, diabetic neuropathy, diabetic nephropathy, diabetic erectile dysfunction, diabetic heart failure, diabetic coronary microvascular heart disorders, diabetic ulcers on the extremities, diabetic foot ulcers, for promoting diabetic wound healing, and for promoting wound healing of diabetic foot ulcers.

The present invention further provides a method for the treatment and/or prophylaxis of comorbidities and/or sequelae of diabetes mellitus, diabetic heart disorders, diabetic coronary heart disorders, diabetic coronary microvascular heart disorders, diabetic heart failure, diabetic cardiomyopathy and myocardial infarction, diabetic microangiopathy, diabetic retinopathy, diabetic neuropathy, diabetic nephropathy, diabetic erectile dysfunction, diabetic ulcers on the extremities, diabetic foot ulcers, for promoting diabetic wound healing, and for promoting wound healing of diabetic foot ulcers, in humans and animals by administration of an effective amount of at least one competitive adrenoreceptor α2C receptor antagonist or of a medicament comprising at least one competitive adrenoreceptor α2C receptor antagonist.

Adrenoreceptor α2C receptor antagonists in the context of the present invention are receptor ligands or compounds that block or inhibit the biological responses induced by adrenoreceptor α2C receptor agonists. Adrenoreceptor α2C receptor antagonists in the context of the present invention can be competitive antagonists, non-competitive antagonists, inverse agonists or allosteric modulators.

The compounds according to the invention can be used alone or, if required, in combination with other active compounds. The present invention further provides medicaments comprising a compound according to the invention and one or more further active compounds, in particular for the treatment and/or prophylaxis of the disorders mentioned above. Suitable active compounds for combination are, by way of example and by way of preference: lipid metabolism-modulating active compounds, antidiabetics, hypotensive agents, agents which lower the sympathetic tone, perfusion-enhancing and/or antithrombotic agents and also antioxidants, aldosterone and mineralocorticoid receptor antagonists, vasopressin receptor antagonists, organic nitrates and NO donors, IP receptor agonists, positive inotropic compounds, calcium sensitizers, ACE inhibitors, cGMP- and cAMP-modulating compounds, natriuretic peptides, NO-independent stimulators of guanylate cyclase, NO-independent activators of guanylate cyclase, inhibitors of human neutrophil elastase, compounds which inhibit the signal transduction cascade, compounds which modulate the energy metabolism of the heart, chemokine receptor antagonists, p38 kinase inhibitors, NPY agonists, orexin agonists, anorectics, PAF-AH inhibitors, antiphlogistics (COX inhibitors, LTB₄ receptor antagonists, inhibitors of LTB₄ synthesis), analgesics (aspirin), antidepressants and other psychopharmaceuticals.

The present invention provides in particular combinations of at least one of the compounds according to the invention and at least one lipid metabolism-modulating active compound, antidiabetic, hypotensive active compound and/or antithrombotic agent.

The compounds according to the invention may preferably be combined with one or more of the active compounds mentioned below:
lipid metabolism-modulating active compounds, by way of example and by way of preference from the group of the HMG-CoA reductase inhibitors from the class of the statins such as, by way of example and by way of preference, lovastatin, simvastatin, pravastatin, fluvastatin, atorvastatin, rosuvastatin, cerivastatin or pitavastatin, inhibitors of HMG-CoA reductase expression, squalene synthesis inhibitors such as, by way of example and by way of preference, BMS-188494 or TAK-475, ACAT inhibitors such as, by way of example and by way of preference, melinamide, pactimibe, eflucimibe or SMP-797, LDL receptor inductors, cholesterol absorption inhibitors such as, by way of example and by way of preference, ezetimibe, tiqueside or pamaqueside, polymeric bile acid adsorbers such as, by way of example and by way of preference, cholestyramine, colestipol, colesolvam, CholestaGel or colestimide, bile acid reabsorption inhibitors such as, by way of example and by way of preference, ASBT (=IBAT) inhibitors such as elobixibat (AZD-7806), S-8921, AK-105, canosimibe (BARI-1741, AVE-5530), SC-435 or SC-635, MTP inhibitors such as, by way of example and by way of preference, implitapide or JTT-130, lipase inhibitors such as, by way of example and by way of preference, orlistat, LpL activators, fibrates, niacin, CETP inhibitors such as, by way of example and by way of preference, torcetrapib, dalcetrapib (JTT-705) or CETP vaccine (Avant), PPAR-γ and/or PPAR-δ agonists such as, by way of example and by way of preference, pioglitazone or rosiglitazone and/or endurobol (GW-501516), RXR modulators, FXR modulators, LXR modulators, thyroid hormones and/or thyroid mimetics such as, by way of example and by way of preference, D-thyroxine or 3,5,3′-triiodothyronine (T3), ATP citrate lyase inhibitors, Lp(a) antagonists, cannabinoid receptor 1-antagonists such as, by way of example and by way of preference, rimonabant or surinabant (SR-147778), leptin receptor agonists, bombesin receptor agonists, histamine receptor agonists, agonists of the niacin receptor such as, by way of example and by way of preference, niacin, acipimox, acifran or radecol, and the antioxidants/radical scavengers such as, by way of example and by way of preference, probucol, succinobucol (AGI-1067), BO-653 or AEOL-10150;
antidiabetics mentioned in Die Rote Liste 2014, chapter 12. Antidiabetics are preferably understood as meaning insulin and insulin derivatives and also orally effective hypoglycaemically active compounds. Here, insulin and insulin derivatives include both insulins of animal, human or biotechnological origin and mixtures thereof. The orally effective hypoglycaemically active compounds preferably include sulphonylureas, biguanides, meglitinide derivatives, glucosidase inhibitors and PPAR-gamma agonists. Sulphonylureas which may be mentioned are, by way of example and by way of preference, tolbutamide, glibenclamide, glimepiride, glipizide or gliclazide, biguanides which may be mentioned are, by way of example and by way of preference, metformin, meglitinide derivatives which may be mentioned are, by way of example and by way of preference, repaglinide or nateglinide, glucosidase inhibitors which may be mentioned are, by way of example and by way of preference, miglitol or acarbose, oxadiazolidinones, thiazolidinediones, GLP 1 receptor agonists, glucagon antagonists, insulin sensitizers, CCK 1 receptor agonists, leptin receptor agonists, inhibitors of liver enzymes involved in the stimulation of gluconeogenesis and/or glycogenolysis, modulators of glucose uptake and potassium channel openers such as, for example, those disclosed in WO 97/26265 and WO 99/03861;
hypotensive active compounds, by way of example and by way of preference from the group of the calcium antagonists such as, by way of example and by way of preference, nifedipine, amlodipine, verapamil or diltiazem, angiotensin AII antagonists such as, by way of example and by way of preference, losartan, valsartan, candesartan, embusartan or telmisartan, ACE inhibitors such as, by way of example and by way of preference, enalapril, captopril, ramipril, delapril, fosinopril, quinopril, perindopril or trandopril, beta receptor blockers such as, by way of example and by way of preference, propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol, alpha receptor blockers such as, by way of example and by way of preference, prazosin, ECE inhibitors, rho kinase inhibitors and of the vasopeptidase inhibitors, and also of the diuretics such as, by way of example and by way of preference, a loop diuretic such as furosemide, bumetanide or torsemide, or a thiazide or thiazide-like diuretic such as chlorothiazide or hydrochlorothiazide or A1 antagonists such as rolofylline, tonopofylline and SLV-320;
agents which lower the sympathetic tone such as, by way of example and by way of preference, reserpin, clonidine or alpha-methyldopa, or in combination with a potassium channel agonist such as, by way of example and by way of preference, minoxidil, diazoxide, dihydralazine or hydralazine;
antithrombotic agents such as, by way of example and by way of preference, from the group of the platelet aggregation inhibitors such as, by way of example and by way of preference, aspirin, clopidogrel, ticlopidine, cilostazol or dipyridamole, or of the anticoagulants such as thrombin inhibitors such as, by way of example and by way of preference, ximelagatran, melagatran, bivalirudin or clexane, a GPIIb/IIIa antagonist such as, by way of example and by way of preference, tirofiban or abciximab, a factor Xa inhibitor such as, by way of example and by way of preference, rivaroxaban, edoxaban (DU-176b), apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428, with heparin or a low molecular weight (LMW) heparin derivative or with a vitamin K antagonist such as, by way of example and by way of preference, coumarin;
aldosterone and mineralocorticoid receptor antagonists such as, by way of example and by way of preference, spironolactone, eplerenone or finerenone;
vasopressin receptor antagonists such as, by way of example and by way of preference, conivaptan, tolvaptan, lixivaptan or satavaptan (SR-121463);
organic nitrates and NO donors such as, by way of example and by way of preference, sodium nitroprusside, nitroglycerol, isosorbide mononitrate, isosorbide dinitrate, molsidomine or SIN-1, or in combination with inhalative NO;
IP receptor agonists, such as, by way of example and by way of preference, iloprost, treprostinil, beraprost and selexipag (NS-304);
positive inotropic compounds, such as, by way of example and by way of preference, cardiac glycosides (digoxin), beta-adrenergic and dopaminergic agonists such as isoproterenol, adrenalinee, noradrenalinee, dopamine and dobutamine;
calcium sensitizers, such as, by way of example and by way of preference, levosimendan;
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, especially PDE 5 inhibitors such as sildenafil, vardenafil and tadalafil, and PDE 3 inhibitors such as milrinone;
natriuretic peptides, for example atrial natriuretic peptide (ANP, anaritide), B-type natriuretic peptide or brain natriuretic peptide (BNP, nesiritide), C-type natriuretic peptide (CNP) and urodilatin;
NO-independent but haem-dependent stimulators of guanylate cyclase, such as especially the compounds described in WO 00/06568, WO 00/06569, WO 02/42301 and WO 03/095451;
NO- and haem-independent activators of guanylate cyclase, such as especially the compounds described in WO 01/19355, WO 01/19776, WO 01/19778, WO 01/19780, WO 02/070462 and WO 02/070510;
inhibitors of human neutrophil elastase (HNE), for example sivelestat and DX-890 (Reltran);
compounds which inhibit the signal transduction cascade, for example tyrosine kinase inhibitors and multikinase inhibitors, especially sorafenib, imatinib, gefitinib and erlotinib; and/or
compounds which influence the energy metabolism of the heart, such as, for example, etomoxir, dichloroacetate, ranolazine and trimetazidine.

In the context of the present invention, particular preference is given to combinations comprising at least one of the compounds according to the invention and one or more further active compounds selected from the group consisting of HMG-CoA reductase inhibitors (statins), diuretics, beta-receptor blockers, organic nitrates and NO donors, ACE inhibitors, angiotensin AII antagonists, aldosterone and mineralocorticoid receptor antagonists, vasopressin receptor antagonists, platelet aggregation inhibitors and anticoagulants, and also their use for the treatment and/or prevention of the disorders mentioned above.

Particular preference in the context of the present invention is given to combinations comprising at least one of the compounds according to the invention and one or more further active compounds selected from the group consisting of heparin, antidiabetics, ACE inhibitors, diuretics and antibiotics, and also to their use in a method for promoting diabetic wound healing and for the treatment and/or prevention of diabetic ulcers on the extremities, in particular for promoting wound healing of diabetic foot ulcers.

Particular preference in the context of the present invention is given to the use of at least one of the compounds according to the invention in a method for promoting diabetic wound healing and for the treatment and/or prevention of diabetic ulcers on the extremities, in particular for promoting wound healing of diabetic foot ulcers, where the compound of the formula (I) is additionally employed for one or more of the following physical and/or topical therapies: wound management such as dressings, wound excision, weight reduction with appropriate footwear, PDGF (Regranex), hyperbaric oxygen therapy, wound therapy with negative pressure.

The compounds of the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds of the invention can be administered in administration forms suitable for these administration routes.

Suitable administration forms for oral administration are those which function according to the prior art and deliver the inventive compounds rapidly and/or in modified fashion, and which contain the inventive compounds in crystalline and/or amorphized and/or dissolved form, for example tablets (uncoated or coated tablets, for example having enteric coatings or coatings which are insoluble or dissolve with a delay, which control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

Oral administration is preferred.

In the exemplary use of the compounds of the formula (I) for promoting diabetic wound healing, in particular for promoting wound healing of diabetic foot ulcers, preference, in addition to oral administration, is also given to administration in the form of a topical formulation.

Suitable administration forms for the other administration routes are, for example, pharmaceutical forms for inhalation (including powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

The compounds of the invention can be converted to the administration forms mentioned. This can be accomplished in a manner known per se by mixing with inert non-toxic pharmaceutically suitable auxiliaries. These auxiliaries include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

The present invention further provides medicaments comprising at least one inventive compound, preferably together with one or more inert non-toxic pharmaceutically suitable auxiliaries, and the use thereof for the purposes mentioned above.

In general, it has been found to be advantageous in the case of oral administration to administer amounts of from about 0.1 to 250 mg per 24 hours, preferably 0.1 to 50 mg per 24 hours, to achieve effective results. The dose may be divided into a plurality of administrations per day. Examples are administrations twice or three times per day.

It may nevertheless be necessary in some cases to deviate from the stated amounts, specifically as a function of body weight, route of administration, individual response to the active compound, nature of the preparation and time or interval over which administration takes place.

The present invention further provides a compound of the formula (I) as described above for use in a method for the treatment and/or prophylaxis of primary and secondary forms of diabetic microangiopathies, diabetic wound healing, diabetic ulcers on the extremities, in particular for promoting wound healing of diabetic foot ulcers, diabetic retinopathy, diabetic nephropathy, diabetic erectile dysfunction, diabetic heart failure, diabetic coronary microvascular heart disorders, peripheral and cardiac vascular disorders, thromboembolic disorders and ischaemias, peripheral circulatory disturbances, Raynaud's phenomenon, CREST syndrome, microcirculatory disturbances, intermittent claudication, and peripheral and autonomous neuropathies.

The present invention further provides a compound of the formula (I) as described above for use in a method for the treatment and/or prophylaxis of primary and secondary forms of heart failure, peripheral and cardiavascular disorders, thromboembolic disorders and ischaemias, peripheral circulatory disturbances, Raynaud's phenomenon, microcirculatory disturbances, intermittent claudication, peripheral and autonomous neuropathies, and CREST syndrome, and also for diabetic wound healing, in particular for promoting wound healing of diabetic foot ulcers.

The present invention further provides a compound of the formula (I) as described above for preparing a medicament for the treatment and/or prophylaxis of primary and secondary forms of diabetic microangiopathies, diabetic wound healing, diabetic ulcers on the extremities, in particular for promoting wound healing of diabetic foot ulcers, diabetic retinopathy, diabetic nephropathy, diabetic erectile dysfunction, diabetic heart failure, diabetic coronary microvascular heart disorders, peripheral and cardiac vascular disorders, thromboembolic disorders and ischaemias, peripheral circulatory disturbances, Raynaud's phenomenon, CREST syndrome, microcirculatory disturbances, intermittent claudication, and peripheral and autonomous neuropathies.

The present invention further provides the use of a compound of the formula (I) as described above for preparing a medicament for the treatment and/or prophylaxis of primary and secondary forms of heart failure, peripheral and cardiavascular disorders, thromboembolic disorders and ischaemias, peripheral circulatory disturbances, Raynaud's phenomenon, microcirculatory disturbances, intermittent claudication, peripheral and autonomous neuropathies, and CREST syndrome, and also for diabetic wound healing, in particular for promoting wound healing of diabetic foot ulcers.

The present invention further provides a medicament comprising a compound of the formula (I) as described above in combination with one or more inert non-toxic pharmaceutically suitable auxiliaries.

The present invention further provides a medicament comprising a compound of the formula (I) as described above in combination with one or more further active compounds selected from the group consisting of lipid metabolism-modulating active compounds, antidiabetics, hypotensive agents, agents which lower the sympathetic tone, perfusion-enhancing and/or antithrombotic agents and also antioxidants, aldosterone and mineralocorticoid receptor antagonists, vasopressin receptor antagonists, organic nitrates and NO donors, IP receptor agonists, positive inotropic compounds, calcium sensitizers, ACE inhibitors, cGMP- and cAMP-modulating compounds, natriuretic peptides, NO-independent stimulators of guanylate cyclase, NO-independent activators of guanylate cyclase, inhibitors of human neutrophil elastase, compounds which inhibit the signal transduction cascade, compounds which modulate the energy metabolism of the heart, chemokine receptor antagonists, p38 kinase inhibitors, NPY agonists, orexin agonists, anorectics, PAF-AH inhibitors, antiphlogistics, analgesics, antidepressants and other psychopharmaceuticals.

The present invention further provides a medicament as described above for the treatment and/or prophylaxis of primary and secondary forms of diabetic microangiopathies, diabetic wound healing, diabetic ulcers on the extremities, in particular for promoting wound healing of diabetic foot ulcers, diabetic retinopathy, diabetic nephropathy, diabetic erectile dysfunction, diabetic heart failure, diabetic coronary microvascular heart disorders, peripheral and cardiac vascular disorders, thromboembolic disorders and ischaemias, peripheral circulatory disturbances, Raynaud's phenomenon, CREST syndrome, microcirculatory disturbances, intermittent claudication, and peripheral and autonomous neuropathies.

The present invention further provides a medicament as described above for the treatment and/or prophylaxis of primary and secondary forms of heart failure, peripheral and cardiavascular disorders, thromboembolic disorders and ischaemias, peripheral circulatory disturbances, Raynaud's phenomenon, microcirculatory disturbances, intermittent claudication, peripheral and autonomous neuropathies, and CREST syndrome, and also for diabetic wound healing, in particular for promoting wound healing of diabetic foot ulcers.

The present invention further provides a method for the treatment and/or prophylaxis of primary and secondary forms of diabetic microangiopathies, diabetic wound healing, diabetic ulcers on the extremities, in particular for promoting wound healing of diabetic foot ulcers, diabetic retinopathy, diabetic nephropathy, diabetic erectile dysfunction, diabetic heart failure, diabetic coronary microvascular heart disorders, peripheral and cardiac vascular disorders, thromboembolic disorders and ischaemias, peripheral circulatory disturbances, Raynaud's phenomenon, CREST syndrome, microcirculatory disturbances, intermittent claudication, and peripheral and autonomous neuropathies in humans and animals by administration of an effective amount of at least one compound of the formula (I) as described above or of a medicament as described above.

The present invention further provides a method for the treatment and/or prophylaxis of primary and secondary forms of heart failure, peripheral and cardiavascular disorders, thromboembolic disorders and ischaemias, peripheral circulatory disturbances, Raynaud's phenomenon, microcirculatory disturbances, intermittent claudication, peripheral and autonomous neuropathies, and CREST syndrome, and also for diabetic wound healing, in particular for promoting wound healing of diabetic foot ulcers, in humans and animals by administration of an effective amount of at least one compound of the formula (I) as described above or of a medicament as described above.

Unless stated otherwise, the percentages in the tests and examples which follow are percentages by weight; parts are parts by weight. Solvent ratios, dilution ratios and concentration data for the liquid/liquid solutions are based in each case on volume. “w/v” means “weight/volume”. For example, “10% w/v” means: 100 ml of solution or suspension comprise 10 g of substance.

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “formate”, “sodium salt” or “x HCl”, “x CF3COOH”, “x CHCOOH”, “x Na+” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

### A) EXAMPLES

### Abbreviations

α specific rotation
Å Angström
br. broad signal (in NMR)
Ex. no. Example number
CDI carbonyldiimidazole
d day(s), doublet (in NMR)
DAD diode array detector (in HPLC and LC-MS)
TLC thin-layer chromatography
DCI direct chemical ionization (in MS)
dd doublet of doublets (in NMR)
DMAP 4-dimethylaminopyridine
DMF N,N-dimethylformamide
DMSO dimethyl sulphoxide
DSC disuccinimidyl carbonate
EDC 1-(3-dimethylaminopropyl)-3-ethylcarbodiimide hydrochloride
EDTA 2,2′,2″,2′″-(ethane-1,2-diyldinitrilo)tetraacetic acid
EI electron impact (ionisation method in MS)
eq. equivalent(s)
ESI electrospray ionization (in MS)
GC-MS gas chromatography-coupled mass spectroscopy
h hour(s)
HATU O-(7-azabenzotriazol-1-yl)-N,N,N′,N′-tetramethyluronium hexafluorophosphate
HOBT 1-hydroxy-1H-benzotriazole hydrate
HPLC high-pressure, high-performance liquid chromatography
HV high vacuum
LC-MS liquid chromatography-coupled mass spectroscopy
LDA lithium diisopropylamide
m multiplet (in NMR)
min minute(s)
MS mass spectroscopy
NMR nuclear magnetic resonance spectroscopy
PYBOP benzotriazol-1-yloxy-tris(pyrrolidino)phosphonium hexafluorophosphate
q quartet (in NMR)
quin. quintet (in NMR)
R_{f} retention factor (in TLC)
RP reversed phase (in HPLC)
RT room temperature
Rₜ retention time (in HPLC)
s singlet (in NMR)
t triplet (in NMR)
T3P propylphosphonic anhydride 50% strength in ethyl acetate or DMF
THF tetrahydrofuran
w/w percent by weight

### LC-MS, GC-MS and HPLC Methods:

Method 1 (LC-MS): instrument: Waters ACQUITY SQD UPLC system; column: Waters Acquity UPLC HSS T3 1.8μ 50 mm×1 mm; elution A: 1 l of water+0.25 ml of 99% strength formic acid, elution B: 1 l of acetonitrile+0.25 ml of 99% strength formic acid; gradient: 0.0 min 90% A→1.2 min 5% A→2.0 min 5% A; oven: 50° C.; flow rate: 0.40 ml/min; UV detection: 210-400 nm.

Method 2 (LC-MS): instrument: Waters ACQUITY SQD UPLC System; column: Waters Acquity UPLC HSS T3 1.8μ 50 mm×1 mm; elution A: 1 l of water+0.25 ml of 99% strength formic acid, elution B: 1 l of acetonitrile+0.25 ml of 99% strength formic acid; gradient: 0.0 min 90% A→1.2 min 5% A→2.0 min 5% A; oven: 50° C.; flow rate: 0.40 ml/min; UV detection: 210-400 nm.

Method 3 (LC-MS): instrument: Micromass Quattro Premier with Waters UPLC Acquity; column: Thermo Hypersil GOLD 1.9μ 50 mm×1 mm; elution A: 1 l of water+0.5 ml 50% strength formic acid, elution B: 1 l of acetonitrile+0.5 ml 50% strength formic acid; gradient: 0.0 min 90% A→0.1 min 90% A→1.5 min 10% A→2.2 min 10% A; oven: 50° C.; flow rate: 0.33 ml/min; UV detection: 210 nm.

Method 4 (LC-MS): MS instrument type: Waters (Micromass) Quattro Micro; HPLC instrument type: Agilent 1100 series; column: Thermo Hypersil GOLD 3μ 20 mm×4 mm; elution A: 1 l of water+0.5 ml 50% strength formic acid, elution B: 1 l of acetonitrile+0.5 ml 50% strength formic acid; gradient: 0.0 min 100% A→3.0 min 10% A→4.0 min 10% A→4.01 min 100% A (flow rate: 2.5 ml)→5.00 min 100% A; oven: 50° C.; flow rate: 2 ml/min; UV detection: 210 nm.

Method 5 (GC-MS): instrument: Micromass GCT, GC6890; column: Restek RTX-35, 15 m×200 μm×0.33 μm; constant helium flow: 0.88 ml/min; oven: 70° C.; inlet: 250° C.; gradient: 70° C., 30° C./min→310° C. (hold for 3 min).

Method 6 (LC-MS): MS instrument type: Waters ZQ; HPLC instrument type: Agilent 1100 series; UV DAD; column: Thermo Hypersil GOLD 3μ 20 mm×4 mm; elution A: 1 l of water+0.5 ml of 50% strength formic acid, elution B: 1 l of acetonitrile+0.5 ml of 50% strength formic acid; gradient: 0.0 min 100% A→3.0 min 10% A→4.0 min 10% A→4.1 min 100%; oven: 55° C.; flow rate: 2 ml/min; UV detection: 210 nm.

Method 7 (LC-MS): MS instrument: Waters ZQ 2000; HPLC instrument: Agilent 1100, 2-column set-up, autosampler: HTC PAL; column: YMC-ODS-AQ, 50 mm×4.6 mm, 3.0 μm; elution A: water+0.1% formic acid, elution B: acetonitrile+0.1% formic acid; gradient: 0.0 min 100% A→0.2 min 95% A→1.8 min 25% A→1.9 min 10% A→2.0 min 5% A→3.2 min 5% A→3.21 min 100% A→3.35 min 100% A; oven: 40° C.; flow rate: 3.0 ml/min; UV detection: 210 nm.

Method 8 (LC-MS): instrument: Micromass Quattro Premier with Waters UPLC Acquity; column: Thermo Hypersil GOLD 1.9μ 50×1 mm; elution A: 1 l of water+0.5 ml 50% strength formic acid, elution B: 1 l of acetonitrile+0.5 ml 50% strength formic acid; gradient: 0.0 min 90% A→0.1 min 90% A→1.5 min 10% A→2.2 min 10% A; oven: 50° C.; flow rate: 0.33 ml/min; UV detection: 210 nm.

Method 9 (LC-MS): instrument: Waters ACQUITY SQD UPLC System; column: Waters Acquity UPLC HSS T3 1.8μ 30×2 mm; elution A: 1 l of water+0.25 ml of 99% strength formic acid, elution B: 1 l of acetonitrile+0.25 ml of 99% strength formic acid; gradient: 0.0 min 90% A→1.2 min 5% A→2.0 min 5% A; oven: 50° C.; flow rate: 0.60 ml/min; UV detection: 208-400 nm.

Method 10 (LC-MS): instrument: Micromass Quattro Premier with Waters UPLC Acquity; column: Thermo Hypersil GOLD 1.9μ 50×1 mm; elution A: 1 l of water+0.5 ml 50% strength formic acid, elution B: 1 l of acetonitrile+0.5 ml 50% strength formic acid; gradient: 0.0 min 97% A→0.5 min 97% A→3.2 min 5% A→4.0 min 5% A; oven: 50° C.; flow rate: 0.3 ml/min; UV detection: 210 nm.

### Preparative HPLC:

Method 10 (Preparative HPLC): column: YMC-ODS C18, 250×20 mm, 10 μm, elution A: 1 l of water+0.5 ml of trifluoroacetic acid, elution B: 1 l of acetonitrile+0.5 ml of trifluoroacetic acid, gradient: 0.0 min 90% A->3.0 min 90% A->24.0 min 50% A->35.0 min 50% A->35.1 min 90% A; flow rate: 20 ml/min; UV detection: 210 nm.

Method 11 (Preparative HPLC): column: Kromasil C18, 250×20 mm, 10 μm, elution A: water+0.1% trifluoroacetic acid, elution B: acetonitrile+0.1% trifluoroacetic acid, gradient: 0.0 min 90% A->3.0 min 90% A->24 min 50% A->35 min 50% A->35.1 min 90% A; flow rate: 20 ml/min; UV detection: 210 nm.

Method 12a (Preparative HPLC): column: Reprosil C18, 250×40 mm, 10 μm, elution A: water+0.1% trifluoroacetic acid, elution B: acetonitrile+0.1% trifluoroacetic acid, gradient: 0.0 min 90% A->3.0 min 90% A->24 min 50% A->35 min 50% A->35.1 min 90% A; flow rate: 40 ml/min; UV detection: 210 nm.

Method 12b: as above, but gradient: 0.0 min 90% A->3.0 min 90% A->25 min 10% A->35 min 90% A.

Method 12c: as above, but gradient: A=water+0.1% trifluoroacetic acid, B=acetonitrile+0.1% trifluoroacetic acid, 3 min=10% B pre-run without substance, then injection, 5 min=10% B, 25 min=50% B, 45 min=50% B, 45.1 min=10% B, 48 min=10% B.

Method 13 (Preparative HPLC): column: Reprosil C18, 250×40 mm, 10 μm, elution A: water+0.5% formic acid, elution B: acetonitrile, gradient: 0.0 min 95% A->3.0 min 95% A->24 min 50% A->35 min 50% A->35.1 min 95% A; flow rate: 20 ml/min; UV detection: 210 nm

Method 14 (Preparative HPLC): column: Reprosil C18, 250×40 mm, 10 μm, elution A: water, elution B: acetonitrile, gradient: 0.0 min 95% A->3.0 min 95% A->24 min 70% A->34 min 70% A->34.1 min 95% A; flow rate: 20 ml/min; UV detection: 210 nm.

Method 15 (Preparative HPLC): column: Reprosil C18, 250×20 mm, 10 μm, elution A: water+0.5% formic acid, elution B: acetonitrile, gradient: 0.0 min 95% A->3.0 min 95% A->24 min 50% A->35 min 50% A->35.1 min 95% A; flow rate: 20 ml/min; UV detection: 210 nm.

Method 16 (Preparative HPLC): column: Reprosil C18, 250×30 mm, 10 μm, elution A: water, elution B: methanol; gradient: 0.0 min 35% B→8 min 35% B→20 min 70% B→40 min 95%; flow rate: 30 ml/min; column temperature: RT; UV detection: 210 nm

Method 17 (Chiral Preparative HPLC): stationary phase Daicel Chiralpak AD-H 5 μm, column: 250 mm×20 mm; temperature: 25° C.; UV detection: 230 nm. Various mobile phases:
Method 17a: mobile phase: isohexane/ethanol (+0.2% diethylamine) 80:20 (v/v); flow rate: 20 ml/min
Method 17b: mobile phase: isohexane/ethanol (+0.2% diethylamine) 50:50 (v/v); flow rate: 15 ml/min

Method 18 (Chiral Analytical HPLC): stationary phase Daicel Chiralpak AD-H 5 μm, column: 250 mm×4.6 mm; temperature: 40° C.; UV detection: 220 nm. Various mobile phases:
Method 18a: mobile phase: isohexane/ethanol (+0.2% diethylamine) 80:20 (v/v); flow rate: 1 ml/min
Method 18b: mobile phase: isohexane/ethanol (+0.2% diethylamine) 50:50 (v/v); flow rate: 1 ml/min

Method 19 (Chiral Preparative HPLC): stationary phase Daicel Chiralpak AY-H 5 μm, column: 250 mm×20 mm; temperature: 40° C.; UV detection: 210 nm. Various mobile phases:
Method 19a: mobile phase: isohexane/ethanol (+0.2% diethylamine) 50:50 (v/v); flow rate: 17 ml/min
Method 19b: mobile phase: isohexane/2-propanol (+0.2% diethylamine) 50:50 (v/v); flow rate: 18 ml/min

Method 20 (Chiral Analytical HPLC): stationary phase Daicel Chiralpak AY-H 5 μm, column: 250 mm×4.6 mm; temperature: 30° C.; UV detection: 220 nm. Various mobile phases:
Method 20a: mobile phase: isohexane/ethanol (+0.2% diethylamine) 50:50 (v/v); flow rate: 1 ml/min
Method 20b: mobile phase: isohexane/2-propanol (+0.2% diethylamine) 50:50 (v/v); flow rate: 1 ml/min

Method 21 (Preparative HPLC): column: Waters XBridge, 50×19 mm, 10 μm, mobile phase A: water+0.5% ammonium hydroxide, mobile phase B: acetonitrile, 5 min=95% A, 25 min=50% A, 38 min=50% A, 38.1 min=5% A, 43 min=5% A, 43.01 min=95% A, 48.0 min=5% A; flow rate 20 ml/min, UV detection: 210 nm.

Method 22 (Preparative HPLC): column: Chromatorex C18, 250×20 mm, 10 μm, mobile phase A: water+0.5% formic acid, mobile phase B: acetonitrile, gradient: 0.0 min 95% A->3.0 min 95% A->25 min 50% A->38 min 50% A->38.1 min 95% A; flow rate: 20 ml/min; UV detection: 210 nm.

The microwave reactor used was an instrument of the CEM Discover™ type.

The NMR data were assigned unless the signals were concealed by solvent.

For H-Cube hydrogenations, use is made of the HC-2.SS instrument from ThalesNano.

### Starting Materials

### Example 1A

### 4-tert-Butyl-2-methoxybenzoic acid

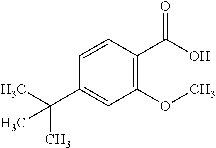

500 mg (2.25 mmol) of methyl 4-tert-butyl-2-methoxybenzoate were initially charged in 10 ml of dioxane, and 161.6 mg (24 mmol) of lithium hydroxide were added. The mixture was stirred at RT overnight and then at 60° C. for 1 h. After cooling, the reaction mixture was concentrated and taken up in ethyl acetate. The organic phase was washed with dilute hydrochloric acid, dried over sodium sulphate and concentrated. This gave 327 mg (65% of theory) of the title compound. The product is described in Shirley et al J. Organometallic Chemistry, 1974, 69, 327-344.

LC-MS [Method 4]: Rₜ=2.05 min; MS (ESIpos): m/z=209 (M+H)⁺

¹H-NMR (400 MHz, DMSO-d₆): δ [ppm]=1.30 (s, 9H), 3.83 (s, 3H), 7.99-7.04 (m, 1H), 7.04-7.08 (m, 1H), 7.57-7.62 (m, 1H), 12.36 (br. s., 1H)

### Example 2A

### 1-[(4-tert-Butylphenyl)carbonyl]piperidin-4-one

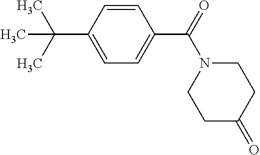 [SMILES: CC(C)(C)c1ccc(C(=O)N2CCC(=O)CC2)cc1]

3 g (0.56 mmol) of 4-tert-butylbenzoic acid were dissolved in 40 ml of DMF, and 3.2 g (16.8 mmol) of EDC, 2.58 g (16.8 mmol) of HOBT and 8.7 g (67.3 mmol) of N,N-diisopropylethylamine were added. The mixture was stirred at RT for 1 h. 2.59 g (16.8 mmol) of piperidin-4-one hydrochloride hydrate were then added, and the mixture was subsequently stirred at RT overnight. The mixture was diluted with ethyl acetate and washed with water and saturated sodium chloride solution. The organic phase was separated off, dried over sodium sulphate, filtered and concentrated. The resulting product was crystallized from cyclohexane, filtered off with suction and air-dried. This gave 2.59 g (59% of theory) of the title compound.

LC-MS [Method 1]: Rₜ=0.97 min; MS (ESIpos): m/z=260 (M+H)⁺

¹H-NMR (400 MHz, DMSO-d₆): δ [ppm]=1.30 (s, 9H), 2.32-2.47 (m, 4H), 3.5-4.0 (m, 4H), 7.39-7.44 (m, 2H), 7.45-7.51 (m, 2H)

### Example 3A

### (4-tert-Butylphenyl)(3-hydroxy-1,4′-bipiperidin-1′-yl)methanone

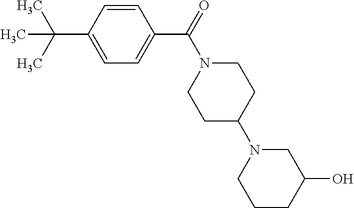 [SMILES: CC(C)(C)c1ccc(C(=O)N2CCC(N3CCCC(O)C3)CC2)cc1]

1.5 g (3.7 mmol) of 1-[(4-tert-butylphenyl)carbonyl]piperidin-4-one were initially charged in 45 ml of 10% strength glacial acetic acid/methanol solution, and 1.52 g (5.55 mmol) of 3-hydroxypiperidine were added. After one hour of stirring at RT, 0.49 g (7.4 mmol) of sodium cyanoborohydride was added, and the mixture was stirred at RT overnight. The reaction mixture was taken up in ethyl acetate and extracted with saturated sodium bicarbonate solution and saturated sodium chloride solution. The organic phase was dried over sodium sulphate, filtered and concentrated. The product was purified by flash chromatography on silica gel, elution: ethyl acetate, gradient ethyl acetate/methanol: 5/1. The product-containing fractions were concentrated and dried under HV. This gave 0.75 g (59% of theory) of the title compound as a solid.

LC-MS [Method 4]: Rₜ=1.41 min; MS (ESIpos): m/z=345 (M+H)⁺

¹H-NMR (400 MHz, DMSO-d₆): δ [ppm]=0.97-1.10 (m, 1H), 1.23-1.43 (m, 4H), 1.29 (s, 9H), 1.54-1.83 (m, 3H), 1.83-1.93 (m, 1H), 1.97-2.09 (m, 1H), 2.04 (t, 1H), 2.62-2.88 (m, 2H), 2.89-3.06 (m. 1H), 3.36-3.45 (m, 1H), 3.5-3.7 (m, 1H), 4.35-4.59 (m, 1H), 4.55 (d, 1H), 7.26-7.36 (m, 2H), 7.41-7.52 (m, 2H)

### Example 4A

### 1′-[(4-tert-Butylphenyl)carbonyl]-1,4′-bipiperidine-3-carbohydrazide

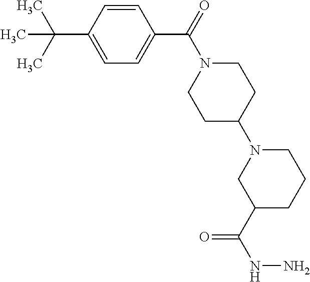 [SMILES: CC(C)(C)c1ccc(C(=O)N2CCC(N3CCCC(C(=O)NN)C3)CC2)cc1]

4.2 g (20 mmol) of hydrazine hydrate in water (24%) were added to 200 mg (0.5 mmol) of ethyl 1′-[(4-tert-butylphenyl)carbonyl]-1,4′-bipiperidine-3-carboxylate, and the mixture was stirred at reflux overnight. 2 ml of ethanol were added, and the mixture was stirred at reflux for another night. After cooling, the mixture was concentrated and the product formed was purified by preparative HPLC [Reprosil, C18 10 μm, 250 mm×30 mm, methanol/water 30:70 to 100/0 over a run time of 23 min, Method 16]. After HPLC control, the product-containing fractions were combined and concentrated. The residue was dried under HV, giving 42 mg (56% of theory) of the title compound.

LC-MS [Method 1]: Rₜ=0.68 min; MS (ESIpos): m/z=387 (M+H)⁺

¹H-NMR (400 MHz, DMSO-d₆): δ [ppm]=1.25-1.47 (m, 4H), 1.29 (s, 9H), 1.55-1.85 (m, 2H), 1.5-1.85 (m, 2H), 2.12 (t, 1H), 2.19-2.30 (m, 2H), 2.46-2.57 (m, 2H), 2.63-2.88 (m, 2H), 2.75-3.05 (m, 1H), 3.5-3.75 (m, 1H), 4.35-4.6 (m, 1H), 4.3-4.6 (m, 1H), 7.24-7.35 (m, 2H), 7.39-7.51 (m, 2H), 8.95 (br. s., 1H)

### Example 5A

### Dimethyl (4-bromophenyl)malonate

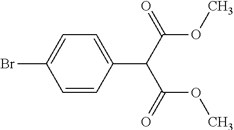 [SMILES: COC(=O)C(C(=O)OC)c1ccc(Br)cc1]

15.0 g (65.5 mmol) of methyl (4-bromophenyl)acetate were dissolved in 300 ml of THF, and sodium hydride in mineral oil (60%) was added at RT. The mixture was stirred at RT for 1 h, after which 23.6 g (262 mmol) of dimethyl carbonate were slowly added dropwise. The reaction was then stirred at RT for 3 d. 1N hydrochloric acid was then added, and the reaction mixture was concentrated. The residue was dissolved in ethyl acetate and washed successively with 1N hydrochloric acid, water and saturated sodium chloride solution. The organic phase was separated off, dried over magnesium sulphate and filtered, and the filtrate was concentrated. The residue obtained was chromatographed on silica gel (0.04-0.063 mm/230-400 mesh ASTM) using cyclohexane/ethyl acetate 4/1. After TLC, the fractions were combined and concentrated. This gave 14.6 g (77% of theory) of a solid.

LC-MS [Method 1]: Rₜ=1.04 min; MS (ESIpos): m/z=286 (M+H)⁺

### Example 6A

### Dimethyl (4-bromophenyl)(methyl)malonate

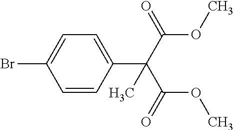 [SMILES: COC(=O)C(C)(C(=O)OC)c1ccc(Br)cc1]

7.2 g (25 mmol) of dimethyl (4-bromophenyl)malonate were dissolved in 200 ml of THF, and 1.5 g (37.6 mmol) of sodium hydride in mineral oil (60%) were added at RT. The mixture was stirred at RT for 30 minutes, after which 7.1 g (50.2 mmol) of iodomethane were added. The mixture was stirred at RT for a further 2 h. Subsequently, the reaction mixture was concentrated, and the residue was taken up in water and extracted with ethyl acetate. The organic phase was separated off, dried over magnesium sulphate and filtered, and the filtrate was concentrated. The oily residue was dried under HV. This gave 5.2 g (67% of theory) of an oil which was used further without purification.

LC-MS [Method 1]: Rₜ=1.11 min; MS (ESIpos): m/z=301 (M+H)⁺

### Example 7A

### Diethyl [4-(tert-butoxycarbonyl)benzyl](methyl)malonate

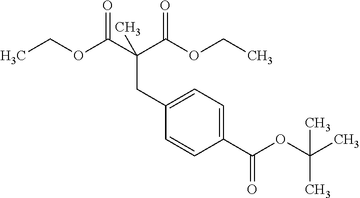 [SMILES: CCOC(=O)C(C)(Cc1ccc(C(=O)OC(C)(C)C)cc1)C(=O)OCC]

3.2 g (18.4 mmol) of diethyl methylmalonate were dissolved in 150 ml of toluene, and 0.9 g (22.1 mmol) of sodium hydride in mineral oil (60%) were added at RT. The mixture was stirred at RT for 1 h. 5.0 g (18.4 mmol) of tert-butyl 4-(bromomethyl)benzoate were then added as a solution in 50 ml of toluene. The mixture was stirred at RT for 4 h. The mixture was diluted with ethyl acetate and washed first with water and then with saturated sodium chloride solution. The organic phase was separated off, dried over magnesium sulphate and filtered, and the filtrate was concentrated. This gave 6.2 g (76% of theory, purity: 83%) of an oil which was used further without purification.

LC-MS [Method 3]: Rₜ=1.38 min; MS (EIpos): m/z=365 (M+H)⁺

### Example 8A

### 2-(4-Bromophenyl)-2-methylpropane-1,3-diol

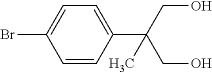

5.2 g (17.3 mmol) of dimethyl (4-bromophenyl)(methyl)malonate were dissolved in 100 ml of ethanol, and 0.98 g (26 mmol) of sodium borohydride was added at RT. The reaction mixture was stirred at RT overnight. 1N hydrochloric acid was then added, and the mixture was extracted with ethyl acetate. The organic phase was separated off, dried over magnesium sulphate and filtered, and the filtrate was concentrated. The residue obtained was chromatographed on silica gel (0.04-0.063 mm/230-400 mesh ASTM) using dichloromethane/methanol 100/1; 50/1; 10/1. After TLC, the fractions were combined and concentrated. This gave 3.26 g (77% of theory) of a solid.

¹H-NMR (400 MHz, DMSO-d₆): δ [ppm]=1.16 (s, 3H), 3.49-3.57 (m, 4H), 4.54 (t, 2H), 7.31-7.35 (m, 2H), 7.42-7.47 (m, 2H)

### Example 9A

### tert-Butyl 4-[3-hydroxy-2-(hydroxymethyl)-2-methylpropyl]benzoate

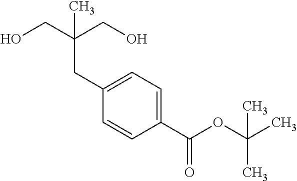 [SMILES: CC(CO)(CO)Cc1ccc(C(=O)OC(C)(C)C)cc1]

6.2 g (82.6 mmol) of diethyl [4-(tert-butoxycarbonyl)benzyl](methyl)malonate (83% pure) were dissolved in 100 ml of ethanol, and 0.97 g (25.5 mmol) of sodium borohydride was added at RT. The reaction mixture was stirred at RT overnight. 1N hydrochloric acid was then added, and the mixture was extracted with ethyl acetate. The organic phase was separated off, dried over magnesium sulphate and filtered, and the filtrate was concentrated. The residue obtained was chromatographed on silica gel (0.04-0.063 mm/230-400 mesh ASTM) using cyclohexane/ethyl acetate 1/1. After TLC, the fractions were combined and concentrated. This gave 2.5 g (55% of theory, purity: 87%) of a solid.

LC-MS [Method 3]: Rₜ=1.10 min; MS (ESIpos): m/z=225 (M-tert-butyl+2H)⁺

¹H-NMR (400 MHz, DMSO-d₆): δ [ppm]=0.63 (s, 3H), 1.54 (s, 9H), 2.57 (s, 2H), 3.10-3.19 (m, 4H), 4.48 (t, 2H), 7.26-7.31 (m, 2H), 7.76-7.81 (m, 2H)

### Example 10A

### 3-(4-Bromophenyl)-3-methyloxetane

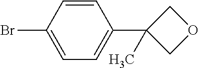

3.3 g (13.3 mmol) of 2-(4-bromophenyl)-2-methylpropane-1,3-diol were dissolved in 60 ml of toluene, and 7.0 g (26.6 mmol) of triphenylphosphine were added. After 10 minutes of stirring at RT, 6.1 g (20.0 mmol) of zink bis(dimethyldithiocarbamate) were added. 11.6 g (26.6 mmol) of diethyl azodicarboxylate (40% strength solution in toluene) were slowly added dropwise to this suspension. After initial spontaneous decolouration of the suspension from yellow to colourless, a slight yellow colouration remained at the end of the dropwise addition. The reaction mixture was stirred at RT overnight. The mixture was filtered through kieselguhr, the filter cake was washed with ethyl acetate and the filtrate was then washed with aqueous ammonia solution (about 5% strength) until no more zink bis(dimethyldithiocarbamate) could be detected in the organic phase by TLC (cyclohexane/ethyl acetate 1/1). The organic phase was separated off, dried over magnesium sulphate and filtered, and the filtrate was concentrated. The residue obtained was chromatographed on silica gel (0.04-0.063 mm/230-400 mesh ASTM) using cyclohexane to cyclohexane/ethyl acetate 2/1. After TLC, the fractions were combined and concentrated. This gave 2.1 g (70% of theory) of an oil.

GC-MS [Method 5]: Rₜ=5.01 min; MS(ESIpos): m/z=226/228 (M⁺)

¹H-NMR (400 MHz, DMSO-d₆): δ [ppm]=1.60 (s, 3H), 4.51-4.55 (m, 2H), 4.75-4.78 (m, 2H), 7.20-7.24 (m, 2H), 7.52-7.56 (m, 2H)

### Example 11A

### tert-Butyl 4-[(3-methyloxetan-3-yl)methyl]benzoate

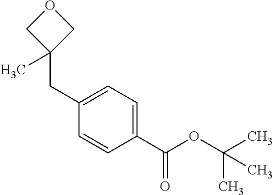 [SMILES: CC1(Cc2ccc(C(=O)OC(C)(C)C)cc2)COC1]

2.0 g (6.2 mmol) of tert-butyl 4-[3-hydroxy-2-(hydroxymethyl)-2-methylpropyl]benzoate (87% pure) were dissolved in 40 ml of toluene, and 3.7 g (14.3 mmol) of triphenylphosphine were added. After 10 minutes of stirring at RT, 3.3 g (10.7 mmol) of zink bis(dimethyldithiocarbamate) (ziram) were added. 6.2 g (14.3 mmol) of diethyl azodicarboxylate (40% strength solution in toluene) were slowly added dropwise to this suspension. After initial spontaneous decolouration of the suspension from yellow to colourless, a slight yellow colouration remained at the end of the dropwise addition. The reaction mixture was stirred at RT overnight. The mixture was filtered through kieselguhr, the filter cake was washed with ethyl acetate and the filtrate was then washed with aqueous ammonia solution (about 5% strength) until no more zink bis(dimethyldithiocarbamate) could be detected in the organic phase by TLC (cyclohexane/ethyl acetate 2/1). The organic phase was separated off, dried over magnesium sulphate and filtered, and the filtrate was concentrated. The residue obtained was chromatographed on silica gel (0.04-0.063 mm/230-400 mesh ASTM) using cyclohexane/ethyl acetate 10/1 to cyclohexane/ethyl acetate 2/1. After TLC, the fractions were combined and concentrated. This gave 1.26 g (77% of theory) of an oil.

¹H-NMR (400 MHz, DMSO-d₆): δ [ppm]=1.17 (s, 3H), 1.54 (s, 9H), 2.96 (s, 2H), 4.15-4.19 (m, 2H), 4.50-4.54 (m, 2H), 7.28-7.32 (m, 2H), 7.79-7.85 (m, 2H)

### Example 12A

### 4-[(3-Methyloxetan-3-yl)methyl]benzoic acid

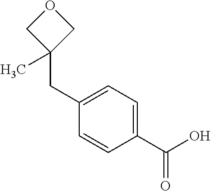

1.4 g (5.3 mmol) of tert-butyl 4-[(3-methyloxetan-3-yl)methyl]benzoate were dissolved in 20 ml of dichloromethane, and 2 ml of trifluoroacetic acid were added dropwise at RT. The mixture was stirred at RT for 5 h. The mixture was then diluted with dichloromethane and extracted first with water and then with saturated sodium chloride solution. The organic phase was separated off, dried over magnesium sulphate and filtered, and the filtrate was concentrated. This gave 1.03 g (83% of theory, purity: 89%) of a solid.

LC-MS [Method 4]: Rₜ=1.54 min; MS (ESIneg): m/z=205 (M−H)⁻

¹H-NMR (400 MHz, DMSO-d₆): δ [ppm]=1.18 (s, 3H), 2.97 (s, 2H), 4.16-4.20 (m, 2H), 4.51-4.55 (m, 2H), 7.28-7.33 (m, 2H), 7.84-7.89 (m, 2H), 12.82 (br. s, 1H)

### Example 13A

### Ethyl 2-(4-bromophenyl)-2-methylpropanoate

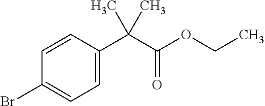

2.0 g (8.2 mmol) of ethyl (4-bromophenyl)acetate were dissolved in 50 ml of DMF, and 0.7 g (18 mmol) of sodium hydride in mineral oil (60%) were added at RT. The mixture was stirred at RT for 30 minutes, after which 2.9 g (20.6 mmol) of iodomethane were added. The mixture was then stirred at RT overnight. The mixture was diluted with ethyl acetate and extracted first with water and then with saturated sodium chloride solution. The organic phase was separated off, dried over magnesium sulphate and filtered, and the filtrate was concentrated. The residue was purified by preparative HPLC [Reprosil C18, 10 μm, 250 mm×40 mm (30% methanol/70% water to 100% methanol) over a run time of 25 min]. After HPLC control, the product-containing fractions were combined and concentrated. This gave 1.75 g (78% of theory) of an oil.

GC-MS [Method 5]: Rₜ=5.10 min; MS(ESIpos): m/z=270/272 (M⁺)

¹H-NMR (400 MHz, DMSO-d₆): δ [ppm]=1.11 (t, 3H), 1.48 (s, 6H), 4.06 (q, 2H), 7.24-7.29 (m, 2H), 7.50-7.54 (m, 2H)

### Example 14A

### Ethyl 1-(4-bromophenyl)cyclopropanecarboxylate

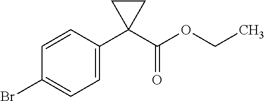

1.45 g (36.2 mmol) of sodium hydride in mineral oil (60% pure) were initially charged in 100 ml of DMF. A mixture of 4.0 g (16.5 mmol) of ethyl (4-bromophenyl)acetate and 6.5 g (34.6 mmol) of 1,2-dibromoethane was dissolved in 50 ml of THF and slowly added dropwise. The mixture was stirred at RT overnight. The reaction mixture was diluted with ethyl acetate and washed with water and saturated sodium chloride solution. The organic phase was separated off, dried over magnesium sulphate and filtered, and the filtrate was concentrated. The residue was purified by preparative HPLC [Reprosil C18, 10 μm, 250 mm×40 mm (30% methanol/70% water to 100% methanol) over a run time of 25 min]. After HPLC control, the product-containing fractions were combined and concentrated. This gave 1.15 g (26% of theory) of a liquid.

GC-MS [Method 5]: Rₜ=5.45 min; MS(ESIpos): m/z=268/270 (M⁺)

¹H-NMR (400 MHz, DMSO-d₆): δ [ppm]=1.09 (t, 3H), 1.16-1.20 (m, 2H), 1.46-1.50 (m, 2H), 4.02 (q, 2H), 7.26-7.31 (m, 2H), 7.47-7.51 (m, 2H)

### Example 15A

### 4-[(3-Methyl-1,4′-bipiperidin-1′-yl)carbonyl]benzoic acid

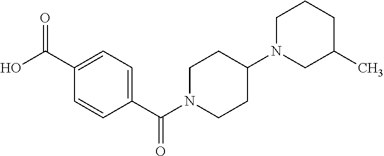 [SMILES: CC1CCCN(C2CCN(C(=O)c3ccc(C(=O)O)cc3)CC2)C1]

1.3 g (3.8 mmol) of methyl 4-[(3-methyl-1,4′-bipiperidin-1′-yl)carbonyl]benzoate were dissolved in 60 ml of dioxane, and a solution of 271.1 mg (11.3 mmol) of lithium hydroxide in 30 ml of water was added. The mixture was warmed to 50° C. and stirred for 2 h. The reaction mixture was concentrated on a rotary evaporator to a volume of 8 ml and acidified with 1N hydrochloric acid. The product was purified by preparative HPLC. [Reprosil C18, 10 μm, 250 mm×30 mm (50% methanol/50% water to 70% methanol/30% water) over a run time of 25 min]. After HPLC control, the product-containing fractions were combined and concentrated. The residue was dried under HV. This gave 482 mg (39% of theory) of a solid.

LC-MS [Method 3]: Rₜ=0.39 min; MS (ESIpos): m/z=331 (M+H)⁺

### Example 16A

### 4-(2-Methoxypropan-2-yl)benzoic acid

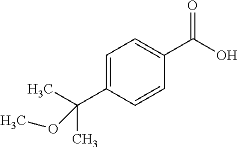

1.00 g (5.55 mmol) of 4-(2-hydroxypropan-2-yl)benzoic acid were dissolved in 40 ml of anhydrous methanol (p.a.). 1.17 g (11.10 mmol) of trimethoxymethane and 73.75 mg (0.22 mmol) of cerium (4⁺) disulphate were added, and the reaction mixture was stirred at 65° C. overnight. 1 ml of water was added to the reaction mixture, which was then filtered through an Extrelut cartridge. The cartridge was rinsed four times with in each case 5 ml of methanol. The solvent was then concentrated on a rotary evaporator and the residue was dried under HV. This gave 1.12 g (86% of theory, purity: 83%) of a crystalline material. This product was reacted further without further purification.

LC-MS [Method 3]: Rₜ=0.89 min; MS (ESIpos): m/z=195 (M+H)⁺

¹H-NMR (400 MHz, DMSO-d₆): δ [ppm]=1.46 (s, 6H), 3.00 (s, 3H), 7.49-7.51 (m, 2H), 7.89-7.92 (m, 2H), 12.88 (br. s, 1H)

### Example 17A

### Methyl 2-(4-bromo-3-fluorophenyl)-2-methylpropanoate

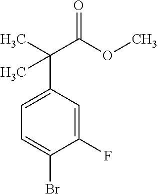

Under argon and at RT, 186.2 mg (4.66 mmol) of sodium hydride (60% in mineral oil) were added to 500 mg (2.02 mmol) of methyl (4-bromo-3-fluorophenyl)acetate. After stirring at 60° C. for one hour, 631.94 mg (4.45 mmol) of iodomethane were slowly added dropwise. The mixture was then stirred at 60° C. for 2 h. The reaction mixture was concentrated and diluted with ethyl acetate and washed with water and saturated sodium chloride solution. The organic phase was separated off, dried over sodium sulphate and filtered, and the filtrate was concentrated. This gave 633 mg (69% of theory, purity: 69%) of an oil, which was reacted further without further purification.

GC-MS [Methode 5]: Rₜ=4.92 min; MS (ESIneg): m/z=274 (M−H)⁻

### Example 18A

### 1-[1-(4-Bromophenyl)cyclobutyl]-N-methylmethanamine

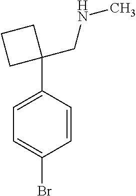

At 0° C. and under argon, 1.00 g (3.7 mmol) of N-{[1-(4-bromophenyl)cyclobutyl]methyl}-N-methylformamide [obtainable in one step from commercially available 1-(4-bromophenylcyclobutanmethanamine by reaction with formic acid in boiling o-xylene with removal of water] were added a little at a time to 1.98 g (26 mmol, 13 ml) of borane/dimethyl sulphide complex in THF (2 M). After stirring at RT overnight, 2 ml of concentrated hydrochloric acid were very slowly added a little at a time with ice cooling. After the exothermic evolution of gas had ceased, the mixture was diluted with water, made basic with aqueous sodium hydroxide solution and extracted twice with ethyl acetate.

The combined organic phases were washed with saturated aqueous sodium chloride solution and then dried over sodium sulphate. The oil obtained after concentration was triturated with a little n-pentane. The pentane phase was concentrated and dried under HV. The oil obtained in this manner (620 mg) was converted into Example 19A without further purification.

LC-MS [Method 1]: Rₜ=0.70 min; MS (ESIpos): m/z=254 (M+H)⁺

### Example 19A

### N-{[1-(4-Bromophenyl)cyclobutyl]methyl}-N-methylmethanesulphonamide

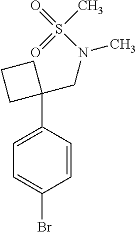 [SMILES: CN(CC1(c2ccc(Br)cc2)CCC1)S(C)(=O)=O]

Under argon, 75 mg (0.9 mmol) of pyridine were added to 200 mg (0.79 mmol) of 1-[1-(4-bromophenyl)cyclobutyl]-N-methylmethanamine in 5 ml of dichloromethane, and 108 mg (0.9 mmol) of methanesulphonyl chloride were then added. The mixture was stirred at RT overnight. After dilution with dichloromethane and water, the organic phase was separated off and washed with saturated aqueous sodium chloride solution. The organic phase was dried over sodium sulphate and concentrated. The resulting solid was triturated with a little isopropanol, filtered off with suction and air-dried. This gave 61 mg (23% of theory) of the target compound as a solid.

LC-MS [Method 8]: Rₜ=2.42 min; MS (ESIpos): m/z=332 (M+H)⁺

### Example 20A

### tert-Butyl 3-(hydrazinocarbonyl)-1,4′-bipiperidine-1′-carboxylate

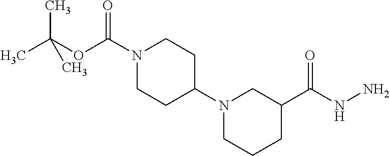 [SMILES: CC(C)(C)OC(=O)N1CCC(N2CCCC(C(=O)NN)C2)CC1]

With addition of 2 ml of ethanol, 2.00 g (5.9 mmol) of 1′-tert-butyl 3-ethyl 1,4′-bipiperidine-1′,3-dicarboxylate (described in US publication No. US 2006/0223792, Butler et al.) and 21 ml (235 mmol) of hydrazine hydrate in water (55%) were heated at reflux overnight. The mixture was concentrated to dryness and the residue was purified by flash chromatography on silica gel (elution: ethyl acetate/methanol: 1/1). The product-containing fractions were concentrated and dried under HV. This gave 0.95 g (99% of theory) of the target compound.

¹H-NMR (400 MHz, DMSO-d₆): δ [ppm]=1.15-1.25 (m, 2H), 1.3-1.4 (m, 11H, including: 1.35, s, about 9H), 1.45-1.7 (m, 4H), 2.05-2.1 (m, 1H), 2.15-2.3 (m, 2H), 2.3-2.4 (m, 1H), 2.6-2.8 (m, 4H), 3.85-3.95 (m, 2H), 4.1 (bs, 2H), 8.95 (bs, 1H).

### Example 21A

### tert-Butyl 3-(3-cyclopropyl-1H-1,2,4-triazol-5-yl)-1,4′-bipiperidine-1′-carboxylate

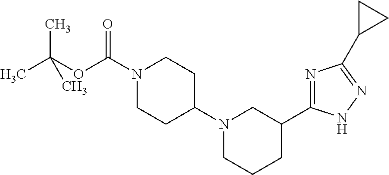 [SMILES: CC(C)(C)OC(=O)N1CCC(N2CCCC(c3nc(C4CC4)n[nH]3)C2)CC1]

Under argon, 74 mg (1.8 mmol) of sodium hydride in paraffin oil (60%) were added to 111 mg (0.92 mmol) of cyclopropanecarboximidamidine hydrochloride in 2 ml of methanol, and the mixture was stirred at RT for 1 h. The mixture was filtered, the filter residue was washed with 1 ml of methanol and the combined filtrate was added to a solution of 200 mg (0.61 mmol) of tert-butyl 3-(hydrazinocarbonyl)-1,4′-bipiperidine-1′-carboxylate in 2 ml of methanol. The solution was heated in the microwave at 140° C. for 2 h. The reaction mixture was concentrated and purified by flash chromatography on silica gel (elution: ethyl acetate/methanol gradient: 5:1 to 3:1). The product-containing fractions were concentrated and dried under HV. This gave 111 mg (48% of theory) of a solid.

¹H-NMR (400 MHz, DMSO-d₆): δ [ppm]=0.70-1.0 (m, 4H), 1.2-1.3 (m, 2H), 1.35 (s, 9H), 1.4-1.5 (m, 2H), 1.6-1.7 (m, 3H), 1.8-1.9 (m, 2H), 2.1-2.45 (m, 3H), 2.6-2.8 (m, 4H), 2.9-3.0 (m, 1H), 3.9-4.0 (m, 2H), about 13 (very broad s, 1H) The following were prepared in the same manner:

### Example 22A

### tert-Butyl 3-(3-cyclobutyl-1H-1,2,4-triazol-5-yl)-1,4′-bipiperidine-1′-carboxylate

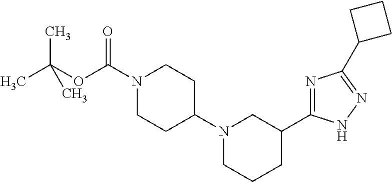 [SMILES: CC(C)(C)OC(=O)N1CCC(N2CCCC(c3nc(C4CCC4)n[nH]3)C2)CC1]

from cyclobutanecarboximidamide hydrochloride; an oil was formed, yield: 84% of theory.

DCI-MS (NH₃): m/z=390 (M+H)⁺

### Example 23A

### tert-Butyl 3-(3-ethyl-1H-1,2,4-triazol-5-yl)-1,4′-bipiperidine-1′-carboxylate

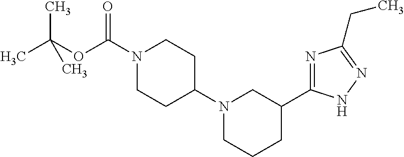 [SMILES: CCc1n[nH]c(C2CCCN(C3CCN(C(=O)OC(C)(C)C)CC3)C2)n1]

from propaneimidamide hydrochloride; an oil was formed, yield: 67% of theory.

DCI-MS (NH₃): m/z=364 (M+H)⁺

### Example 24A

### tert-Butyl 3-(3-methyl-1H-1,2,4-triazol-5-yl)-1,4′-bipiperidine-1′-carboxylate

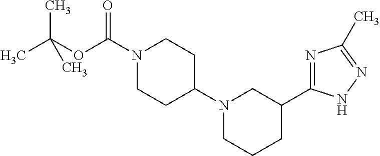 [SMILES: Cc1n[nH]c(C2CCCN(C3CCN(C(=O)OC(C)(C)C)CC3)C2)n1]

from ethaneimidamide acetate; a foam was formed, yield: 82% of theory.

DCI-MS (NHC): m/z=350 (M+H)⁺

### Example 25A

### tert-Butyl 3-(1H-1,2,4-triazol-5-yl)-1,4′-bipiperidine-1′-carboxylate

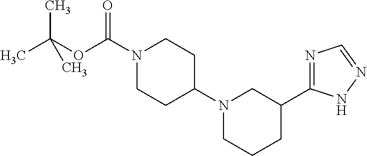 [SMILES: CC(C)(C)OC(=O)N1CCC(N2CCCC(c3ncn[nH]3)C2)CC1]

from formamidine acetate; an oil was formed, yield: 72% of theory.

LC-MS [Method 4]: Rₜ=1.10 min; MS (ESIpos): m/z=336 (M+H)⁺

### Example 26A

### tert-Butyl 3-(3-cyclobutylmethyl-1H-1,2,4-triazol-5-yl)-1,4′-bipiperidine-1′-carboxylate

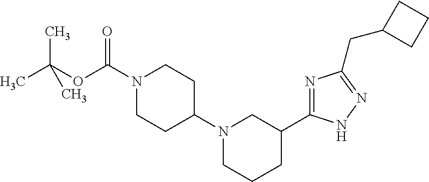 [SMILES: CC(C)(C)OC(=O)N1CCC(N2CCCC(c3nc(CC4CCC4)n[nH]3)C2)CC1]

from 2-cyclobutylethaneimidamide hydrochloride; a solid was formed, yield: 44% of theory.

DCI-MS (NH₃): m/z=404 (M+H)⁺

### Example 27A

### 3-(5-Cyclopropyl-4H-1,2,4-triazol-3-yl)-4′-bipiperidine

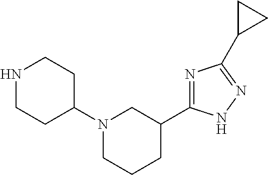 [SMILES: C1CC(c2nc(C3CC3)n[nH]2)CN(C2CCNCC2)C1]

105 mg (0.28 mmol) of tert-butyl 3-(3-cyclopropyl-1H-1,2,4-triazol-5-yl)-1,4′-bipiperidine-1′-carboxylate were dissolved in dichloromethane, and 1 ml of trifluoroacetic acid was added with ice cooling. The mixture was stirred at RT for 2 h. The reaction mixture was neutralized with sodium bicarbonate solution and the resulting two-phase mixture was concentrated. The residue was stirred with methanol, and insolubles were then filtered off. The filtrate was concentrated, the residue was chromatographed on silica gel (0.04-0.063 mm/230-400 mesh ASTM) (methanol/25% strength ammonia solution 20/1). After TLC check (methanol/25% strength ammonia solution 20/1, staining with potassium permanganate solution), the product-containing fractions were combined and concentrated. The residue was dried under HV. This gave 53 mg (69% of theory) of a solid.

¹H-NMR (400 MHz, DMSO-d₆): δ [ppm]=0.70-1.0 (m, 4H), 1.2-1.3 (m, 2H), 1.3-1.5 (m, 2H), 1.6-1.7 (m, 3H), 1.8-1.9 (m, 2H), 2.1-2.45 (m, 6H), 2.65-2.8 (m, 2H), 2.85-3.0 (m, 3H), about 13.2 (br. s, 1H)

The following were prepared in the same manner:

### Example 28A

### 3-(5-Cyclobutyl-4H-1,2,4-triazol-3-yl)-1,4′-bipiperidine

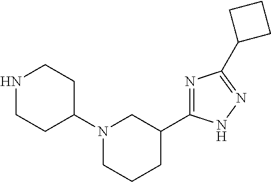 [SMILES: C1CC(c2n[nH]c(C3CCCN(C4CCNCC4)C3)n2)C1]

from tert-butyl 3-(3-cyclobutyl-1H-1,2,4-triazol-5-yl)-1,4′-bipiperidine-1′-carboxylate; a solid was formed, yield: 69% of theory.

DCI-MS (NH₃): m/z=290 (M+H)⁺

### Example 29A

### 3-(3-Ethyl-1H-1,2,4-triazol-5-yl)-1,4′-bipiperidine

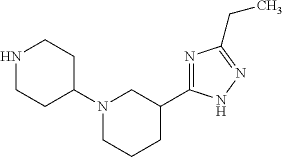 [SMILES: CCc1n[nH]c(C2CCCN(C3CCNCC3)C2)n1]

from tert-butyl 3-(3-ethyl-1H-1,2,4-triazol-5-yl)-1,4′-bipiperidine-1′-carboxylate; a solid was formed, yield: 80% of theory.

DCI-MS (NH₃): m/z=264 (M+H)⁺

### Example 30A

### 3-(3-Methyl-1H-1,2,4-triazol-5-yl)-1,4′-bipiperidine

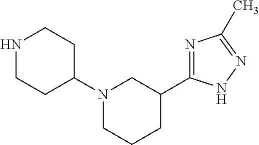 [SMILES: Cc1n[nH]c(C2CCCN(C3CCNCC3)C2)n1]

from tert-butyl 3-(3-methyl-1H-1,2,4-triazol-5-yl)-1,4′-bipiperidine-1′-carboxylate; a foam was formed, yield: 81% of theory.

DCI-MS (NH₃): m/z=250 (M+H)⁺

### Example 31A

### 3-(1H-1,2,4-Triazol-5-yl)-1,4′-bipiperidine

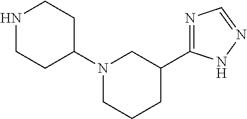 [SMILES: c1n[nH]c(C2CCCN(C3CCNCC3)C2)n1]

from tert-butyl 3-(1H-1,2,4-triazol-5-yl)-1,4′-bipiperidine-1′-carboxylate; a foam was formed, yield: 56% of theory.

DCI-MS (NH₃): m/z=236 (M+H)⁺

### Example 32A

### 3-[5-(Cyclobutylmethyl)-4H-1,2,4-triazol-3-yl]-1,4′-bipiperidine

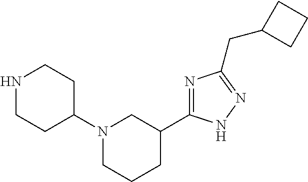 [SMILES: C1CC(Cc2n[nH]c(C3CCCN(C4CCNCC4)C3)n2)C1]

from tert-butyl 3-[5-(cyclobutylmethyl)-4H-1,2,4-triazol-3-yl]-1,4′-bipiperidine-1′-carboxylate; a solid was formed, yield: 81% of theory.

DCI-MS (NH₃): m/z=304 (M+H)⁺

### Example 33A

### 2-(4-Bromo-3-fluorophenyl)-N-tert-butyl-2-methylpropanamide

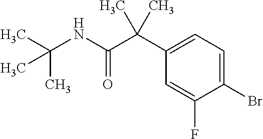 [SMILES: CC(C)(C)NC(=O)C(C)(C)c1ccc(Br)c(F)c1]

Under argon, 87 mg (0.43 mol) of EDC, 66 mg (0.43 mmol) of HOBT and 0.19 ml (1.08 mmol) of N,N-diisopropylethylamine were added to 174 mg (content 54%; 0.36 mmol) of 2-(4-bromo-3-fluorophenyl)-2-methylpropanoic acid in 3 ml of DMF. After stirring at RT for 10 minutes, 32 mg (0.43 mmol) of tert-butylamine were added. The mixture was stirred at RT overnight. After dilution with water, the mixture was extracted with ethyl acetate. The organic phase was washed with saturated aqueous sodium chloride solution and dried over sodium sulphate. The crude product obtained in this manner, having a content of 46.5% (GC/MS, Method 5), was reacted further without further purification.

DCI-MS (NH₃): m/z=316 (M+H)⁺

### Example 34A

### tert-Butyl {[1-(4-bromophenyl)cyclobutyl]methyl}methylcarbamate

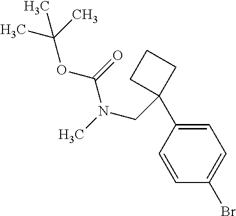 [SMILES: CN(CC1(c2ccc(Br)cc2)CCC1)C(=O)OC(C)(C)C]

639 mg (2.93 mmol) of di-tert-butyl dicarbonate were added to 372 mg (1.46 mmol) of 1-[1-(4-bromophenyl)cyclobutyl]-N-methylmethanamine and 178 mg (1.46 mmol) of DMAP in 10 ml of dichloromethane, and the mixture was stirred at RT overnight. The mixture was then heated at 60° C. for 2 h. After cooling to RT, water was added and the organic phase was washed with saturated aqueous sodium chloride solution. The organic phase was dried over sodium sulphate and concentrated. The solid obtained was stirred with diethyl ether. The filtrate was, after concentration, taken up in ethyl acetate, washed repeatedly with water and dried over sodium sulphate. After concentration, 245 mg of an oil remained which, according to analysis, still contained about 15% DMAP. It was reacted further as such.

¹H-NMR (400 MHz, DMSO-d₆): δ [ppm]=1.2 and 1.3 (2 br. s, together about 9H), 1.65-1.8 (m, 1H, 2.0 (m, 1H), 2.15-2.3 (m, 5H), 3.3 and 3.5 (2 s, together 2H), 7.1 (m, 2H), 7.5 (m, 2H) and DMAP signals

### Example 35A

### N-{[1-(4-Bromophenyl)cyclobutyl]methyl}-N-methylformamide

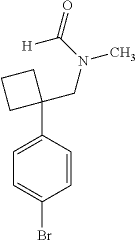 [SMILES: CN(C=O)CC1(c2ccc(Br)cc2)CCC1]

Under argon, 36 mg (0.90 mmol) of sodium hydride in paraffin oil (60%) were added to 0.20 g (0.75 mmol) of N-{[1-(4-bromophenyl)cyclobutyl]methyl}-N-methylformamide [obtainable in one step from commercially available 1-(4-bromophenylcyclobutanmethanamine by reaction with formic acid in boiling o-xylene with removal of water] in 10 ml THF. After 1 h of stirring at RT, 116 mg (0.82 mmol) of iodomethane were added. After stirring overnight at RT, another 40 mg of iodomethane were added. The mixture was once more stirred at RT overnight. The reaction mixture was concentrated, and saturated ammonium chloride solution and ethyl acetate were added. The organic phase was washed with saturated aqueous sodium chloride solution, dried over sodium sulphate and concentrated. This gave 126 mg (60% of theory) of an oil.

GC-MS [Methode 5]: Rₜ=7.26 min; MS (ESIpos): m/z=281/283 (M⁺)

### Example 36A

### Methyl 4-[(4-oxopiperidin-1-yl)carbonyl]benzoate

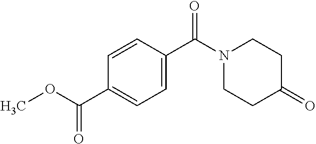 [SMILES: COC(=O)c1ccc(C(=O)N2CCC(=O)CC2)cc1]

At 0° C., 11.7 ml (20.0 mmol) of T3P (50% by weight strength solution in DMF) were added to a solution of 3.00 g (16.7 mmol) of monomethyl terephthalate, 2.48 g (18.3 mmol) of 4-piperidinone hydrochloride and 7.3 ml (42 mmol) of N,N-diisopropylethylamine in 175 ml of acetonitrile, and the mixture was then stirred at RT overnight. For work-up, the volatile constituents were removed under reduced pressure and the mixture was adjusted with aqueous ammonia to pH 8-9 and then extracted repeatedly with ethyl acetate. The combined organic phases were washed with saturated sodium bicarbonate solution and saturated sodium chloride solution, dried over magnesium sulphate, filtered and concentrated. The crude product obtained (3.59 g, 83% of theory) was reacted without further purification.

LC-MS [Method 1]: Rₜ=0.61 min; MS (ESIpos): m/z=262 (M+H)⁺

### Example 37A

### 4-[(4-Oxopiperidin-1-yl)carbonyl]benzoic acid

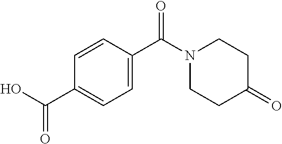 [SMILES: O=C1CCN(C(=O)c2ccc(C(=O)O)cc2)CC1]

A solution of 1.50 g (5.74 mmol) of the compound from Example 36A was stirred in a mixture of 29 ml of 1 N lithium hydroxide solution, 50 ml of THF and 10 ml of methanol for 2 h at 40° C. The mixture was then acidified to pH 3 using 6 N hydrochloric acid and substantially concentrated. The residue was extracted repeatedly with dichloromethane. The combined organic phases were washed once with saturated sodium chloride solution, dried over magnesium sulphate, filtered and concentrated. The crude product obtained (480 mg, 33% of theory) was directly reacted further.

LC-MS [Method 4]: Rₜ=1.06 min; MS (ESIpos): m/z=248 (M+H)⁺

### Example 38A

### Present as a Mixture with the Hydrate

### N-[(3,5-Difluoropyridin-2-yl)methyl]-4-[(4-oxopiperidin-1-yl)carbonyl]benzamide

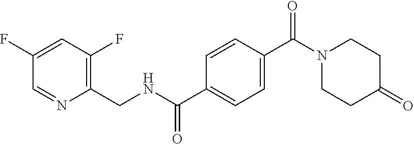 [SMILES: O=C1CCN(C(=O)c2ccc(C(=O)NCc3ncc(F)cc3F)cc2)CC1]

200 mg (0.526 mmol) of HATU were added to a solution of 100 mg (0.404 mmol) of the compound from Example 37A, 87.6 mg (0.485 mmol) of 1-(3,5-difluoropyridin-2-yl)methanamine hydrochloride and 0.35 ml (2.0 mmol) of N,N-diisopropylethylamine in 4.0 ml of DMF, and the mixture was stirred at RT overnight. For work-up, water was added and the mixture was extracted repeatedly with ethyl acetate. The combined organic phases were washed with saturated sodium bicarbonate solution and saturated sodium chloride solution, dried over magnesium sulphate, filtered and concentrated. The crude product was purified by column chromatography (25 g silica gel cartridge, cyclohexane/ethyl acetate gradient). This gave 94 mg (61% of theory) of the title compound.

LC-MS [Method 2]: Rₜ=0.65 min; MS (ESIpos): m/z=374 (M+H)⁺

¹H-NMR (400 MHz, DMSO-d₆): δ [ppm]=2.35-2.55 (m, 2H), 3.50-3.70 (m, 2H), 3.85-4.00 (m, 2H), 4.67 (d, 2H), 7.59 (d, 2H), 7.90-8.00 (m, 3H), 8.49 (m, 1H), 9.10-9.18 (m, 1H).

### Example 39A

### Present as a Mixture with the Hydrate

### N-(2,6-Difluorobenzyl)-N-methyl-4-[(4-oxopiperidin-1-yl)carbonyl]benzamide

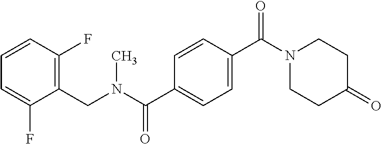 [SMILES: CN(Cc1c(F)cccc1F)C(=O)c1ccc(C(=O)N2CCC(=O)CC2)cc1]

280 mg (0.736 mmol) of HATU were added to a solution of 280 mg (0.566 mmol) of the compound from Example 37A, 107 mg (0.679 mmol) of 1-(2,6-difluorophenyl)-N-methylmethanamine and 0.49 ml (2.8 mmol) of N,N-diisopropylethylamine in 6.0 ml of DMF, and the mixture was stirred at RT overnight. For work-up, water was added and the mixture was extracted repeatedly with ethyl acetate. The combined organic phases were washed with saturated sodium bicarbonate solution and saturated sodium chloride solution, dried over magnesium sulphate, filtered and concentrated. The crude product was purified by column chromatography (25 g silica gel cartridge, ethyl acetate/methanol gradient) and by HPLC [Method 12a]. This gave 190 mg (86% of theory) of the title compound.

LC-MS [Method 2]: Rₜ=0.80 min; MS (ESIpos): m/z=387 (M+H)⁺

¹H-NMR (400 MHz, DMSO-d₆): δ [ppm]=2.30-2.65 (m, 4H), 3.50-4.00 (m, 4H), 4.5-4.90 (m, 4H), 7.00-7.25 (m, 2H); 7.35-7.62 (m, 5H).

### Example 40A

### 2-(4-Bromo-2-chlorophenyl)propan-2-ol

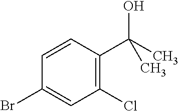

At 0° C., 4.0 ml of a 3 M methylmagnesium chloride solution (12 mmol) were added dropwise to a solution of 1.00 g (4.00 mmol) of methyl 4-bromo-2-chlorobenzoate in 37 ml of THF. The reaction mixture was slowly warmed to RT and stirred overnight. For work-up, saturated sodium chloride solution was added, the mixture was diluted with ethyl acetate and the phases were separated. The aqueous phase was extracted with ethyl acetate, and the combined organic phases were dried over magnesium sulphate, filtered and concentrated. The crude product obtained (quantitative) was directly reacted further.

GC-MS [Method 5]: Rₜ=4.67 min; MS (EI+): m/z=230 (M−H₂O)⁺

¹H-NMR (400 MHz, DMSO-d₆): δ [ppm]=1.56 (s, 6H), 5.41 (s, 1H), 7.54 (dd, 1H), 7.61 (d, 1H), 7.76 (d, 2H).

### Example 41A

### 4-Bromo-N-tert-butyl-2-chlorobenzamide

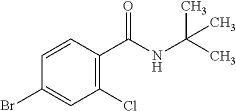

At 0° C., 1.49 g (2.34 mmol) of T3P (50% by weight solution in ethyl acetate) were added to a solution of 500 mg (2.12 mmol) of 4-bromo-2-chlorobenzoic acid, 186 mg (2.55 mmol) of tert-butylamine and 1.3 ml (7.4 mmol) of N,N-diisopropylethylamine in 7.0 ml of acetonitrile, and the mixture was then stirred at RT overnight. For work-up, the volatile constituents were removed under reduced pressure and the residue was taken up in ethyl acetate. The organic phase was washed three times with saturated sodium bicarbonate solution and saturated sodium chloride solution, dried over magnesium sulphate, filtered and concentrated. This gave 384 mg of the title compound (62% of theory) which were directly reacted further.

LC-MS [Method 2]: Rₜ=1.05 min; MS (ESI+): m/z=290 (M+H)⁺

¹H-NMR (400 MHz, DMSO-d₆): δ [ppm]=1.34 (s, 9H), 7.31 (d, 1H), 7.57 (dd, 1H), 7.76 (d, 1H), 8.03-8.12 (m, 1H).

### Example 42A

### 4-Bromo-N-tert-butyl-3-chlorobenzamide

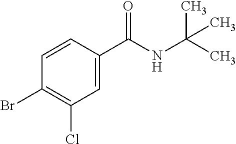

Prepared analogously to Example 41A from 1.00 g (4.25 mmol) of 4-bromo-N-tert-butyl-3-chlorobenzoic acid. This gave 1.05 g of the title compound (95% of theory).

LC-MS [Method 9]: Rₜ=1.15 min; MS (ESI+): m/z=290 (M+H)⁺

¹H-NMR (400 MHz, DMSO-d₆): δ [ppm]=1.37 (s, 9H), 7.69 (dd, 1H), 7.85 (d, 1H), 7.98 (br. s, 1H), 8.03 (d, 1H).

### Example 43A

### 4-(tert-Butylcarbamoyl)-2-chlorobenzoic acid

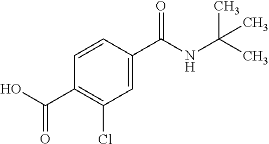 [SMILES: CC(C)(C)NC(=O)c1ccc(C(=O)O)c(Cl)c1]

At −78° C., 1.2 ml (1.9 mmol) of 1.6 M methyllithium solution in diethyl ether were added dropwise to a solution of 500 mg (1.72 mmol) of 4-bromo-N-tert-butyl-3-chlorobenzamide in 17 ml of THF. After 15 min, 2.3 ml (3.6 mmol) of 1.6 M tert-butyllithium solution in pentane were added dropwise. After 10 min, the reaction was quenched by addition of dry ice. The reaction mixture was warmed to 0° C., water was added and the mixture was then extracted three times with ethyl acetate. The combined organic phases were dried over magnesium sulphate, filtered and concentrated. The residue was triturated with n-pentane und the white solid obtained was filtered off and dried under HV. The crude product (478 mg, 93% of theory) was reacted without further purification.

LC-MS [Method 1]: Rₜ=0.72 min; MS (ESI+): m/z=256 (M+H)⁺

¹H-NMR (400 MHz, DMSO-d₆): δ [ppm]=1.38 (s, 9H), 7.77-7.83 (m, 2H), 7.92 (s, 1H), 8.03 (s, 1H).

### Example 44A

### 1-[4-(2-Hydroxypropan-2-yl)benzoyl]piperidin-4-one

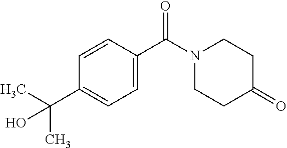 [SMILES: CC(C)(O)c1ccc(C(=O)N2CCC(=O)CC2)cc1]

3.00 g (16.6 mmol) of 4-(1-hydroxy-1-methylethyl)benzoic acid, 2.48 g (18.3 mmol) of piperidin-4-one hydrochloride hydrate and 6.38 ml of N,N-diisopropylethylamine were dissolved in 80 ml of acetonitrile, and 10.7 ml (18.3 mmol) of T3P (50% by weight strength solution in ethyl acetate) were added at 0° C. The mixture was stirred at RT for 18 h. For work-up, the reaction mixture was concentrated, 25 ml of water were added and the mixture was extracted four times with in each case 25 ml of ethyl acetate. The combined organic phases were washed successively with 20 ml of saturated sodium bicarbonate solution and with 20 ml of saturated sodium chloride solution. The mixture was dried over sodium sulphate, filtered and concentrated under reduced pressure. Drying under HV gave 2.43 g (56% of theory) of the title compound.

LC-MS [Method 2]: Rₜ=0.58 min; MS (ESI+): m/z=262 (M+H)⁺, 280 (M+H+H₂O)⁺

¹H-NMR (400 MHz, DMSO-d₆): δ [ppm]=1.43 (s, 6H), 2.34-2.48 (m, 4H), 3.50-4.0 (m, 4H), 5.11 (s, 1H), 7.42 (d, 2H), 7.54 (d, 2H).

### Example 45A

### Methyl 4-[(3,5-dimethyl-1,2-oxazol-4-yl)carbamoyl]benzoate

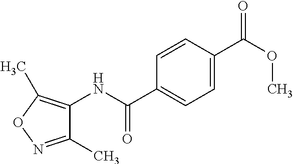 [SMILES: COC(=O)c1ccc(C(=O)Nc2c(C)noc2C)cc1]

300 mg (1.67 mmol) of monomethyl terephthalate were dissolved in 3 ml of DMF, and 377 mg (1.97 mol) of EDC and 266 mg (1.97 mmol) of HOBT were added. The mixture was left to stir at RT for 20 min and then 170 mg (1.51 mmol) of 4-amino-3,5-dimethylisoxazole were added. The mixture was stirred at RT overnight. After dilution with water, the mixture was extracted three times with in each case 10 ml of ethyl acetate. The combined organic phases were dried over sodium sulphate, filtered and concentrated under reduced pressure. The crude product was purified chromatographically [Method 16]. This gave 342 mg (75% of theory) of the target compound.

LC-MS [Method 8]: Rₜ=0.88 min; MS (ESI+): m/z=275 (M+H)⁺.

### Example 46A

### 4-[(3,5-Dimethyl-1,2-oxazol-4-yl)carbamoyl]benzoic acid

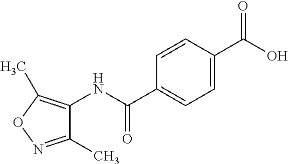 [SMILES: Cc1noc(C)c1NC(=O)c1ccc(C(=O)O)cc1]

341 mg (1.24 mmol) of the compound from Example 45A were dissolved in 3 ml of methanol, and 2.80 ml (2.80 mol) of a 1 molar solution of lithium hydroxide in water were added. The crude mixture was left to stir at RT for 2 h and concentrated under reduced pressure. 5 ml of water were added to the residue and the mixture was extracted with 10 ml of ethyl acetate. The aqueous phase was neutralized with 2.8 ml (2.8 mmol) of a 1 molar hydrochloric acid solution and extracted three times with in each case 10 ml of ethyl acetate. The combined organic phases were dried over sodium sulphate, filtered and concentrated under reduced pressure. This gave 318 mg (97% of theory) of the target compound.

LC-MS [Method 1]: Rₜ=0.76 min; MS (ESI+): m/z=261 (M+H)⁺.

### Example 47A

### N-(3,5-Dimethyl-1,2-oxazol-4-yl)-4-[(4-oxopiperidin-1-yl)carbonyl]benzamide

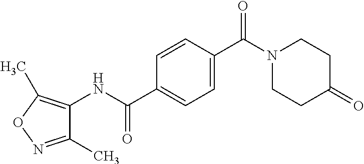 [SMILES: Cc1noc(C)c1NC(=O)c1ccc(C(=O)N2CCC(=O)CC2)cc1]

250 mg (0.96 mmol) of the compound from Example 46A were dissolved in 5 ml of DMF, and 203 mg (1.06 mol) of EDC and 162 mg (1.06 mmol) of HOBT were added. The mixture was left to stir at RT for 20 min and then 256 mg (0.96 mmol) of piperidin-4-one hydrochloride hydrate and 0.54 ml (3.84 mmol) of triethylamine were added. The mixture was stirred at RT overnight. After dilution with water, the mixture was extracted three times with in each case 10 ml of ethyl acetate. The combined organic phases were dried over sodium sulphate, filtered and concentrated under reduced pressure. The crude product was purified chromatographically [Method 16]. This gave 160 mg (49% of theory) of the target compound.

LC-MS [Method 2]: Rₜ=0.58 min; MS (ESI+): m/z=342 (M+H)⁺.

¹H-NMR (400 MHz, DMSO-d₆): δ [ppm]=2.14 (s, 3H), 2.31 (s, 3H), 2.36-2.47 (m, 2H), 2.47-2.57 (m, 2H under DMSO signal), 3.53-3.64 (m, 2H), 3.84-3.96 (m, 2H), 7.64 (d, 2H), 8.05 (d, 2H), 9.90 (s, 1H).

### Example 48A

### tert-Butyl 3-(tert-butoxymethyl)piperidine-1-carboxylate

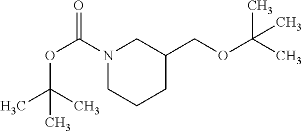 [SMILES: CC(C)(C)OCC1CCCN(C(=O)OC(C)(C)C)C1]

1.00 g (4.65 mmol) of tert-butyl 3-(hydroxymethyl)piperidine-1-carboxylate was dissolved in 10 ml of dichloroethane, and 104 mg (0.46 mmol) of magnesium perchlorate and 2.33 g (10.7 mmol) of di-tert-butyl dicarbonate were added. The mixture was stirred at 50° C. for 1 h, and a further 2.33 g (10.7 mmol) of di-tert-butyl dicarbonate were then added. The mixture was stirred at 50° C. for 6 h and left to stand at RT for 18 h. For work-up, 10 ml of water were added and the mixture was extracted twice with in each case 20 ml of dichloromethane. The organic phase was washed once with 10 ml of saturated sodium chloride solution, dried over sodium sulphate, filtered and concentrated under reduced pressure. The crude product was reacted further without further purification. This gave 1.23 g (68% of theory) in a purity of 70%.

LC-MS [Method 1]: Rₜ=1.27 min; MS (ESI+): m/z=272 (M+H)⁺.

### Example 49A

### 3-(tert-Butoxymethyl)piperidine hydrochloride

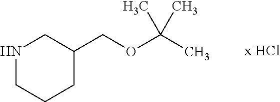

1.23 g (4.53 mmol) of the compound from Example 48A were dissolved in 5 ml of dichloromethane, and 12.1 ml (48.6 mmol) of a 4N solution of hydrogen chloride in dioxane were added. The mixture was stirred at RT for 1 h and then concentrated to dryness and dried under HV. The crude product was reacted further without further purification. This gave 0.79 g (59% of theory) in a purity of 70%.

LC-MS [Method 4]: Rₜ=0.89 min; MS (ESI+): m/z=172 (M+H)⁺.

### Example 50A

### tert-Butyl 3-[(3-fluorophenoxy)methyl]piperidine-1-carboxylate

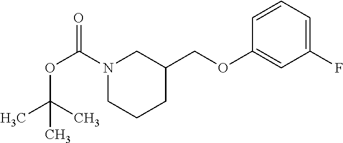 [SMILES: CC(C)(C)OC(=O)N1CCCC(COc2cccc(F)c2)C1]

200 mg (0.93 mmol) of tert-butyl 3-(hydroxymethyl)piperidine-1-carboxylate were dissolved in 8 ml of THF, and 104 mg (0.93 mmol) of 3-fluorophenol and 268 mg (1.02 mmol) of triphenylphosphine were added. At 0° C., 203 μl (1.02 mmol) of diisopropyl azodicarboxylate were added and the mixture was stirred at 0° C. for about 10 min. The mixture was then stirred at RT for 18 h. For work-up, 10 ml of water were added and the mixture was extracted twice with in each case 15 ml of ethyl acetate. The organic phase was dried over sodium sulphate, filtered and concentrated under reduced pressure. The crude product was purified chromatographically [Method 16]. This gave 220 mg (77% of theory) of the target compound.

LC-MS [Method 8]: Rₜ=1.50 min; MS (ESI+): m/z=310 (M+H)⁺.

### Example 51A

### 3-[(3-Fluorophenoxy)methyl]piperidine hydrochloride

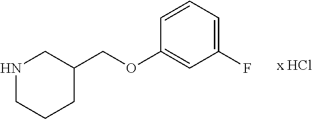 [SMILES: Fc1cccc(OCC2CCCNC2)c1]

220 mg (0.71 mmol) of the compound from Example 50A were reacted analogously to the compound from Example 49A. This gave 155 mg (89% of theory) of the target compound.

LC-MS [Method 1]: Rₜ=0.54 min; MS (ESI+): m/z=210 (M+H)⁺.

### Example 52A

### 1′-(4-tert-Butylbenzoyl)-1,4′-bipiperidine-3-carboxylic acid

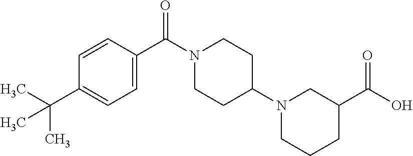 [SMILES: CC(C)(C)c1ccc(C(=O)N2CCC(N3CCCC(C(=O)O)C3)CC2)cc1]

80 ml of semiconcentrated hydrochloric acid were added to 2.00 g (5.00 mmol) of the compound from Example 1, and the mixture was stirred at RT overnight. The mixture was concentrated under reduced pressure, and twice in each case 10 ml of acetonitrile were added and the mixture was concentrated again. The crude mixture was taken up in 10 ml of dichloromethane and the solution was dried over sodium sulphate, filtered and concentrated under reduced pressure. Drying under HV gave 1.20 g (57% of theory) of the target compound.

LC-MS [Method 1]: Rₜ=0.72 min; MS (ESIpos): m/z=373 (M+H)⁺

### Example 53A

### 4-Bromo-N-tert-butyl-3-fluorobenzamide

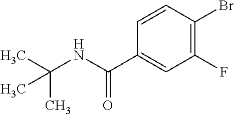

200 mg (0.91 mmol) of 4-bromo-3-fluorobenzoic acid were dissolved in 6 ml of DMF, and 175 mg (0.91 mol) of EDC and 140 mg (0.91 mmol) of HOBT were added. The mixture was left to stir at RT for 10 min and then 73 mg (1.00 mmol) of tert-butylamine and 0.48 ml (2.74 mmol) of N,N-diisopropylethylamine were added. The mixture was stirred at RT overnight. After dilution with water, the mixture was extracted three times with in each case 10 ml of ethyl acetate. The combined organic phases were washed with saturated sodium chloride solution, dried over sodium sulphate, filtered and concentrated under reduced pressure. Drying under HV gave 125 mg (50% of theory) of the target compound which were reacted further without further purification.

GC-MS [Methode 5]: Rₜ=5.52 min; MS (ESI+): m/z=273 and 275 (M+H)⁺.

### Example 54A

### 4-Bromo-N-tert-butyl-2-fluorobenzamide

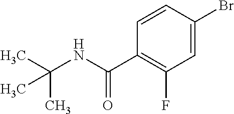

300 mg (1.26 mmol) of 4-bromo-2-fluorobenzoyl chloride, dissolved in 5 ml of dichloromethane were added dropwise to a solution of 101 mg (1.39 mmol) of tert-butylamine and 0.53 ml (3.79) mmol of triethylamine in 5 ml of dichloromethane. The mixture was stirred at RT overnight. After dilution with 10 ml of dichloromethane, the mixture was washed with saturated sodium bicarbonate solution. The organic phase was washed with saturated sodium chloride solution, dried over sodium sulphate, filtered and concentrated under reduced pressure. Drying under HV gave 278 g (80% of theory) of the target compound which were reacted further without further purification.

GC-MS [Method 5]: Rₜ=5.16 min; MS (ESI+): m/z=273 and 275 (M+H)⁺.

### Example 55A

### tert-Butyl (3R)-3-methyl-1,4′-bipiperidine-1′-carboxylate hydrochloride

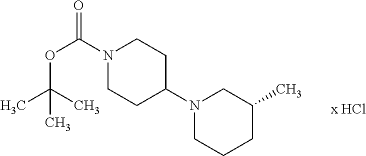 [SMILES: C[C@@H]1CCCN(C2CCN(C(=O)OC(C)(C)C)CC2)C1]

12.89 g (64.7 mmol) of tert-butyl 4-oxopiperidine-1-carboxylate together with 7.70 g (77.6 mmol) of (3R)-3-methylpiperidine and about 2 g of molecular sieve 3 Å in 220 ml of dichloromethane were stirred at RT for 1 h. 20.60 g (97.0 mmol) of sodium triacetoxyborohydride were then added to this suspension, and the mixture was stirred at RT for a further 16 h. For work-up, the mixture was diluted with 200 ml of dichloromethane and washed twice with in each case 100 ml of saturated sodium bicarbonate solution. The aqueous phase was extracted once with 100 ml of dichloromethane and the combined organic phases were washed twice with in each case 100 ml of saturated sodium chloride solution. The organic phase was dried over sodium sulphate, filtered and concentrated under reduced pressure. The residue obtained was dissolved using about 50 ml of dichloromethane, and 20 ml of a 4N solution of hydrogen chloride in dioxane were added. The mixture was stirred for another 10 min approximately and then concentrated by evaporation, and the solid residue obtained was triturated with diethyl ether. The product was filtered off with suction, washed with ether and dried under HV. This gave 10.7 g (49% of theory) of the target compound.

LC-MS [Method 2]: Rₜ=0.54 min; MS (ESIpos): m/z=283 (M+H)⁺

### Example 56A

### (3R)-3-Methyl-1,4′-bipiperidine

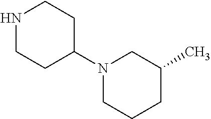

10.7 g (31.9 mmol) of the compound from example 55A were suspended in 72 ml of a mixture of dichloromethane and TFA (5:1) and stirred at RT for 3 h. After concentration of the mixture, about 100 ml of diethyl ether and 15 ml of water were added to the residue and, with ice cooling, the pH was adjusted to pH=12 using 45% strength sodium hydroxide solution. The organic phase was separated off and the aqueous phase was extracted three times with in each case 50 ml of diethyl ether. The combined organic phases were washed once with about 20 ml of saturated sodium chloride solution. The organic phase was dried over magnesium sulphate, filtered and concentrated under reduced pressure. The residue obtained was purified by kugelrohr distillation. At a pressure of 0.29 mbar in a boiling range of 135-150° C., 4.40 g (70% of theory) of the target compound were obtained.

LC-MS [Method 5]: Rₜ=4.31 min; MS (ESIpos): m/z=182 (M)⁺

### Example 57A

### Methyl 4-{[(3R)-3-methyl-1,4′-bipiperidin-1′-yl]carbonyl}benzoate

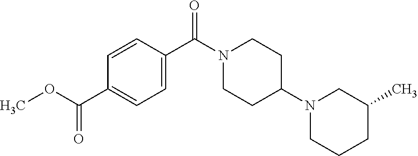

1.70 g (9.42 mmol) of monomethyl terephthalate and 1.89 g (10.37 mmol) of the compound from Example 56A were suspended in 100 ml of acetonitrile, and 1.22 g (9.42 mmol) of N,N-diisopropylethylamine were added. At 0° C., 7.20 g (11.31 mmol) of T3P (50% by weight strength solution in DMF) were added, and the mixture was stirred at RT for 22 h. For work-up, the volatile constituents were removed under reduced pressure and the residue was taken up in about 20 ml of water and made alkaline with ammonia solution. The mixture was extracted three times with 20 ml of dichloromethane each time. The combined organic phases were washed with saturated sodium chloride solution, dried over magnesium sulphate, filtered and concentrated under reduced pressure. The crude product was dissolved in about 20 ml of ethyl acetate and the mixture was washed twice with in each case 10 ml of saturated sodium bicarbonate solution and once with 10 ml of saturated sodium chloride solution. After drying over magnesium sulphate, the mixture was filtered and concentrated under reduced pressure. The product was dried under HV. This gave 3.70 g of the title compound (>100% of theory) which were reacted further without further purification.

LC-MS[Method 2]: Rₜ=0.56 min; MS (ESI+): m/z=345 (M+H)⁺

¹H-NMR (400 MHz, DMSO-d₆): δ [ppm]=0.75-0.87 (m, 1H), 0.82 (d, 3H), 1.30-1.68 (m, 7H), 1.69-1.86 (m, 2H), 2.05 (t, 1H), 2.66-2.84 (m, 4H), 3.00 (t, 1H), 3.26-3.37 (m, 1H), 3.48 (d, 1H), 3.87 (s, 3H), 4.50 (d, 1H), 7.52 (d, 2H), 8.00 (d, 2H).

### Example 58A

### 4-{[(3R)-3-Methyl-1,4′-bipiperidin-1′-yl]carbonyl}benzoic acid trifluoroacetic acid salt

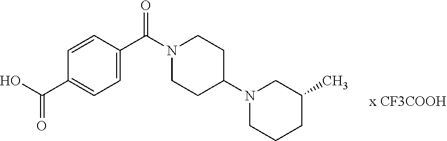 [SMILES: C[C@@H]1CCCN(C2CCN(C(=O)c3ccc(C(=O)O)cc3)CC2)C1]

3.70 g (10.74 mmol) of the compound from Example 57A were dissolved in 140 ml of THF/methanol (5:1), and 53.7 ml (53.7 mmol) of a 1N lithium hydroxide solution in water were added. The mixture was stirred at 40° C. for 5 h. For work-up, the mixture was, with ice-cooling, acidified to pH=4 using 6N hydrochloric acid, and the mixture was concentrated under reduced pressure. The residue obtained was dissolved using 25 ml of water and 5 ml of ammonia solution and purified chromatographically in 6 portions [Method 12c]. This gave 3.05 g of the title compound (64% of theory).

Rotation: α_{D}²⁰ (methanol): −0.9°

LC-MS[Method 8]: Rₜ=0.32 min; MS (ESI+): m/z=331 (M+H)⁺

¹H-NMR (400 MHz, DMSO-d₆): δ [ppm]=0.91 (d, 3H), 1.02-1.17 (m, 1H), 1.60-2.19 (m, 8H), 2.55-2.67 (m, 1H), 2.72-2.96 (m, 2H), 3.02-3.21 (m, 1H), 3.27-3.69 (m, 4H), 4.56-4.72 (m, 1H), 7.54 (d, 2H), 8.01 (d, 2H), 9.64 (br. s., 1H).

### Example 59A

### O-[(1-Benzylpiperidin-3-yl)methyl]S-methyl dithiocarbonate

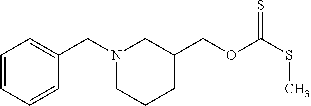 [SMILES: CSC(=S)OCC1CCCN(Cc2ccccc2)C1]

With ice cooling, 296 mg (7.4 mmol) of sodium hydride (60% in mineral oil) were added to a solution of 1.01 g (4.93 mmol) of (1-benzylpiperidin-3-yl)methanol in 11.6 ml of DMF, and the mixture was stirred at RT for 50 min. Subsequently, with ice cooling, 0.59 ml (9.9 mmol) of carbon disulphide were added dropwise and the mixture was stirred at RT for 4.5 h. The mixture was cooled once more to 5° C., 0.46 ml (7.4 mmol) of iodomethane was then added dropwise and the reaction mixture was stirred at RT overnight. For work-up, saturated ammonium chloride solution was added, the mixture was extracted with ethyl acetate and the organic phase was washed with saturated sodium chloride solution, dried over magnesium sulphate and concentrated. The crude product was purified chromatographically on silica gel (elution with cyclohexane/ethyl acetate 95:5-70:30), which gave 823 mg (56% of theory) of the title compound.

R_{f} value (cyclohexane/ethyl acetate 5:1): 0.24

LC-MS [Method 9]: Rₜ=0.71 min; MS (ESIpos): m/z=296 (M+H)⁺

¹H-NMR (400 MHz, DMSO-d₆): δ [ppm]=1.03-1.20 (m, 1H), 1.40-1.55 (m, 1H), 1.57-1.72 (m, 2H), 1.85-1.96 (m, 1H), 1.97-2.15 (m, 2H), 2.47 (s, 3H), 2.58-2.67 (m, 1H), 2.67-2.76 (m, 1H), 3.39-3.51 (m, 2H), 4.41-4.53 (m, 2H), 7.19-7.34 (m, 5H).

### Example 60A

### 1-Benzyl-3-[(trifluoromethoxy)methyl]piperidine

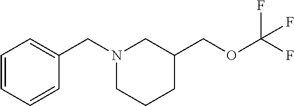 [SMILES: FC(F)(F)OCC1CCCN(Cc2ccccc2)C1]

In a 250 ml Teflon flask, a suspension of 1.21 g (4.24 mmol) of 1,3-dibromo-5,5-dimethylhydantoin in 40 ml of dichloromethane was cooled to −75° C. (internal temperature). 2.8 ml (113 mmol) of hydrogen fluoride/pyridine complex (65-70%) were added over about 5 min. After 10 min, a solution of 829 mg (2.78 mmol) of the compound from Example 59A in 10 ml of dichloromethane was added dropwise over 5 min. After the addition had ended, the mixture was stirred at this temperature for 10 min and then for 45 min in an ice/sodium chloride bath (−20° C.). For work-up, 60 ml of diethyl ether were added and the reaction mixture was poured onto a cooled mixture of 60 ml of saturated sodium bicarbonate solution, 60 ml of saturated sodium thiosulphate solution and 40 ml of 1M sodium hydroxide solution. Using 50% strength sodium hydroxide solution, the pH was once more adjusted to pH 10, and the aqueous phase was extracted three times with 60 ml of diethyl ether. The combined organic phases were washed with saturated sodium chloride solution, dried over magnesium sulphate and concentrated. The crude product was purified chromatographically on silica gel (elution with cyclohexane/ethyl acetate 95:5-70:30), which gave 147 mg (36% of theory) of the title compound in a purity of 94% (based on GC-MS area %).

R_{f} value (silica gel, cyclohexane/ethyl acetate 2:1): 0.52

GC-MS [Method 5]Rₜ=4.27 min; MS (EI): m/z=273 (M)⁺

¹H-NMR (400 MHz, CDCl₃): δ [ppm]=0.99-1.24 (m, 1H), 1.50-1.78 (m, 3H), 1.86-2.14 (m, 3H), 2.64-2.74 (m, 1H), 2.74-2.84 (m, 1H), 3.49 (q, 2H), 3.79-3.89 (m, 2H), 7.21-7.36 (m, 5H).

### Example 61A

### 3-[(Trifluoromethoxy)methyl]piperidine hydrochloride

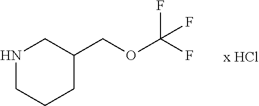 [SMILES: FC(F)(F)OCC1CCCNC1]

15 mg of palladium 10% on carbon were added to a solution of 138 mg (0.505 mmol) of the compound from Example 60A in methanol, and the mixture was hydrogenated in a Parr apparatus at RT and a hydrogen pressure of 2.8 bar overnight. Owing to incomplete conversion, palladium hydroxide 20% on carbon was added and the mixture was hydrogenated at a hydrogen pressure of 2.8 bar for 3 days. For workup, the reaction mixture was filtered through kieselguhr, washed with ethyl acetate, and the filtrate was concentrated. The crude product obtained was converted into the corresponding hydrochloride using a 4N solution of hydrogen chloride in dioxane. This gave 58.0 mg (52% of theory, 85% pure based on GC-MS area %) of the title compound which was reacted without further purification.

GC-MS [Method 5]: Rₜ=1.61 min; MS (EI): m/z=183 (M)⁺

¹H-NMR (400 MHz, DMSO-d₆): δ [ppm]=1.17-1.40 (m, 1H), 1.54-1.70 (m, 1H), 1.70-1.86 (m, 2H), 2.03-2.20 (m, 1H), 2.62-2.84 (m, 2H), 3.18-3.30 (m, 2H), 3.95-4.12 (m, 2H), 8.43-8.90 (m, 2H).

### Example 62A

### 3-(Cyclobutyloxy)pyridine hydrochloride

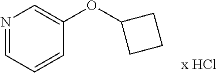 [SMILES: c1cncc(OC2CCC2)c1]

With ice cooling, 2.59 g (13.7 mmol) of triphenylphosphine were added to a solution of 1.00 g (10.5 mmol) of 3-hydroxypyridine and 1.3 ml (13.7 mmol) of cyclobutylmethanol in 20 ml of THF, and the mixture was stirred for 5 min. 2.7 ml (13.7 mmol) of diisopropyl azodicarboxylate were then added dropwise and the reaction mixture was warmed to RT overnight. For work-up, water was added and the mixture was extracted twice with in each case 50 ml of ethyl acetate. The combined organic phases were washed with saturated sodium chloride solution, dried over magnesium sulphate and concentrated. The crude product was stirred with 50 ml of cyclohexane and the white solid was filtered off with suction and washed three times with in each case 20 ml of cyclohexane. The filtrate was concentrated and dissolved in 40 ml of diethyl ether, and 3 ml (12 mmol) of a 4N solution of hydrogen chloride in dioxane were added with ice cooling. The resulting beige precipitate was filtered off, washed twice with in each case 20 ml of diethyl ether and dried under HV. This gave 1.67 g (76% of theory) of the target compound.

LC-MS [Method 10]: Rₜ=1.46 min; MS (ESIpos): m/z=164 (M+H)⁺

¹H-NMR (400 MHz, DMSO-d₆): δ [ppm]=1.77-2.01 (m, 4H), 2.03-2.17 (m, 2H), 2.68-2.84 (m, 1H), 4.19 (d, 2H), 7.90 (dd, 1H), 8.10 (dd, 1H), 8.47 (d, 1H), 8.65 (d, 1H).

### Example 63A

### 3-(Cyclobutyloxy)piperidine hydrochloride

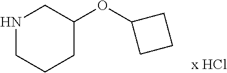 [SMILES: C1CC(OC2CCCNC2)C1]

20.5 mg (0.090 mmol) of platinum(IV) oxide were added to a solution of 205 mg (1.03 mmol) of the compound from Example 62A in 10 ml of methanol, and the mixture was hydrogenated in a Parr apparatus at RT and a hydrogen pressure of 2.9 bar overnight. For workup, the mixture was filtered through kieselguhr, washed with methanol, and the filtrate was concentrated. This gave 192 mg of crude product, which was converted further without further purification.

GC-MS [Method 5]: Rₜ=3.72 min; MS (EI): m/z=169 (M)⁺

### Example 64A

### 3-(Cyclopropyloxy)pyridine hydrochloride

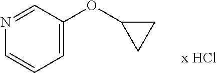

A mixture of 1.00 g (10.5 mmol) of 3-hydroxypyridine, 2.4 ml (30.5 mmol) of cyclopropyl bromide, 261 mg (1.56 mmol) of potassium iodide and 10.3 g (31.5 mmol) of caesium carbonate in 15 ml of DMF was stirred in a microwave at 180° C. for 7.5 h. After cooling to RT, water was added and the mixture was extracted repeatedly with tert-butyl methyl ether. The combined organic phases were washed with saturated sodium chloride solution, dried over magnesium sulphate, filtered and concentrated. The crude product was purified chromatographically on silica gel (elution with cyclohexane/ethyl acetate 95:5-70:30). The isolated product was taken up in dichloromethane, 1 N hydrochloric acid was added, the mixture was concentrated and then extracted with diethyl ether, concentrated and dried under HV. This gave 336 mg (17% of theory) of the title compound.

R_{f} value (cyclohexane/ethyl acetate 2:1, free base): 0.26

LC-MS [Method 9]: Rₜ=0.38 min; MS (ESIpos): m/z=136 (M+H)⁺

¹H-NMR (400 MHz, DMSO-d₆): δ [ppm]=0.72-0.79 (m, 2H), 0.84-0.92 (m, 2H), 4.07-4.14 (m, 1H), 7.82 (dd, 1H), 8.06 (br. d, 1H), 8.46 (d, 1H), 8.65 (d, 1H).

### Example 65A

### 3-(Cyclopropyloxy)piperidine hydrochloride

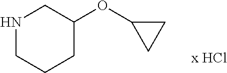

36 mg (0.160 mmol) of platinum(IV) oxide were added to a solution of 336 mg (1.82 mmol) of the compound from Example 64A in 8.9 ml of methanol, and the mixture was hydrogenated in a Parr apparatus at RT and a hydrogen pressure of 2.9 bar overnight. For workup, the mixture was filtered through kieselguhr, washed with methanol, and the filtrate was concentrated. This gave 290 mg of crude product, which was converted further without further purification.

### Example 66A

### 2-(4-Bromo-2-fluorophenyl)propan-2-ol

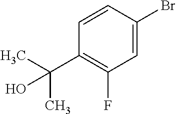

At 0° C., 2.86 ml (8.59 mmol) of methylmagnesium bromide (3M solution in diethyl ether) were added to a solution of 500 mg (2.15 mmol) of methyl 4-bromo-2-fluorobenzoate in 10 ml of dry THF, and the mixture was stirred at this temperature for one hour. After a further hour at RT, about 10 ml of saturated ammonium chloride solution and 10 ml of ethyl acetate were added. After separation of the phases, the aqueous phase was extracted once more with 10 ml of ethyl acetate. The combined organic phases were dried over sodium sulphate, filtered and concentrated. The crude product obtained was dried under HV and reacted further without further purification. This gave 431 mg (86% of theory) of the target compound.

GC-MS [Method 5]: Rₜ=3.70 min; MS (ESIpos): m/z=232 and 234 (M).

### Example 67A

### tert-Butyl 3-({[(4-methylphenyl) sulphonyl]oxy}methyl)piperidine-1-carboxylate

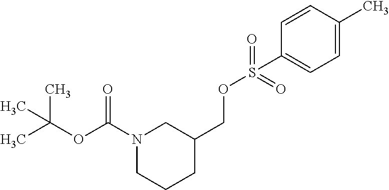 [SMILES: Cc1ccc(S(=O)(=O)OCC2CCCN(C(=O)OC(C)(C)C)C2)cc1]

At 0° C., 974 mg (5.11 mmol) of toluene-4-sulphonyl chloride and 0.71 ml (5.11 mmol) of triethylamine were added to a solution of 1.00 g (4.65 mmol) of tert-butyl (3S)-3-(hydroxymethyl)piperidine-1-carboxylate in 15 ml of dichloromethane, and the mixture was subsequently stirred at RT for 18 h. The mixture was then diluted with about 15 ml of dichloromethane and washed once with 10 ml of saturated sodium bicarbonate solution and 10 ml of saturated sodium chloride solution. The organic phase was dried over sodium sulphate, filtered and concentrated. The crude product was purified by chromatography on silica gel (mobile phase: cyclohexane/ethyl acetate 10:1-4:1). This gave 1.38 g of product (80% of theory).

LC-MS[Method 1]: Rₜ=1.18 min; MS (ESI+): m/z=370 (M+H)⁺

### Example 68A

### tert-Butyl 3-[(cyclobutyloxy)methyl]piperidine-1-carboxylate

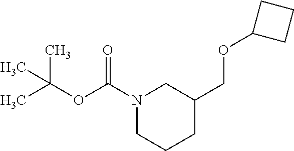 [SMILES: CC(C)(C)OC(=O)N1CCCC(COC2CCC2)C1]

0.11 ml (1.43 mmol) of cyclobutanol was added to 57 mg (1.43 mmol) of sodium hydride (60% suspension in mineral oil) in 3 ml of dry DMF. The mixture was stirred at RT for 30 min. 176 mg (0.48 mmol) of the compound from Example 67A, dissolved in 3 ml of DMF, were added to the now clear solution, and the mixture was subsequently stirred in a preheated oil bath at 55° C. for 9 h. After cooling to RT, 10 ml of water were added and the mixture was extracted twice with in each case 25 ml of ethyl acetate. The combined organic phases were dried over sodium sulphate, filtered and concentrated. The crude product was purified by chromatography on silica gel (mobile phase: cyclohexane/ethyl acetate 10:1). This gave 71 mg of product (51% of theory).

LC-MS[Method 9]: Rₜ=1.33 min; MS (ESI+): m/z=270 (M+H)⁺

### Example 69A

### 3-[(Cyclobutyloxy)methyl]piperidine hydrochloride

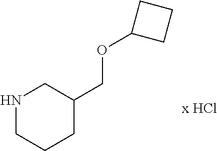 [SMILES: C1CNCC(COC2CCC2)C1]

At RT, 0.26 ml (1.05 mmol) of a 4N solution of hydrogen chloride in dioxane were added to a solution of 71 mg (0.26 mmol) of the compound from Example 68A in 1 ml of dichloromethane, and the mixture was subsequently stirred at RT for 2 h. The mixture was concentrated to dryness and the crude product was reacted further without further purification. This gave 64 mg of the target compound (80% of theory).

LC-MS[Method 2]: Rₜ=0.45 min; MS (ESI+): m/z=170 (M+H)⁺

### Example 70A

### Benzyl 3-[(vinyloxy)methyl]piperidine-1-carboxylate

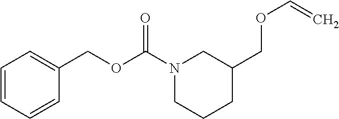 [SMILES: C=COCC1CCCN(C(=O)OCc2ccccc2)C1]

At RT, 23 ml (241 mmol) of ethyl vinyl ether were added to 321 mg (0.65 mmol) of chloro(triphenylphosphine)gold(I) and 108 mg of silver(I) acetate. After 10 min of stirring, 6.00 g (24.07 mmol) of benzyl 3-(hydroxymethyl)piperidine-1-carboxylate were added. The mixture was stirred at 50° C. for 5 h. This was followed by concentration under reduced pressure. The crude product was purified by chromatography on silica gel (mobile phase: cyclohexane/ethyl acetate gradient 100:0-100:1-20:1-10:1). This gave 4.40 g of product (66% of theory).

LC-MS[Method 1]: Rₜ=1.15 min; MS (ESI+): m/z=276 (M+H)⁺

### Example 71A

### Benzyl 3-[(cyclopropyloxy)methyl]piperidine-1-carboxylate

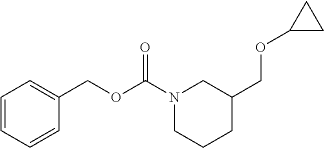 [SMILES: O=C(OCc1ccccc1)N1CCCC(COC2CC2)C1]

In a flask which had been dried by heating, a solution of 3.80 g (13.8 mmol) of the compound from Example 70A was initially charged under argon in 80 ml of dry diethyl ether, 41.4 ml (41.4 mmol) of diethylzinc (1M in hexane) were added at RT. 3.45 ml (42.8 mmol) of diiodomethane were then slowly added dropwise. The mixture was stirred under reflux for 18 h. After cooling to RT, 150 ml of saturated ammonium hydrochloride solution were added. The solid was filtered off with suction and washed thoroughly with diethyl ether. After separation of the phases, the organic phase was extracted three more times with in each case 50 ml of diethyl ether. The combined organic phases were washed with about 50 ml of saturated sodium chloride solution, dried over sodium sulphate, filtered and concentrated. Drying under HV gave 3.7 g (86% of theory) of the target compound.

LC-MS [Method 10]: Rₜ=2.47 min; MS (ESI+): m/z=290 (M+H)⁺

### Example 72A

### 3-[(Cyclopropyloxy)methyl]piperidine

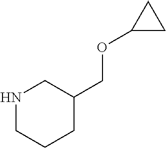 [SMILES: C1CNCC(COC2CC2)C1]

101 mg (0.14 mmol) of palladium(II) hydroxide (20% on activated carbon) were added to a solution of 209 mg (0.72 mmol) of the compound from Example 71A in 500 ml of ethanol, and the mixture was hydrogenated at RT and a hydrogen pressure of 3-4 bar for 18 h. For work-up, the catalyst was filtered off and washed with a little ethanol and the filtrate was carefully concentrated under reduced pressure. 112 mg (97% of theory) of the target compound were obtained.

LC-MS [Method 2]: Rₜ=0.27 min; MS (ESI+): m/z=156 (M+H)⁺

¹H-NMR (400 MHz, DMSO-d₆): δ [ppm]=0.35-0.50 (m, 4H), 1.08-1.20 (m, 1H), 1.47-1.61 (m, 2H), 1.62-1.78 (m, 2H), 1.79-1.93 (m, 2H), 2.57-2.69 (m, 1H), 3.04-3.13 (m, 2H), 3.19-3.36 (m, 4H), 8.38 (s, 1H).

### Example 73A

### 1-[4-(2-Methoxypropan-2-yl)benzoyl]piperidin-4-one

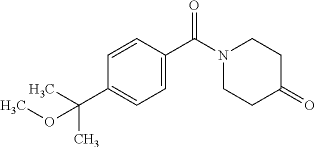 [SMILES: COC(C)(C)c1ccc(C(=O)N2CCC(=O)CC2)cc1]

Analogously to the compound from Example 44A, 0.67 g (2.84 mmol) of the compound from Example 12A, 0.46 g (3.41 mmol) of piperidin-4-one hydrochloride hydrate and 1.24 ml of N,N-diisopropylethylamine were dissolved in 12 ml of acetonitrile, and 1.82 ml (3.12 mmol) of T3P (50% by weight strength solution in ethyl acetate) were added at 0° C. The mixture was stirred at RT for 18 h. For work-up, the reaction mixture was concentrated, 10 ml of water were added and the mixture was extracted four times with in each case 10 ml of ethyl acetate. The combined organic phases were washed successively with 10 ml of saturated sodium bicarbonate solution and with 10 ml of saturated sodium chloride solution. The mixture was dried over sodium sulphate, filtered and concentrated under reduced pressure. Drying under HV gave 0.416 g (52% of theory) of the title compound.

LC-MS [Method 9]: Rₜ=0.72 min; MS (ESI+): m/z=276 (M+H)⁺

¹H-NMR (400 MHz, DMSO-d₆): δ [ppm]=1.48 (s, 6H), 2.34-2.48 (m, 4H), 3.00 (s, 3H), 3.50-4.0 (m, 4H), 7.48 (s, 4H).

### Example 74A

### 4-(3-Hydroxyoxetan-3-yl)benzoic acid

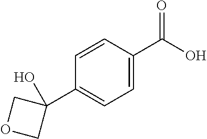 [SMILES: O=C(O)c1ccc(C2(O)COC2)cc1]

0.8 ml of 25% strength sodium hydroxide solution was added to 440 mg (2.51 mmol) of 4-(3-hydroxyoxetan-3-yl)benzonitrile, and the mixture was heated under reflux for 1 h. After cooling to RT, the mixture was acidified with 10% strength sulphuric acid (pH 4). The precipitate was filtered off, washed with water and dried under HV. This gave 435 mg (89% of theory) of the target compound.

LC-MS [Method 10]: Rₜ=0.93 min; MS (ESI+): m/z=195 (M+H)⁺

### Example 75A

### 1-[4-(3-Hydroxyoxetan-3-yl)benzoyl]piperidin-4-one

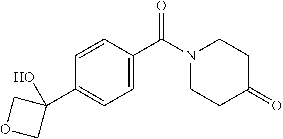 [SMILES: O=C1CCN(C(=O)c2ccc(C3(O)COC3)cc2)CC1]

At 0° C., a solution of 350 mg (1.80 mmol) of the compound from Example 74A, 268 mg (1.98 mmol) of 4-piperidone hydrochloride and 0.75 ml (4.51 mmol) of diisopropylethylamine in 7.1 ml of acetonitrile was reacted with 1.26 ml (2.16 mmol) of T3P (50% by weight strength solution in DMF) analogously to the compound from Example 36A. The crude product was purified by chromatography on silica gel (mobile phase: cyclohexane/ethyl acetate gradient 5:1-1:1, ethyl acetate, ethyl acetate/methanol 5:1). This gave 219 mg of the target compound (43% of theory).

LC-MS [Method 9]: Rₜ=0.42 min; MS (ESI+): m/z=276 (M+H)⁺

¹H-NMR (400 MHz, DMSO-d₆): δ [ppm]=2.34-2.56 (m, 4H), 3.56-3.96 (m, 2H), 4.70 (d, 2H), 4.79 (d, 2H), 6.46 (br. s., 1H), 7.49-7.55 (m, 2H), 7.66-7.71 (m, 2H).

### Target Compounds

### Example 1

### Ethyl 1′-[(4-tert-butylphenyl)carbonyl]-1,4′-bipiperidine-3-carboxylate

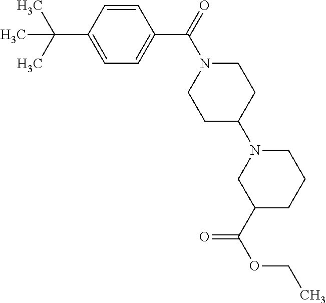 [SMILES: CCOC(=O)C1CCCN(C2CCN(C(=O)c3ccc(C(C)(C)C)cc3)CC2)C1]

0.6 g (3.37 mmol) of 4-tert-butylbenzoic acid were dissolved in 25 ml of DMF, and 0.65 g (3.37 mmol) of EDC, 0.52 g (3.37 mmol) of HOBT and 2.2 g (16.8 mmol) of N,N-diisopropylethylamine were added. The mixture was stirred at RT for 1 h. 1.1 g (3.37 mmol) of ethyl 1,4′-bipiperidine-3-carboxylate dihydrochloride were then added, and the mixture was subsequently stirred at RT overnight. The resulting product was separated by preparative HPLC [Reprosil, C18 10 μm, 250 mm×30 mm, acetonitrile/water 10:90 to 90:10 over a run time of 38 min]. After HPLC control, the product-containing fractions were combined and concentrated. The residue was dried under HV. This gave 0.706 g (52% of theory) of the racemate as an oil.

LC-MS [Method 1]: Rₜ=0.82 min; MS (ESIpos): m/z=401 (M+H)⁺

¹H-NMR (400 MHz, DMSO-d₆): δ [ppm]=1.17 (t, 3H), 1.25-1.49 (m, 4H), 1.29 (s, 9H), 1.54-1.87 (m, 4H), 2.17-2.28 (m, 1H), 2.34-2.47 (m, 1H), 2.61-3.07 (m, 4H), 3.5-3.8 (m, 1H), 4.05 (q, 2H), 4.3-4.65 (m, 1H), 7.24-7.34 (m, 2H), 7.4-7.47 (m, 2H)

### Example 2

### 3-(Ethoxycarbonyl)-1-{1-[4-(ethoxycarbonyl)benzoyl]piperidin-4-yl}piperidine trifluoroacetic acid salt

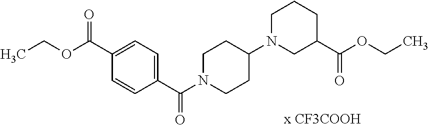 [SMILES: CCOC(=O)c1ccc(C(=O)N2CCC(N3CCCC(C(=O)OCC)C3)CC2)cc1]

200 mg (0.87 mmol) of ethyl 4-bromobenzoate, 136.8 mg (0.44 mmol) of ethyl 1,4′-bipiperidine-3-carboxylate dihydrochloride, 57.6 mg (0.22 mmol) of molybdenum hexacarbonyl, 20.5 mg (0.02 mmol) of trans-bis(acetate)bis[o-(di-o-tolylphosphine)benzyl]dipalladium(II) (Herrmann's palladacycle) and 231.3 mg (2.18 mmol) of sodium carbonate were suspended in 1 ml of water and heated in a microwave at 150° C. for 15 minutes. After cooling, the mixture was extracted with ethyl acetate and then filtered through kieselguhr. The organic phase was removed from the filtrate, dried over magnesium sulphate and filtered, and the filtrate was concentrated. The residue was purified by preparative HPLC. [Reprosil C18, 10 μm, 250 mm×30 mm (50% methanol/50% water (+0.05% trifluoroacetic acid) to 70% methanol/30% water (+0.05% trifluoroacetic acid)) over a run time of 25 min]. The product-containing fractions were combined, concentrated and dried under HV. This gave 59 mg (12% of theory) of an oil.

LC-MS [Method 4]: Rₜ=1.30 min; MS (ESIpos): m/z=417 (M−CF₃COOH+H)⁺

¹H-NMR (400 MHz, DMSO-d₆): δ [ppm]=1.13-1.24 (t, 3H), 1.33 (t, 3H), 1.45-2.23 (m, 9H), 2.63-3.31 (m, 7H), 4.08-4.18 (m, 2H), 4.34 (q, 2H), 4.56-4.71 (m, 1H), 7.49-7.63 (m, 2H), 8.00-8.05 (m, 2H), 9.22-9.45 (m, 1H).

### Example 3

### (3-Chloro-4-tert-butylphenyl)(3-methyl-1,4′-bipiperidin-1′-yl)methanone trifluoroacetic acid salt

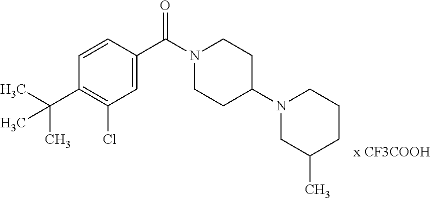 [SMILES: CC1CCCN(C2CCN(C(=O)c3ccc(C(C)(C)C)c(Cl)c3)CC2)C1]

100 mg (0.47 mmol) of 3-chloro-4-tert-butylbenzoic acid were dissolved in 10 ml of dichloromethane, and 597 mg (4.7 mmol) of oxalyl chloride and 1 drop of DMF were added. After 1 h of stirring, the mixture was concentrated on a rotary evaporator and dried under HV. 86 mg (0.47 mmol) of 3-methyl-1,4′-bipiperidine dissolved in dichloromethane were initially charged, 238 mg (2.35 mmol) of triethylamine were added and the above acid chloride, obtained after drying under HV, was added dissolved in dichloromethane. The mixture was stirred at RT overnight. The mixture was concentrated and the resulting product was then separated by preparative HPLC [Reprosil, C18 10 μm, 250 mm×30 mm, acetonitrile/water (+0.05% trifluoroacetic acid) 10:90 to 90:10 over a run time of 38 min]. After HPLC control, the product-containing fractions were combined and concentrated. The residue was dried under HV, giving 33 mg (14% of theory) of an oil.

LC-MS [Method 3]: Rₜ=1.02 min; MS (ESIpos): m/z=377 (M−CF₃COOH+H)⁺

¹H-NMR (400 MHz, DMSO-d₆): δ [ppm]=0.87-0.94 (d, 3H), 1.01-1.18 (m, 1H), 1.46 (s, 9H), 1.60-1.76 (m, 3H), 1.77-1.90 (m, 1H), 1.91-2.15 (m, 1H), 2.75-3.3 (m, 4H), 3.25-3.46 (m, 3H), 4.46-4.69 (m, 1H), 7.31-7.36 (m, 1H), 7.43-7.46 (m, 1H), 7.52-7.56 (m, 1H), 9.08 (br. s., 1H)

### Example 4

### (4-Isopropylphenyl)(3-methyl-1,4′-bipiperidin-1′-yl)methanone

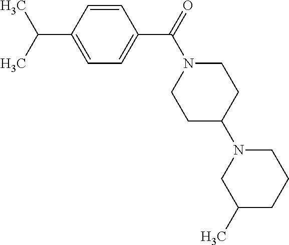 [SMILES: CC1CCCN(C2CCN(C(=O)c3ccc(C(C)C)cc3)CC2)C1]

100 mg (0.61 mmol) of 4-isopropylbenzoic acid were dissolved in 3 ml of DMF, and 128.4 mg (0.67 mmol) of EDC, 103 mg (0.67 mmol) of HOBT and 236 mg (1.83 mmol) of N,N-diisopropylethylamine were added. The mixture was stirred at RT for 1 h. 111 mg (0.61 mmol) of 3-methyl-1,4′-bipiperidine were then added, and the mixture was subsequently stirred at RT overnight. The mixture was diluted with ethyl acetate and washed with water and saturated sodium chloride solution. The organic phase was separated off, dried over sodium sulphate, filtered and concentrated. The resulting product was separated by preparative HPLC [Reprosil, C18 10 μm, 250 mm×30 mm, acetonitrile/water 10:90 to 90:10 over a run time of 38 min]. After HPLC control, the product-containing fractions were combined and concentrated. The residue was dried under HV. This gave 117 mg (57% of theory) of an oil.

LC-MS [Method 1]: Rₜ=0.81 min; MS (ESIpos): m/z=343 (M+H)⁺

¹H-NMR (400 MHz, DMSO-d₆): δ [ppm]=0.75-0.85 (m, 1H) 0.82 (d, 3H), 1.21 (d, 6H), 1.75 (s, 9H), 2.05 (t, 1H), 2.4-2.49 (m, 2H) 2.7-2.8 (m, 2H), 2.86-2.99 (m, 2H), 3.45-3.81 (m, 1H), 4.23-4.65 (m, 1H), 7.27-7.3 (m, 4H)

### Example 5

### (4-tert-Butylphenyl)(3-methyl-1,4′-bipiperidin-1′-yl)methanone

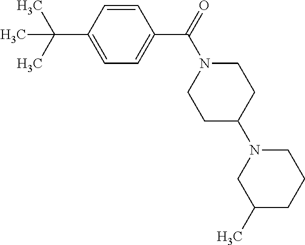 [SMILES: CC1CCCN(C2CCN(C(=O)c3ccc(C(C)(C)C)cc3)CC2)C1]

100 mg (0.56 mmol) of 4-tert-butylbenzoic acid were dissolved in 3 ml of dichloromethane, and 108 mg (0.56 mmol) of EDC, 86 mg (0.56 mmol) of HOBT and 145 mg (1.12 mmol) of N,N-diisopropylethylamine were added. The mixture was stirred at RT for 1 h. 307 mg (1.68 mmol) of 4-(3-methyl)piperidinopiperidine were added, and the mixture was stirred at RT overnight. The mixture was diluted with ethyl acetate and washed with water and saturated sodium chloride solution. The organic phase was separated off, dried over sodium sulphate, filtered and concentrated. The resulting product was separated by preparative HPLC [Reprosil, C18 10 μm, 250 mm×30 mm, acetonitrile/water 10:90 to 90:10 over a run time of 38 min]. After HPLC control, the product-containing fractions were combined and concentrated. The residue was dried under HV. This gave 102 mg (53% of theory) of an oil.

LC-MS [Method 1]: Rₜ=0.79 min; MS (ESIpos): m/z=343 (M+H)⁺

¹H-NMR (400 MHz, DMSO-d₆): δ [ppm]=0.75-0.85 (m, 1H), 0.82 (d, 3H), 1.29 (s, 9H), 1.32-1.83 (m, 8H), 2.05 (t, 1H), 2.4-2.6 (m, 2H), 2.63-2.81 (m, 3H), 2.8-3.1 (m, 1H), 3.59-3.69 (m, 1H), 4.42-4.55 (m, 1H), 7.27-7.34 (m, 2H), 7.4-7.47 (m, 2H)

### Example 6

### (4-tert-Butylphenyl)(3-methyl-1,4′-bipiperidin-1′-yl)methanone, (−)-enantiomer

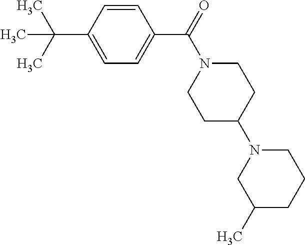 [SMILES: CC1CCCN(C2CCN(C(=O)c3ccc(C(C)(C)C)cc3)CC2)C1]

93 mg (0.27 mmol) of the racemate (4-tert-butylphenyl)(3-methyl-1,4′-bipiperidin-1′-yl)methanone were separated into its enantiomers by preparative HPLC [Daicel Chiralpak AS-H, 5 μm 250 mm×20 mm, 90% isohexane/10% ethanol/0.2% diethylamine, flow rate: 1.0 ml/min, temperature: 30° C.]. After HPLC control, the enantiomerically pure fractions were combined and concentrated. The residue was dried under HV. In this manner, the (−)-enantiomer was isolated with a retention time of 5.8 min under the given conditions. This gave 29 mg (30% of theory) of an oil.

Rotation: α_{D}²⁰ (methanol): −5.3°

LC-MS [Method 3]: Rₜ=0.93 min; MS (ESIpos): m/z=343 (M+H)⁺

¹H-NMR (400 MHz, DMSO-d₆): δ [ppm]=0.75-0.85 (m, 1H), 0.82 (d, 3H), 1.29 (s, 9H), 1.32-1.83 (m, 8H), 2.05 (t, 1H), 2.4-2.6 (m, 2H), 2.63-2.81 (m, 3H), 2.8-3.1 (m, 1H), 3.59-3.69 (m, 1H), 4.42-4.55 (m, 1H), 7.27-7.34 (m, 2H), 7.4-7.47 (m, 2H)

The (+)-enantiomer separated off in this manner had a retention time of 6.19 min [Daicel Chiralpak AS-H, 5 μm 250 mm×20 mm, 90% isohexane/10% ethanol/0.2% diethylamine, flow rate: 1.0 ml/min, temperature: 30° C., rotation: α_{D}²⁰ (methanol): +4.20].

### Example 7

### [4-(1-Hydroxy-1-methylethyl)phenyl)](3-methyl-1,4′-bipiperidin-1′-yl)methanone

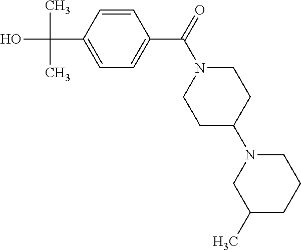 [SMILES: CC1CCCN(C2CCN(C(=O)c3ccc(C(C)(C)O)cc3)CC2)C1]

900 mg (5.0 mmol) of 4-(1-hydroxy-1-methylethyl)benzoic acid were dissolved in 20 ml of DMF, and 957 mg (5.0 mmol) of EDC, 765 mg (5.0 mmol) of HOBT and 1291 mg (10 mmol) of N,N-diisopropylethylamine were added. The mixture was stirred at RT for 10 min. 1002 mg (5.5 mmol) of 3-methyl-1,4′-bipiperidine were then added, and the mixture was subsequently stirred at RT overnight. The mixture was diluted with ethyl acetate and washed with water and saturated sodium chloride solution. The organic phase was separated off, dried over sodium sulphate, filtered and concentrated. The resulting product was separated by preparative HPLC [Reprosil, C18 10 μm, 250 mm×30 mm, acetonitrile/water 10:90 to 90:10 over a run time of 38 min]. After HPLC control, the product-containing fractions were combined and concentrated. The residue was dried under HV. This gave 996 mg (58% of theory) of a crystalline compound.

LC-MS [Method 2]: Rₜ=0.53 min; MS (ESIpos): m/z=345 (M+H)⁺

¹H-NMR (400 MHz, DMSO-d₆): δ [ppm]=0.74-0.88 (m, 1H), 0.82 (d, 3H), 1.30-1.45 (m, 4H), 1.46 (s, 6H) 1.46-1.68 (m, 2H), 1.75 (t, 1H), 2.05 (t, 1H), 2.4-2.55 (m, 4H) 2.6-3.1 (m, 2H), 2.69-2.78 (m, 2H), 3.5-3.75 (m, 1H), 4.38-4.55 (m, 1H), 5.06 (s, 1H), 7.27-7.33 (m, 2H), 7.48-7.53 (m, 2H)

### Example 8

### [4-(1-Hydroxy-1-methylethyl)phenyl)](3-methyl-1,4′-bipiperidin-1′-yl)methanone, (−)-enantiomer

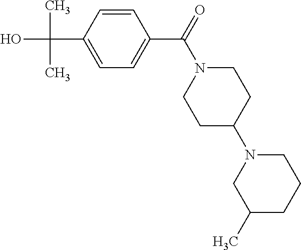 [SMILES: CC1CCCN(C2CCN(C(=O)c3ccc(C(C)(C)O)cc3)CC2)C1]

996 mg (2.9 mmol) of the racemate [4-(1-hydroxy-1-methylethyl)phenyl)](3-methyl-1,4′-bipiperidin-1′-yl)methanone were separated by preparative HPLC [Daicel Chiralpak AS-H, 5 μm 250 mm×20 mm, 90% isohexane/10% ethanol/0.2% diethylamine, flow rate: 1.0 ml/min, temperature: 30° C.]. After HPLC control, the enantiomerically pure fractions were combined and concentrated. The residue was taken up in ethyl acetate and washed twice with water and with saturated sodium chloride solution. The organic phase was separated off, dried over sodium sulphate, filtered and concentrated. The residue was dried under HV. In this manner, the (−)-enantiomer was isolated with a retention time of 9.4 min under the given conditions. This gave 367.8 mg (37% of theory) of an oil.

Rotation: α_{D}²⁰ (methanol): −5.7°

LC-MS [Method 3]: Rₜ=0.93 min; MS (ESIpos): m/z=343 (M+H)⁺

¹H-NMR (400 MHz, DMSO-d₆): δ [ppm]=0.74-0.88 (m, 1H), 0.82 (d, 3H), 1.30-1.45 (m, 4H), 1.46 (s, 6H) 1.46-1.68 (m, 2H), 1.75 (t, 1H), 2.05 (t, 1H), 2.4-2.55 (m, 4H) 2.6-3.1 (m, 2H), 2.69-2.78 (m, 2H), 3.5-3.75 (m, 1H), 4.38-4.55 (m, 1H), 5.06 (s, 1H), 7.27-7.33 (m, 2H), 7.48-7.53 (m, 2H)

The (+)-enantiomer separated off in this manner had a retention time of 10.29 min [Daicel Chiralpak AS-H, 5 μm 250 mm×20 mm, 90% isohexane/10% ethanol/0.2% diethylamine, flow rate: 1.0 ml/min, temperature: 30° C., rotation: α_{D}²⁰ (methanol): +5.30].

### Example 9

### Ethyl 1′-[4-(2-hydroxypropan-2-yl)benzoyl]-1,4′-bipiperidine-3-carboxylate

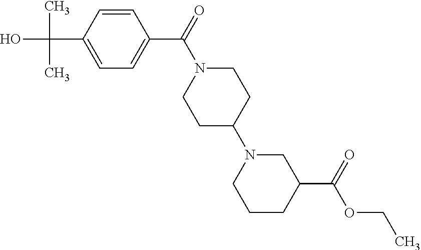 [SMILES: CCOC(=O)C1CCCN(C2CCN(C(=O)c3ccc(C(C)(C)O)cc3)CC2)C1]

600 mg (3.33 mmol) of 4-(1-hydroxy-1-methylethyl)benzoic acid were dissolved in 25 ml of DMF, and 638 mg (3.33 mmol) of EDC, 510 mg (3.33 mmol) of HOBT and 2152 mg (16.6 mmol) of N,N-diisopropylethylamine were added. The mixture was stirred at RT for 10 min. 1043 mg (3.33 mmol) of ethyl 1,4′-bipiperidine-3-carboxylate dihydrochloride were then added, and the mixture was subsequently stirred at RT overnight. The resulting product was separated by preparative HPLC [Reprosil, C18 10 μm, 250 mm×40 mm, acetonitrile/water 10:90 to 90:10 over a run time of 54 min]. After HPLC control, the product-containing fractions were combined and concentrated. The residue was dried under HV. This gave 571 mg (43% of theory) of the title compound.

LC-MS [Method 1]: Rₜ=0.58 min; MS (ESIpos): m/z=403 (M+H)⁺

¹H-NMR (400 MHz, DMSO-d₆): δ [ppm]=1.17 (t, 3H), 1.26-1.49 (m, 1H), 1.43 (s, 3H), 1.54-1.83 (m, 4H), 2.15-2.30 (m, 1H), 2.35-2.47 (m, 2H), 2.48-2.58 (m, 4H), 2.59-3.08 (m, 2H), 2.62-2.72 (m, 2H), 2.79-2.88 (m, 2H), 3.4-3.85 (m, 1H), 4.3-4.6 (m, 1H), 4.05 (d, 2H), 5.06 (s, 1H), 7.27-7.34 (m, 2H), 7.48-7.53 (m, 2H)

### Example 10

### {4-[(2-Methoxyphenoxy)methyl]phenyl}[3-methyl-1,4′-bipiperidin-1′-yl]methanone

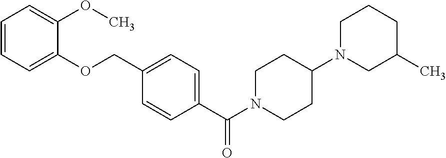 [SMILES: COc1ccccc1OCc1ccc(C(=O)N2CCC(N3CCCC(C)C3)CC2)cc1]

100 mg (0.39 mmol) of 4-[(2-methoxyphenoxy)methyl]benzoic acid were dissolved in 2 ml of DMF, and 74 mg (0.39 mmol) of EDC, 59 mg (0.39 mmol) of HOBT and 200 mg (1.5 mmol) of N,N-diisopropylethylamine were added. The mixture was stirred at RT for 10 min. 71 mg (0.39 mmol) of 3-methyl-1,4′-bipiperidine were then added, and the mixture was subsequently stirred at RT overnight. The resulting product was separated by preparative HPLC [Reprosil, C18 10 μm, 250 mm×30 mm, acetonitrile/water 10:90 to 90:10 over a run time of 38 min]. After HPLC control, the product-containing fractions were combined and concentrated. The residue was dried under HV. This gave 88 mg (52% of theory) of an oil.

LC-MS [Method 2]: Rₜ=0.8 min; MS (ESIpos): m/z=423 (M+H)⁺

¹H-NMR (400 MHz, DMSO-d₆): δ [ppm]=0.73-0.89 (m, 1H), 0.82 (d, 3H), 1.32-1.68 (m, 8H), 1.75 (t, 1H), 1.99-2.12 (m, 1H), 2.43-2.59 (m, 2H), 2.71-2.74 (m., 2H), 2.85-3.1 (m, 1H), 3.45-3.7 (m, 1H), 4.06-4.6 (m, 1H), 3.77 (s, 3H), 5.11 (s, 2H), 6.84-6.95 (m, 2H), 6.96-7.07 (m, 2H), 7.40 (m, 2H), 7.46-7.52 (m, 2H)

### Example 11

### {4-[(3,5-Dimethyl-1H-pyrazol-1-yl)methyl]phenyl}(3-methyl-1,4′-bipiperidin-1′-yl)methanone

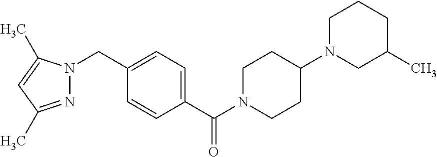 [SMILES: Cc1cc(C)n(Cc2ccc(C(=O)N3CCC(N4CCCC(C)C4)CC3)cc2)n1]

100 mg (0.43 mmol) of 4-[(3,5-dimethyl-1H-pyrazol-1-yl)methyl]benzoic acid were dissolved in 2.3 ml of DMF, and 83 mg (0.43 mmol) of EDC, 67 mg (0.43 mmol) of HOBT and 225 mg (1.74 mmol) of N,N-diisopropylethylamine were added. The mixture was stirred at RT for 10 min. 79 mg (0.43 mmol) of 3-methyl-1,4′-bipiperidine were then added, and the mixture was subsequently stirred at RT overnight. The resulting product was separated by preparative HPLC [Reprosil, C18 10 μm, 250 mm×30 mm, acetonitrile/water 10:90 to 90:10 over a run time of 38 min]. After HPLC control, the product-containing fractions were combined and concentrated. The residue was dried under HV. This gave 52 mg (30% of theory) of an oil.

LC-MS [Method 1]: Rₜ=0.67 min; MS (ESIpos): m/z=395 (M+H)⁺

¹H-NMR (400 MHz, DMSO-d₆): δ [ppm]=0.73-0.88 (m, 1H), 0.82 (d, 3H), 1.29-1.84 (m, 8H), 1.98-2.11 (t, 1H), 2.00-2.19 (m, 1H), 2.1 (s, 3H), 2.16 (s, 3H), 2.41-2.96 (m, 2H), 2.64-2.83 (m, 2H), 2.87-2.94 (m, 1H), 3.05-3.65 (m, 1H), 4.35-4.6 (m, 1H), 5.22 (s, 2H), 5.86 (s, 1H), 7.08-7.14 (m, 2H), 7.31-7.35 (m, 2H)

### Example 12

### (2-Hydroxy-4-tert-butylphenyl)(3-methyl-1,4′-bipiperidin-1′-yl)methanone

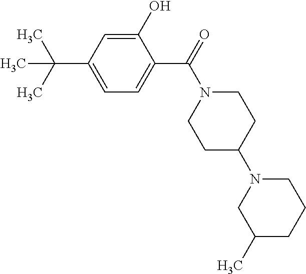 [SMILES: CC1CCCN(C2CCN(C(=O)c3ccc(C(C)(C)C)cc3O)CC2)C1]

100 mg (0.52 mmol) of (2-hydroxy-4-tert-butyl)benzoic acid were dissolved in 2.7 ml of DMF, and 99 mg (0.52 mmol) of EDC, 79 mg (0.52 mmol) of HOBT and 266 mg (2.1 mmol) of N,N-diisopropylethylamine were added. The mixture was stirred at RT for 10 min. 94 mg (0.52 mmol) of 3-methyl-1,4′-bipiperidine were then added, and the mixture was subsequently stirred at RT overnight. The resulting product was separated by preparative HPLC [Reprosil, C18 10 μm, 250 mm×30 mm, acetonitrile/water 10:90 to 90:10 over a run time of 38 min]. After HPLC control, the product-containing fractions were combined and concentrated. The residue was dried under HV. This gave 35 mg (19% of theory) of the title compound.

LC-MS [Method 1]: Rₜ=0.85 min; MS (ESIpos): m/z=359 (M+H)⁺

¹H-NMR (400 MHz, DMSO-d₆): δ [ppm]=0.71-0.91 (m, 1H), 0.82 (d, 3H), 1.24 (s, 9H), 1.3-1.8 (m, 8H), 1.98-2.09 (m, 1H), 2.30-2.46 (m, 2H), 2.64-2.78 (m, 5H), 2.9-2.96 (m, 1H), 6.82-6.87 (m, 2H), 6.99-7.04 (m, 2H)

### Example 13

### [4-(Ethoxymethyl)phenyl](3-methyl-1,4′-bipiperidin-1′-yl)methanone

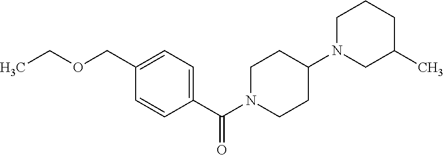 [SMILES: CCOCc1ccc(C(=O)N2CCC(N3CCCC(C)C3)CC2)cc1]

100 mg (0.52 mmol) of 4-(ethoxymethyl)benzoic acid were dissolved in 2.7 ml of DMF, and 96 mg (0.5 mmol) of EDC, 77 mg (0.5 mmol) of HOBT and 258 mg (2 mmol) of N,N-diisopropylethylamine were added. The mixture was stirred at RT for 10 min. 91 mg (0.5 mmol) of 3-methyl-1,4′-bipiperidine were then added, and the mixture was subsequently stirred at RT overnight. The resulting product was separated by preparative HPLC [Reprosil, C18 10 μm, 250 mm×30 mm, acetonitrile/water 10:90 to 90:10 over a run time of 38 min]. After HPLC control, the product-containing fractions were combined and concentrated. The residue was dried under HV. This gave 115 mg (67% of theory) of an oil.

LC-MS [Method 2]: Rₜ=0.64 min; MS (ESIpos): m/z=345 (M+H)⁺

¹H-NMR (400 MHz, DMSO-d₆): δ [ppm]=0.74-0.89 (m, 1H), 0.82 (d, 3H), 1.16 (t, 3H), 1.29-1.82 (m, 10H), 1.99-2.14 (m, 1H), 2.45-2.55 (m, 1H), 2.63-2.82 (m, 2H), 2.89-3.05 (m, 1H), 3.46-3.7 (m, 1H), 3.50 (q, 2H), 4.38-4.62 (m 1H), 4.48 (s, 2H), 7.28-7.43 (m, 4H)

### Example 14

### [4-(Phenoxymethyl)phenyl](3-methyl-1,4′-bipiperidin-1′-yl)methanone

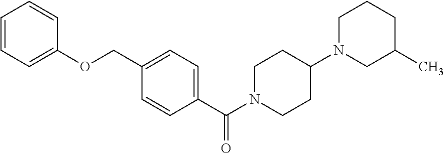 [SMILES: CC1CCCN(C2CCN(C(=O)c3ccc(COc4ccccc4)cc3)CC2)C1]

90 mg (0.39 mmol) of (4-phenoxymethyl)benzoic acid were dissolved in 3 ml of DMF, and 76 mg (0.39 mmol) of EDC, 60 mg (0.39 mmol) of HOBT and 204 mg (1.6 mmol) of N,N-diisopropylethylamine were added. The mixture was stirred at RT for 10 min. 72 mg (0.39 mmol) of 3-methyl-1,4′-bipiperidine were then added, and the mixture was subsequently stirred at RT overnight. The resulting product was separated by preparative HPLC [Reprosil, C18 10 μm, 250 mm×30 mm, acetonitrile/water 10:90 to 90:10 over a run time of 38 min]. After HPLC control, the product-containing fractions were combined and concentrated. The residue was dried under HV. This gave 29 mg (19% of theory) of an oil.

LC-MS [Method 2]: Rₜ=0.83 min; MS (ESIpos): m/z=393 (M+H)⁺

¹H-NMR (400 MHz, DMSO-d₆): δ [ppm]=0.74-0.87 (m, 1H), 0.82 (d, 3H), 1.31-1.69 (m, 8H), 1.75 (t, 1H), 2.05 (t, 1H), 2.43-2.56 (m, 2H), 2.70-2.80 (m, 2H), 2.85-3.1 (m, 1H), 3.5-3.7 (m, 1H), 4.4-4.6 (m, 1H), 5.14 (s, 2H), 6.95 (t, 1H), 6.99-7.05 (m, 2H), 7.27-7.34 (m, 2H), 7.34-7.44 (m, 2H), 7.47-7.53 (m, 2H)

### Example 15

### (4-tert-Butyl-2-methoxyphenyl)(3-methyl-1,4′-bipiperidin-1′-yl)methanone

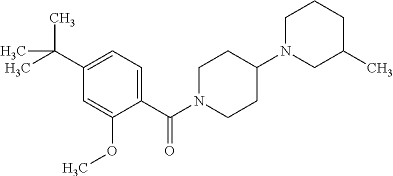 [SMILES: COc1cc(C(C)(C)C)ccc1C(=O)N1CCC(N2CCCC(C)C2)CC1]

100 mg (0.48 mmol) of (4-tert-butyl-2-methoxy)benzoic acid were dissolved in 3 ml of DMF, and 92 mg (0.48 mmol) of EDC, 74 mg (0.48 mmol) of HOBT and 248 mg (1.9 mmol) of N,N-diisopropylethylamine were added. The mixture was stirred at RT for 10 min. 88 mg (0.48 mmol) of 3-methyl-1,4′-bipiperidine were then added, and the mixture was subsequently stirred at RT overnight. The resulting product was separated by preparative HPLC [Reprosil, C18 10 μm, 250 mm×30 mm, acetonitrile/water 10:90 to 90:10 over a run time of 38 min]. After HPLC control, the product-containing fractions were combined and concentrated. The residue was dried under HV. This gave 106 mg (59% of theory) of an oil.

LC-MS [Method 2]: Rₜ=0.83 min; MS (ESIpos): m/z=373 (M+H)⁺

¹H-NMR (400 MHz, DMSO-d₆): δ [ppm]=0.82 (t, 3H), 1.12-1.67 (m, 6H), 1.3 (s, 9H), 1.76 (d, 4H), 2.04 (m, 2H), 2.38-2.57 (m, 2H), 2.61-2.78 (m, 2H), 2.80-2.98 (m, 2H), 3.76-3.83 (m, 2H), 4.51 (d, 1H), 6.96-7.06 (m, 2H), 7.07-7.13 (m, 1H)

### Example 16

### 1′-(4-tert-Butylbenzoyl)-1,4′-bipiperidin-3-yl cyclopropylcarbamate

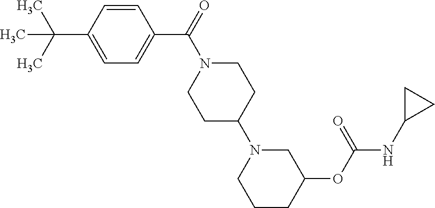 [SMILES: CC(C)(C)c1ccc(C(=O)N2CCC(N3CCCC(OC(=O)NC4CC4)C3)CC2)cc1]

29 mg (0.35 mmol) of cyclopropyl isocyanate and a catalytic amount of N,N-dimethylaminopyridine were added to 60 mg (0.17 mmol) of (4-tert-butylphenyl)(3-hydroxy-1,4′-bipiperidin-1′-yl)methanone. In a microwave oven, this mixture was heated at 150° C. for 30 min. Another 29 mg (0.35 mmol) of cyclopropyl isocyanate were added and the mixture was heated in the microwave at 150° C. for a further 15 min. The mixture was dissolved in a little methanol and separated by preparative HPLC [Reprosil, C18 10 μm, 250 mm×30 mm, methanol/water 30:70 to 100/0 over a run time of 23 min]. After HPLC control, the product-containing fractions were combined and concentrated. The residue was dried under HV. This gave 42 mg (56% of theory) of an oil.

LC-MS [Method 2]: Rₜ=0.8 min; MS (ESIpos): m/z=428 (M+H)⁺

¹H-NMR (400 MHz, DMSO-d₆): δ [ppm]=0.34-0.40 (m, 2H), 0.51-0.57 (m, 2H), 1.17-1.49 (m, 4H), 1.29 (s, 9H), 1.60-1.87 (m, 4H), 2.07-2.24 (m, 2H), 2.39-2.47 (m, 1H), 2.49-2.59 (m, 1H), 2.59-3.05 (m, 3H), 2.64 (d, 1H), 2.89 (dd, 1H), 3.5-3.56 (m, 1H), 4.42-4.55 (m, 1H), 7.21-7.27 (m, 1H), 7.28-7.34 (m, 2H), 7.41-7.47 (m, 2H)

### Example 17

### 1-[1-(4-tert-Butylbenzoyl)piperidin-4-yl]-4,5-dimethyl-1,2,3,6-tetrahydropyridine

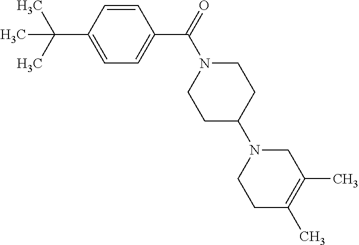 [SMILES: CC1=C(C)CN(C2CCN(C(=O)c3ccc(C(C)(C)C)cc3)CC2)CC1]

150 mg (0.58 mmol) of 1-[(4-tert-butylphenyl)carbonyl]piperidin-4-one were initially charged in 3 ml of 10% strength glacial acetic acid/methanol solution, and 97 mg (0.87 mmol) of 4,5-dimethyl-1,2,3,6-tetrahydropyridine were added. After one hour of stirring at RT, 77 mg (1.16 mmol) of sodium cyanoborohydride were added, and the mixture was stirred at RT overnight. The mixture was concentrated. The reaction mixture was taken up in ethyl acetate and washed with saturated sodium bicarbonate solution and saturated sodium chloride solution. The organic phase was dried over sodium sulphate, filtered and concentrated. The product was purified by flash chromatography on silica gel (elution ethyl acetate, then gradient ethyl acetate/methanol 5/1). The product-containing fractions were concentrated and dried under HV. This gave 18 mg (9% of theory) of an oil.

LC-MS [Method 1]: Rₜ=0.82 min; MS (ESIpos): m/z=355 (M+H)⁺

¹H-NMR (400 MHz, DMSO-d₆): δ [ppm]=1.2-1.5 (m, 2H), 1.29 (s, 9H), 1.53 (s, 3H), 1.6-1.9 (m, 2H), 1.57 (s, 3H), 1.62-1.98 (m, 1H), 1.91-1.99 (m, 2H), 2.25-3.1 (m, 1H), 2.73-3.07 (m, 2H), 2.85 (br. s, 2H), 3.45-3.75 (m, 1H), 4.3-4.55 (m, 1H), 7.27-7.35 (m, 2H), 7.42-7.49 (m, 2H)

### Example 18

### (4-tert-Butylphenyl) [3-(5-methyl-1,3,4-oxadiazol-2-yl)-1,4′-bipiperidin-1′-yl]methanone

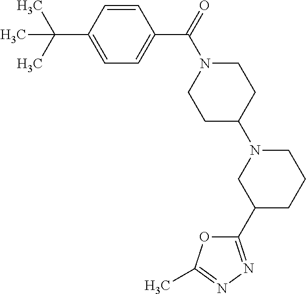 [SMILES: Cc1nnc(C2CCCN(C3CCN(C(=O)c4ccc(C(C)(C)C)cc4)CC3)C2)o1]

63 mg (0.16 mmol) of 1′-[(4-tert-butylphenyl)carbonyl]-1,4′-bipiperidine-3-carbohydrazide were initially charged in 2 ml of glacial acetic acid, and 823 mg (5.36 mmol) of phosphoryl chloride were added. The mixture was stirred at RT overnight. To bring the reaction to completion, the mixture was heated at 120° C. for 1 h. After cooling, the reaction mixture was, with ice cooling, poured into dilute aqueous sodium hydroxide solution and taken up in ethyl acetate. The organic phase was washed with saturated sodium bicarbonate solution and saturated sodium chloride solution, dried over sodium sulphate and concentrated. The residue was separated by flash chromatography on silica gel (elution:ethyl acetate/methanol 10/1). The product-containing fractions gave, after concentration and drying under HV, 41 mg (60% of theory) of an oil.

LC-MS [Method 4]: Rₜ=1.44 min; MS (ESIpos): m/z=411 (M+H)⁺

¹H-NMR (400 MHz, DMSO-d₆): δ [ppm]=1.29 (s, 9H), 1.32-1.48 (m, 2H), 1.54 (m, 2H), 1.64-1.78 (m, 2H), 1.92-2.03 (m, 1H), 2.23-2.34 (m, 1H), 2.45 (s, 3H), 2.4-2.6 (m, 4H), 2.6-3.2 (m, 1H), 2.72-2.78 (m, 1H), 2.98-3.07 (m, 2H), 3.5-3.8 (m, 1H), 4.35-4.6 (m, 1H), 7.28-7.34 (m, 2H), 7.41-7.47 (m, 2H)

### Example 19

### (4-tert-Butylphenyl)[3-(methoxymethyl)-1,4′-bipiperidin-1′-yl]methanone

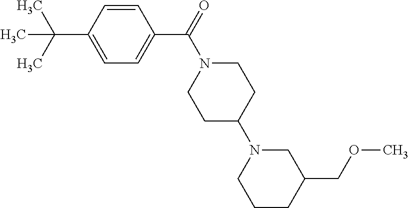 [SMILES: COCC1CCCN(C2CCN(C(=O)c3ccc(C(C)(C)C)cc3)CC2)C1]

80 mg (0.31 mmol) of 1-[(4-tert-butylphenyl)carbonyl]piperidin-4-one were initially charged in 2.5 ml of 10% strength glacial acetic acid/methanol solution, and 60 mg (0.46 mmol) of 3-(methoxymethyl)piperidine were added. After one hour of stirring at RT, 41 mg (0.62 mmol) of sodium cyanoborohydride were added, and the mixture was stirred at RT overnight. The reaction mixture was taken up in ethyl acetate and extracted with saturated sodium bicarbonate solution and saturated sodium chloride solution. The organic phase was dried over sodium sulphate, filtered and concentrated. The product was purified by flash chromatography on silica gel (elution: ethyl acetate, gradient ethyl acetate/methanol 5/1). The product-containing fractions were concentrated. The resulting residue was crystallized with ethyl acetate and filtered off with suction. After drying in the air, 5 mg (4% of theory) of a solid were obtained.

LC-MS [Method 3]: Rₜ=0.97 min; MS (ESIpos): m/z=373 (M+H)⁺

¹H-NMR (400 MHz, DMSO-d₆): δ [ppm]=1.1-1.3 (m, 1H), 1.3 (s, 3H), 1.5-1.9 (m, 6H), 1.95-2.25 (m, 2H), 2.5-3.2 (m, 2H), 2.65-2.95 (m, 2H), 3.15-4.55 (m, 8H), 3.22 (s, 3H), 3.5-3.95 (m, 1H), 4.45-4.8 (m, 1H), 7.3-7.4 (m, 2H), 7.4-7.5 (m, 2H), 9.55-9.75 (m, 1H)

### Example 20

### cis-1-[1-(4-tert-Butylbenzoyl)piperidin-4-yl]-3,4-dimethylpiperidine trifluoroacetic acid salt

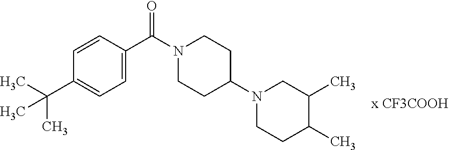 [SMILES: CC1CCN(C2CCN(C(=O)c3ccc(C(C)(C)C)cc3)CC2)CC1C]

75 mg (0.16 mmol) of (4-tert-butylphenyl)[4-(4,5-dimethyl-3,6-dihydropyridin-1(2H)-yl)piperidin-1-yl]methanone trifluoroacetate were dissolved in ethanol and hydrogenated using an H-Cube (catalyst: Pd/C 10%, solvent: ethanol, cartridge pressure: 1 bar, flow rate: 1 ml/min, temperature: 70° C.). The reaction solution was concentrated and dried under HV. This gave 67 mg (97% of theory) of an oil.

LC-MS [Method 3]: Rₜ=0.96 min; MS (ESIpos): m/z=357 (M+H)⁺

¹H-NMR (400 MHz, DMSO-d₆): δ [ppm]=0.85-0.95 (m, 3H), 0.86 (s, 2H), 1.06 (t, 1H), 1.1-1.35 (m, 2H), 1.17 (t, 1H), 1.30 (s, 9H), 1.56-1.71 (m, 2H), 1.65 (d, 4H), 1.99-2.33 (m, 2H), 2.24-2.27 (m, 1H), 3.4-3.57 (m, 5H), 7.32-7.37 (m, 2H), 7.45-7.49 (m, 2H)

### Example 21

### 1-[1-(4-tert-Butylbenzoyl)piperidin-4-yl]-3-(3-methyl-1H-1,2,4-triazol-5-yl)piperidine trifluoroacetic acid salt

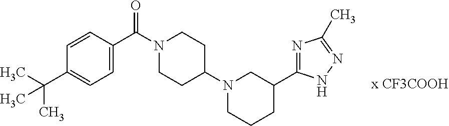 [SMILES: Cc1n[nH]c(C2CCCN(C3CCN(C(=O)c4ccc(C(C)(C)C)cc4)CC3)C2)n1]

Under argon, 19 mg (0.48 mmol) of sodium hydride (60% in mineral oil) and 36 mg (0.38 mmol) of acetamidine hydrochloride were added to 2 ml of methanol. After 20 minutes of stirring at RT, the mixture was filtered through a microfilter. This solution was added dropwise to 123 mg (0.37 mmol) of 1′-[(4-tert-butylphenyl)carbonyl]-1,4′-bipiperidine-3-carbohydrazide dissolved in 1 ml of methanol. After one hour of heating at 120° C. in a microwave, the mixture was separated by preparative HPLC [Reprosil, C18 10 μm, 250 mm×30 mm, methanol/water (with 0.05% trifluoroacetic acid) 30:70 to 100:0 over a run time of 23 min]. After HPLC control, the product-containing fractions were combined and freeze-dried. This gave 53 mg (30% of theory) of an oil.

LC-MS [Method 3]: Rₜ=1.46 min; MS (ESIpos): m/z=410 (M−CF₃COOH+H)⁺

¹H-NMR (400 MHz, DMSO-d₆): δ [ppm]=1.3 (s, 9H), 1.5-1.75 (m, 3H), 1.75-1.95 (m, 1H), 1.9-2.1 (m, 4H), 2.3 (s, 3H), 2.65-3.3 (m, 6H), 3.4-4.0 (m, 5H), 7.3-7.4 (m, 2H), 7.4-7.5 (m, 2H), 9.5 (br.s., 1H)

### Example 22

### Methyl 4-[(3-methyl-1,4′-bipiperidin-1′-yl)carbonyl]benzoate

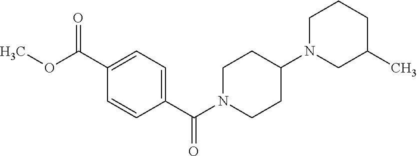

2.7 g (15 mmol) of 4-(methoxycarbonyl)benzoic acid, 2.75 g (17.95 mmol) of HOBT, 3.44 g (17.95 mmol) of EDC and 7.82 ml (25 mmol) of N,N-diisopropylethylamine were dissolved in 60 ml of DMF, and the mixture was stirred at RT for 1 h. 3.0 g (16.45 mmol) of 4-(4-methylpiperidin-1-yl)piperidine were then added, and the mixture was stirred at RT overnight. The reaction mixture was allowed to stand at RT for 2 days. The mixture was poured into water and extracted with ethyl acetate. The organic phase was separated off, dried over magnesium sulphate and filtered, and the filtrate was concentrated. The residue was purified by preparative HPLC [Reprosil C18, 10 μm, 250 mm×40 mm (15% methanol/85% water (isocratic to 15 min) then gradient to 100% methanol) over a run time of 35 min]. After HPLC control, the product-containing fractions were combined and concentrated. The residue was dried under HV. This gave 1.7 g (32% of theory) of an oil.

LC-MS [Method 1]: Rₜ=0.59 min; MS (ESIpos): m/z=345 (M+H)⁺

¹H-NMR (400 MHz, DMSO-d₆): δ [ppm]=0.82 (d, 3H), 0.71-0.94 (m, 1H), 1.28-1.86 (m, 10H), 1.98-2.14 (m, 1H), 2.65-2.84 (m, 4H), 2.93-3.06 (m, 1H), 3.42-3.55 (m, 1H), 3.87 (s, 3H), 4.43-4.97 (m, 1H), 7.46-7.58 (m, 2H), 7.96-8.06 (m, 2H).

### Example 23

### (3-Methyl-1,4′-bipiperidin-1′-yl){4-[(3-methyloxetan-3-yl)methyl]phenyl}methanone

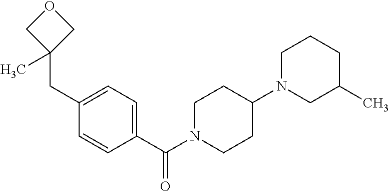

150 mg (0.64 mmol) of 4-[(3-methyloxetan-3-yl)methyl]benzoic acid (89% pure) were dissolved in 2 ml of DMF, and 99 mg (0.64 mmol) of HOBT, 124 mg (0.64 mmol) of EDC and 0.45 ml (2.6 mmol) of N,N-diisopropylethylamine were added. The mixture was stirred at RT for 15 minutes, and 118 mg (0.64 mmol) of 4-(3-methylpiperidin-1-yl)piperidine were then added. The mixture was stirred at RT overnight. The mixture was subsequently diluted with ethyl acetate and washed first with water and then with saturated sodium chloride solution. The organic phase was separated off, dried over magnesium sulphate and filtered, and the filtrate was concentrated. The residue was purified by preparative HPLC. [Reprosil C18, 10 μm, 250 mm×30 mm (10% acetonitrile/90% water to 95% acetonitrile/5% water) over a run time of 38 min]. The product-containing fractions were combined and concentrated. This gave 102 mg (43% of theory) of a solid.

LC-MS [Method 1]: Rₜ=0.64 min; MS (ESIpos): m/z=371 (M+H)⁺

¹H-NMR (400 MHz, DMSO-d₆): δ [ppm]=0.75-0.87 (m, 4H, including d, 3H), 1.19 (s, 3H), 1.30-1.46 (m, 3H), 1.46-1.58 (m, 2H), 1.58-1.70 (m, 2H), 1.71-1.80 (m, 2H), 2.01-2.11 (m, 1H), 2.43-2.48 (m, 1H), 2.64-2.71 (m, 1H), 2.69-2.79 (m, 2H), 2.93 (s, 2H), 2.95-3.05 (m, 1H), 3.49-3.71 (m, 1H), 4.16-4.21 (m, 2H), 4.37-4.50 (m, 1H), 4.50-4.55 (m, 2H), 7.21-7.25 (m, 2H), 7.27-7.32 (m, 2H)

### Example 24

### Ethyl 1′-{4-[(3-methyloxetan-3-yl)methyl]benzoyl}-1,4′-bipiperidine-3-carboxylate

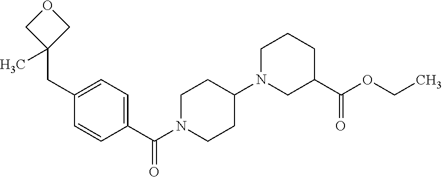 [SMILES: CCOC(=O)C1CCCN(C2CCN(C(=O)c3ccc(CC4(C)COC4)cc3)CC2)C1]

150 mg (0.64 mmol) of 4-[(3-methyloxetan-3-yl)methyl]benzoic acid (89% pure) were dissolved in 2 ml of DMF, and 111 mg (0.73 mmol) of HOBT, 139 mg (0.73 mmol) of EDC and 0.63 ml (3.6 mmol) of N,N-diisopropylethylamine were added. The mixture was stirred at RT for 1 h, and 191 mg (0.64 mmol) of ethyl 1,4′-bipiperidine-3-carboxylate dihydrochloride were then added. The mixture was stirred at RT overnight. The mixture was subsequently diluted with ethyl acetate and washed first with water and then with saturated sodium chloride solution. The organic phase was separated off, dried over magnesium sulphate and filtered, and the filtrate was concentrated. The residue was purified by preparative HPLC. [Reprosil C18, 10 μm, 250 mm×30 mm (10% acetonitrile/90% water to 95% acetonitrile/5% water) over a run time of 54 min]. The product-containing fractions were combined, concentrated and dried under HV. This gave 81 mg (29% of theory) of an oil.

LC-MS [Method 4]: Rₜ=1.27 min; MS (ESIpos): m/z=429 (M+H)⁺

¹H-NMR (400 MHz, DMSO-d₆): δ [ppm]=1.13-1.22 (m, 6H), 1.32-1.45 (m, 4H), 1.59-1.80 (m, 4H), 2.17-2.27 (m, 1H), 2.31-2.34 (m, 1H), 2.35-2.47 (m, 2H), 2.55-2.59 (m, 1H), 2.61-2.72 (m, 1H), 2.80-2.87 (m, 1H), 2.93 (s, 2H), 3.46-3.75 (m, 1H), 4.05 (q, 2H), 4.17-4.21 (m, 2H), 4.39-4.51 (m, 1H), 4.51-4.54 (m, 2H), 7.21-7.25 (m, 2H), 7.28-7.32 (m, 2H)

### Example 25

### (3-Methyl-1,4′-bipiperidin-1′-yl) [4-(3-methyloxetan-3-yl)phenyl]methanone

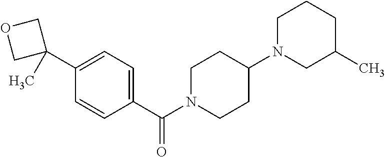 [SMILES: CC1CCCN(C2CCN(C(=O)c3ccc(C4(C)COC4)cc3)CC2)C1]

60 mg (0.26 mmol) of 3-(4-bromophenyl)-3-methyloxetane, 48 mg (0.26 mmol) of 4-(3-methylpiperidin-1-yl)piperidine, 35 mg (0.13 mmol) of molybdenum hexacarbonyl, 12 mg (0.013 mmol) of trans-bis(acetate)bis[o-(di-o-tolylphosphine)benzyl]dipalladium(II) (Herrmann's palladacycle) and 84 mg (0.79 mmol) of sodium carbonate were suspended in 0.5 ml of water and heated in a microwave at 130° C. for 5 minutes. After cooling, the mixture was diluted with 2 ml of water and extracted with ethyl acetate. The organic phase was separated off, dried over magnesium sulphate and filtered, and the filtrate was concentrated. The residue was purified by preparative HPLC. [Reprosil C18, 10 μm, 250 mm×30 mm (10% acetonitrile/90% water to 95% acetonitrile/5% water) over a run time of 54 min]. The product-containing fractions were combined, concentrated and dried under HV. This gave 20.4 mg (20% of theory) of an oil.

LC-MS [Method 4]: Rₜ=1.12 min; MS (ESIpos): m/z=357 (M+H)⁺

¹H-NMR (400 MHz, DMSO-d₆): δ [ppm]=0.76-0.87 (m, 4H, including d, 3H), 1.32-1.45 (m, 3H), 1.56-1.58 (m., 3H), 1.59-1.67 (m, 4H), 1.70-1.82 (m, 3H), 1.99-2.12 (m, 1H), 2.73-2.77 (m, 1H), 2.71-2.81 (m, 2H), 2.93-3.09 (m, 1H), 3.47-3.75 (m, 1H), 4.33-4.51 (m, 1H), 4.54-4.57 (m, 2H), 4.79-4.82 (m, 2H), 7.28-7.32 (m, 2H), 7.35-7.38 (m, 2H)

### Example 26

### Ethyl 1′-[4-(3-methyloxetan-3-yl)benzoyl]-1,4′-bipiperidine-3-carboxylate

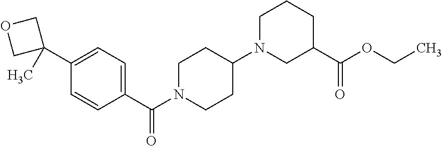 [SMILES: CCOC(=O)C1CCCN(C2CCN(C(=O)c3ccc(C4(C)COC4)cc3)CC2)C1]

100 mg (0.44 mmol) of 3-(4-bromophenyl)-3-methyloxetane, 276 mg (0.88 mmol) of ethyl 1,4′-bipiperidine-3-carboxylate dihydrochloride, 58 mg (0.22 mmol) of molybdenum hexacarbonyl, 21 mg (0.022 mmol) of trans-bis(acetate)bis[o-(di-o-tolylphosphine)benzyl]dipalladium(II) (Herrmann's palladacycle) and 233 mg (2.2 mmol) of sodium carbonate were suspended in 1 ml of water and heated in a microwave at 130° C. for 5 minutes. After cooling, the mixture was diluted with 2 ml of water and extracted with ethyl acetate. The organic phase was separated off, dried over magnesium sulphate and filtered, and the filtrate was concentrated. The residue was purified by preparative HPLC. [Reprosil C18, 10 μm, 250 mm×30 mm (10% acetonitrile/90% water to 95% acetonitrile/5% water) over a run time of 34 min]. The product-containing fractions were combined, concentrated and dried under HV. This gave 50 mg (27% of theory) of an oil.

LC-MS [Method 4]: Rₜ=1.21 min; MS (ESIpos): m/z=415 (M+H)⁺

¹H-NMR (400 MHz, DMSO-d₆): δ [ppm]=1.17 (t, 3H), 1.37-1.44 (m, 4H), 1.63 (s, 4H), 1.71-1.79 (m, 2H), 2.18-2.27 (m, 1H), 2.30-2.47 (m, 2H), 2.63-2.69 (m, 2H), 2.81-2.87 (m, 1H), 2.94-3.05 (m, 1H), 3.52-3.72 (m, 1H), 4.05 (q, 2H), 4.37-4.53 (m, 1H), 4.54-4.57 (m, 2H), 4.79-4.82 (m, 2H), 7.28-7.32 (m, 2H), 7.35-7.39 (m, 2H)

### Example 27

### 1-{1-[4-(1-Ethoxy-2-methyl-1-oxopropan-2-yl)benzoyl]piperidin-4-yl}-3-methylpiperidine trifluoroacetic acid salt

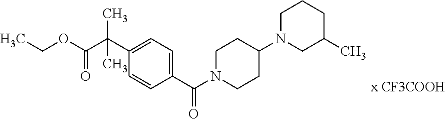 [SMILES: CCOC(=O)C(C)(C)c1ccc(C(=O)N2CCC(N3CCCC(C)C3)CC2)cc1]

300 mg (1.1 mmol) of ethyl 2-(4-bromophenyl)-2-methylpropanoate, 403 mg (2.2 mmol) of 4-(3-methylpiperidin-1-yl)piperidine, 146 mg (0.55 mmol) of molybdenum hexacarbonyl, 52 mg (0.055 mmol) of trans-bis(acetate)bis[o-(di-o-tolylphosphine)benzyl]dipalladium(II) (Herrmann's palladacycle) and 586 mg (5.5 mmol) of sodium carbonate were suspended in 3 ml of water and heated in a microwave at 150° C. for 10 minutes. After cooling, the mixture was extracted with ethyl acetate and then filtered. The organic phase was removed from the filtrate, dried over magnesium sulphate and filtered, and the filtrate was concentrated. The residue was purified by preparative HPLC. [Reprosil C18, 10 μm, 250 mm×30 mm (50% methanol/50% water (+0.05% trifluoroacetic acid) to 70% methanol/30% water (+0.05% trifluoroacetic acid)) over a run time of 25 min]. The product-containing fractions were combined, concentrated and dried under HV. This gave 254 mg (45% of theory) of an oil.

LC-MS [Method 1]: Rₜ=0.73 min; MS (ESIpos): m/z=401 (M−CF₃COOH+H)⁺

¹H-NMR (400 MHz, DMSO-d₆): δ [ppm]=0.91 (d, 3H), 1.07-1.12 (m, 1H), 1.13 (t, 3H), 1.51 (s, 6H), 1.57-1.77 (m, 4H), 1.77-1.91 (m, 2H), 1.92-2.18 (m, 2H), 2.55-2.65 (m, 2H), 2.75-2.94 (m, 2H), 2.95-3.13 (m, 1H), 3.36-3.52 (m, 2H), 3.55-3.90 (m, 1H), 4.08 (q, 2H), 4.37-4.79 (m, 1H), 7.39 (s, 4H), 9.15 (m, 1H)

### Example 28

### 1-{1-[4-(1-Hydroxy-2-methylpropan-2-yl)benzoyl]piperidin-4-yl}-3-methylpiperidine trifluoroacetic acid salt

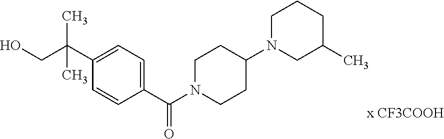 [SMILES: CC1CCCN(C2CCN(C(=O)c3ccc(C(C)(C)CO)cc3)CC2)C1]

301 mg (0.58 mmol) of 1-{1-[4-(1-ethoxy-2-methyl-1-oxopropan-2-yl)benzoyl]piperidin-4-yl}-3-methylpiperidine trifluoroacetic acid salt were dissolved in 10 ml of ethanol. At RT, 44 mg (1.17 mmol) of sodium borohydride were added, and the mixture was stirred for 3 h. The mixture was warmed up to 50° C. and stirred overnight. After cooling, the reaction mixture was acidified with 1N hydrochloric acid and extracted with ethyl acetate. The organic phase was separated off, dried over magnesium sulphate and filtered, and the filtrate was concentrated. The residue was purified by preparative HPLC. [Reprosil C18, 10 μm, 250 mm×30 mm (50% methanol/50% water (+0.05% trifluoroacetic acid) to 70% methanol/30% water (+0.05% trifluoroacetic acid)) over a run time of 25 min]. The product-containing fractions were combined, concentrated and dried under HV. This gave 67 mg (24% of theory, purity: 97%) of a foam.

LC-MS [Method 3]: Rₜ=0.67 min; MS (ESIpos): m/z=359 (M−CF₃COOH+H)⁺

¹H-NMR (400 MHz, DMSO-d₆): δ [ppm]=0.90 (d, 3H), 1.01-1.14 (m, 1H), 1.24 (s, 6H), 1.60-1.95 (m, 5H), 1.97-2.22 (m, 3H), 2.75-2.87 (m, 1H), 3.27-3.42 (m, 3H), 4.38-4.52 (m, 3H), 7.31-7.35 (m, 2H), 7.42-7.46 (m, 2H), 10.39-10.53 (m, 1H) More product was obtained by analogous purification of the aqueous phase by preparative HPLC. This gave 62 mg (20% of theory; purity: 88%) of product.

LC-MS [Method 2]: Rₜ=0.61 min; MS (ESIpos): m/z=359 (M−CF₃COOH+H)⁺

### Example 29

### 3-(Ethoxycarbonyl)-1-{1-[4-(1-ethoxy-2-methyl-1-oxopropan-2-yl)benzoyl]piperidin-4-yl}piperidine trifluoroacetic acid salt

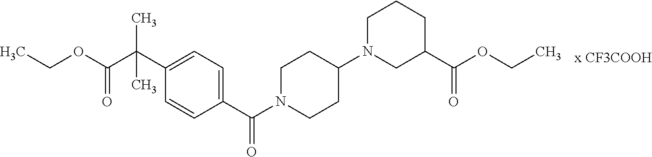 [SMILES: CCOC(=O)C1CCCN(C2CCN(C(=O)c3ccc(C(C)(C)C(=O)OCC)cc3)CC2)C1]

300 mg (1.1 mmol) of ethyl 2-(4-bromophenyl)-2-methylpropanoate, 403 mg (1.3 mmol) of ethyl 1,4′-bipiperidine-3-carboxylate dihydrochloride, 146 mg (0.55 mmol) of molybdenum hexacarbonyl, 52 mg (0.055 mmol) of trans-bis(acetate)bis[o-(di-o-tolylphosphine)benzyl]dipalladium(II) (Herrmann's palladacycle) and 586 mg (5.5 mmol) of sodium carbonate were suspended in 3 ml of water and heated in a microwave at 150° C. for 10 minutes. After cooling, the mixture was extracted with ethyl acetate and then filtered. The organic phase was removed from the filtrate, dried over magnesium sulphate and filtered, and the filtrate was concentrated. The residue was purified by preparative HPLC. [Reprosil C18, 10 μm, 250 mm×30 mm (50% methanol/50% water (+0.05% trifluoroacetic acid) to 70% methanol/30% water (+0.05% trifluoroacetic acid)) over a run time of 25 min]. The product-containing fractions were combined, concentrated and dried under HV. This gave 237 mg (37% of theory) of an oil.

LC-MS [Method 4]: Rₜ=1.46 min; MS (ESIpos): m/z=459 (M−CF₃COOH+H)⁺

¹H-NMR (400 MHz, DMSO-d₆): δ [ppm]=1.13 (t, 3H), 1.21 (t, 3H), 1.52 (s, 6H), 1.60-1.76 (m, 3H), 1.89-2.06 (m, 4H), 2.61-3.43 (m, 6H), 3.50-3.57 (m, 2H), 4.04-4.19 (m, 4H), 4.42-4.79 (m, 1H), 7.39 (s, 4H), 9.25-9.55 (m, 1H)

### Example 30

### 1-(1-{4-[1-(Ethoxycarbonyl)cyclopropyl]benzoyl}piperidin-4-yl)-3-methylpiperidine trifluoroacetic acid salt

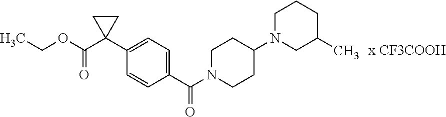 [SMILES: CCOC(=O)C1(c2ccc(C(=O)N3CCC(N4CCCC(C)C4)CC3)cc2)CC1]

600 mg (2.2 mmol) of ethyl 1-(4-bromophenyl)cyclopropanecarboxylate, 813 mg (4.5 mmol) of 4-(3-methylpiperidin-1-yl)piperidine, 294 mg (1.12 mmol) of molybdenum hexacarbonyl, 105 mg (0.11 mmol) of trans-bis(acetate)bis[o-(di-o-tolylphosphine)benzyl]dipalladium(II) (Herrmann's palladacycle) and 709 mg (6.7 mmol) of sodium carbonate were suspended in 3 ml of water and heated in a microwave at 150° C. for 10 minutes. After cooling, the mixture was extracted with ethyl acetate and then filtered. The organic phase was removed from the filtrate, dried over magnesium sulphate and filtered, and the filtrate was concentrated. The residue was purified by preparative HPLC. [Reprosil C18, 10 μm, 250 mm×30 mm (50% methanol/50% water (+0.05% trifluoroacetic acid) to 70% methanol/30% water (+0.05% trifluoroacetic acid)) over a run time of 25 min]. The product-containing fractions were combined, concentrated and dried under HV. This gave 348 mg (30% of theory) of an oil.

LC-MS [Method 2]: Rₜ=0.74 min; MS (ESIpos): m/z=399 (M−CF₃COOH+H)⁺

¹H-NMR (400 MHz, DMSO-d₆): δ [ppm]=0.88-0.94 (m, 3H), 1.11 (t, 3H), 1.20-1.26 (m, 2H), 1.49-1.55 (m, 2H), 1.59-2.19 (m, 8H), 2.58-3.14 (m, 5H), 3.33-3.55 (m, 3H), 3.58-3.88 (m, 1H), 4.04 (q, 2H), 4.29-4.81 (m, 1H), 7.32-7.38 (m, 2H), 7.39-7.43 (m, 2H), 8.98-9.38 (m, 1H)

### Example 31

### 1-(1-{4-[1-(Hydroxymethyl)cyclopropyl]benzoyl}piperidin-4-yl)-3-methylpiperidine trifluoroacetic acid salt

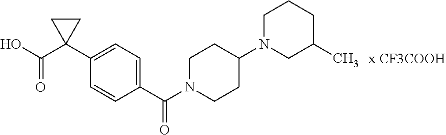 [SMILES: CC1CCCN(C2CCN(C(=O)c3ccc(C4(C(=O)O)CC4)cc3)CC2)C1]

225 mg (0.44 mmol) of 1-(1-{4-[1-(ethoxycarbonyl)cyclopropyl]benzoyl}piperidin-4-yl)-3-methylpiperidine trifluoroacetic acid salt were dissolved in 10 ml of ethanol, and 83 mg (2.2 mmol) of sodium borohydride were added. The mixture was stirred at RT overnight, and a further 17 mg (0.44 mmol) of sodium borohydride were then added and the mixture was warmed to 50° C. The mixture was stirred overnight at 50° C. Another 17 mg (0.44 mmol) of sodium borohydride were then added, and the mixture was stirred at 70° C. overnight. The mixture was cooled, 1N hydrochloric acid was added and the mixture was extracted with ethyl acetate. The organic phase was separated off, dried over magnesium sulphate and filtered, and the filtrate was concentrated. The residue was purified by preparative HPLC. [Reprosil C18, 10 μm, 250 mm×30 mm (50% methanol/50% water (+0.05% trifluoroacetic acid) to 70% methanol/30% water (+0.05% trifluoroacetic acid)) over a run time of 25 min]. The product-containing fractions were combined, concentrated and dried under HV. This gave 115 mg of an oil. This oil was dissolved in 5 ml of THF, and 24 mg (0.24 mmol) of triethylamine were added. At −10° C., 26 mg (0.24 mmol) of ethyl chloroformate were added. After 1 h of stirring at RT, 0.96 ml (23.4 mmol) of methanol and 16 mg (0.71 mmol) of lithium borohydride were added. The mixture was then stirred at 0° C. for 1 h and at RT for 1 h. The mixture was acidified with hydrochloric acid and extracted with ethyl acetate. The organic phase was separated off, dried over magnesium sulphate, filtered and concentrated. The residue was purified by preparative HPLC [Reprosil C18, 10 μm, 250 mm×30 mm (50% methanol/50% water (+0.05% trifluoroacetic acid) to 70% methanol/30% water (+0.05% trifluoroacetic acid)) over a run time of 25 min]. The product-containing fractions were combined, concentrated and dried under HV. This gave 25 mg (11% of theory) of a foam.

LC-MS [Method 1]: Rₜ=0.57 min; MS (ESIpos): m/z=357 (M−CF₃COOH+H)⁺

¹H-NMR (400 MHz, DMSO-d₆): δ [ppm]=0.75-0.79 (m, 2H), 0.86-0.92 (m, 5H), 1.02-1.22 (m, 2H), 1.57-1.88 (m, 6H), 1.90-2.25 (m, 4H), 2.71-2.91 (m, 2H), 2.96-3.11 (m, 1H), 3.37-3.47 (m, 2H), 3.54-3.57 (m, 2H), 4.45-4.80 (m, 2H), 7.29-7.33 (m, 2H), 7.34-7.37 (m, 2H), 9.99-10.20 (m, 1H)

### Example 32

### 1-{1-[4-(Ethoxycarbonyl)benzoyl]piperidin-4-yl}-3-methylpiperidine trifluoroacetic acid salt

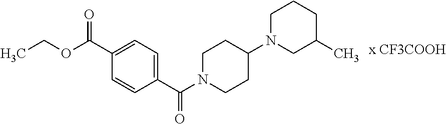 [SMILES: CCOC(=O)c1ccc(C(=O)N2CCC(N3CCCC(C)C3)CC2)cc1]

600 mg (2.6 mmol) of ethyl 4-bromobenzoate, 478 mg (2.6 mmol) of 4-(3-methylpiperidin-1-yl)piperidine, 346 mg (1.3 mmol) of molybdenum hexacarbonyl, 123 mg (0.13 mmol) of trans-bis(acetate)bis[o-(di-o-tolylphosphine)benzyl]dipalladium(II) (Herrmann's palladacycle) and 832.8 mg (7.9 mmol) of sodium carbonate were suspended in 3 ml of water and heated in a microwave at 150° C. for 10 minutes. After cooling, the mixture was extracted with ethyl acetate and then filtered through kieselguhr. The organic phase was removed from the filtrate, dried over magnesium sulphate and filtered, and the filtrate was concentrated. The residue was purified by preparative HPLC. [Reprosil C18, 10 μm, 250 mm×30 mm (50% methanol/50% water (+0.05% trifluoroacetic acid) to 70% methanol/30% water (+0.05% trifluoroacetic acid)) over a run time of 25 min]. The product-containing fractions were combined, concentrated and dried under HV. This gave 370 mg (30% of theory) of the title compound.

LC-MS [Method 1]: Rₜ=0.67 min; MS (ESIpos): m/z=359 (M−CF₃COOH+H)⁺

¹H-NMR (400 MHz, DMSO-d₆): δ [ppm]=0.91 (d, 3H), 1.02-1.17 (m, 1H), 1.33 (t, 3H), 1.57-1.98 (m, 7H), 2.05-2.15 (m, 1H), 2.56-2.69 (m, 1H), 2.74-2.95 (m, 2H), 2.99-3.19 (m, 1H), 3.36-3.52 (m, 3H), 3.54-3.68 (m, 1H), 4.34 (q, 2H), 4.55-4.71 (m, 1H), 7.53-7.58 (m, 2H), 8.00-8.05 (m, 2H), 9.05-9.21 (m, 1H)

### Example 33

### 1-{1-[4-(2-Ethoxy-2-oxoethyl)benzoyl]piperidin-4-yl}-3-methylpiperidine trifluoroacetic acid salt

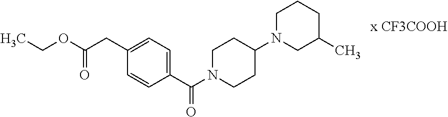 [SMILES: CCOC(=O)Cc1ccc(C(=O)N2CCC(N3CCCC(C)C3)CC2)cc1]

100 mg (0.41 mmol) of ethyl (4-bromophenyl)acetate, 150 mg (0.82 mmol) of 4-(3-methylpiperidin-1-yl)piperidine, 54 mg (0.21 mmol) of molybdenum hexacarbonyl, 19 mg (0.02 mmol) of trans-bis(acetate)bis[o-(di-o-tolylphosphine)benzyl]dipalladium(II) (Herrmann's palladacycle) and 131 mg (1.23 mmol) of sodium carbonate were suspended in 1 ml of water and heated in a microwave at 150° C. for 15 minutes. After cooling, the mixture was extracted with ethyl acetate and then filtered through kieselguhr. The organic phase was removed from the filtrate, dried over magnesium sulphate and filtered, and the filtrate was concentrated. The residue was purified by preparative HPLC. [Reprosil C18, 10 μm, 250 mm×30 mm (50% methanol/50% water (+0.05% trifluoroacetic acid) to 70% methanol/30% water (+0.05% trifluoroacetic acid)) over a run time of 25 min]. The product-containing fractions were combined, concentrated and dried under HV. This gave 145 mg (68% of theory) of a foam.

LC-MS [Method 1]: Rₜ=0.66 min; MS (ESIpos): m/z=373 (M−CF₃COOH+H)⁺

¹H-NMR (400 MHz, DMSO-d₆): δ [ppm]=0.91 (d, 3H), 1.01-1.15 (m, 1H), 1.20 (t, 3H), 1.58-1.77 (m, 4H), 1.79-1.90 (m, 2H), 1.91-2.15 (m, 2H), 2.56-2.69 (m, 2H), 2.73-2.94 (m, 2H), 2.96-3.17 (m 2H), 3.39-3.53 (m, 2H), 3.71-3.75 (m, 2H), 4.09 (q, 2H), 4.39-4.78 (m, 1H), 7.32-7.40 (m, 4H), 9.04-9.22 (m, 1H)

### Example 34

### 3-(Ethoxycarbonyl)-1-{1-[4-(2-ethoxy-2-oxoethyl)benzoyl]piperidin-4-yl}piperidine trifluoroacetic acid salt

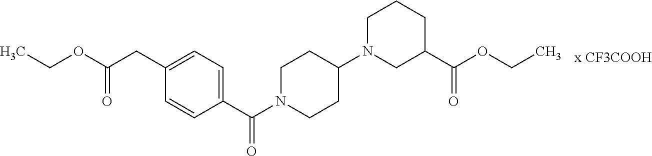 [SMILES: CCOC(=O)Cc1ccc(C(=O)N2CCC(N3CCCC(C(=O)OCC)C3)CC2)cc1]

100 mg (0.41 mmol) of ethyl (4-bromophenyl)acetate, 258 mg (0.82 mmol) of ethyl 1,4′-bipiperidine-3-carboxylate dihydrochloride, 54 mg (0.21 mmol) of molybdenum hexacarbonyl, 19 mg (0.02 mmol) of trans-bis(acetate)bis[o-(di-o-tolylphosphine)benzyl]dipalladium(II) (Herrmann's palladacycle) and 218 mg (2.06 mmol) of sodium carbonate were suspended in 1 ml of water and heated in a microwave at 150° C. for 15 minutes. After cooling, the mixture was extracted with ethyl acetate and then filtered through kieselguhr. The organic phase was removed from the filtrate, dried over magnesium sulphate and filtered, and the filtrate was concentrated. The residue was purified by preparative HPLC. [Reprosil C18, 10 μm, 250 mm×30 mm (50% methanol/50% water (+0.05% trifluoroacetic acid) to 70% methanol/30% water (+0.05% trifluoroacetic acid)) over a run time of 25 min]. The product-containing fractions were combined, concentrated and dried under HV. This gave 141 mg (62% of theory) of an oil.

LC-MS [Method 4]: Rₜ=1.26 min; MS (ESIpos): m/z=431 (M−CF₃COOH+H)⁺

¹H-NMR (400 MHz, DMSO-d₆): δ [ppm]=1.11-1.28 (m, 6H), 1.44-1.60 (m, 1H), 1.60-1.86 (m, 3H), 1.87-2.21 (m, 4H), 2.64-3.31 (m, 6H), 3.44-3.69 (m, 5H), 4.04-4.20 (m, 4H), 4.46-4.79 (m, 1H), 7.33-7.40 (m, 4H), 9.36-9.55 (m, 1H)

### Example 35

### N-Butyl-N-methyl-4-[(3-methyl-1,4′-bipiperidin-1′-yl)carbonyl]benzamide

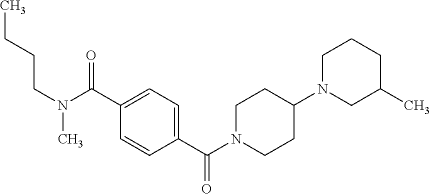 [SMILES: CCCCN(C)C(=O)c1ccc(C(=O)N2CCC(N3CCCC(C)C3)CC2)cc1]

40 mg (0.12 mmol) of 4-[(3-methyl-1,4′-bipiperidin-1′-yl)carbonyl]benzoic acid were dissolved in 5 ml of dichloromethane, and 77 mg (0.61 mmol) of oxalyl chloride were added. The reaction mixture was stirred at RT for 2 h and then concentrated and dried under HV. The residue was dissolved in 3 ml of dichloromethane and, at RT, added dropwise to an initially charged solution of 21 mg (0.24 mmol) of N-methyl-N-butylamine and 61 mg (0.61 mmol) of triethylamine in 2 ml of dichloromethane. The mixture was stirred at RT for 2 h and then concentrated and, without any further work-up, purified by preparative HPLC. [Reprosil C18, 10 μm, 250 mm×30 mm (50% methanol/50% water to 70% methanol/30% water) over a run time of 25 min]. The product-containing fractions were combined, concentrated and dried under HV. This gave 12 mg (24% of theory) of an oil.

LC-MS [Method 4]: Rₜ=1.29 min; MS (ESIpos): m/z=400 (M+H)⁺

¹H-NMR (400 MHz, DMSO-d₆): δ [ppm]=0.68-0.82 (m, 5H, including d, 3H), 0.86-0.98 (m, 2H), 1.03-1.14 (m, 1H), 1.20-1.69 (m, 11H), 1.71-1.82 (m, 2H), 2.00-2.11 (m, 1H), 2.65-2.82 (m, 3H), 2.83-3.03 (m, 3H), 3.12-3.22 (m, 1H), 3.34-3.40 (m, 1H), 3.40-3.49 (m, 1H), 3.49-3.65 (m, 1H), 4.42-4.56 (m, 1H), 7.37-7.45 (m, 4H)

### Example 36

### N-(2-Methoxyethyl)-4-[(3-methyl-1,4′-bipiperidin-1′-yl)carbonyl]benzamide

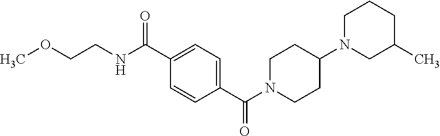

40 mg (0.12 mmol) of 4-[(3-methyl-1,4′-bipiperidin-1′-yl)carbonyl]benzoic acid were dissolved in 5 ml of dichloromethane, and 77 mg (0.61 mmol) of oxalyl chloride were added. The reaction mixture was stirred at RT for 2 h and then concentrated and dried under HV. The residue was dissolved in 3 ml of dichloromethane and, at RT, added dropwise to an initially charged solution of 18 mg (0.24 mmol) of 2-methoxyethylamine and 61 mg (0.61 mmol) of triethylamine in 2 ml of dichloromethane. The mixture was stirred at RT for 2 h and then concentrated and, without any further work-up, purified by preparative HPLC. [Reprosil C18, 10 μm, 250 mm×30 mm (50% methanol/50% water to 70% methanol/30% water) over a run time of 25 min]. The product-containing fractions were combined, concentrated and dried under HV. This gave 31 mg (66% of theory) of an oil.

LC-MS [Method 1]: Rₜ=0.48 min; MS (ESIpos): m/z=388 (M+H)⁺

¹H-NMR (400 MHz, DMSO-d₆): δ [ppm]=0.76-0.87 (m, 4H, including d, 3H), 1.31-1.46 (m, 3H), 1.47-1.69 (m, 4H), 1.70-1.85 (m, 2H), 2.00-2.10 (m, 1H), 2.43-2.48 (m, 1H), 2.69-2.80 (m, 3H), 2.92-3.07 (m, 1H), 3.24-3.28 (m, 3H), 3.39-3.49 (m, 3H), 3.38-3.56 (m, 2H), 4.42-4.57 (m, 1H), 7.41-7.49 (m, 2H), 7.85-7.92 (m, 2H), 8.56-8.64 (m, 1H)

### Example 37

### 1-(1-{4-[Ethyl-(2-methoxyethyl)carbamoyl]benzoyl}piperidin-4-yl)-3-methylpiperidine trifluoroacetic acid salt

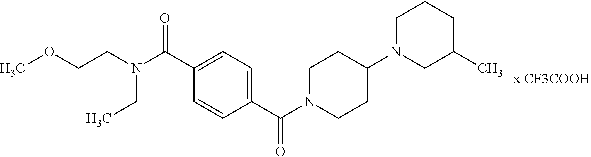

100 mg (0.3 mmol) of 4-[(3-methyl-1,4′-bipiperidin-1′-yl)carbonyl]benzoic acid were dissolved in 10 ml of DMF, and 56 mg (0.36 mmol) of HOBT, 70 mg (0.36 mmol) of EDC and 0.16 ml (0.91 mmol) of N,N-diisopropylethylamine were added. The mixture was stirred at RT for 1 h. 38 mg (0.36 mmol) of N-ethyl-2-methoxyethanamine were then added, and the mixture was stirred at RT overnight. The mixture was diluted with ethyl acetate and washed successively with water and saturated sodium chloride solution. The organic phase was separated off, dried over magnesium sulphate and filtered, and the filtrate was concentrated. The residue was purified by preparative HPLC. [Reprosil C18, 10 μm, 250 mm×30 mm (50% methanol/50% water (+0.05% trifluoroacetic acid) to 70% methanol/30% water (+0.05% trifluoroacetic acid)) over a run time of 25 min]. The product-containing fractions were combined, concentrated and dried under HV. This gave 63 mg (39% of theory) of an oil.

LC-MS [Method 1]: Rₜ=0.60 min; MS (ESIpos): m/z=416 (M−CF₃COOH+H)⁺

¹H-NMR (400 MHz, DMSO-d₆): δ [ppm]=0.86-0.94 (d, 3H), 0.99-1.19 (m, 4H), 1.60-1.76 (m, 4H), 1.77-1.91 (m, 2H), 1.92-2.16 (m, 2H), 2.57-2.72 (m, 1H), 2.73-2.94 (m, 2H), 2.97-3.36 (m, 9H), 3.61-3.78 (m, 2H), 4.41-4.78 (m, 1H), 7.35-7.52 (m, 4H), 9.03-9.24 (m, 1H)

### Example 38

### 1-{1-[4-(tert-Butylcarbamoyl)benzoyl]piperidin-4-yl}-3-methylpiperidine trifluoroacetic acid salt

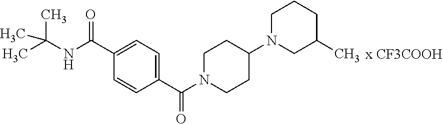 [SMILES: CC1CCCN(C2CCN(C(=O)c3ccc(C(=O)NC(C)(C)C)cc3)CC2)C1]

100 mg (0.39 mmol) of 4-bromo-N-tert-butylbenzamide, 142 mg (0.78 mmol) of 4-(3-methylpiperidin-1-yl)piperidine, 52 mg (0.2 mmol) of molybdenum hexacarbonyl, 18 mg (0.02 mmol) of trans-bis(acetate)bis[o-(di-o-tolylphosphine)benzyl]dipalladium(II) (Herrmann's palladacycle) and 124 mg (1.2 mmol) of sodium carbonate were suspended in 1 ml of water and heated in a microwave at 150° C. for 15 minutes. After cooling, the mixture was extracted with ethyl acetate and then filtered through kieselguhr. The organic phase was removed from the filtrate, dried over magnesium sulphate and filtered, and the filtrate was concentrated. The residue was purified by preparative HPLC. [Reprosil C18, 10 μm, 250 mm×30 mm (50% methanol/50% water (+0.05% trifluoroacetic acid) to 70% methanol/30% water (+0.05% trifluoroacetic acid)) over a run time of 25 min]. The product-containing fractions were combined, concentrated and dried under HV. This gave 43 mg (22% of theory) of the title compound.

LC-MS [Method 4]: Rₜ=1.23 min; MS (ESIpos): m/z=386 (M−CF₃COOH+H)⁺

¹H-NMR (400 MHz, DMSO-d₆): δ [ppm]=0.88-0.93 (m, 4H), 1.02-1.06 (m, 1H), 1.07-1.12 (m, 1H), 1.33-1.42 (m, 9H), 1.58-1.90 (m, 10H), 2.03-2.18 (m, 1H), 2.70-2.96 (m, 2H), 3.55-3.65 (m, 1H), 4.55-4.68 (m, 1H), 7.42-7.48 (m, 2H), 7.82-7.88 (m, 2H), 7.86-7.89 (m, 1H), 9.20-9.68 (m, 1H)

### Example 39

### (3-Methyl-1,4′-bipiperidin-1′-yl) [4-(1,3-oxazol-5-yl)phenyl]methanone

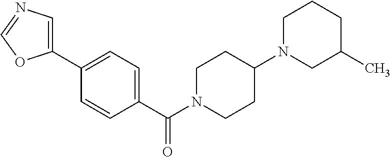 [SMILES: CC1CCCN(C2CCN(C(=O)c3ccc(-c4cnco4)cc3)CC2)C1]

100 mg (0.45 mmol) of 5-(4-bromophenyl)-1,3-oxazole, 163 mg (0.89 mmol) of 4-(3-methylpiperidin-1-yl)piperidine, 59 mg (0.22 mmol) of molybdenum hexacarbonyl, 21 mg (0.022 mmol) of trans-bis(acetate)bis[o-(di-o-tolylphosphine)benzyl]dipalladium(II) (Herrmann's palladacycle) and 142 mg (1.34 mmol) of sodium carbonate were suspended in 1 ml of water and 1 ml of 1,2-dimethoxyethane and heated in a microwave at 150° C. for 15 minutes. After cooling, the mixture was extracted with ethyl acetate and then filtered through kieselguhr. The organic phase was removed from the filtrate, dried over magnesium sulphate and filtered, and the filtrate was concentrated. The residue was purified by preparative HPLC. [Reprosil C18, 10 μm, 250 mm×30 mm (50% methanol/50% water (+0.05% trifluoroacetic acid) to 70% methanol/30% water (+0.05% trifluoroacetic acid)) over a run time of 25 min]. The product-containing fractions were combined, concentrated and dried under HV. For further purification, the product was then chromatographed on silica gel (0.04-0.063 mm/230-400 mesh ASTM), using methanol. After TLC control, the product-containing fractions were combined and concentrated. The residue was dried under HV. This gave 15 mg (10% of theory) of a solid.

LC-MS [Method 3]: Rₜ=0.67 min; MS (ESIpos): m/z=354 (M+H)⁺

¹H-NMR (400 MHz, DMSO-d₆): δ [ppm]=0.75-0.89 (m, 4H, including d, 3H), 1.12-1.92 (m, 10H), 2.01-2.11 (m, 1H), 2.71-2.81 (m, 3H), 3.01 (m, 1H), 3.49-3.71 (m, 1H), 4.53-3.70 (m, 1H), 7.46-7.52 (m, 2H), 7.75-7.81 (m, 3H, including s, 1H), 8.49 (s, 1H)

### Example 40

### 1′-(4-tert-Butylbenzoyl)-1,4′-bipiperidin-3-yl-pyrrolidin-1-carboxylate trifluoroacetic acid salt

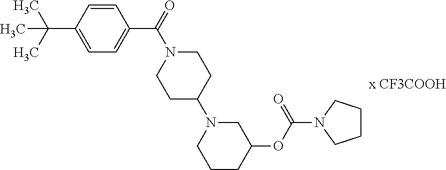 [SMILES: CC(C)(C)c1ccc(C(=O)N2CCC(N3CCCC(OC(=O)N4CCCC4)C3)CC2)cc1]

Under argon, 75 mg (0.22 mmol) of 1′-[(4-tert-butylphenyl)carbonyl]-1,4′-bipiperidin-3-yl-pyrrolidin-1-carboxylate trifluoroacetate were initially charged in THF, and 21 mg (0.54 mmol) of sodium hydride (60% in mineral oil) were added. The mixture was stirred under reflux for 1 h. After addition of 64 mg (0.48 mmol) of N-pyrrolidinecarbonyl chloride, the mixture was stirred at 60° C. overnight. After concentration, the mixture was separated by preparative HPLC. [Reprosil C18, 10 μm, 250 mm×40 mm (30% methanol/70% water (+0.05% trifluoroacetic acid) to 100% methanol) over a run time of 23 min]. The product-containing fractions were combined, concentrated and dried under HV. This gave 25 mg (21% of theory) of an oil.

LC-MS [Method 6]: Rₜ=1.62 min; MS (ESIpos): m/z=442 (M+H)⁺

¹H-NMR (400 MHz, DMSO-d₆): δ [ppm]=1.30 (s, 9H), 1.47-2.18 (m, 12H), 3.26 (m, 8H), 3.30-3.66 (m, 5H), 7.30-7.40 (m, 2H), 7.47 (m, 2H), 8.76 (br. s., 1H), 9.58 (br. s., 1H).

### Example 41

### Methyl 2-{3-fluoro-4-[(3-methyl-1,4′-bipiperidin-1′-yl)carbonyl]phenyl}-2-methylpropanoate trifluoroacetic acid salt

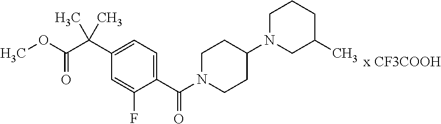

633 mg (1.59 mmol, 69% pure) of methyl 2-(4-bromo-3-fluorophenyl)-2-methylpropanoate, 210 mg (0.5 mmol) of molybdenum hexacarbonyl, 75 mg (0.08 mmol) of trans-bis(acetate)bis[o-(di-o-tolylphosphine)benzyl]dipalladium(II) (Herrmann's palladacycle) and 505 mg (4.76 mmol) of sodium carbonate were suspended in 3 ml of water and stirred in a microwave at 150° C. and 200 Watt for 10 min. After cooling, the mixture was diluted with 2 ml of water and shaken with ethyl acetate. The mixture was filtered through a little Celite. The organic phase was separated off, dried over magnesium sulphate and filtered, and the filtrate was concentrated. The residue was purified by preparative HPLC. [Reprosil C18, 10 μm, 250 mm×30 mm (50% methanol/50% water (+0.05% trifluoroacetic acid) to 100% methanol) over a run time of 25 min]. The product-containing fractions were combined, concentrated and dried under HV. This gave 65 mg (8% of theory, purity: 91%) of an oil.

LC-MS [Method 2]: Rₜ=0.67 min; MS (ESIpos): m/z=405 (M+H)⁺

¹H-NMR (400 MHz, DMSO-d₆): δ [ppm]=0.91 (d, 3H), 0.86-0.99 (m, 1H), 1.02-1.19 (m, 2H), 1.53 (s, 4H), 1.47-1.91 (m, 4H), 1.93-2.24 (m, 4H), 2.5-2.65 (m, 2H), 2.72-2.98 (m, 2H), 3.11 (t, 1H), 3.33-3.55 (m, 4H), 3.62 (s, 3H), 4.65 (d, 1H), 7.14-7.30 (m, 2H), 7.32-7.48 (m, 1H).

### Example 42

### Ethyl 1′-[4-(2-methoxypropan-2-yl)benzoyl]-1,4′-bipiperidine-3-carboxylate

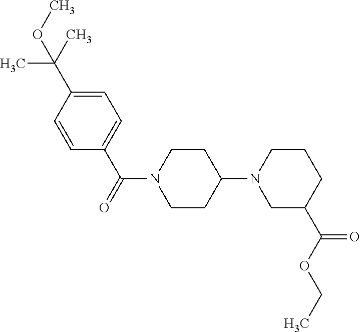 [SMILES: CCOC(=O)C1CCCN(C2CCN(C(=O)c3ccc(C(C)(C)OC)cc3)CC2)C1]

150 mg (0.77 mmol) of 4-(2-methoxypropan-2-yl)benzoic acid and 363 mg (1.16 mmol) of ethyl 1,4′-bipiperidine-3-carboxylate dihydrochloride were dissolved in 6 ml of DMF, and 499 mg (3.86 mmol) of N,N-diisopropylethylamine and 440 mg (1.16 mmol) of N-[(dimethylamino)(3H-[1,2,3]triazolo[4,5-b]pyridin-3-yloxy)methylene]-N-methylmethanaminium hexafluorophosphate were added. The reaction mixture was stirred at RT overnight. 50 ml of ethyl acetate were added, and the mixture was washed three times with in each case 20 ml of water and once with 30 ml of saturated aqueous sodium chloride solution. The organic phase was separated off, dried over sodium sulphate and then filtered and concentrated. The residue was purified by preparative HPLC [Reprosil C18, 10 μm, 250 mm×40 mm (30% methanol/70% water to 100% methanol) over a run time of 35 min]. The product-containing fractions were combined, concentrated and dried under HV. This gave 60 mg (18% of theory) of an oil.

LC-MS [Method 1]: Rₜ=0.69 min; MS (ESIpos): m/z=417 (M+H)⁺

¹H-NMR (400 MHz, DMSO-d₆): δ [ppm]=1.17 (t, 3H), 1.34-1.41 (m., 2H), 1.46 (s, 6H), 1.57-1.80 (m, 4H), 2.18-2.25 (m, 1H), 2.30-2.47 (m, 2H), 2.52-2.56 (m, 2H), 2.62-2.70 (m, 1H), 2.82-2.88 (m, 2H), 2.99 (s, 3H), 3.25 (s, 2H), 3.55-3.65 (m, 1H), 4.05 (q, 2H), 4.45-4.55 (m, 1H), 7.33-7.39 (m, 2H), 7.40-7.48 (m, 2H).

### Example 43

### N-Methyl-4-{[(3R)-3-methyl-1,4′-bipiperidin-1′-yl]carbonyl}-N-(1-phenylethyl)benzamide

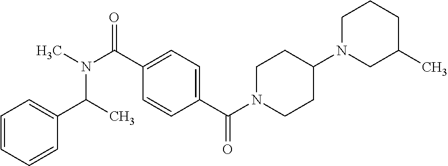 [SMILES: CC1CCCN(C2CCN(C(=O)c3ccc(C(=O)N(C)C(C)c4ccccc4)cc3)CC2)C1]

13 mg (0.1 mmol) of (1R)—N-methyl-1-phenylethanamine were initially charged in a well of a 96-well microtitre plate having deep wells, and a solution of 26 mg (0.08 mmol) of 4-[(3-methyl-1,4′-bipiperidin-1′-yl)carbonyl]benzoic acid in 0.4 ml of DMSO was added. A solution of 33 mg (0.1 mmol) of 2-(1H-benzotriazol-1-yl)-1,1,3,3-tetramethylaminium tetrafluoroborate in 0.2 ml of DMSO and 70 μl of diisopropylethylamine were added successively to this mixture. The microtitre plate was covered and shaken at RT overnight. The mixture was then filtered and the filtrate was purified directly by preparative LC-MS (MS instrument: Waters, Instrument HPLC: Waters; column Waters X-Bridge C18, 18 mm×50 mm, 5 μm, elution A: water+0.05% triethylamine, elution B: acetonitrile (ULC)+0.05% triethylamine or methanol (ULC)+0.05% triethylamine, gradient: 0.0 min 95% A→0.15 min 95% A→8.0 min 5% A→9.0 min 5% A; flow rate: 40 ml/min; UV detection: DAD; 210-400 nm). The product-containing fractions were concentrated under reduced pressure using a centrifugal dryer. The residues of the individual fractions were in each case dissolved in 0.6 ml of DMSO and combined. The solvent was then evaporated completely in a centrifugal drier. This gave 16.8 mg (44% of theory) of the target product.

LC-MS [Method 7]: Rₜ=1.38 min; MS (ESIpos): m/z=448 (M+H)⁺

The following compounds were synthesized analogously:

| | | | Yield | |
|---|---|---|---|---|
| Ex. | | | [of | |
| No. | Structure | Name | theory] | Analytical data |
| 44 | 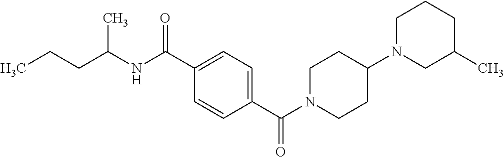 [SMILES: CCCC(C)NC(=O)c1ccc(C(=O)N2CCC(N3CCCC(C)C3)CC2)cc1] | 4-[(3-methyl-1,4′- bipiperidin-1′-yl) carbonyl]-N-(pentan- 2-yl)benzamide | 7.8 mg 24% | LC-MS [Method 7]: Rₜ = 1.32 min MS (ESIpos): m/z = 400 (M + H)⁺ |
| 45 | 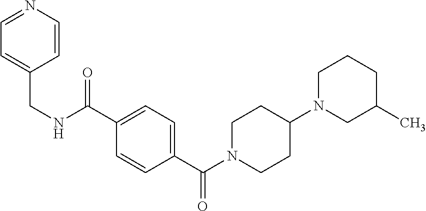 [SMILES: CC1CCCN(C2CCN(C(=O)c3ccc(C(=O)NCc4ccncc4)cc3)CC2)C1] | 4-[(3-methyl-1,4′- bipiperidin-1′-yl) carbonyl]-N-(pyridin- 4-yl-methyl) benzamide | 6.3 mg 19% | LC-MS [Method 7]: Rₜ = 0.34 min MS (ESIpos): m/z = 421 (M + H)⁺ |
| 46 | 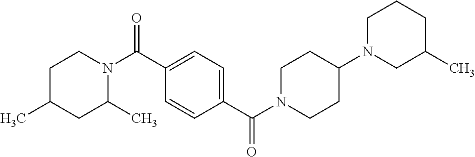 | (2,4- dimethylpiperidin- 1-yl){4-[(3-methyl- 1,4′-bipiperidin-1′- yl)carbonyl]phenyl} methanone | 16.5 mg 48% | LC-MS [Method 7]: Rₜ = 1.37 min MS (ESIpos): m/z = 426 (M + H)⁺ |
| 47 | 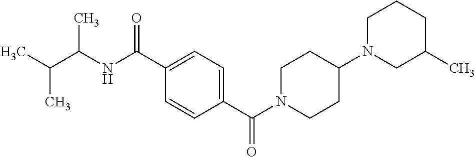 [SMILES: CC1CCCN(C2CCN(C(=O)c3ccc(C(=O)NC(C)C(C)C)cc3)CC2)C1] | 4-[(3-methyl-1,4′- bipiperidin-1′- yl)carbonyl]-N-(3- methylbutan-2- yl)benzamide | 10.3 mg 32% | LC-MS [Method 7]: Rₜ = 1.30 min MS (ESIpos): m/z = 400 (M + H)⁺ |
| 48 | 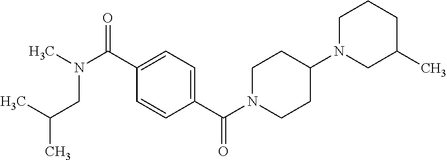 [SMILES: CC(C)CN(C)C(=O)c1ccc(C(=O)N2CCC(N3CCCC(C)C3)CC2)cc1] | N-methyl-4-[(3- methyl-1,4′- bipiperidin-1′- yl)carbonyl]-N-(2- methylpropyl) benzamide | 12.6 mg 39% | LC-MS [Method 7]: Rₜ = 1.28 min MS (ESIpos): m/z = 400 (M + H)⁺ |
| 49 | 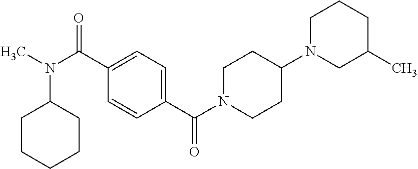 | N-cyclohexyl-N- methyl-4-[(3-methyl- 1,4′-bipiperidin-1′- yl)carbonyl] benzamide | 10.9 mg 32% | LC-MS [Method 7]: Rₜ = 1.37 min MS (ESIpos): m/z = 426 (M + H)⁺ |
| 50 | 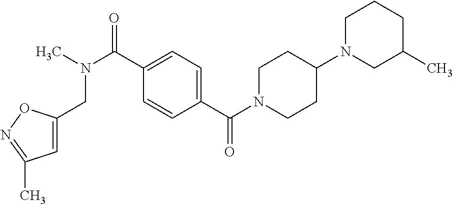 [SMILES: Cc1cc(CN(C)C(=O)c2ccc(C(=O)N3CCC(N4CCCC(C)C4)CC3)cc2)on1] | N-methyl-4-[(3- methyl-1,4′- bipiperidin-1′- yl)carbonyl]-N-[(3- methyl-1,2-oxazol-5- yl)methyl]benzamide | 22.3 mg 63% | LC-MS [Method 7]: Rₜ = 1.18 min MS (ESIpos): m/z = 439 (M + H)⁺ |
| 51 | 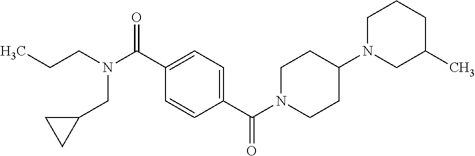 [SMILES: CCCN(CC1CC1)C(=O)c1ccc(C(=O)N2CCC(N3CCCC(C)C3)CC2)cc1] | N-(cyclopropyl- methyl)-4-[(3- methyl-1,4′- bipiperidin-1′-yl) carbonyl]-N- propylbenzamide | 6.5 mg 19% | LC-MS [Method 7]: Rₜ = 1.38 min MS (ESIpos): m/z = 426 (M + H)⁺ |
| 52 | 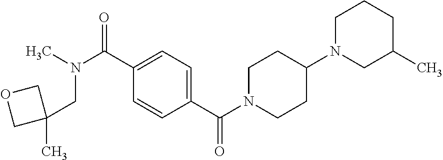 [SMILES: CC1CCCN(C2CCN(C(=O)c3ccc(C(=O)N(C)CC4(C)COC4)cc3)CC2)C1] | N-methyl-4-[(3- methyl-1,4′- bipiperidin-1′- yl)carbonyl]-N-[(3- methyloxetan-3- yl)methyl]benzamide | 4.0 mg 12% | LC-MS [Method 7]: Rₜ = 1.11 min MS (ESIpos): m/z = 428 (M + H)⁺ |
| 53 | 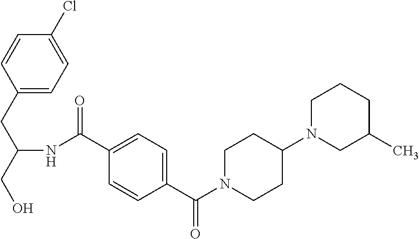 [SMILES: CC1CCCN(C2CCN(C(=O)c3ccc(C(=O)NC(CO)Cc4ccc(Cl)cc4)cc3)CC2)C1] | N-[1-(4-chlorophenyl)- 3-hydroxypropan- 2-yl]-4-[(3-methyl- 1,4′-bipiperidin-1′-yl) carbonyl]benzamide | 16.6 mg 42% | LC-MS [Method 7]: Rₜ = 1.34 min MS (ESIpos): m/z = 498 (M + H)⁺ |
| 54 | 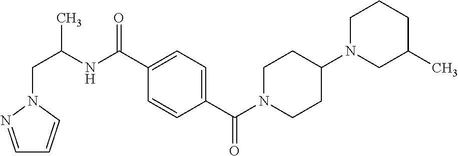 [SMILES: CC1CCCN(C2CCN(C(=O)c3ccc(C(=O)NC(C)Cn4cccn4)cc3)CC2)C1] | 4-[(3-methyl-1,4′- bipiperidin-1′-yl) carbonyl]-N-[1-(1H- pyrazol-1-yl)propan- 2-yl]benzamide | 13.9 mg 40% | LC-MS [Method 7]: Rₜ = 1.14 min MS (ESIpos): m/z = 438 (M + H)⁺ |
| 55 | 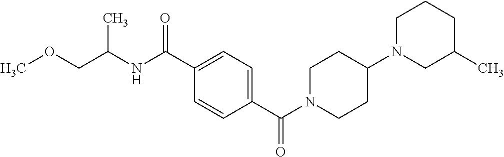 | N-(1- methoxypropan-2- yl)-4-[(3-methyl-1,4′- bipiperidin-1′-yl) carbonyl]benzamide | 5.4 mg 17% | LC-MS [Method 7]: Rₜ = 1.12 min MS (ESIpos): m/z = 402 (M + H)⁺ |
| 56 | 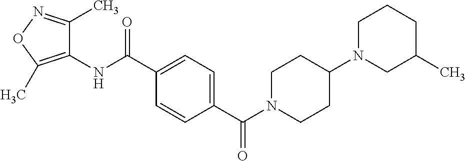 [SMILES: Cc1noc(C)c1NC(=O)c1ccc(C(=O)N2CCC(N3CCCC(C)C3)CC2)cc1] | N-(3,5-dimethyl-1,2- oxazol-4-yl)-4-[(3- methyl-1,4′- bipiperidin-1′-yl) carbonyl]benzamide | 14.5 mg 43% | LC-MS [Method 7]: Rₜ = 1.14 min MS (ESIpos): m/z = 425 (M + H)⁺ |

### Example 57

### N-Methyl-N-[(1-{4-[(3-methyl-1,4′-bipiperidin-1′yl)carbonyl]phenyl}cyclobutyl)methyl]methanesulphonamide

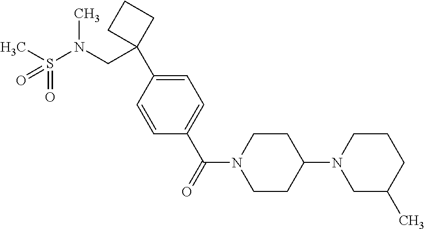

61 mg (0.18 mmol) of N-{[1-(4-bromophenyl)cyclobutyl]methyl}-N-methylmethanesulphonamide, 40 mg (0.22 mmol) of 4-(3-methylpiperidin-1-yl)piperidine, 24 mg (0.09 mmol) of molybdenum hexacarbonyl, 9 mg (0.01 mmol) of trans-bis(acetate)bis[o-(di-o-tolylphosphine)benzyl]dipalladium(II) (Herrmann's palladacycle) and 58 mg (0.55 mmol) of sodium carbonate in 1 ml of water were stirred in a microwave at 150° C. for 10 min. After cooling, the mixture was diluted with a little water and shaken with ethyl acetate. The mixture was filtered through Celite. The organic phase was separated off from the filtrate and dried over sodium sulphate. Concentration gave 65 mg of a crude product which was purified by flash chromatography on silica gel (elution: ethyl acetate, gradient ethyl acetate/methanol: 3/1). The product-containing fractions were concentrated and dried under HV. This gave 29 mg (33% of theory) of an oil.

LC-MS [Method 3]: Rₜ=0.82 min; MS (ESIpos): m/z=462 (M+H)⁺

¹H-NMR (400 MHz, DMSO-d₆): δ [ppm]=0.75-0.89 (m, 4H, including d, 3H), 1.32-1.82 (m, 12H), 2.01-2.11 (m, 1H), 2.15-2.45 (m, 4H, including s, 3H), 2.81 (m, 3H), 3.0 (m, 1H), 3.45 (m, 2H), 3.55 (m, 1H) 4.53 (m, 1H), 7.2 (m, 2H), 7.45 (m, 2H)

### Example 58

### [4-(2-Hydroxypropan-2-yl)phenyl][3-(methoxymethyl)-1,4′-bipiperidin-1′-yl]methanone

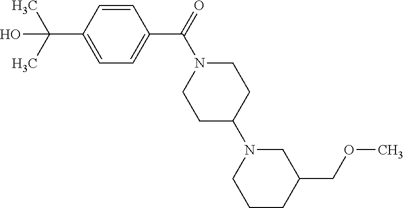 [SMILES: COCC1CCCN(C2CCN(C(=O)c3ccc(C(C)(C)O)cc3)CC2)C1]

Under argon, 106 mg (0.55 mol) of EDC, 85 mg (0.55 mmol) of HOBT and 0.21 g (1.67 mmol) of N,N-diisopropylethylamine were added to 100 mg (0.56 mmol) of 4-(1-hydroxy-1-methylethyl)benzoic acid in 3.6 ml of DMF. After stirring at RT for 10 minutes, 130 mg (0.61 mmol) of 3-(methoxymethyl)-1,4′-bipiperidine were added. The mixture was stirred at RT overnight. After dilution with water, the mixture was extracted with ethyl acetate. The organic phase was washed with saturated aqueous sodium chloride solution and dried over sodium sulphate. The oil obtained after concentration was purified by flash chromatography on silica gel (elution: cyclohexane/ethyl acetate: 1/1, then methanol). The product-containing fractions were concentrated and dried under HV. Further purification was by preparative HPLC [Reprosil, C18 10 μm, 250 mm×30 mm, methanol/water 10:90 to 100:0 over a run time of 23 min]. After HPLC control, the product-containing fractions were combined and concentrated. The residue was dried under HV. This gave 53 mg (25% of theory) of an oil.

LC-MS [Method 3]: Rₜ=0.59 min; MS (ESIpos): m/z=375 (M+H)⁺

¹H-NMR (400 MHz, DMSO-d₆): δ [ppm]=0.8-0.9 (m, 1H), 1.30-1.45 (m, 8H, including: s, 6H), 1.5-2.1 (m, 10H), 2.6-3.0 (m, 4H), 2.95 (m, 1H), 3.1-3.2 (m, 2H), 3.25 (s, 3H), 3.5 (m, 1H), 4.45 (m, 1H), 5.1 (m, 1H), 7.3-7.4 (m, 2H), 7.4-7.5 (m, 2H), 9.55-9.75 (m, 2H)

### Example 59

### (4-tert-Butylphenyl) [3-(5-methyl-1,2,4-oxadiazol-2-yl)-1,4′-bipiperidin-1′-yl]methanone

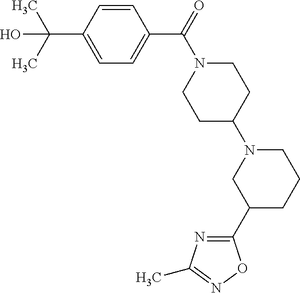 [SMILES: Cc1noc(C2CCCN(C3CCN(C(=O)c4ccc(C(C)(C)O)cc4)CC3)C2)n1]

Under argon, 103 mg (0.54 mol) of 1-(3-dimethylaminopropyl)-3-ethylcarbodiimide hydrochloride, 82 mg (0.54 mmol) of HOBT and 0.43 ml (2.45 mmol) of N,N-diisopropylethylamine were added to 200 mg (0.49 mmol) of 1′-(4-tert-butylbenzoyl)-1,4′-bipiperidine-3-carboxylic acid in 5 ml DMF. After stirring at RT for 10 minutes, 130 mg (0.61 mmol) of acetamidoxime were added. The mixture was stirred at RT overnight. After dilution with water, the mixture was extracted with ethyl acetate. The organic phase was washed with saturated aqueous sodium chloride solution and dried over sodium sulphate. The oil obtained after concentration was heated undiluted at 130° C. for 1 h. The residue was purified by preparative HPLC [Reprosil, C18 10 μm, 250 mm×30 mm, methanol/water (+0.05% trifluoroacetic acid) 50:50 to 100:0 over a run time of 25 min]. After HPLC control, the product-containing fractions were combined and concentrated. The residue was dried under HV. The residue was taken up in ethyl acetate, washed with saturated sodium bicarbonate solution and dried over sodium sulphate, and the solution was concentrated. The product obtained in this manner was purified by flash chromatography on silica gel, elution: ethyl acetate. The product-containing fractions were concentrated and dried under HV. This gave 27 mg (13% of theory) of an oil.

LC-MS [Method 1]: Rₜ=0.80 min; MS (ESIpos): m/z=411 (M+H)⁺

¹H-NMR (400 MHz, DMSO-d₆): δ [ppm]=1.4-1.9 (m, 17H, including: 1.5, s, 9H), 1.9-2.0 (m, 1H), 2.25-2.35 (m, 4H, 2.6, s, 3H), 2.65-2.8 (m, 1H), 2.85-3.2 (m, 3H), 3.5-3.8 (m, 1H), 4.35-4.6 (m, 1H), 7.3-7.4 (m, 2H), 7.4-7.45 (m, 2H)

### Example 60

### [3-(5-Cyclopropyl-4H-1,2,4-triazol-3-yl)-1,4′-bipiperidin-1′-yl][4-(2-hydroxypropan-2-yl)phenyl]methanone

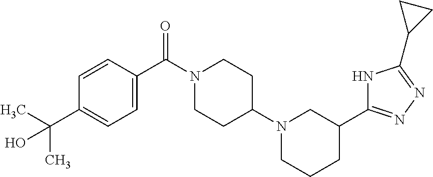 [SMILES: CC(C)(O)c1ccc(C(=O)N2CCC(N3CCCC(c4nnc(C5CC5)[nH]4)C3)CC2)cc1]

Under argon, 37 mg (0.19 mol) of EDC, 29 mg (0.19 mmol) of HOBT and 74 mg (0.58 mmol) of N,N-diisopropylethylamine were added to 35 mg (0.19 mmol) of 4-(1-hydroxy-1-methylethyl)benzoic acid in 1 ml of DMF. After 1 h of stirring at RT, 53 mg (0.19 mmol) of 3-(5-cyclopropyl-4H-1,2,4-triazol-3-yl)-1,4′-bipiperidine, dissolved in 1 ml of DMF, were added. The mixture was stirred at RT overnight. Without work-up, the reaction mixture was chromatographed on an RP column [Reprosil, C18 10 μm, 250 mm×30 mm, methanol/water 10:90 to 100:0 over a run time of 23 min]. After HPLC control, the product-containing fractions were combined and concentrated. The residue was dried under HV. This gave 28 mg (34% of theory) of a solid.

LC-MS [Method 4]: Rₜ=1.10 min; MS (ESIpos): m/z=438 (M+H)⁺

¹H-NMR (400 MHz, DMSO-d₆): δ [ppm]=0.65-1.0 (m, 4H), 1.35-2.3 (m, 17H, including: 1.45, s, 6H), 2.65-3.0 (m, 5H), 3.6 (m, 1H), 4.5 (m, 1H), 7.3-7.4 (m, 2H), 7.4-7.5 (m, 2H), 13.1 and 13.2 (bs, together 1H)

### Example 61

### [3-(5-Cyclobutyl-4H-1,2,4-triazol-3-yl)-1,4′-bipiperidin-1′-yl][4-(2-hydroxypropan-2-yl)phenyl]methanone

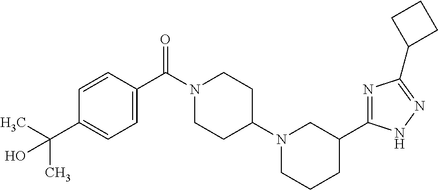 [SMILES: CC(C)(O)c1ccc(C(=O)N2CCC(N3CCCC(c4nc(C5CCC5)n[nH]4)C3)CC2)cc1]

Under argon, 66 mg (0.35 mol) of EDC, 53 mg (0.35 mmol) of HOBT and 134 mg (1.04 mmol) of N,N-diisopropylethylamine were added to 62 mg (0.35 mmol) of 4-(1-hydroxy-1-methylethyl)benzoic acid in 1 ml of DMF. After 1 h of stirring at RT, 100 mg (0.35 mmol) of 3-(5-cyclobutyl-4H-1,2,4-triazol-3-yl)-1,4′-bipiperidine, dissolved in 1 ml of DMF, were added. The mixture was stirred at RT overnight. Without work-up, the reaction mixture was chromatographed on an RP column [Reprosil, C18 10 μm, 250 mm×30 mm, methanol/water 10:90 to 100:0 over a run time of 23 min]. After HPLC control, the product-containing fractions were combined and concentrated. The residue was dried under HV. This gave 43 mg (28% of theory) of a solid.

LC-MS [Method 1]: Rₜ=0.59 min; MS (ESIpos): m/z=452 (M+H)⁺

¹H-NMR (400 MHz, DMSO-d₆): δ [ppm]=1.35-1.55 (m, 10H, including: 1.4, s, 6H), 1.6-2.3 (m, 9H), 2.65-3.0 (m, 5H), 3.6 (m, 2H), 4.5 (m, 1H), 5.0 (m, 1H), 7.3-7.4 (m, 2H), 7.4-7.5 (m, 2H); further signals concealed by DMSO/water.

### Example 62

### [3-(5-Ethyl-1H-1,2,4-triazol-3-yl)-1,4′-bipiperidin-1′-yl][4-(2-hydroxypropan-2-yl)phenyl]methanone

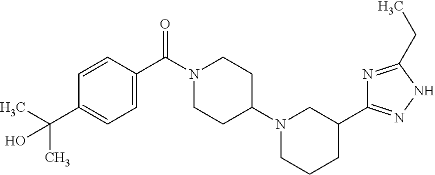 [SMILES: CCc1nc(C2CCCN(C3CCN(C(=O)c4ccc(C(C)(C)O)cc4)CC3)C2)n[nH]1]

Under argon, 62 mg (0.32 mol) of EDC, 49 mg (0.32 mmol) of HOBT and 125 mg (0.97 mmol) of N,N-diisopropylethylamine were added to 58 mg (0.32 mmol) of 4-(1-hydroxy-1-methylethyl)benzoic acid in 1 ml of DMF. After 1 h of stirring at RT, 85 mg (0.32 mmol) of 3-(5-ethyl-4H-1,2,4-triazol-3-yl)-1,4′-bipiperidine, dissolved in 1 ml of DMF, were added. The mixture was stirred at RT overnight. Without work-up, the reaction mixture was chromatographed on an RP column [Reprosil, C18 10 μm, 250 mm×30 mm, methanol/water 10:90 to 100:0 over a run time of 23 min]. After HPLC control, the product-containing fractions were combined and concentrated. The residue was dried under HV. This gave 81 mg (54% of theory) of a solid.

LC-MS [Method 1]: Rₜ=0.47 min; MS (ESIpos): m/z=426 (M+H)⁺

¹H-NMR (400 MHz, DMSO-d₆): δ [ppm]=1.15 (t, 3H), 1.35-2.0 (m, 15H, including: 1.45, s, 6H), 2.1-2.3 (m, 3H), 2.5-3.0 (m, 6H), 3.6 (m, 1H), 4.5 (m, 1H), 5.05 (s, 1H), 7.25-7.3 (m, 2H), 7.45-7.5 (m, 2H), 13.1 (bs, 1H)

### Example 63

### [4-(2-Hydroxypropan-2-yl)phenyl][3-(3-methyl-1H-1,2,4-triazol-5-yl)-1,4′-bipiperidin-1′-yl]methanone

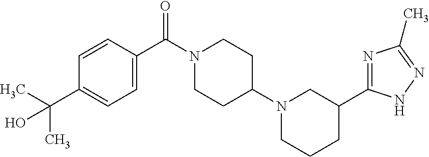 [SMILES: Cc1n[nH]c(C2CCCN(C3CCN(C(=O)c4ccc(C(C)(C)O)cc4)CC3)C2)n1]

Under argon, 75 mg (0.39 mol) of EDC, 60 mg (0.39 mmol) of HOBT and 152 mg (1.18 mmol) of N,N-diisopropylethylamine were added to 71 mg (0.39 mmol) of 4-(1-hydroxy-1-methylethyl)benzoic acid in 1 ml of DMF. After 1 h of stirring at RT, 98 mg (0.39 mmol) of 3-(5-methyl-4H-1,2,4-triazol-3-yl)-1,4′-bipiperidine, dissolved in 1 ml of DMF, were added. The mixture was stirred at RT overnight. Without work-up, the reaction mixture was chromatographed on an RP column [Reprosil, C18 10 μm, 250 mm×30 mm, methanol/water 10:90 to 100:0 over a run time of 23 min]. After HPLC control, the product-containing fractions were combined and concentrated. The residue was dried under HV. This gave 44 mg (27% of theory) of a solid.

LC-MS [Method 1]: Rₜ=0.41 min; MS (ESIpos): m/z=412 (M+H)⁺

¹H-NMR (400 MHz, DMSO-d₆): δ [ppm]=1.35-2.0 (m, 15H, including: 1.4, s, 6H), 2.1-2.3 (m, 5H), 2.6-3.0 (m, 6H), 3.6 (m, 1H), 4.5 (m, 1H), 5.05 (s, 1H), 7.25-7.3 (m, 2H), 7.45-7.5 (m, 2H), 13.2 (br. s, 1H)

### Example 64

### [4-(2-Hydroxypropan-2-yl)phenyl][3-(1H-1,2,4-triazol-5-yl)-1,4′-bipiperidin-1′-yl]yl]methanone

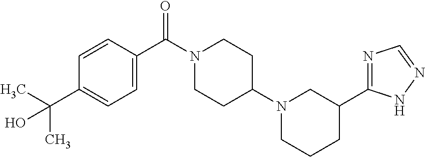 [SMILES: CC(C)(O)c1ccc(C(=O)N2CCC(N3CCCC(c4ncn[nH]4)C3)CC2)cc1]

Under argon, 49 mg (0.25 mol) of EDC, 39 mg (0.25 mmol) of HOBT and 66 mg (0.51 mmol) of N,N-diisopropylethylamine were added to 46 mg (0.25 mmol) of 4-(1-hydroxy-1-methylethyl)benzoic acid in 1 ml of DMF. After 1 h of stirring at RT, 60 mg (0.25 mmol) of 3-(5-methyl-4H-1,2,4-triazol-3-yl)-1,4′-bipiperidine, dissolved in 1 ml of DMF, were added. The mixture was stirred at RT overnight. Without work-up, the reaction mixture was purified on a Biotage cartridge 25M (elution: ethyl acetate/methanol 1:1). After HPLC control, the product-containing fractions were combined and concentrated. The crude product thus obtained was again separated chromatographically (Analogix cartridge 12M, ethyl acetate/methanol gradient 5:1 to 3:1). After HPLC control, the product-containing fractions were combined and concentrated. The residue was dried under HV. This gave 25 mg (25% of theory) of a solid. As a second fraction, a further 28 mg (26% of theory) of target product were obtained in a purity of 91%.

LC-MS [Method 1]: Rₜ=0.31 min; MS (ESIpos): m/z=398 (M+H)⁺

¹H-NMR (400 MHz, DMSO-d₆): δ [ppm]=1.35-2.0 (m, 14H, including: 1.4, s, 6H), 2.1-2.3 (m, 3H), 2.6-3.0 (m, 6H), 3.6 (m, 1H), 4.5 (m, 1H), 5.05 (s, 1H), 7.25-7.3 (m, 2H), 7.45-7.5 (m, 2H), 7.8 and 8.4 (two bs, together 1H), 13.6-13.8 (br. m, about 1H) (The spectrum shows the presence of a tautomer mixture.)

### Example 65

### N-tert-Butyl-2-{3-fluoro-4-[(3-methyl-1,4′-bipiperidin-1′-yl)carbonyl]phenyl}-2-methylpropanamide

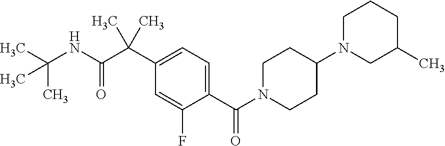 [SMILES: CC1CCCN(C2CCN(C(=O)c3ccc(C(C)(C)C(=O)NC(C)(C)C)cc3F)CC2)C1]

256 mg (content: 46%, 0.36 mmol) of 2-(4-bromo-3-fluorophenyl)-N-tert-butyl-2-methylpropanamide, 48 mg (0.18 mmol) of molybdenum hexacarbonyl, 34 mg (0.04 mmol) of trans-bis(acetate)bis[o-(di-o-tolylphosphine)benzyl]dipalladium(II) (Herrmann's palladacycle) and 116 mg (1.10 mmol) of sodium carbonate were suspended in 1.5 ml of water and stirred in a microwave at 150° C. and 200 Watt for 10 min. After cooling, the mixture was diluted with water and shaken with ethyl acetate. The mixture was filtered through a little Celite. The organic phase was separated off, dried over magnesium sulphate and filtered, and the filtrate was concentrated. The residue was purified by flash chromatography on silica gel, elution:ethyl acetate, gradient ethyl acetate/methanol: 3/1. The product-containing fractions were concentrated. The crude product was purified by preparative HPLC, Method: Axia Gemini C18, 5 μm, 50 mm×21.5 mm [30% acetonitrile/70% water (+0.1% ammonium hydroxide) to 100% acetonitrile]. The product-containing fractions were combined, concentrated and dried under HV. This gave 9 mg (5% of theory, purity: 92%) of a syrup.

LC-MS [Method 3]: Rₜ=0.88 min; MS (ESIpos): m/z=446 (M+H)⁺

¹H-NMR (400 MHz, DMSO-d₆): δ [ppm]=0.8 (m, 4H), 1.25 (s, 9H), 1.3-1.8 (m, 16, including: 1.4, s, 6H), 1.53 (s, 4H), 2.0 (m, 1H), 2.65-2.75 (m, ca 3H), 2.95-3.05 (m, 1H), 3.4 (m, 1H), 4.5 (d, 1H), 6.55 (s, 1H), 7.14-7.30 (m, 2H), 7.35 (m, 1H).

### Example 66

### N-[(1-{4-[(3-Methyl-1,4′-bipiperidin-1′-yl)carbonyl]phenyl}cyclobutyl)methyl]formamide hydrochloride

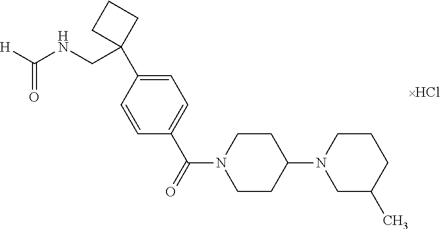 [SMILES: CC1CCCN(C2CCN(C(=O)c3ccc(C4(CNC=O)CCC4)cc3)CC2)C1]

150 mg (0.56 mmol) of N-{[1-(4-bromophenyl)cyclobutyl]methyl}-N-methylformamide [obtainable in one step from commercially available 1-(4-bromophenylcyclobutanmethanamine by reaction with formic acid in boiling o-xylene with removal of water], 122 mg (0.67 mmol) of 4-(3-methylpiperidin-1-yl)piperidine, 74 mg (0.28 mmol) of molybdenum hexacarbonyl, 26 mg (0.03 mmol) of trans-bis(acetate)bis[o-(di-o-tolylphosphine)benzyl]dipalladium(II) (Herrmann's palladacycle) and 178 mg (1.7 mmol) of sodium carbonate in 2.9 ml of water were stirred in a microwave at 150° C. for 10 min. After cooling, the mixture was diluted with a little water and shaken with ethyl acetate. The mixture was filtered through Celite. The organic phase was separated off from the filtrate and dried over sodium sulphate. Concentration gave 109 mg of a crude product which was purified by flash chromatography on silica gel (elution: ethyl acetate, gradient ethyl acetate/methanol 3:1). Flash-chromatography was repeated using the crude product obtained after concentration of the product-containing fractions (elution: ethyl acetate/methanol 10:1). The product-containing fractions were concentrated and dried under HV. The residue obtained was stirred with etheral hydrogen chloride in diethyl ether. The hygroscopic salt was taken up in methanol, concentrated and dried under high vacuum. This gave 17 mg (6.8% of theory) of a solid.

LC-MS [Method 3]: Rₜ=0.71 min; MS (ESIpos): m/z=398 (M+H)⁺

¹H-NMR (400 MHz, DMSO-d₆): δ [ppm]=0.85-0.9 (d, 3H), 1.0-1.15 (m, 1H), 1.6-2.3 (m, about 14H), 2.81 (m, 3H), 3.0 (m, 1H), 3.5 (m, 1H), 4.5 (m, 1H), 7.2 (m, 2H), 7.35 (m, 2H), 7.85-8 (m, 2H), 9.2 (m, 1H)

### Example 67

### N-Methyl-N-[(1-{4-[(3-methyl-1,4′-bipiperidin-1′-yl)carbonyl]phenyl}cyclobutyl)methyl]formamide trifluoroacetic acid salt

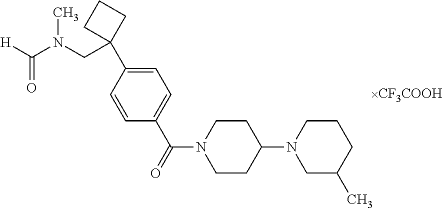 [SMILES: CC1CCCN(C2CCN(C(=O)c3ccc(C4(CN(C)C=O)CCC4)cc3)CC2)C1]

126 mg (0.45 mmol) of N-{[1-(4-bromophenyl)cyclobutyl]methyl}-N-methylformamide, 98 mg (0.54 mmol) of 4-(3-methylpiperidin-1-yl)piperidine, 59 mg (0.22 mmol) of molybdenum hexacarbonyl, 32 mg (0.03 mmol) of trans-bis(acetate)bis[o-(di-o-tolylphosphine)benzyl]dipalladium(II) (Herrmann's palladacycle) and 142 mg (1.34 mmol) of sodium carbonate in 1 ml of water were stirred in a microwave at 150° C. for 10 min. After cooling, the mixture was diluted with a little water and shaken with ethyl acetate. The mixture was filtered through Celite. The organic phase was separated off from the filtrate and dried over sodium sulphate. Concentration gave 101 mg of a crude product which was purified by flash chromatography on silica gel (elution: ethyl acetate, gradient ethyl acetate/methanol 1:1). The product-containing fractions were combined and concentrated. Addition of etheral hydrogen chloride did not result in the formation of a solid. The solvent was removed by evaporation and the residue was subjected to an RP HPLC separation [Reprosil, C18 10 μm, 250 mm×30 mm, methanol/water (+0.05% trifluoroacetic acid) 30:70 to 100:0 over a run time of 23 min]. After HPLC control, the product-containing fractions were combined and concentrated. The residue was dried under HV. This gave 59 mg (25% of theory) of a syrup.

LC-MS [Method 4]: Rₜ=0.71 min; MS (ESIpos): m/z=412 (M+H)⁺

¹H-NMR (400 MHz, DMSO-d₆): δ [ppm]=0.85-0.9 (d, 3H), 1.0-1.15 (m, 1H), 1.6-2.4 (m, about 16H), 2.7-3.6 (m, about 7H), 3.0 (m, 1H), 3.5 (m, 1H), 4.5 (m, 1H), 7.2 (m, 2H), 7.4 (m, 2H), 7.55 and 7.9 (2 d, together 1H), 9.3 (m, 1H) [some signals doubled owing to amide E/Z isomerism]

### Example 68

### (4-{1-[(Methylamino)methyl]cyclobutyl}phenyl) (3-methyl-1,4′-bipiperidin-1′-yl)methanone trifluoroacetic acid salt

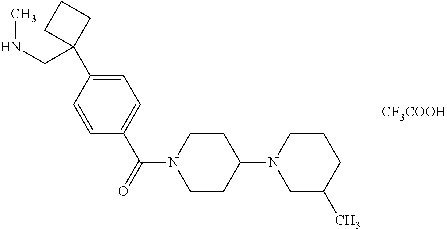 [SMILES: CNCC1(c2ccc(C(=O)N3CCC(N4CCCC(C)C4)CC3)cc2)CCC1]

44 mg (0.08 mmol) of tert-butyl methyl[(1-{4-[(3-methyl-1,4′-bipiperidin-1′-yl)carbonyl]phenyl}cyclobutyl)methyl]carbamate (content: 88%) were dissolved in 10 ml of dichloromethane, and 0.68 ml (8.8 mmol) of trifluoroacetic acid were added with ice cooling. The mixture was stirred at RT overnight. The two-phase mixture was concentrated and the residue was dried under HV. The residue was subjected to an RP HPLC separation [Reprosil, C18 10 μm, 250 mm×30 mm, methanol/water (+0.05% trifluoroacetic acid) 30:70 to 100:0 over a run time of 23 min]. After HPLC control, the product-containing fractions were combined and concentrated. The residue was dried under HV. This gave 40 mg (71% of theory) of a solid.

LC-MS [Method 2]: Rₜ=0.71 min; MS (ESIpos): m/z=384 (M+H)⁺

¹H-NMR (400 MHz, DMSO-d₆): δ [ppm]=0.9-0.95 (d, 3H), 1.0-1.15 (m, 1H), 1.5-2.2 (m, about 10H), 2.25-2.35 (m, about 4H), 2.6-3.2 (m, 4H), 3.3-3.5 (m, 5H), 7.2 (m, 2H), 7.4 (m, 2H), 8.1 and 9.35 (m, together 2H).

The intermediate tert-butyl methyl[(1-{4-[(3-methyl-1,4′-bipiperidin-1′-yl)carbonyl]phenyl}cyclobutyl)methyl]carbamate required for this purpose is accessible as follows: 245 mg (about 0.69 mmol) of tert-butyl {[1-(4-bromophenyl)cyclobutyl]methyl}methylcarbamate (as crude product, still contained about 15% DMAP), 152 mg (0.83 mmol) of 4-(3-methylpiperidin-1-yl)piperidine, 91 mg (0.35 mmol) of molybdenum hexacarbonyl, 32 mg (0.03 mmol) of trans-bis(acetate)bis[o-(di-o-tolylphosphine)benzyl]dipalladium(II) (Herrmann's palladacycle) and 220 mg (2.08 mmol) of sodium carbonate with 3.6 ml of water were stirred in a microwave at 150° C. for 10 min. After cooling, the mixture was diluted with a little water and shaken with ethyl acetate. The mixture was filtered through Celite. The organic phase was separated off from the filtrate and dried over sodium sulphate. Concentration gave a crude product which was purified by flash chromatography on silica gel (elution: ethyl acetate, gradient ethyl acetate/methanol 2/1). The product-containing fractions were combined and concentrated. This gave 44 mg of intermediate in a purity of 88% which were reacted further in this form.

### Example 69

### {3-[5-(Cyclobutylmethyl)-4H-1,2,4-triazol-3-yl]-1,4′-bipiperidin-1′-yl}[4-(2-hydroxypropan-2-yl)phenyl]methanone

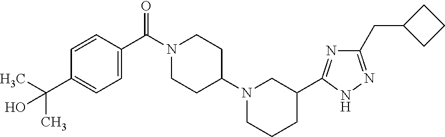 [SMILES: CC(C)(O)c1ccc(C(=O)N2CCC(N3CCCC(c4nc(CC5CCC5)n[nH]4)C3)CC2)cc1]

Under argon, 42 mg (0.22 mol) of EDC, 33 mg (0.22 mmol) of HOBT and 84 mg (0.65 mmol) of N,N-diisopropylethylamine were added to 39 mg (0.22 mmol) of 4-(1-hydroxy-1-methylethyl)benzoic acid in 1 ml of DMF. After 1 h of stirring at RT, 66 mg (0.22 mmol) of 3-(5-cyclobutylmethyl-4H-1,2,4-triazol-3-yl)-1,4′-bipiperidine, dissolved in 1 ml of DMF, were added. The mixture was stirred at RT overnight. Without work-up, the reaction mixture was chromatographed on an RP column [Reprosil, C18 10 μm, 250 mm×30 mm, methanol/water 10:90 to 100:0 over a run time of 23 min]. After HPLC control, the product-containing fractions were combined and concentrated. Purification by RP chromatography was repeated once. After HPLC control, the product-containing fractions were combined and concentrated. The residue was dried under HV. This gave 35 mg (35% of theory) of a solid.

LC-MS [Method 2]: Rₜ=0.63 min; MS (ESIpos): m/z=466 (M+H)⁺

¹H-NMR (400 MHz, DMSO-d₆): δ [ppm]=1.35-1.5 (m, 10H, including: 1.4, s, 6H), 1.6-2.05 (m, 10H), 2.1-2.3 (m, 2H), 2.6-3.0 (m, 8H), 3.6 (m, 1H), 4.45 (m, 1H), 5.05 (s, 1H), 7.25-7.3 (m, 2H), 7.45-7.5 (m, 2H), 13.15 (bs, 1H)

### Example 70

### 4-[(3-Methyl-1,4′-bipiperidin-1′-yl)carbonyl]-N-[3-(trifluoromethoxy)benzyl]benzamide trifluoroacetic acid salt

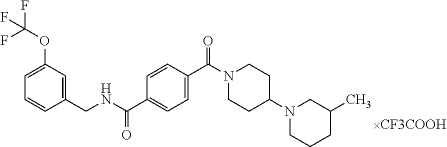 [SMILES: CC1CCCN(C2CCN(C(=O)c3ccc(C(=O)NCc4cccc(OC(F)(F)F)c4)cc3)CC2)C1]

0.26 ml (1.5 mmol) of diisopropylethylamine and 139 mg (0.364 mmol) of HATU were added to a mixture of 102 mg (0.309 mmol) of the compound from Example 15A and 71 mg (0.370 mmol) of 1-[3-(trifluoromethoxy)phenyl]methanamine in 1.0 ml of DMF, and the mixture was stirred at RT overnight. For work-up, water was added and the mixture was extracted repeatedly with ethyl acetate. The combined organic phases were dried over magnesium sulphate, filtered and concentrated. Chromatographic separation using Isolera (10 g, silica gel cartridge, ethyl acetate/methanol gradient) gave no clean product and the residue was purified again by preparative HPLC [Method 10]. After HPLC control, the product-containing fractions were combined and concentrated. The residue was dried under HV. This gave 4 mg of the title compound (3% of theory).

LC-MS [Method 1]: Rₜ=0.75 min; MS (ESIpos): m/z=504 (M−CF₃COOH+H)⁺

¹H-NMR (400 MHz, DMSO-d₆): δ [ppm]=0.92 (d, 3H), 1.02-1.17 (m, 1H), 1.59-1.77 (m, 4H), 1.78-2.01 (m, 3H), 2.01-2.18 (m, 1H), 2.72-2.98 (m, 2H), 2.99-3.19 (m, 1H), 3.55-3.72 (m, 1H), 4.54 (d, 2H), 4.59-4.71 (m, 1H), 7.25 (d, 1H), 7.28-7.33 (m, 1H), 7.36 (d, 1H), 7.44-7.51 (m, 1H), 7.53 (d, 2H), 7.96 (d, 2H), 8.95-9.11 (m, 1H), 9.17-9.27 (m, 1H).

The following were prepared analogously:

### Example 71

### 4-[(3-Methyl-1,4′-bipiperidin-1′-yl)carbonyl]-N-(2-phenylpropan-2-yl)benzamide trifluoroacetic acid salt

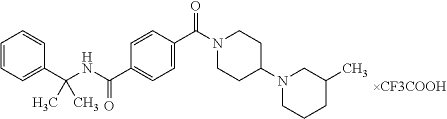 [SMILES: CC1CCCN(C2CCN(C(=O)c3ccc(C(=O)NC(C)(C)c4ccccc4)cc3)CC2)C1]

Reaction of 50 mg (0.151 mmol) of the compound from Example 15A with 25 mg (0.182 mmol) of 2-phenylpropane-2-amine, 75 mg (0.197 mmol) of HATU and 0.13 ml (0.76 mmol) of N,N-diisopropylethylamine in 0.5 ml of DMF and separation by preparative HPLC [Method 10] gave the title compound as trifluoroacetic acid salt (57 mg, 65% of theory)

LC-MS [Method 2]: Rₜ=0.75 min; MS (ESIpos): m/z=448 (M−CF₃COOH+H)⁺

¹H-NMR (400 MHz, DMSO-d₆): δ [ppm]=0.92 (d, 3H), 1.01-1.18 (m, 1H), 1.60-1.77 (m, 10H), 1.78-2.00 (m, 3H), 2.00-2.18 (m, 1H), 2.55-2.66 (m, 1H), 2.72-2.96 (m, 2H), 2.99-3.20 (m, 1H), 3.28-3.52 (m, 3H), 3.54-3.74 (m, 1H), 4.55-4.71 (m, 1H), 7.14-7.20 (m, 1H), 7.25-7.32 (m, 2H), 7.35-7.41 (m, 2H), 7.49 (d, 2H), 7.91 (d, 2H), 8.54 (br. s, 1H), 9.13-9.23 (m, 1H).

### Example 72

### 4-[(3-Methyl-1,4′-bipiperidin-1′-yl)carbonyl]-N-{2-[4-(trifluoromethyl)phenyl]propan-2-yl}benzamide trifluoroacetic acid salt

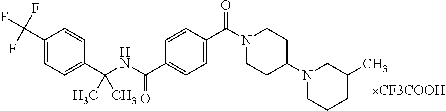 [SMILES: CC1CCCN(C2CCN(C(=O)c3ccc(C(=O)NC(C)(C)c4ccc(C(F)(F)F)cc4)cc3)CC2)C1]

Reaction of 50 mg (0.151 mmol) of the compound from Example 15A with 37 mg (0.182 mmol) of 2-[4-(trifluoromethyl)phenyl]propane-2-amine, 75 mg (0.197 mmol) of HATU and 0.13 ml (0.76 mmol) of N,N-diisopropylethylamine in 0.5 ml of DMF and separation by preparative HPLC [Method 10] gave the title compound as trifluoroacetic acid salt (66 mg, 68% of theory)

LC-MS [Method 2]: Rₜ=0.85 min; MS (ESIpos): m/z=516 (M−CF₃COOH+H)⁺

¹H-NMR (400 MHz, DMSO-d₆): δ [ppm]=0.92 (d, 3H), 1.03-1.17 (m, 1H), 1.60-1.77 (m, 10H), 1.78-1.97 (m, 3H), 1.99-2.17 (m, 1H), 2.55-2.68 (m, 1H), 2.72-2.98 (m, 2H), 2.99-3.21 (m, 1H), 3.29-3.52 (m, 3H), 3.56-3.68 (m, 1H), 4.58-4.70 (m, 1H), 7.49 (d, 2H), 7.59 (d, 2H), 7.66 (d, 2H), 7.92 (d, 2H), 8.71 (br. s, 1H), 9.10-9.20 (m, 1H).

### Example 73

### N-[2-(3,4-Dichlorophenyl)propan-2-yl]-4-[(3-methyl-1,4′-bipiperidin-1′-yl)carbonyl]benzamide trifluoroacetic acid salt

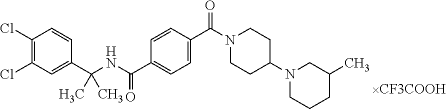 [SMILES: CC1CCCN(C2CCN(C(=O)c3ccc(C(=O)NC(C)(C)c4ccc(Cl)c(Cl)c4)cc3)CC2)C1]

Reaction of 50 mg (0.151 mmol) of the compound from Example 15A with 44 mg (0.182 mmol) of 2-(3,4-dichlorophenyl)propane-2-amine hydrochloride, 75 mg (0.197 mmol) of HATU and 0.19 ml (1.06 mmol) of N,N-diisopropylethylamine in 0.5 ml of DMF and separation by preparative HPLC [Method 10] gave the title compound as trifluoroacetic acid salt (74 mg, 73% of theory)

LC-MS [Method 2]: Rₜ=0.86 min; MS (ESIpos): m/z=516 (M−CF₃COOH+H)⁺

¹H-NMR (400 MHz, DMSO-d₆): δ [ppm]=0.92 (d, 3H), 1.02-1.17 (m, 1H), 1.60-1.77 (m, 10H), 1.78-1.99 (m, 3H), 2.01-2.19 (m, 1H), 2.56-2.66 (m, 1H), 2.71-2.99 (m, 2H), 3.00-3.19 (m, 1H), 3.28-3.54 (m, 3H), 3.55-3.74 (m, 1H), 4.61-4.72 (m, 1H), 7.37 (dd, 1H), 7.49 (d, 2H), 7.55 (d, 1H), 7.57 (s, 1H), 7.91 (d, 2H), 8.65 (br s, 1H), 9.15-9.25 (m, 1).

### Example 74

### 4-{[4-(3-Methylcyclohexyl)piperidin-1-yl]carbonyl}-N-[(3-methylpyridin-2-yl)methyl]benzamide trifluoroacetic acid salt

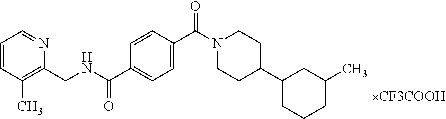 [SMILES: Cc1cccnc1CNC(=O)c1ccc(C(=O)N2CCC(C3CCCC(C)C3)CC2)cc1]

0.26 ml (1.5 mmol) of diisopropylethylamine and 75 mg (0.197 mmol) of HATU were added to a mixture of 50 mg (0.151 mmol) of the compound from Example 15A and 35 mg (0.182 mmol) of 1-(3-methylpyridin-2-yl)methanamine dihydrochloride in 0.5 ml of DMF, and the mixture was stirred at RT overnight. The reaction mixture was separated directly by preparative HPLC [Method 10]. After HPLC control, the product-containing fractions were combined and concentrated. The residue was dried under HV. This gave 62 mg of the title compound (72% of theory).

LC-MS [Method 2]: Rₜ=0.40 min; MS (ESIpos): m/z=435 (M−CF₃COOH+H)⁺

¹H-NMR (400 MHz, DMSO-d₆): δ [ppm]=0.92 (d, 3H), 1.03-1.15 (m, 1H), 1.60-1.77 (m, 4H), 1.78-2.20 (m, 4H), 2.45 (s, 3H), 2.55-2.65 (m, 1H), 2.72-2.97 (m, 2H), 3.03-3.18 (m, 1H), 3.28-3.42 (m, 2H), 3.43-3.53 (m, 1H), 3.53-3.71 (m, 1H), 4.55-4.71 (m, 1H), 4.72 (d, 2H), 7.53 (d, 2H), 7.57-7.64 (m, 1H), 7.98 (d, 2H), 8.02-8.09 (m, 1H), 8.54 (d, 1H), 9.17-9.30 (m, 2H).

### Example 75

### N-[(2-Chloropyridin-4-yl)methyl]-4-[(3-methyl-1,4′-bipiperidin-1′-yl)carbonyl]benzamide trifluoroacetic acid salt

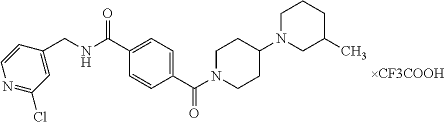 [SMILES: CC1CCCN(C2CCN(C(=O)c3ccc(C(=O)NCc4ccnc(Cl)c4)cc3)CC2)C1]

0.13 ml of diisopropylethylamine (0.76 mmol) and 75 mg of HATU (0.197 mmol) were added to a mixture of 50 mg (0.151 mmol) of the compound from Example 15A and 26 mg of 1-(2-chloropyridin-4-yl)methanamine (0.182 mmol) in 0.5 ml of DMF, and the mixture was stirred at RT overnight. The reaction mixture was separated by preparative HPLC [Method 10]. After HPLC control, the product-containing fractions were combined and concentrated and the residue was dried under HV. This gave 71 mg of the title compound (81% of theory).

LC-MS [Method 1]: Rₜ=0.55 min; MS (ESIpos): m/z=455 (M−CF₃COOH+H)⁺

¹H-NMR (400 MHz, DMSO-d₆): δ [ppm]=0.91 (d, 3H), 1.03-1.27 (m, 1H), 1.52-2.21 (m, 8H), 2.57-2.70 (m, 1H), 2.75-2.97 (m, 2H), 3.02-3.19 (m, 1H), 3.28-3.54 (m, 3H), 3.57-3.71 (m, 1H), 4.50-4.56 (d, 2H), 4.58-4.69 (m, 1H), 7.35 (d, 1H), 7.43 (s, 1H), 7.54 (d, 2H), 7.98 (d, 2H), 8.36 (d, 2H), 9.08-9.19 (m, 1H), 9.24-9.28 (m, 1H).

The following were prepared analogously:

### Example 76

### N-[(6-Chloropyridin-2-yl)methyl]-4-[(3-methyl-1,4′-bipiperidin-1′-yl)carbonyl]benzamide trifluoroacetic acid salt

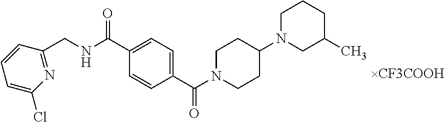 [SMILES: CC1CCCN(C2CCN(C(=O)c3ccc(C(=O)NCc4cccc(Cl)n4)cc3)CC2)C1]

Reaction of 50 mg (0.151 mmol) of the compound from Example 15A with 33 mg (0.182 mmol) of 1-(6-chloropyridin-2-yl)methanamine hydrochloride, 75 mg (0.197 mmol) of HATU and 0.26 ml (1.5 mmol) of N,N-diisopropylethylamine in 0.50 ml of DMF and separation by preparative HPLC [Method 10] gave the title compound as trifluoroacetic acid salt (78 mg, 89% of theory)

LC-MS [Method 1]: Rₜ=0.58 min; MS (ESIpos): m/z=455 (M−CF₃COOH+H)⁺

¹H-NMR (400 MHz, DMSO-d₆): δ [ppm]=0.92 (d, 3H), 1.03-1.17 (m, 1H), 1.59-1.77 (m, 4H), 1.78-2.01 (m, 3H), 2.02-2.19 (m, 1H), 2.55-2.67 (m, 1H), 2.72-2.98 (m, 2H), 3.01-3.19 (m, 1H), 3.28-3.54 (m, 3H), 3.55-3.74 (m, 1H), 4.55 (d, 2H), 4.59-4.70 (m, 1H), 7.35 (d, 1H), 7.41 (d, 1H), 7.53 (d, 2H), 7.84 (t, 1H), 7.98 (d, 2H), 9.07-9.20 (m, 1H), 9.25-9.34 (m, 1H).

### Example 77

### N-[2-(4-Chlorophenyl)propan-2-yl]-4-[(3-methyl-1,4′-bipiperidin-1′-yl)carbonyl]benzamide trifluoroacetic acid salt

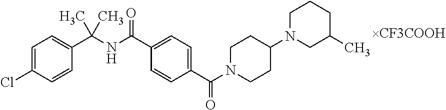 [SMILES: CC1CCCN(C2CCN(C(=O)c3ccc(C(=O)NC(C)(C)c4ccc(Cl)cc4)cc3)CC2)C1]

Reaction of 50 mg (0.151 mmol) of the compound from Example 15A with 31 mg (0.182 mmol) of 2-(4-chlorophenyl)propane-2-amine, 75 mg (0.197 mmol) of HATU and 0.13 ml (0.76 mmol) of N,N-diisopropylethylamine in 0.50 ml of DMF and separation by preparative HPLC [Method 10] gave the title compound as trifluoroacetic acid salt (70 mg, 75% of theory)

LC-MS [Method 1]: Rₜ=0.75 min; MS (ESIpos): m/z=482 (M−CF₃COOH+H)⁺

¹H-NMR (400 MHz, DMSO-d₆): δ [ppm]=0.92 (d, 3H), 1.03-1.19 (m, 1H), 1.60-1.77 (m, 10H), 1.78-2.00 (m, 3H), 2.00-2.19 (m, 1H), 2.55-2.65 (m, 1H), 2.72-2.97 (m, 2H), 3.01-3.19 (m, 1H), 3.28-3.53 (m, 3H), 3.56-3.71 (m, 1H), 4.56-4.71 (m, 1H), 7.34 (d, 2H), 7.39 (d, 2H), 7.48 (d, 2H), 7.91 (d, 2H), 8.59 (br. s, 1H), 9.08-9.17 (m, 1H).

### Example 78

### N-[2-(2-Chlorophenyl)propan-2-yl]-4-[(3-methyl-1,4′-bipiperidin-1′-yl)carbonyl]benzamide trifluoroacetic acid salt

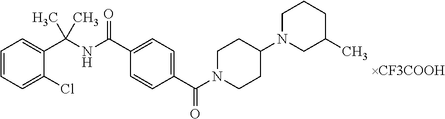 [SMILES: CC1CCCN(C2CCN(C(=O)c3ccc(C(=O)NC(C)(C)c4ccccc4Cl)cc3)CC2)C1]

Reaction of 50 mg (0.151 mmol) of the compound from Example 15A with 31 mg (0.182 mmol) of 2-(4-chlorophenyl)propane-2-amine, 75 mg (0.197 mmol) of HATU and 0.13 ml (0.76 mmol) of N,N-diisopropylethylamine in 0.50 ml of DMF and separation by preparative HPLC [Method 10] gave the title compound as trifluoroacetic acid salt (42.7 mg, 47% of theory)

LC-MS [Method 1]: Rₜ=0.71 min; MS (ESIpos): m/z=482 (M−CF₃COOH+H)⁺

¹H-NMR (400 MHz, DMSO-d₆): δ [ppm]=0.91 (d, 3H), 1.02-1.16 (m, 1H), 1.60-2.20 (m, 14H), 2.55-2.66 (m, 1H), 2.71-2.95 (m, 2H), 3.00-3.20 (m, 1H), 3.28-3.52 (m, 3H), 3.56-3.68 (m, 1H), 4.57-4.70 (m, 1H), 7.19-7.25 (m, 1H), 7.28-7.35 (m, 2H), 7.43-7.50 (m, 2H), 7.53-7.59 (m, 1H), 7.90 (d, 2H), 8.64 (br. s, 1H), 9.13-9.23 (m, 1H).

### Example 79

### N-[2-(3-Chlorophenyl)propan-2-yl]-4-[(3-methyl-1,4′-bipiperidin-1′-yl)carbonyl]benzamide trifluoroacetic acid salt

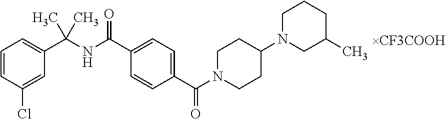 [SMILES: CC1CCCN(C2CCN(C(=O)c3ccc(C(=O)NC(C)(C)c4cccc(Cl)c4)cc3)CC2)C1]

Reaction of 50 mg (0.151 mmol) of the compound from Example 15A with 31 mg (0.182 mmol) of 2-(3-chlorophenyl)propane-2-amine, 75 mg (0.197 mmol) of HATU and 0.13 ml (0.76 mmol) of N,N-diisopropylethylamine in 0.50 ml of DMF and separation by preparative HPLC [Method 10] gave the title compound as trifluoroacetic acid salt (77 mg, 85% of theory)

LC-MS [Method 1]: Rₜ=0.76 min; MS (ESIpos): m/z=482 (M−CF₃COOH+H)⁺

¹H-NMR (400 MHz, DMSO-d₆): δ [ppm]=0.92 (d, 3H), 1.03-1.16 (m, 1H), 1.60-2.20 (m, 14H), 2.55-2.65 (m, 1H), 2.72-2.95 (m, 2H), 3.02-3.17 (m, 1H), 3.28-3.52 (m, 3H), 3.59-3.67 (m, 1H), 4.56-4.72 (m, 1H), 7.23-7.27 (m, 1H), 7.30-7.40 (m, 3H), 7.47-7.53 (m, 2H), 7.91 (d, 2H), 8.61 (br. s, 1H), 9.02-9.13 (m, 1H).

### Example 80

### N-[2-(3,5-Dichlorophenyl)propan-2-yl]-4-[(3-methyl-1,4′-bipiperidin-1′-yl)carbonyl]benzamide trifluoroacetic acid salt

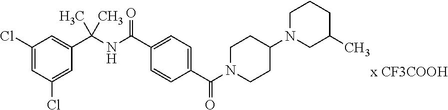 [SMILES: CC1CCCN(C2CCN(C(=O)c3ccc(C(=O)NC(C)(C)c4cc(Cl)cc(Cl)c4)cc3)CC2)C1]

Reaction of 50 mg (0.151 mmol) of the compound from Example 15A with 37 mg (0.182 mmol) of 2-(3,5-dichlorophenyl)propane-2-amine, 75 mg (0.197 mmol) of HATU and 0.13 ml (0.76 mmol) of N,N-diisopropylethylamine in 0.50 ml of DMF and separation by preparative HPLC [Method 10] gave the title compound as trifluoroacetic acid salt (65.0 mg, 67% of theory)

LC-MS [Method 8]: Rₜ=1.07 min; MS (ESIpos): m/z=516 (M−CF₃COOH+H)⁺

¹H-NMR (400 MHz, DMSO-d₆): δ [ppm]=0.92 (d, 3H), 1.03-1.18 (m, 1H), 1.59-1.77 (m, 10H), 1.77-1.99 (m, 3H), 2.01-2.18 (m, 1H), 2.56-2.66 (m, 1H), 2.72-2.96 (m, 2H), 3.02-3.20 (m, 1H), 3.29-3.53 (m, 3H), 3.57-3.72 (m, 1H), 4.58-4.70 (m, 1H), 7.38 (d, 2H), 7.42-7.45 (m, 1H), 7.50 (d, 2H), 7.92 (d, 2H), 8.66 (br. s, 1H), 9.11-9.23 (m, 1H).

### Example 81

### 4-[(3-Methyl-1,4′-bipiperidin-1′-yl)carbonyl]-N-[2-(trifluoromethyl)benzyl]benzamide trifluoroacetic acid salt

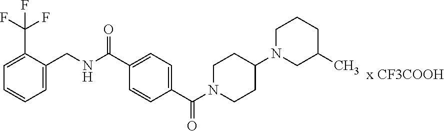 [SMILES: CC1CCCN(C2CCN(C(=O)c3ccc(C(=O)NCc4ccccc4C(F)(F)F)cc3)CC2)C1]

Reaction of 50 mg (0.151 mmol) of the compound from Example 15A with 32 mg (0.182 mmol) of 2-(trifluoromethyl)benzylamine, 75 mg (0.197 mmol) of HATU and 0.13 ml (0.76 mmol) of N,N-diisopropylethylamine in 0.50 ml of DMF and two separations by preparative HPLC [Method 11] gave the title compound as trifluoroacetic acid salt (52 mg, 56% of theory)

LC-MS [Method 1]: Rt=0.71 min; MS (ESIpos): m/z=488 (M−CF₃COOH+H)⁺

¹H-NMR (400 MHz, DMSO-d₆): δ [ppm]=0.92 (d, 3H), 1.01-1.19 (m, 1H), 1.60-1.78 (m, 4H), 1.78-2.20 (m, 4H), 2.56-2.68 (m, 1H), 2.72-2.96 (m, 2H), 3.02-3.21 (m, 1H), 3.29-3.54 (m, 3H), 3.54-3.74 (m, 1H), 4.55-4.74 (m, 3H), 7.45-7.56 (m, 4H), 7.62-7.70 (m, 1H), 7.72-7.78 (m, 1H), 8.01 (d, 2H), 9.20-9.34 (m, 2H).

### Example 82

### (R)—N-[(3,5-Difluoropyridin-2-yl)methyl]-4-{[(3R)-3-methyl-1,4′-bipiperidin-1′-yl]carbonyl}benzamide trifluoroacetic acid salt

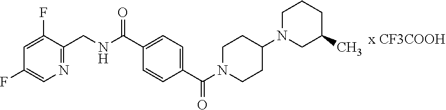 [SMILES: C[C@@H]1CCCN(C2CCN(C(=O)c3ccc(C(=O)NCc4ncc(F)cc4F)cc3)CC2)C1]

Reaction of 100 mg (0.225 mmol) of the compound from Example 58A with 49 mg (0.270 mmol) of 1-(3,5-difluoropyridin-2-yl)methanamine hydrochloride, 111 mg (0.292 mmol) of HATU and 0.39 ml (2.3 mmol) of N,N-diisopropylethylamine in 1.0 ml of DMF and subsequent separation of the reaction mixture by preparative HPLC [Method 12a] gave the title compound as trifluoroacetic acid salt (104 mg, 80% of theory)

LC-MS [Method 1]: Rₜ=0.55 min; MS (ESIpos): m/z=457 (M−CF₃COOH+H)⁺

¹H-NMR (400 MHz, DMSO-d₆): δ [ppm]=0.92 (d, 3H), 1.01-1.18 (m, 1H), 1.59-1.77 (m, 4H), 1.77-2.15 (m, 4H), 2.55-2.66 (m, 1H), 2.70-2.95 (m, 2H), 2.97-3.20 (m, 1H), 3.25-3.53 (m, 3H), 4.52-4.71 (m, 3H), 7.45-7.56 (m, 2H), 7.88-8.00 (m, 3H), 8.46 (d, 1H), 9.05-9.18 (m, 2H).

### Example 83

### (R)—N-[(2-Chloropyridin-3-yl)methyl]-4-{[(3R)-3-methyl-1,4′-bipiperidin-1′-yl]carbonyl}benzamide trifluoroacetic acid salt

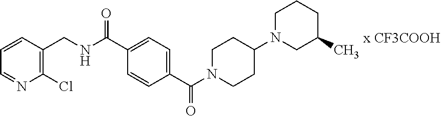 [SMILES: C[C@@H]1CCCN(C2CCN(C(=O)c3ccc(C(=O)NCc4cccnc4Cl)cc3)CC2)C1]

Reaction of 100 mg (0.225 mmol) of the compound from Example 58A with 38.5 mg (0.270 mmol) of 1-(2-chloropyridin-3-yl)methanamine, 111 mg (0.292 mmol) of HATU and 0.27 ml (1.6 mmol) of N,N-diisopropylethylamine in 1.0 ml of DMF and subsequent separation of the reaction mixture by preparative HPLC [Method 12a] gave the title compound as trifluoroacetic acid salt (114 mg, 88% of theory)

LC-MS [Method 1]: Rₜ=0.54 min; MS (ESIpos): m/z=455 (M−CF₃COOH+H)⁺

¹H-NMR (400 MHz, DMSO-d₆): δ [ppm]=0.92 (d, 3H), 1.00-1.18 (m, 1H), 1.59-1.77 (m, 4H), 1.78-2.17 (m, 4H), 2.56-2.65 (m, 1H), 2.71-2.97 (m, 2H), 2.98-3.20 (m, 1H), 3.26-3.53 (m, 3H), 3.54-3.72 (m, 1H), 4.54 (d, 2H), 4.58-4.71 (m, 1H), 7.43 (dd, 1H), 7.53 (d, 2H), 7.80 (dd, 1H), 7.99 (d, 2H), 8.34 (dd, 1H), 9.18-9.28 (m, 2H).

### Example 84

### (R)—N-(2,6-Difluorobenzyl)-N-methyl-4-{[(3R)-3-methyl-1,4′-bipiperidin-1′-yl]carbonyl}benzamide trifluoroacetic acid salt

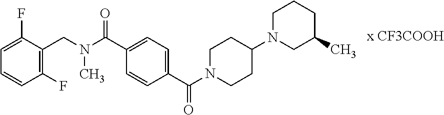 [SMILES: C[C@@H]1CCCN(C2CCN(C(=O)c3ccc(C(=O)N(C)Cc4c(F)cccc4F)cc3)CC2)C1]

Reaction of 100 mg (0.225 mmol) of the compound from Example 58A with 42 mg (0.270 mmol) of 1-(2,6-difluorophenyl)-N-methylmethanamine, 11 mg (0.292 mmol) of HATU and 0.27 ml (1.6 mmol) of N,N-diisopropylethylamine in 1.0 ml of DMF and separation by preparative HPLC [Method 12a] gave the title compound as trifluoroacetic acid salt (116 mg, 87% of theory)

LC-MS [Method 1]: Rₜ=0.67 min; MS (ESIpos): m/z=470 (M−CF₃COOH+H)⁺

¹H-NMR (400 MHz, DMSO-d₆): δ [ppm]=0.91 (d, 3H), 1.02-1.18 (m, 1H), 1.60-1.76 (m, 4H), 1.77-2.19 (m, 4H), 2.55-2.67 (m, 1H), 2.70-2.98 (m, 5H), 3.00-3.20 (m, 1H), 3.27-3.54 (m, 3H), 3.55-3.81 (m, 1H), 4.50-4.87 (m, 3H), 7.01-7.26 (m, 2H), 7.34-7.63 (m, 5H), 9.07-9.41 (m, 1H).

### Example 85

### 4-{[(3R)-3-Methyl-1,4′-bipiperidin-1′-yl]carbonyl}-N-{2,2,2-trifluoro-1-[4-(trifluoromethyl)phenyl]ethyl}benzamide trifluoroacetic acid salt

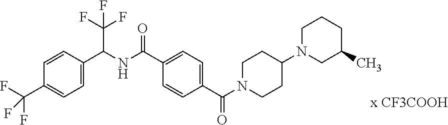 [SMILES: C[C@@H]1CCCN(C2CCN(C(=O)c3ccc(C(=O)NC(c4ccc(C(F)(F)F)cc4)C(F)(F)F)cc3)CC2)C1]

Reaction of 100 mg (0.225 mmol) of the compound from Example 58A with 66 mg (0.270 mmol) of 2,2,2-trifluoro-1-[4-(trifluoromethyl)phenyl]ethanamine, 111 mg (0.292 mmol) of HATU and 0.27 ml (1.6 mmol) of N,N-diisopropylethylamine in 1.0 ml of DMF and subsequent separation of the reaction mixture by preparative HPLC [Method 12b] gave the title compound as trifluoroacetic acid salt (41 mg, 27% of theory)

LC-MS [Method 1]: Rₜ=0.85 min; MS (ESIpos): m/z=556 (M−CF₃COOH+H)⁺

¹H-NMR (400 MHz, DMSO-d₆): δ [ppm]=0.92 (d, 3H), 1.02-1.17 (m, 1H), 1.57-1.77 (m, 4H), 1.78-2.20 (m, 4H), 2.56-2.65 (m, 1H), 2.72-2.94 (m, 2H), 2.99-3.15 (m, 1H), 3.26-3.52 (m, 3H), 3.54-3.68 (m, 1H), 4.58-4.70 (m, 1H), 6.26 (quin., 1H), 7.55 (d, 2H), 7.85 (d, 2H), 7.94-8.01 (m, 4H), 9.13-9.29 (m, 1H).

### Example 86

### (R)—N-[3-(Difluoromethoxy)benzyl]-4-[(3-methyl-1,4′-bipiperidin-1′-yl)carbonyl]benzamide trifluoroacetic acid salt

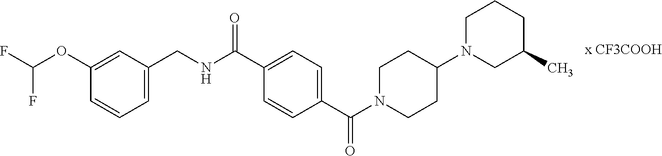 [SMILES: C[C@@H]1CCCN(C2CCN(C(=O)c3ccc(C(=O)NCc4cccc(OC(F)F)c4)cc3)CC2)C1]

Reaction of 100 mg (0.225 mmol) of the compound from Example 58A with 47 mg (0.270 mmol) of 1-[3-(difluoromethoxy)phenyl]methanamine, 111 mg (0.292 mmol) of HATU and 0.27 ml (1.6 mmol) of N,N-diisopropylethylamine in 1.0 ml of DMF and subsequent separation of the reaction mixture by preparative HPLC [Method 12a] gave the title compound as trifluoroacetic acid salt (64 mg, 47% of theory)

LC-MS [Method 1]: Rₜ=0.69 min; MS (ESIpos): m/z=486 (M−CF₃COOH+H)⁺

¹H-NMR (400 MHz, DMSO-d₆): δ [ppm]=0.92 (d, 3H), 1.02-1.17 (m, 1H), 1.60-1.77 (m, 4H), 1.77-2.18 (m, 4H), 2.56-2.66 (m, 1H), 2.72-2.98 (m, 2H), 2.98-3.19 (m, 1H), 3.30-3.53 (m, 3H), 3.55-3.69 (m, 1H), 4.51 (d, 2H), 4.56-4.69 (m, 1H), 7.04-7.09 (m, 1H), 7.11-7.14 (m, 1H), 7.19 (dd, 1H) 7.21-7.23 (m, 1H), 7.38-7.42 (m, 1H), 7.52 (d, 2H), 7.96 (d, 2H), 9.15-9.26 (m, 2H).

### Example 87

### N-[1-(2,6-Difluorophenyl)ethyl]-4-{[(3R)-3-methyl-1,4′-bipiperidin-1′-yl]carbonyl}benzamide trifluoroacetic acid salt

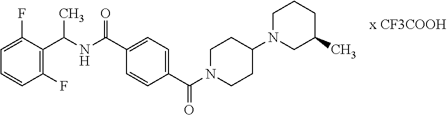 [SMILES: CC(NC(=O)c1ccc(C(=O)N2CCC(N3CCC[C@@H](C)C3)CC2)cc1)c1c(F)cccc1F]

Reaction of 50 mg (0.112 mmol) of the compound from Example 58A with 21 mg (0.135 mmol) of 1-(2,6-difluorophenyl)ethanamine, 56 mg (0.146 mmol) of HATU and 0.14 ml (0.78 mmol) of N,N-diisopropylethylamine in 1.0 ml of DMF and separation by preparative HPLC [Method 12a] gave the title compound as trifluoroacetic acid salt (44 mg, 67% of theory)

LC-MS [Method 1]: Rₜ=0.69 min; MS (ESIpos): m/z=470 (M−CF₃COOH+H)⁺

¹H-NMR (400 MHz, DMSO-d₆): δ [ppm]=0.91 (d, 3H), 1.01-1.17 (m, 1H), 1.58 (d, 3H), 1.61-1.77 (m, 4H), 1.77-2.17 (m, 4H), 2.56-2.65 (m, 1H), 2.72-2.97 (m, 2H), 2.98-3.18 (m, 1H), 3.28-3.52 (m, 3H), 4.55-4.71 (m, 1H), 5.32-5.45 (quin., 1H), 6.99-7.09 (m, 2H), 7.26-7.38 (m, 1H), 7.49 (d, 2H), 7.92 (d, 2H), 8.98 (d, 1H), 9.05-9.15 (m, 1H).

### Example 88

### N-(2,6-Difluorobenzyl)-4-{[(3R)-3-methyl-1,4′-bipiperidin-1′-yl]carbonyl}benzamide trifluoroacetic acid salt

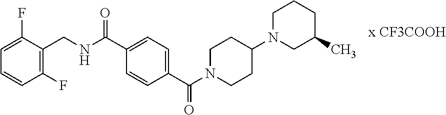 [SMILES: C[C@@H]1CCCN(C2CCN(C(=O)c3ccc(C(=O)NCc4c(F)cccc4F)cc3)CC2)C1]

Reaction of 50 mg (0.112 mmol) of the compound from Example 58A with 19 mg (0.135 mmol) of 1-(2,6-difluorophenyl)methanamine, 56 mg (0.146 mmol) of HATU and 0.14 ml (0.78 mmol) of N,N-diisopropylethylamine in 1.0 ml of DMF and separation by preparative HPLC [Method 12a] gave the title compound as trifluoroacetic acid salt (44 mg, 65% of theory)

LC-MS [Method 1]: Rₜ=0.64 min; MS (ESIpos): m/z=456 (M−CF₃COOH+H)⁺

¹H-NMR (400 MHz, DMSO-d₆): δ [ppm]=0.91 (d, 3H), 1.02-1.16 (m, 1H), 1.58-1.77 (m, 4H), 1.77-2.18 (m, 4H), 2.56-2.66 (m, 1H), 2.72-3.00 (m, 2H), 3.01-3.16 (m, 1H), 3.27-3.51 (m, 3H), 3.54-3.69 (m, 1H), 4.53 (d, 2H), 4.58-4.68 (m, 1H), 7.05-7.14 (m, 2H), 7.35-7.45 (m, 1H), 7.48 (d, 2H), 7.91 (d, 2H), 8.95-9.04 (m, 1H), 9.10-9.20 (m, 1H).

### Example 89

### N-[1-(2-Fluorophenyl)-3-hydroxypropyl]-4-{[(3R)-3-methyl-1,4′-bipiperidin-1′-yl]carbonyl}benzamide trifluoroacetic acid salt

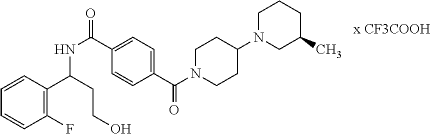 [SMILES: C[C@@H]1CCCN(C2CCN(C(=O)c3ccc(C(=O)NC(CCO)c4ccccc4F)cc3)CC2)C1]

Reaction of 50 mg (0.112 mmol) of the compound from Example 58A with 23 mg (0.135 mmol) of 3-amino-3-(2-fluorophenyl)propan-1-ol, 56 mg (0.146 mmol) of HATU and 0.14 ml (0.78 mmol) of N,N-diisopropylethylamine in 1.0 ml of DMF and separation by preparative HPLC [Method 12a] gave the title compound as trifluoroacetic acid salt (48 mg, 67% of theory)

LC-MS [Method 1]: Rₜ=0.59 min; MS (ESIpos): m/z=482 (M−CF₃COOH+H)⁺

¹H-NMR (400 MHz, DMSO-d₆): δ [ppm]=0.91 (d, 3H), 1.03-1.16 (m, 1H), 1.60-1.77 (m, 4H), 1.77-1.97 (m, 4H), 1.97-2.17 (m, 2H), 2.56-2.65 (m, 1H), 2.72-2.97 (m, 2H), 2.98-3.19 (m, 1H), 3.28-3.42 (m, 2H), 3.42-3.53 (m, 3H), 4.57-4.71 (m, 1H), 5.39-5.48 (m, 1H), 7.11-7.21 (m, 2H), 7.24-7.32 (m, 1H), 7.44-7.55 (m, 3H), 7.94 (d, 2H), 8.92 (d, 1H), 9.09-9.19 (m, 1H).

### Example 90

### N-[(4-Chloropyridin-2-yl)methyl]-4-{[(3R)-3-methyl-1,4′-bipiperidin-1′-yl]carbonyl}benzamide

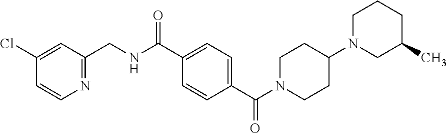 [SMILES: C[C@@H]1CCCN(C2CCN(C(=O)c3ccc(C(=O)NCc4cc(Cl)ccn4)cc3)CC2)C1]

Reaction of 50 mg (0.112 mmol) of the compound from Example 58A with 29 mg (0.135 mmol) of 1-(4-chloropyridin-2-yl)methanamine dihydrochloride, 56 mg (0.146 mmol) of HATU and 0.20 ml (1.1 mmol) of N,N-diisopropylethylamine in 1.0 ml of DMF and subsequent separation of the reaction mixture by preparative HPLC [Method 12a] and subsequent purification by column chromatography (10 g, silica gel cartridge, methanol) gave the title compound (6 mg, 11% of theory)

LC-MS [Method 1]: Rₜ=0.58 min; MS (ESIpos): m/z=455 (M+H)⁺

¹H-NMR (400 MHz, DMSO-d₆): δ [ppm]=0.75-0.89 (m, 4H), 1.32-1.69 (m, 7H), 1.70-1.84 (m, 2H), 2.00-2.12 (m, 1H), 2.70-2.81 (m, 3H), 2.94-3.07 (m, 1H), 3.46-3.60 (m, 1H), 4.43-4.56 (m, 1H), 4.56-4.63 (m, 2H), 7.40-7.46 (m, 2H), 7.49 (d, 2H), 7.96 (d, 2H), 8.50 (d, 1H), 9.19-9.27 (m, 1H).

### Example 91

### N-{[5-Chloro-3-(trifluoromethyl)pyridin-2-yl]methyl}-4-{[(3R)-3-methyl-1,4′-bipiperidin-1′-yl]carbonyl}benzamide

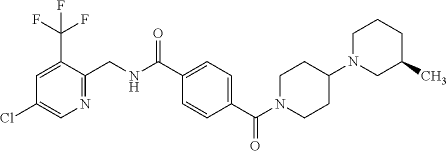 [SMILES: C[C@@H]1CCCN(C2CCN(C(=O)c3ccc(C(=O)NCc4ncc(Cl)cc4C(F)(F)F)cc3)CC2)C1]

Reaction of 50 mg (0.112 mmol) of the compound from Example 58A with 33 mg (0.135 mmol) of 1-[5-chloro-3-(trifluoromethyl)pyridin-2-yl]methanamine hydrochloride, 56 mg (0.146 mmol) of HATU and 0.20 ml (1.1 mmol) of N,N-diisopropylethylamine in 1.0 ml of DMF and subsequent separation of the reaction mixture by preparative HPLC [Method 12a] and subsequent purification by column chromatography (10 g, silica gel cartridge, methanol) gave the title compound (18 mg, 31% of theory)

LC-MS [Method 1]: Rₜ=0.71 min; MS (ESIpos): m/z=523 (M+H)⁺

¹H-NMR (400 MHz, DMSO-d₆): δ [ppm]=0.74-0.89 (m, 4H), 1.31-1.70 (m, 7H), 1.70-1.90 (m, 2H), 2.00-2.13 (m, 1H), 2.68-2.86 (m, 3H), 2.92-3.07 (m, 1H), 3.45-3.63 (m, 1H), 4.38-4.60 (m, 1H), 4.73 (d, 2H), 7.48 (d, 2H), 7.93 (d, 2H), 8.38 (d, 1H), 8.88 (d, 1H), 9.10-9.16 (m, 1H).

### Example 92

### N-{[5-Chloro-4-(trifluoromethyl)pyridin-2-yl]methyl}-4-{[(3R)-3-methyl-1,4′-bipiperidin-1′-yl]carbonyl}benzamide

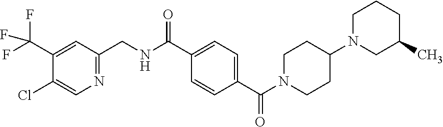 [SMILES: C[C@@H]1CCCN(C2CCN(C(=O)c3ccc(C(=O)NCc4cc(C(F)(F)F)c(Cl)cn4)cc3)CC2)C1]

Reaction of 50 mg (0.112 mmol) of the compound from Example 58A with 33 mg (0.135 mmol) of 1-[5-chloro-4-(trifluoromethyl)pyridin-2-yl]methanamine hydrochloride, 56 mg (0.146 mmol) of HATU and 0.20 ml (1.1 mmol) of N,N-diisopropylethylamine in 1.0 ml of DMF and subsequent separation of the reaction mixture by preparative HPLC [Method 12a] and subsequent purification by column chromatography (10 g, silica gel cartridge, methanol) gave the title compound (16 mg, 27% of theory).

LC-MS [Method 1]: Rₜ=0.70 min; MS (ESIpos): m/z=523 (M+H)⁺

¹H-NMR (400 MHz, DMSO-d₆): δ [ppm]=0.73-0.90 (m, 4H), 1.29-1.70 (m, 7H), 1.70-1.85 (m, 2H), 1.99-2.11 (m, 1H), 2.69-2.82 (m, 3H), 2.92-3.08 (m, 1H), 3.45-3.60 (m, 1H), 4.42-4.59 (m, 1H), 4.62-4.70 (m, 2H), 7.49 (d, 2H), 7.80 (s, 1H), 7.94 (d, 2H), 8.89 (s, 1H), 9.24-9.34 (m, 1H).

### Example 93

### 4-{[(3R)-3-Methyl-1,4′-bipiperidin-1′-yl]carbonyl}-N-[1-(pyridin-4-yl)ethyl]benzamide

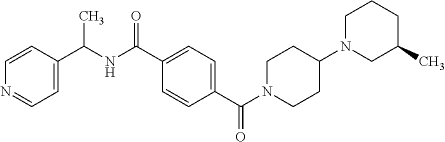 [SMILES: CC(NC(=O)c1ccc(C(=O)N2CCC(N3CCC[C@@H](C)C3)CC2)cc1)c1ccncc1]

A mixture of 50 mg (0.112 mmol) of the compound from Example 58A with 21 mg (0.135 mmol) of 1-(pyridin-4-yl)ethanamine hydrochloride, 56 mg (0.146 mmol) of HATU and 0.20 ml (1.1 mmol) of N,N-diisopropylethylamine in 1.0 ml of DMF was stirred at RT overnight. For work-up, 1 ml of saturated sodium bicarbonate solution and 5 ml of ethyl acetate were added and the mixture was filtered through an Extrelut® cartridge. The filtrate was concentrated giving, after purification of the crude product by column chromatography (10 g, silica gel cartridge, ethyl acetate/methanol gradient), the title compound (16 mg, 27% of theory).

LC-MS [Method 8]: Rₜ=0.28 min; MS (ESIpos): m/z=435 (M+H)⁺

¹H-NMR (400 MHz, DMSO-d₆): δ [ppm]=0.73-0.89 (m, 4H), 1.29-1.69 (m, 10H), 1.70-1.85 (m, 2H), 2.00-2.10 (m, 1H), 2.69-2.81 (m, 3H), 2.93-3.07 (m, 1H), 3.45-3.59 (m, 1H), 4.45-4.60 (m, 1H), 5.09-5.19 (m, 1H), 7.38 (d, 2H), 7.48 (d, 2H), 7.94 (d, 2H), 8.48-8.54 (m, 2H), 8.98 (d, 1H).

### Example 94

### N-[1-(2-Fluorophenyl)-2-hydroxyethyl]-4-{[(3R)-3-methyl-1,4′-bipiperidin-1′-yl]carbonyl}benzamide

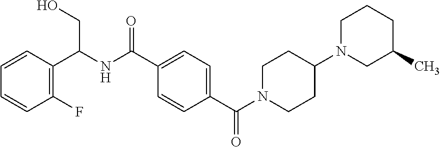 [SMILES: C[C@@H]1CCCN(C2CCN(C(=O)c3ccc(C(=O)NC(CO)c4ccccc4F)cc3)CC2)C1]

A mixture of 50 mg (0.112 mmol) of the compound from Example 58A with 20.9 mg (0.135 mmol) of 2-amino-2-(2-fluorophenyl)ethanol, 56 mg (0.146 mmol) of HATU and 0.20 ml (1.1 mmol) of N,N-diisopropylethylamine in 1.0 ml of DMF was stirred at RT overnight. For work-up, 1 ml of saturated sodium bicarbonate solution and 5 ml of ethyl acetate were added and the mixture was filtered through an Extrelut® cartridge. The filtrate was concentrated giving, after purification of the crude product by column chromatography (10 g, silica gel cartridge, ethyl acetate/methanol gradient), the title compound (42 mg, 74% of theory).

LC-MS [Method 8]: Rₜ=0.76 min; MS (ESIpos): m/z=468 (M+H)⁺

¹H-NMR (400 MHz, DMSO-d₆): δ [ppm]=0.73-0.89 (m, 4H), 1.32-1.86 (m, 9H), 1.99-2.11 (m, 1H), 2.69-2.82 (m, 3H), 2.92-3.07 (m, 1H), 3.44-3.58 (m, 1H), 3.60-3.75 (m, 2H), 4.45-4.56 (m, 1H), 5.08-5.17 (m, 1H), 5.34-5.42 (m, 2H), 7.00-7.54 (m, 6H), 7.94-7.94 (d, 2H), 8.89 (d, 1H). (diastereomer mixture)

### Example 95

### 4-{[(3R)-3-Methyl-1,4′-bipiperidin-1′-yl]carbonyl}-N-[(2-methylpyridin-3-yl)methyl]benzamide

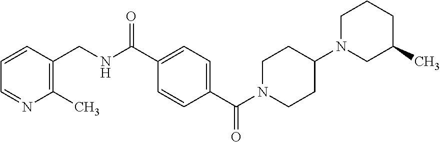 [SMILES: Cc1ncccc1CNC(=O)c1ccc(C(=O)N2CCC(N3CCC[C@@H](C)C3)CC2)cc1]

0.32 ml (1.8 mmol) of N,N-diisopropylethylamine and 131 mg (0.345 mmol) of HATU were added to a mixture of 118 mg (0.265 mmol) of the compound from Example 58A and 81 mg (0.530 mmol) of 1-(2-methylpyridin-3-yl)methanamine in 2.4 ml of DMF, and the mixture was stirred at RT overnight. For work-up, the mixture was diluted with ethyl acetate and the organic phase was washed repeatedly with saturated sodium bicarbonate solution and saturated sodium chloride solution. The organic phase was dried over magnesium sulphate, filtered and concentrated. Purification of the crude product by column chromatography (10 g, silica gel cartridge, ethyl acetate/methanol gradient) gave 24 mg of the title compound (21% of theory).

LC-MS [Method 8]: Rₜ=0.43 min; MS (ESIpos): m/z=435 (M+H)⁺

¹H-NMR (400 MHz, DMSO-d₆): δ [ppm]=0.75-0.87 (m, 4H), 1.31-1.68 (m, 7H), 1.70-1.85 (m, 2H), 1.98-2.11 (m, 1H), 2.52 (s, 3H), 2.69-2.81 (m, 3H), 2.92-3.08 (m, 1H), 3.45-3.58 (m, 1H), 4.42-4.59 (m, 3H), 7.19 (dd, 1H), 7.48 (d, 2H), 7.59 (dd, 1H), 7.94 (d, 2H), 8.33 (dd, 1H), 9.06-9.12 (m, 1H).

### Example 96

### N-(2, 6-Difluorobenzyl)-4-{[3-(methoxymethyl)-1,4′-bipiperidin-1′-yl]carbonyl}-N-methylbenzamide trifluoroacetic acid salt

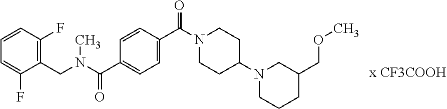 [SMILES: COCC1CCCN(C2CCN(C(=O)c3ccc(C(=O)N(C)Cc4c(F)cccc4F)cc3)CC2)C1 ; "x CF3COOH"]

0.12 ml (0.12 mmol) of a 1 M solution of titanium tetrachloride in dichloromethane were added to a solution of 90 mg (0.233 mmol) of the compound from Example 39A and 90 mg (0.699 mmol) of 3-(methoxymethyl)piperidine in 2.0 ml of dichloromethane, and the mixture was stirred at RT overnight. A solution of 44 mg (0.70 mmol) of sodium cyanoborohydride in 2.0 ml of methanol was then added, and the mixture was stirred for 15 min. For work-up, 2.0 ml of a 1N EDTA solution were added, the mixture was stirred briefly and then filtered through a kieselguhr pad and the pad was washed with dichloromethane. The filtrate was washed with saturated sodium chloride solution and the organic phase was dried over magnesium sulphate, filtered and concentrated. Separation of the residue by preparative HPLC [Method 11] gave 55 mg (38% of theory) of the title compound as trifluoroacetic acid salt.

LC-MS [Method 2]: Rₜ=0.71 min; MS (ESIpos): m/z=500 (M−CF₃COOH+H)⁺

¹H-NMR (400 MHz, DMSO-d₆): δ [ppm]=1.11-1.30 (m, 1H), 1.59-1.78 (m, 3H), 1.84-2.14 (m, 4H), 2.69-2.94 (m, 5H), 2.99-3.17 (m, 1H), 3.17-3.23 (m, 5H), 3.35-3.46 (m, 2H), 3.47-3.58 (m, 1H), 4.52-4.71 (m, 2H), 4.72-4.86 (m, 1H), 7.05-7.22 (m, 2H), 7.36-7.58 (m, 5H), 9.10-9.24 (m, 1H).

### Example 97

### N-[(3,5-Difluoropyridin-2-yl)methyl]-4-{[3-(methoxymethyl)-1,4′-bipiperidin-1′-yl]carbonyl}benzamide trifluoroacetic acid salt

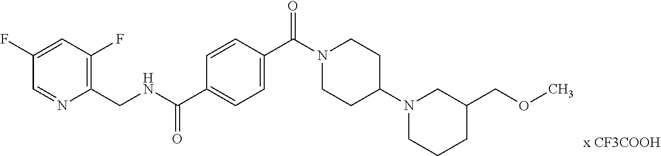 [SMILES: COCC1CCCN(C2CCN(C(=O)c3ccc(C(=O)NCc4ncc(F)cc4F)cc3)CC2)C1]

0.12 ml (0.12 mmol) of a 1 M solution of titanium tetrachloride in dichloromethane were added to a solution of 88 mg (0.236 mmol) of the compound from Example 38A and 91 mg (0.707 mmol) of 3-methoxymethylpiperidine in 2.0 ml of dichloromethane, and the mixture was stirred at RT overnight. A solution of 44 mg (0.707 mmol) of sodium cyanoborohydride in 2.0 ml of methanol was then added, and the mixture was stirred for 15 min. For work-up, 2.0 ml of a 1N EDTA solution were added and the mixture was stirred briefly and then filtered through a kieselguhr pad which was washed with dichloromethane. The filtrate was washed with saturated sodium chloride solution and the organic phase was dried over magnesium sulphate, filtered and concentrated. Separation of the residue by preparative HPLC [Method 11] gave 44 mg (28% of theory) of the title compound as trifluoroacetic acid salt.

LC-MS [Method 2]: Rₜ=0.59 min; MS (ESIpos): m/z=487 (M−CF₃COOH+H)⁺

¹H-NMR (400 MHz, CDCl₃): δ [ppm]=1.26-1.45 (m, 1H), 1.77-1.89 (m, 1H), 1.90-2.00 (m, 1H), 2.01-2.26 (m, 3H), 2.28-2.44 (m, 1H), 2.77-2.92 (m, 1H), 2.95-3.22 (m, 1H), 3.24-3.43 (m, 5H), 3.44-3.58 (m, 2H), 4.82 (d, 2H), 4.87-5.02 (m, 1H), 7.26-7.31 (m, 1H), 7.49 (d, 2H), 7.54-7.62 (m, 1H), 7.93 (d, 2H), 8.33 (d, 1H), 12.05-12.46 (m, 1H).

### Example 98

### N-[(3-Fluoropyridin-2-yl)methyl]-4-{[(3R)-3-methyl-1,4′-bipiperidin-1′-yl]carbonyl}benzamide

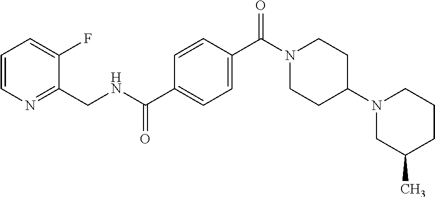 [SMILES: C[C@@H]1CCCN(C2CCN(C(=O)c3ccc(C(=O)NCc4ncccc4F)cc3)CC2)C1]

At 0° C., 0.19 ml (0.324 mmol) of T3P (50% by weight solution in DMF) was added to a solution of 150 mg (0.270 mmol) of the compound from Example 58A with 64.4 mg (0.324 mmol) of 1-(3-fluoropyridin-2-yl)methanamine dihydrochloride and 0.47 ml (2.7 mmol) of N,N-diisopropylethylamine in 2.7 ml of acetonitrile, and the mixture was then stirred at RT overnight. For work-up, the volatile constituents were removed under reduced pressure and the residue was taken up in ethyl acetate and washed repeatedly with saturated sodium bicarbonate solution and saturated sodium chloride solution. The organic phase was dried over magnesium sulphate, filtered and concentrated. Purification by column chromatography (10 g, silica gel cartridge, ethyl acetate/methanol gradient) gave 53 mg (45% of theory) of the title compound.

LC-MS [Method 1]: Rₜ=0.52 min; MS (ESIpos): m/z=439 (M+H)⁺

¹H-NMR (400 MHz, DMSO-d₆): δ [ppm]=0.73-0.88 (m, 4H), 1.30-1.68 (m, 8H), 1.70-1.85 (m, 2H), 1.99-2.11 (m, 1H), 2.69-2.81 (m, 3H), 2.86-3.07 (m, 1H), 3.43-3.61 (m, 1H), 4.42-4.57 (m, 1H), 4.63-4.68 (m, 2H), 7.37-7.43 (m, 1H), 7.46 (d, 2H), 7.67-7.73 (m, 1H), 7.91-7.95 (m, 2H), 8.36-8.39 (m, 1H), 9.03-9.09 (m, 1H).

### Example 99

### [3-Chloro-4-(2-hydroxypropan-2-yl)phenyl][(3R)-3-methyl-1,4′-bipiperidin-1′-yl]methanone

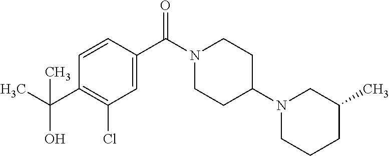 [SMILES: C[C@@H]1CCCN(C2CCN(C(=O)c3ccc(C(C)(C)O)c(Cl)c3)CC2)C1]

132 mg (0.402 mmol) of the compound from Example 40A, 147 mg (0.804 mmol) of the compound from Example 56A, 53.1 mg (0.201 mmol) of molybdenum hexacarbonyl, 19 mg (0.020 mmol) of trans-bis(acetato)bis[o-(di-o-tolylphosphine)benzyl]dipalladium(II) (Herrmann's Palladacycle) and 128 mg (1.21 mmol) of sodium carbonate were suspended in 4 ml of water and heated in a CEM microwave (300 W) at 150° C. for 10 min. After cooling, the mixture was extracted with ethyl acetate and filtered through an Extrelut® cartridge, and the filtrate was concentrated. The residue was purified by preparative HPLC [Method 14]. The product-containing fractions were combined, concentrated and dried under HV. This gave 31 mg of the title compound as a white foam (21% of theory).

LC-MS [Method 1]: Rₜ=0.62 min; MS (ESIpos): m/z=379 (M+H)⁺

¹H-NMR (400 MHz, DMSO-d₆): δ [ppm]=0.72-0.92 (m, 4H), 1.35-1.90 (m, 16H), 1.98-2.12 (m, 1H), 2.65-2.84 (m, 3H), 2.90-3.09 (m, 1H), 3.46-3.68 (m, 1H), 4.37-4.57 (m, 1H), 5.38 (s, 1H), 7.33 (dd, 1H), 7.37 (d, 1H), 7.87 (d, 1H).

### Example 100

### N-tert-Butyl-2-chloro-4-{[(3R)-3-methyl-1,4′-bipiperidin-1′-yl]carbonyl}benzamide formic acid salt

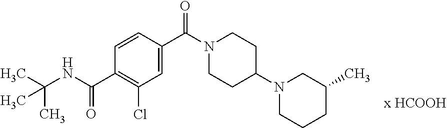 [SMILES: C[C@@H]1CCCN(C2CCN(C(=O)c3ccc(C(=O)NC(C)(C)C)c(Cl)c3)CC2)C1]

110 mg (0.379 mmol) of the compound from Example 41A, 138 mg (0.757 mmol) of the compound from Example 56A, 50 mg (0.189 mmol) of molybdenum hexacarbonyl, 18 mg (0.019 mmol) of trans-bis(acetato)bis[o-(di-o-tolylphosphine)benzyl]dipalladium(II) (Herrmann's Palladacycle) and 120 mg (1.14 mmol) of sodium carbonate were suspended in 1.3 ml of water and heated in a CEM microwave (300 W) at 150° C. for 10 min. After cooling, the mixture was extracted with ethyl acetate and filtered through an Extrelut® cartridge, and the filtrate was concentrated. The crude product was purified by preparative HPLC [Method 14]. This gave 30 mg of the title compound as formic acid salt (17% of theory).

LC-MS [Method 2]: Rₜ=0.71 min; MS (ESIpos): m/z=420 (M+H)⁺

¹H-NMR (400 MHz, DMSO-d₆): δ [ppm]=0.78-0.91 (m, 4H), 1.35 (s, 9H), 1.39-1.93 (m, 8H), 2.10-2.22 (m, 1H), 2.57-2.68 (m, 1H), 2.69-2.87 (m, 3H), 2.97-3.09 (m, 1H), 3.48-3.58 (m, 2H), 4.42-4.54 (m, 1H), 7.35 (dd, 1H), 7.39 (d, 1H), 7.47 (d, 1H), 8.09 (s, 1H), 8.17 (s, 1H).

### Example 101

### N-tert-Butyl-3-chloro-4-{[(3R)-3-methyl-1,4′-bipiperidin-1′-yl]carbonyl}benzamide formic acid salt

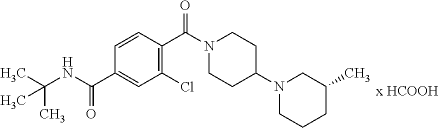 [SMILES: C[C@@H]1CCCN(C2CCN(C(=O)c3ccc(C(=O)NC(C)(C)C)cc3Cl)CC2)C1]

At 0° C., 0.23 ml (0.38 mmol) of T3P (50% by weight solution in ethyl acetate) was added to a solution of 114 mg (0.380 mmol) of the compound from Example 43A with 58 mg (0.316 mmol) of (3R)-3-methyl-1,4′-bipiperidine and 0.27 ml (1.6 mmol) of N,N-diisopropylethylamine in 2.6 ml of acetonitrile, and the mixture was then stirred at RT overnight. For work-up, the volatile constituents were removed under reduced pressure and the residue was taken up in ethyl acetate and washed repeatedly with saturated sodium bicarbonate solution and saturated sodium chloride solution. The organic phase was dried over magnesium sulphate, filtered and concentrated. Purification of the crude product by preparative HPLC [Method 15] gave 45 mg of the title compound as formic acid salt (23% of theory).

LC-MS [Method 8]: Rₜ=0.84 min; MS (ESIpos): m/z=420 (M+H)⁺

¹H-NMR (400 MHz, DMSO-d₆): δ [ppm]=0.75-0.89 (m, 4H), 1.27-1.48 (m, 12H), 1.48-1.72 (m, 4H), 1.74-1.89 (m, 2H), 2.01-2.15 (m, 1H), 2.72-2.82 (m, 3H), 2.91-3.06 (m, 2H), 4.46-4.59 (m, 1H), 7.39 (d, 0.5H), 7.49 (d, 0.5H), 7.77-7.83 (m, 1H), 7.89-7.94 (m, 1H), 7.95-8.00 (m, 1H), 8.16 (s, 1H) (rotamers).

### Example 102

### N-tert-Butyl-3-fluoro-4-[(3-methyl-1,4′-bipiperidin-1′-yl)carbonyl]benzamide hydrochloride

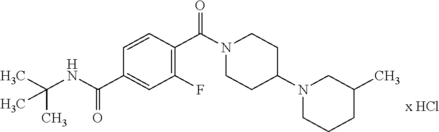 [SMILES: CC1CCCN(C2CCN(C(=O)c3ccc(C(=O)NC(C)(C)C)cc3F)CC2)C1]

125 mg (0.46 mmol) of the compound from Example 53A, 100 mg (0.55 mmol) of 3-methyl-1,4′-bipiperidine, 60 mg (0.23 mmol) of molybdenum hexacarbonyl, 21 mg (0.032 mmol) of trans-bis(acetato)bis[o-(di-o-tolylphosphine)benzyl]dipalladium(II) (Herrmann's Palladacycle) and 145 mg (1.37 mmol) of sodium carbonate were suspended in 3 ml of water and heated in a CEM microwave (300 W) at 150° C. for 10 min. After cooling, the mixture was diluted with water and extracted with ethyl acetate and filtered through Celite. The organic phase was removed from the filtrate, dried over sodium sulphate, filtered and concentrated under reduced pressure. The crude product was purified by chromatography on silica gel (elution: 1. ethyl acetate, 2. ethyl acetate/methanol 3:1). After concentration and drying of the product fractions under HV, the product was stirred with etheral hydrogen chloride, and a little 2-propoanol was added. The solid was decanted off. The residue was dissolved in methanol, concentrated and dried under HV. This gave 37 mg (17% of theory) of the target compound.

LC-MS [Method 2]: Rₜ=0.66 min; MS (ESIpos): m/z=404 (M+H)⁺

¹H-NMR (400 MHz, DMSO-d₆): δ [ppm]=0.90 (d, 3H), 1.01-1.14 (m, 1H), 1.34-1.42 (m, 9H), 1.47-1.89 (m, 5H), 1.91-2.10 (m, 2H), 2.16-2.27 (m, 1H), 2.74-2.89 (m, 2H), 3.04-3.20 (m, 2H), 3.24-3.51 (m, 4H), 4.65 (d, 1H), 7.50 (br. s., 1H), 7.68-7.76 (m, 2H), 7.94 (s, 1H).

### Example 103

### N-tert-Butyl-2-fluoro-4-[(3-methyl-1,4′-bipiperidin-1′-yl)carbonyl]benzamide

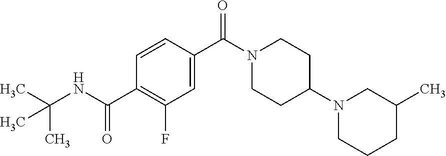 [SMILES: CC1CCCN(C2CCN(C(=O)c3ccc(C(=O)NC(C)(C)C)c(F)c3)CC2)C1]

255 mg (0.93 mmol) of the compound from Example 54A were reacted analogously to the compound from Example 102. Chromatography on silica gel (elution: 1. ethyl acetate, 2. ethyl acetate/methanol 3:1) gave 53 mg of the title compound (13% of theory).

LC-MS [Method 2]: Rₜ=0.67 min; MS (ESIpos): m/z=404 (M+H)⁺

¹H-NMR (400 MHz, DMSO-d₆): δ [ppm]=0.74-0.88 (m, 4H), 1.30-1.68 (m, 17H), 1.70-1.83 (m, 2H), 2.07 (t, 1H), 2.68-2.81 (m, 4H), 2.94-3.05 (m, 1H), 3.12-3.20 (m, 1H), 3.45-3.57 (m, 1H), 7.22 (dd, 1H), 7.28 (dd, 1H), 7.53 (t, 1H), 7.95 (s, 1H).

### Example 104

### 4-{[4-(4,5-Dimethyl-3,6-dihydropyridin-1 (2H)-yl)piperidin-1-yl]carbonyl}-N-(3,5-dimethyl-1,2-oxazol-4-yl)benzamide

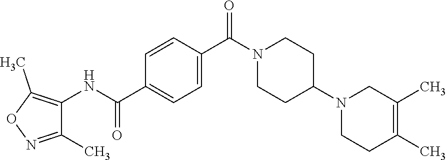 [SMILES: CC1=C(C)CN(C2CCN(C(=O)c3ccc(C(=O)Nc4c(C)noc4C)cc3)CC2)CC1]

27 mg (0.079 mmol) of the compound from Example 47A together with 18 mg (0.158 mmol) of 4,5-dimethyl-1,2,3,6-tetrahydropyridine were stirred in 2 ml of dichloromethane at RT for 1 h. 25 mg (0.119 mmol) of sodium triacetoxyborohydride were then added and the mixture was stirred at RT for a further 18 h. For workup, 1 ml of saturated sodium bicarbonate solution was added and the mixture was extracted three times with dichloromethane. The combined organic phases were dried over sodium sulphate, filtered and concentrated under reduced pressure. The crude product was purified chromatographically [Method 16]. This gave 26 mg (76% of theory) of the target compound.

LC-MS [Method 2]: Rₜ=0.58 min; MS (ESIpos): m/z=437 (M+H)⁺

¹H-NMR (400 MHz, DMSO-d₆): δ [ppm]=1.35-1.50 (m, 2H), 1.53 (s, 3H), 1.57 (s, 3H), 1.64-1.75 (m, 1H), 1.81-1.91 (m, 1H), 1.92-2.00 (m, 2H), 2.13 (s, 3H), 2.31 (s, 3H), 2.48-2.58 (3H, m), 2.76-2.90 (m, 3H), 2.97-3.10 (m, 1H), 3.48-3.58 (m, 1H), 4.41-4.51 (m, 1H), 7.54 (d, 2H), 8.01 (d, 2H), 9.88 (s, 1H).

### Example 105

### N-(3,5-Dimethyl-1,2-oxazol-4-yl)-4-{[3-(methoxymethyl)-1,4′-bipiperidin-1′-yl]carbonyl}benzamide

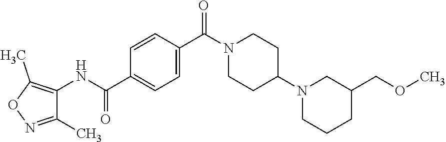 [SMILES: COCC1CCCN(C2CCN(C(=O)c3ccc(C(=O)Nc4c(C)noc4C)cc3)CC2)C1]

40 mg (0.117 mmol) of the compound from Example 47A were reacted analogously to the compound from Example 104. The crude product was purified chromatographically [Method 16]. This gave 21 mg (39% of theory) of the target compound.

LC-MS [Method 1]: Rₜ=0.49 min; MS (ESIpos): m/z=454 (M+H)⁺

¹H-NMR (400 MHz, DMSO-d₆): δ [ppm]=0.88-0.98 (m, 2H), 1.32-1.49 (m, 3H), 1.54-1.86 (m, 5H), 1.91 (t, 1H), 2.13 (s, 3H), 2.31 (s, 3H), 2.69-2.87 (m, 3H), 2.97-3.06 (m, 1H), 3.14-3.19 (m, 2H), 3.21 (s, 3H), 3.49-3.56 (m, 1H), 4.47-4.55 (m, 1H), 7.53 (d, 2H), 8.01 (d, 2H), 9.87 (s, 1H).

### Example 106

### [3-(tert-Butoxymethyl)-1,4′-bipiperidin-1′-yl][4-(2-hydroxypropan-2-yl)phenyl]methanone

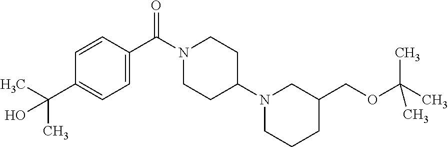 [SMILES: CC(C)(C)OCC1CCCN(C2CCN(C(=O)c3ccc(C(C)(C)O)cc3)CC2)C1]

49 mg (0.188 mmol) of the compound from Example 44A together with 82 mg (0.394 mmol) of the compound from Example 49A and 65 μl (0.375 mmol) of N,N-diisopropylethylamine were stirred in 3 ml of dichloromethane at RT for 1 h. 25 mg (0.119 mmol) of sodium triacetoxyborohydride were then added and the mixture was stirred at RT for a further 18 h. For workup, 1 ml of water was added and the mixture was extracted twice with dichloromethane. The combined organic phases were dried over sodium sulphate, filtered and concentrated under reduced pressure. The crude product was purified chromatographically [Method 16]. This gave 17 mg (21% of theory) of the target compound.

LC-MS [Method 2]: Rₜ=0.68 min; MS (ESIpos): m/z=417 (M+H)⁺

¹H-NMR (400 MHz, DMSO-d₆): δ [ppm]=0.88-0.97 (m, 1H), 1.10 (s, 9H), 1.35-1.46 (m, 9H), 1.52-1.67 (m, 5H), 1.86-1.96 (m, 1H), 2.07-2.17 (m, 1H), 2.65-3.05 (m, 5H), 3.11-3.16 (m, 2H), 3.50-3.71 (m, 1H), 4.34-4.59 (m, 1H), 5.07 (s, 1H), 7.30 (d, 2H), 7.51 (d, 2H).

### Example 107

### [3-(tert-Butoxymethyl)-1,4′-bipiperidin-1′-yl](4-tert-butylphenyl)methanone

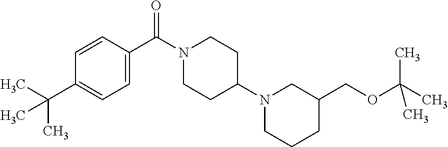 [SMILES: CC(C)(C)OCC1CCCN(C2CCN(C(=O)c3ccc(C(C)(C)C)cc3)CC2)C1]

49 mg (0.189 mmol) of the compound from Example 2A were reacted analogously to the compound from Example 106. This gave 40 mg (51% of theory) of the target compound.

LC-MS [Method 2]: Rₜ=0.88 min; MS (ESIpos): m/z=415 (M+H)⁺

¹H-NMR (400 MHz, DMSO-d₆): δ [ppm]=0.85-0.99 (m, 1H), 1.05-1.15 (m, 9H), 1.22-1.46 (m, 13H), 1.51-1.80 (m, 5H), 1.90 (t, 1H), 2.11 (t, 1H), 2.65-2.85 (m, 3H), 2.90-3.08 (m, 1H), 3.09-3.18 (m, 2H), 3.50-3.74 (m, 1H), 4.32-4.60 (m, 1H), 7.30 (d, 2H), 7.44 (d, 2H).

### Example 108

### {3-[(3-Fluorophenoxy)methyl]-1,4′-bipiperidin-1′-yl}[4-(2-hydroxypropan-2-yl)phenyl]methanone

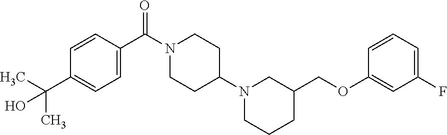 [SMILES: CC(C)(O)c1ccc(C(=O)N2CCC(N3CCCC(COc4cccc(F)c4)C3)CC2)cc1]

Analogously to the compound from Example 104, 47 mg (0.168 mmol) of the compound from Example 44A were reacted with the compound from Example 51A. This gave 20 mg (26% of theory) of the target compound.

LC-MS [Method 1]: Rₜ=0.65 min; MS (ESIpos): m/z=455 (M+H)⁺

¹H-NMR (400 MHz, DMSO-d₆): δ [ppm]=1.01-1.14 (m, 1H), 1.33-1.51 (m, 9H), 1.59-1.79 (m, 4H), 1.87-1.97 (m, 1H), 2.08 (t, 1H), 2.19 (t, 1H), 2.68-2.78 (m, 2H), 2.86-3.09 (m, 3H), 3.52-3.70 (m, 1H), 3.81-3.89 (m, 2H), 4.37-4.59 (m, 1H), 5.08 (s, 1H), 6.69-6.84 (m, 3H), 7.25-7.34 (m, 3H), 7.50 (d, 2H).

### Example 109

### (4-tert-Butylphenyl){3[3-(2-methoxyethyl)-1,2,4-oxadiazol-5-yl]-1,4′-bipiperidin-1′-}methanone

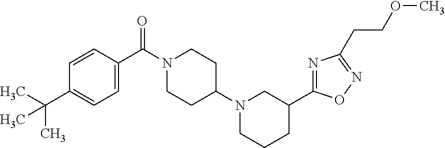 [SMILES: COCCc1noc(C2CCCN(C3CCN(C(=O)c4ccc(C(C)(C)C)cc4)CC3)C2)n1]

70 mg (0.171 mmol) of the compound from Example 52A were dissolved in 5 ml of DMF, the solution was heated to 60° C. and 42 mg (0.257 mmol) of CDI were added at this temperature. The mixture was stirred at this temperature for 1 h and then, after cooling to RT, 30 mg (0.257 mmol) of N′-hydroxy-3-methoxypropanimidamide were added. The mixture was stirred initially at 40° C. for 2 h and then at 115° C. for 2 h. For work-up, the mixture was cooled to RT and diluted with 1 ml of methanol. The crude mixture was purified directly chromatographically [Method 16]. This gave 30 mg (39% of theory) of the target compound.

LC-MS [Method 8]: Rₜ=0.92 min; MS (ESIpos): m/z=455 (M+H)⁺

¹H-NMR (400 MHz, DMSO-d₆): δ [ppm]=1.29 (s, 9H), 1.32-1.47 (m, 2H), 1.48-1.86 (m, 5H), 1.92-2.03 (m, 1H), 2.26-2.35 (m, 1H), 2.46-2.64 (m, 2H), 2.66-2.78 (m, 2H), 2.91 (t, 2H), 2.94-3.07 (m, 2H), 3.09-3.19 (m, 1H), 3.23 (s, 3H), 3.66 (t, 2H), 7.31 (d, 2H), 7.44 (d, 2H).

### Example 110

### [4-(2-Hydroxypropan-2-yl)phenyl]{3-[(trifluoromethoxy)methyl]-1,4′-bipiperidin-1′-yl}methanone

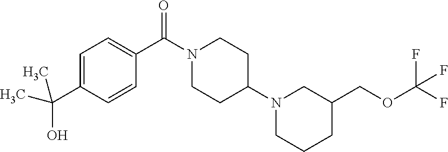 [SMILES: CC(C)(O)c1ccc(C(=O)N2CCC(N3CCCC(COC(F)(F)F)C3)CC2)cc1]

56 μl (0.26 mmol) of N,N-diisopropylethylamine and a spatula of molecular sieves were added to a mixture of 58 mg (0.264 mmol) of the compound from Example 61A and 166 mg (0.634 mmol) of the compound from Example 44A in 2.9 ml of dichloromethane, and the mixture was stirred at RT for 1 h. 112 mg (0.528 mmol) of sodium triacetoxyborohydride were then added, and the reaction was stirred at RT overnight. For work-up, 1 ml of water was added, the mixture was filtered through an Extrelut cartridge eluted with ethyl acetate and the filtrate was concentrated and purified by preparative HPLC [Method 13]. This gave 24 mg (19% of theory) of the title compound.

LC-MS [Method 1]: Rₜ=0.62 min; MS (ESIpos): m/z=429 (M+H)⁺

¹H-NMR (400 MHz, DMSO-d₆): δ [ppm]=0.98-1.13 (m, 1H), 1.30-1.50 (m, 9H), 1.52-1.91 (m, 5H), 2.02-2.14 (m, 1H), 2.14-2.26 (m, 1H), 2.63-2.82 (m, 2H), 2.89-3.13 (m, 1H), 3.47-3.73 (m, 1H), 3.93-4.01 (m, 2H), 4.40-4.58 (m, 1H), 5.09 (s, 1H), 7.30 (d, 2H), 7.51 (d, 2H).

### Example 111

### [3-(Cyclobutylmethoxy)-1,4′-bipiperidin-1-yl][4-(2-hydroxypropan-2-yl)phenyl]methanone

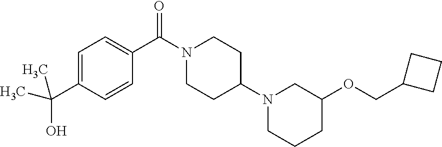 [SMILES: CC(C)(O)c1ccc(C(=O)N2CCC(N3CCCC(OCC4CCC4)C3)CC2)cc1]

100 μl (0.574 mmol) of N,N-diisopropylethylamine and a spatula tip of molecular sieves were added to a mixture of 118 mg (0.574 mmol) of 3-(cyclobutylmethoxy)piperidine hydrochloride and 75.0 mg (0.287 mmol) of the compound from Example 44A in 3.1 ml of dichloromethane, and the mixture was stirred at RT for 1 h. 121 mg (0.574 mmol) of sodium triacetoxyborohydride were then added, and the reaction was stirred at RT overnight. For work-up, 1 ml of water was added, the mixture was filtered through an Extrelut cartridge and eluted with ethyl acetate and the filtrate was concentrated. The crude product obtained was purified by preparative HPLC [Method 21]. This gave 29 mg (24% of theory) of the title compound.

LC-MS [Method 2]: Rₜ=0.69 min; MS (ESIpos): m/z=415 (M+H)⁺

¹H-NMR (400 MHz, DMSO-d₆): δ [ppm]=0.92-1.12 (m, 1H), 1.29-1.50 (m, 9H), 1.54-2.01 (m, 11H), 2.02-2.16 (m, 1H), 2.38-2.46 (m, 1H), 2.60-2.82 (m, 2H), 2.82-3.09 (m, 2H), 3.12-3.27 (m, 1H), 3.50-3.79 (m, 1H), 4.26-4.76 (m, 1H), 4.84-5.25 (m, 1H), 7.31 (d, 2H), 7.51 (d, 2H).

### Example 112

### 3-(Cyclopropyloxy)-1,4′-bipiperidin-1′-yl][4-(2-hydroxypropan-2-yl)phenyl]methanone formic acid salt

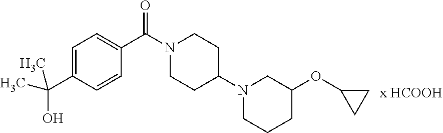 [SMILES: CC(C)(O)c1ccc(C(=O)N2CCC(N3CCCC(OC4CC4)C3)CC2)cc1]

34.3 μl (0.197 mmol) of N,N-diisopropylethylamine and a spatula of molecular sieves were added to a mixture of 35 mg (0.20 mmol) of the compound from Example 65A and 34 mg (0.13 mmol) of the compound from Example 44A in 1.0 ml of dichloromethane, and the mixture was stirred at RT for 1 h. 56 mg (0.263 mmol) of sodium triacetoxyborohydride were then added, and the reaction was stirred at RT overnight. For work-up, 1 ml of water was added, the mixture was filtered through an Extrelut cartridge and eluted with dichloromethane and the filtrate was concentrated. The crude product obtained was purified by preparative HPLC [Method 22]. This gave 12 mg (21% of theory) of the title compound.

LC-MS [Method 1]: Rₜ=0.56 min; MS (ESIpos): m/z=387 (M+H)⁺

¹H-NMR (400 MHz, DMSO-d₆): δ [ppm]=0.36-0.46 (m, 4H), 1.02-1.18 (m, 1H), 1.30-1.46 (m, 9H), 1.56-1.95 (m, 4H), 1.99-2.19 (m, 2H), 2.62-2.80 (m, 2H), 2.82-3.09 (m, 2H), 3.49-3.71 (m, 1H), 4.38-4.64 (m, 1H), 5.08 (br. s, 1H), 7.31 (d, 2H), 7.51 (d, 2H), 8.14 (br. s, 1H).

### Example 113

### [3-Fluoro-4-(2-hydroxypropan-2-yl)phenyl][(3R)-3-methyl-1,4′-bipiperidin-1′-yl]methanone

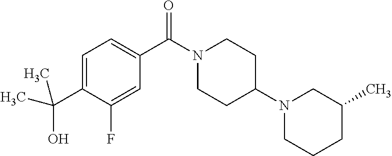 [SMILES: C[C@@H]1CCCN(C2CCN(C(=O)c3ccc(C(C)(C)O)c(F)c3)CC2)C1]

59 mg (0.25 mmol) of the compound from Example 66A were reacted analogously to the compound from Example 99. This gave 71 mg (77% of theory) of the target compound.

LC-MS [Method 9]: Rₜ=0.50 min; MS (ESIpos): m/z=363 (M+H)⁺

¹H-NMR (400 MHz, DMSO-d₆): δ [ppm]=0.83 (d, 3H), 1.31-1.87 (m, 10H), 1.49 (s, 6H), 2.06 (br. s., 1H), 2.61-2.87 (m, 4H), 3.00 (br. s., 1H), 3.59 (br. s., 1H), 4.49 (br. s., 1H), 5.35 (s, 1H), 7.10-7.22 (m, 2H), 7.67 (t, 1H).

### Example 114

### [3-Ethyl-1,4′-bipiperidin-1′-yl][4-(2-hydroxypropan-2-yl)phenyl]methanone

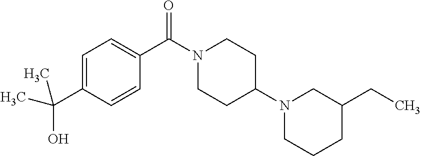 [SMILES: CCC1CCCN(C2CCN(C(=O)c3ccc(C(C)(C)O)cc3)CC2)C1]

Analogously to the compound from Example 110, 83 mg (0.32 mmol) of the compound from Example 44A were reacted with 100 mg (0.67 mmol) of 3-ethylpiperidine hydrochloride. The crude product was purified chromatographically [Method 16]. This gave 62 mg (54% of theory) of the target compound.

LC-MS [Method 9]: Rₜ=0.50 min; MS (ESIpos): m/z=359 (M+H)⁺

¹H-NMR (400 MHz, DMSO-d₆): δ [ppm]=0.70-0.82 (m, 1H), 0.70-0.89 (m, 1H), 0.85 (t, 3H), 1.06-1.25 (m, 2H), 1.27-1.46 (m, 4H), 1.43 (s, 6H), 1.52-1.87 (m, 5H), 1.98-2.16 (m, 1H), 2.60-2.85 (m, 2H), 2.90-3.04 (m, 1H), 3.55-3.67 (m, 1H), 4.42-4.53 (m, 1H), 5.08 (s, 1H), 7.30 (d, 2H), 7.50 (d, 2H).

### Example 115

### [3-Ethyl-1,4′-bipiperidin-1′-yl][4-(2-hydroxypropan-2-yl)phenyl]methanone (enantiomer 1)

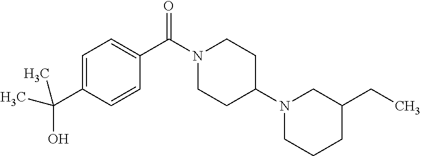 [SMILES: CCC1CCCN(C2CCN(C(=O)c3ccc(C(C)(C)O)cc3)CC2)C1]

530 mg (2.03 mmol) of the compound from Example 44A were reacted analogously to the compound from Example 114. The crude product obtained after work-up was separated into its enantiomers by preparative chiral chromatography [Method 17A].

Enantiomer 1: 150 mg (21% of theory) of the first eluting isomer were obtained.

Chiral analytical HPLC [Method 18a]: Rₜ=5.34 min

LC-MS [Method 10]: Rₜ=1.33 min; MS (ESIpos): m/z=359 (M+H)⁺

Enantiomer 2: 197 mg (27% of theory) of the last eluting isomer were obtained.

Chiral analytical HPLC [Method 18a]: Rₜ=5.94 min

### Example 116

### {3-[(Cyclobutyloxy)methyl]-1,4′-bipiperidin-1′-yl}[4-(2-hydroxypropan-2-yl)phenyl]methanone (racemate)

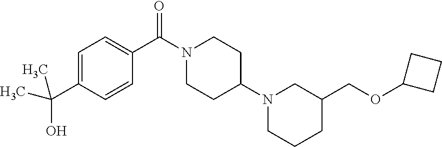 [SMILES: CC(C)(O)c1ccc(C(=O)N2CCC(N3CCCC(COC4CCC4)C3)CC2)cc1]

Analogously to the compound from Example 110, 38 mg (0.15 mmol) of the compound from Example 44A were reacted with 60 mg (0.29 mmol) of the compound from Example 69A. The crude product was purified chromatographically [Method 16]. This gave 16 mg (26% of theory) of the target compound.

LC-MS [Method 1]: Rₜ=0.59 min; MS (ESIpos): m/z=415 (M+H)⁺

¹H-NMR (400 MHz, DMSO-d₆): δ [ppm]=0.84-0.97 (m, 1H), 1.29-1.50 (m, 5H), 1.43 (s, 6H), 1.51-1.84 (m, 8H), 1.90 (t, 1H), 2.05-2.18 (m, 3H), 2.63-2.85 (m, 3H), 2.88-3.02 (m, 1H), 3.03-3.16 (m, 2H), 3.57-3.69 (m, 1H), 3.82 (quin, 1H), 4.44-4.54 (m, 1H), 5.08 (s, 1H), 7.30 (d, 2H), 7.50 (d, 2H).

### Example 117

### {3-[(Cyclobutyloxy)methyl]-1,4′-bipiperidin-1′-yl}[4-(2-hydroxypropan-2-yl)phenyl]methanone (enantiomer 2)

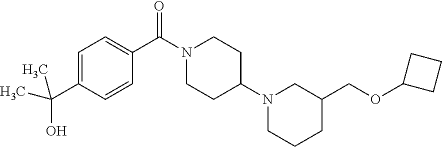 [SMILES: CC(C)(O)c1ccc(C(=O)N2CCC(N3CCCC(COC4CCC4)C3)CC2)cc1]

210 mg (0.80 mmol) of the compound from Example 44A were reacted analogously to the compound from Example 116. The crude product obtained after work-up was separated into its enantiomers by preparative chiral chromatography [Method 19A].

Enantiomer 1: 121 mg (36% of theory) of the first eluting isomer were obtained.

Chiral analytical HPLC [Method 20a]: Rₜ=4.84 min

Enantiomer 2: 133 mg (37% of theory) of the last eluting isomer were obtained.

Chiral analytical HPLC [Method 20a]: Rₜ=6.40 min

LC-MS [Method 9]: Rₜ=0.61 min; MS (ESIpos): m/z=415 (M+H)⁺

### Example 118

### {3-[(Cyclopropyloxy)methyl]-1,4′-bipiperidin-1′-yl}[4-(2-hydroxypropan-2-yl)phenyl]methanone

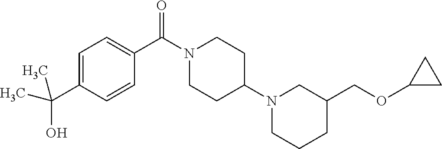 [SMILES: CC(C)(O)c1ccc(C(=O)N2CCC(N3CCCC(COC4CC4)C3)CC2)cc1]

201 mg (0.77 mmol) of the compound from Example 44A and 150 mg of molecular sieves were added to a solution of 179 mg (1.15 mmol) of the compound from Example 72A in 6.0 ml of dichloromethane, and the mixture was stirred at RT for 2 h. 244 mg (1.15 mmol) of sodium triacetoxyborohydride were then added, and the reaction was stirred at RT for 18 h. For work-up, the molecular sieves were filtered off and washed with a little dichloromethane, and 10 ml of saturated sodium bicarbonate solution were added. After separation of the phases, the aqueous phase was extracted two more times with in each case 10 ml of dichloromethane. The combined organic phases were dried over sodium sulphate, filtered and concentrated. The crude product was purified chromatographically [Method 16]. This gave 16 mg (25% of theory) of the target compound.

LC-MS [Method 9]: Rₜ=0.54 min; MS (ESIpos): m/z=401 (M+H)⁺

¹H-NMR (400 MHz, DMSO-d₆): δ [ppm]=0.33-0.47 (m, 4H), 0.82-0.98 (m, 1H), 1.29-1.48 (m, 4H), 1.43 (s, 6H), 1.53-1.74 (m, 5H), 1.89 (t, 1H), 2.12 (t, 1H), 2.63-2.83 (m, 3H), 2.81-3.02 (m, 1H), 3.56-3.67 (m, 1H), 3.16-3.30 (m, 3H), 4.44-4.52 (m, 1H), 5.08 (s, 1H), 7.30 (d, 2H), 7.50 (d, 2H).

### Example 119

### [3-(tert-Butoxymethyl)-1,4′-bipiperidin-1′-yl][4-(2-methoxypropan-2-yl)phenyl]methanone

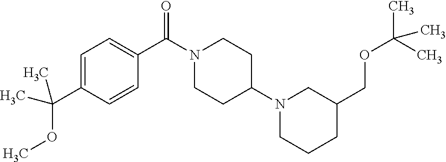 [SMILES: COC(C)(C)c1ccc(C(=O)N2CCC(N3CCCC(COC(C)(C)C)C3)CC2)cc1]

Analogously to the compound from Example 110, 50 mg (0.18 mmol) of the compound from Example 73A were reacted with 30 mg (0.15 mmol) of the compound from Example 49A. The crude product was purified chromatographically [Method 16]. This gave 10 mg (13% of theory) of the target compound.

LC-MS [Method 10]: Rₜ=1.73 min; MS (ESIpos): m/z=431 (M+H)⁺

¹H-NMR (400 MHz, DMSO-d₆): δ [ppm]=0.87-0.99 (m, 1H), 1.10 (s, 9H), 1.27-1.49 (m, 3H), 1.46 (s, 6H), 1.51-1.81 (m, 5H), 1.91 (br. s., 1H), 2.12 (br. s., 1H), 2.62-2.88 (m, 3H), 2.99 (s, 3H), 3.08-3.19 (m, 2H), 3.57-3.66 (m, 1H), 4.44-4.54 (m, 1H), 7.32-7.38 (m, 2H), 7.43 (d, 2H).

### Example 120

### [3-(Ethoxymethyl)-1,4′-bipiperidin-1′-yl][4-(2-hydroxypropan-2-yl)phenyl]methanone

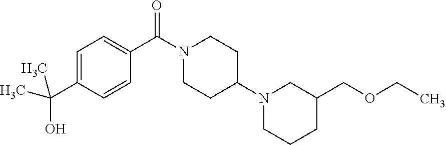 [SMILES: CCOCC1CCCN(C2CCN(C(=O)c3ccc(C(C)(C)O)cc3)CC2)C1]

Analogously to the compound from Example 110, 300 mg (1.15 mmol) of the compound from Example 44A were reacted with 413 mg (2.30 mmol) of 3-(ethoxymethyl)piperidine hydrochloride. The crude product was purified chromatographically [Method 16]. This gave 352 mg (69% of theory) of the target compound.

LC-MS [Method 9]: Rₜ=0.50 min; MS (ESIpos): m/z=389 (M+H)⁺

¹H-NMR (400 MHz, DMSO-d₆): δ [ppm]=0.84-0.97 (m, 1H), 1.09 (t, 3H), 1.26-1.47 (m, 4H), 1.43 (s, 6H), 1.53-1.72 (m, 5H), 1.90 (t, 1H), 2.11 (t, 1H), 2.65-2.86 (m, 3H), 2.93-3.03 (m, 1H), 3.14-3.24 (m, 2H), 3.34-3.42 (m, 2H), 3.57-3.67 (m, 1H), 4.44-4.54 (m, 1H), 5.08 (s, 1H), 7.30 (d, 2H), 7.51 (d, 2H).

### Example 121

### [3-(Ethoxymethyl)-1,4′-bipiperidin-1′-yl][4-(2-hydroxypropan-2-yl)phenyl]methanone (enantiomer 2)

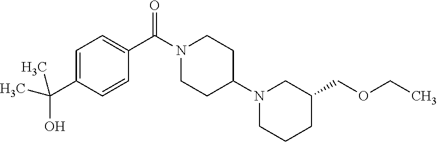 [SMILES: CCOC[C@@H]1CCCN(C2CCN(C(=O)c3ccc(C(C)(C)O)cc3)CC2)C1]

342 mg (0.88 mmol) of the compound from Example 120 were separated into its enantiomers by preparative chiral chromatography [Method 19b].

Enantiomer 1: 154 mg (35% of theory) of the first eluting isomer were obtained.

Chiral analytical HPLC [Method 20b]: Rₜ=5.17 min

Enantiomer 2: 139 mg (31% of theory) of the last eluting isomer were obtained.

Chiral analytical HPLC [Method 20b]: Rₜ=8.79 min

### Example 122

### [3-(Cyclopropylmethoxy)-1,4′-bipiperidin-1′-yl][4-(2-methoxypropan-2-yl)phenyl]methanone

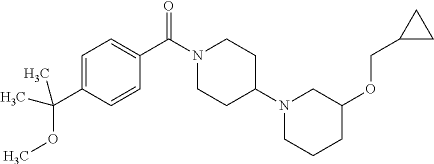 [SMILES: COC(C)(C)c1ccc(C(=O)N2CCC(N3CCCC(OCC4CC4)C3)CC2)cc1]

Analogously to the compound from Example 110, 50 mg (0.18 mmol) of the compound from Example 73A were reacted with 52 mg (0.27 mmol) of 3-(cyclopropylmethoxy)piperidine hydrochloride. The crude product was purified chromatographically [Method 16]. This gave 38 mg (50% of theory) of the target compound.

LC-MS [Method 1]: Rₜ=0.63 min; MS (ESIpos): m/z=415 (M+H)⁺

¹H-NMR (400 MHz, DMSO-d₆): δ [ppm]=0.07-0.17 (m, 2H), 0.38-0.48 (m, 2H), 0.88-1.11 (m, 2H), 1.27-1.51 (m, 4H), 1.46 (s, 6H), 1.54-1.83 (m, 3H), 1.85-1.99 (m, 2H), 2.09 (t, 1H), 2.59-2.79 (m, 2H), 2.90-3.05 (m, 5H), 3.18-3.30 (m, 3H), 3.61 (br. s., 1H), 4.49 (br. s., 1H), 7.36 (d, 2H), 7.44 (d, 2H).

### Example 123

### [4-(2-Hydroxypropan-2-yl)phenyl][(3R)-3-(methoxymethyl)-1,4′-bipiperidin-1′-yl]methanone (enantiomer 2)

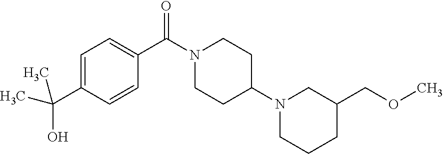 [SMILES: COCC1CCCN(C2CCN(C(=O)c3ccc(C(C)(C)O)cc3)CC2)C1]

294 mg (0.79 mmol) of the compound from Example 58 were separated into its enantiomers by preparative chiral chromatography [Method 19b].

Enantiomer 1: 141 mg (48% of theory) of the first eluting isomer were obtained.

Chiral analytical HPLC [Method 20b]: Rₜ=6.25 min

Enantiomer 2: 147 mg (49% of theory) of the last eluting isomer were obtained.

Chiral analytical HPLC [Method 20b]: Rₜ=14.12 min

LC-MS [Method 9]: Rₜ=0.44 min; MS (ESIpos): m/z=375 (M+H)⁺

### Example 124

### [3-(Cyclopropylmethoxy)-1,4′-bipiperidin-1′-yl][4-(3-hydroxyoxetan-3-yl)phenyl]methanone

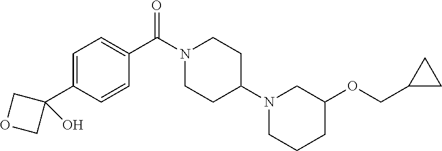 [SMILES: O=C(c1ccc(C2(O)COC2)cc1)N1CCC(N2CCCC(OCC3CC3)C2)CC1]

Analogously to the compound from Example 110, 70 mg (0.25 mmol) of the compound from Example 75A were reacted with 97 mg (0.51 mmol) of 3-(cyclopropylmethoxy)piperidine hydrochloride. The crude product was purified chromatographically [Method 16]. This gave 62 mg (55% of theory) of the target compound.

LC-MS [Method 9]: Rₜ=0.44 min; MS (ESIpos): m/z=415 (M+H)⁺

¹H-NMR (400 MHz, DMSO-d₆): δ [ppm]=0.06-0.17 (m, 2H), 0.38-0.46 (m, 2H), 0.87-1.13 (m, 2H), 1.25-1.48 (m, 3H), 1.53-1.83 (m, 3H), 1.85-1.98 (m, 2H), 2.09 (t, 1H), 2.59-2.82 (m, 2H), 2.91-3.07 (m, 2H), 3.21-3.33 (m, 4H), 3.57-3.65 (m, 1H), 4.46-4.54 (m, 1H), 4.66-4.81 (m, 2H), 6.44 (s, 1H), 7.42 (d, 2H), 7.65 (d, 2H).

### Example 125

### {3-[(Cyclobutyloxy)methyl]-1,4′-bipiperidin-1′-yl}[4-(3-hydroxyoxetan-3-yl)phenyl]methanone

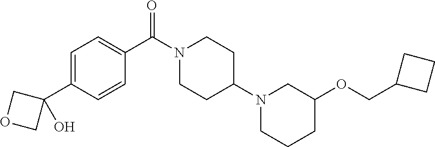 [SMILES: O=C(c1ccc(C2(O)COC2)cc1)N1CCC(N2CCCC(OCC3CCC3)C2)CC1]

Analogously to the compound from Example 110, 70 mg (0.25 mmol) of the compound from Example 75A were reacted with 105 mg (0.51 mmol) of the compound from Example 69A. The crude product was purified chromatographically [Method 16]. This gave 14 mg (12% of theory) of the target compound.

LC-MS [Method 1]: Rₜ=0.55 min; MS (ESIpos): m/z=429 (M+H)⁺

¹H-NMR (400 MHz, DMSO-d₆): δ [ppm]=0.80-0.97 (m, 1H), 1.34-1.49 (m, 4H), 1.53-1.66 (m, 3H), 1.70-1.82 (m, 3H), 1.90 (t, 1H), 2.03-2.17 (m, 5H), 2.64-2.75 (m, 2H), 2.77-3.16 (m, 4H), 3.57-3.67 (m, 1H), 3.82 (quin, 1H), 4.44-4.54 (m, 1H), 4.65-4.81 (m, 4H), 6.44 (s, 1H), 7.41 (d, 2H), 7.65 (d, 2H).

### Example 126

### (3-Cyclopropyl-1,4′-bipiperidin-1′-yl)[4-(2-hydroxypropan-2-yl)phenyl]methanone

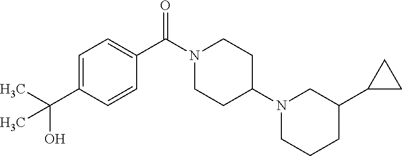 [SMILES: CC(C)(O)c1ccc(C(=O)N2CCC(N3CCCC(C4CC4)C3)CC2)cc1]

Analogously to the compound from Example 110, 210 mg (0.80 mmol) of the compound from Example 44A were reacted with 260 mg (1.61 mmol) of 3-cyclopropylpiperidine hydrochloride. The crude product was purified chromatographically [Method 16]. This gave 68 mg (23% of theory) of the target compound.

LC-MS [Method 2]: Rₜ=0.56 min; MS (ESIpos): m/z=371 (M+H)⁺

¹H-NMR (400 MHz, DMSO-d₆): δ [ppm]=0.01-0.07 (m, 2H), 0.26-0.38 (m, 2H), 0.41-0.56 (m, 1H), 0.59-0.76 (m, 1H), 0.99 (qd, 1H), 1.26-1.48 (m, 10H), 1.53-1.80 (m, 4H), 1.98 (t, 1H), 2.09 (t, 1H), 2.62-2.86 (m, 3H), 2.93-3.02 (m, 1H), 3.57-3.67 (m, 1H), 4.44-4.54 (m, 1H), 5.09 (s, 1H), 7.31 (d, 2H), 7.51 (d, 2H).

### Example 127

### [4-(2-Hydroxypropan-2-yl)phenyl]{3-[2-(trifluoromethoxy)ethoxy]-1,4′-bipiperidin-1′-yl}methanone

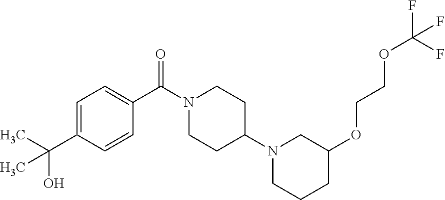 [SMILES: CC(C)(O)c1ccc(C(=O)N2CCC(N3CCCC(OCCOC(F)(F)F)C3)CC2)cc1]

Analogously to the compound from Example 110, 100 mg (0.38 mmol) of the compound from Example 44A were reacted with 191 mg (0.77 mmol) of 3-[2-(trifluoromethoxy)ethoxy]piperidine. The crude product was purified chromatographically [Method 16]. This gave 25 mg (14% of theory) of the target compound.

LC-MS [Method 2]: Rₜ=0.66 min; MS (ESIpos): m/z=459 (M+H)⁺

¹H-NMR (400 MHz, DMSO-d₆): δ [ppm]=0.99-1.16 (m, 1H), 1.27-1.46 (m, 5H), 1.43 (s, 6H), 1.49-2.03 (m, 5H), 2.11 (t, 1H), 2.59-2.77 (m, 2H), 2.97 (d, 2H), 3.56-3.73 (m, 3H), 4.13 (t, 2H), 4.44-4.54 (m, 1H), 5.09 (s, 1H), 7.31 (d, 2H), 7.51 (d, 2H).

### Example 128

### [3-(Cyclopropylmethoxy)-1,4′-bipiperidin-1′-yl][4-(2-hydroxypropan-2-yl)phenyl]methanone

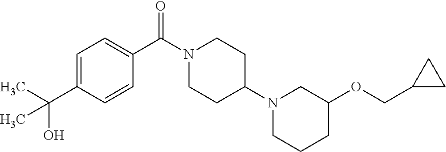 [SMILES: CC(C)(O)c1ccc(C(=O)N2CCC(N3CCCC(OCC4CC4)C3)CC2)cc1]

Analogously to the compound from Example 110, 750 mg (2.87 mmol) of the compound from Example 44A were reacted with 1.10 g (5.74 mmol) of 3-(cyclopropylmethoxy)piperidine hydrochloride. The crude product was purified chromatographically [Method 16]. This gave 693 mg (60% of theory) of the target compound.

LC-MS [Method 2]: Rₜ=0.62 min; MS (ESIpos): m/z=401 (M+H)⁺

¹H-NMR (400 MHz, DMSO-d₆): δ [ppm]=0.09-0.16 (m, 2H), 0.38-0.48 (m, 2H), 0.86-1.14 (m, 2H), 1.22-1.47 (m, 4H), 1.43 (s, 6H), 1.54-1.99 (m, 5H), 2.08 (t, 1H), 2.60-2.76 (m, 2H), 2.97 (d, 2H), 3.21-3.29 (m, 3H), 3.62 (br. s., 1H), 4.49 (br. s., 1H), 5.09 (s, 1H), 7.31 (d, 2H), 7.51 (d, 2H).

### Example 129

### [3-(Cyclopropylmethoxy)-1,4′-bipiperidin-1′-yl][4-(2-hydroxypropan-2-yl)phenyl]methanone hydrochloride (enantiomer 1)

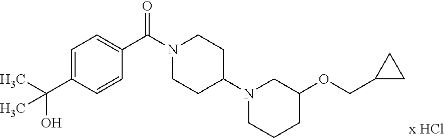 [SMILES: CC(C)(O)c1ccc(C(=O)N2CCC(N3CCCC(OCC4CC4)C3)CC2)cc1]

690 mg (1.72 mmol) of the compound from Example 128 were separated into its enantiomers by preparative chiral chromatography [Method 17b].

Enantiomer 1: 288 mg (41% of theory) of the first eluting isomer were obtained.

Chiral analytical HPLC [Method 18b]: Rₜ=4.20 min

Enantiomer 2: 282 mg (41% of theory) of the last eluting isomer were obtained.

Chiral analytical HPLC [Method 18b]: Rₜ=4.88 min

170 mg (0.42 mmol) of enantiomer 1 were dissolved in 2 ml of diethyl ether and 0.5 ml of a saturated solution of hydrogen chloride in diethyl ether was added with stirring. The resulting solution was concentrated and dried under HV. This gave 155 mg (84% of theory) of the target compound.

LC-MS [Method 9]: Rₜ=0.54 min; MS (ESIpos): m/z=401 (M+H, free base)

### B) ASSESSMENT OF PHYSIOLOGICAL EFFICACY

The suitability of the compounds according to the invention for treating cardiovascular disorders can be demonstrated in the following assay systems:

### B-1) In Vitro Assays

### B-1a) Antagonism Against Adrenoreceptors

Antagonism against the adrenoreceptor α_{1A} was tested using a recombinant human α_{1A} receptor CHO cell line which additionally also recombinantly expresses mtAeq (mitochondrial aequorin). Antagonism against the adrenoreceptor α_{2A} was tested using a recombinant human α_{2A}-Gα16 receptor fusion protein CHO cell line (PerkinElmer Life Sciences) which additionally also recombinantly expresses mtAeq. Antagonism against the adrenoreceptor α_{2B} was tested using a recombinant human α_{2B} receptor CHO cell line (PerkinElmer Life Sciences) which additionally also recombinantly expresses mtAeq. Antagonism against the adrenoreceptor α_{2C} was tested using a recombinant human α_{2C} receptor CHO cell line which additionally also recombinantly expresses a chimaric G protein (Gαqi3) and mtOb (mitochondrial obelin).

The cells were cultivated at 37° C. and 5% CO₂ in Dulbecco's modified Eagle's Medium/NUT mix F12 with L-glutamine which additionally contains 10% (v/v) inactivated foetal calf serum, 1 mM sodium pyruvate, 0.9 mM sodium bicarbonate, 50 U/ml penicillin, 50 μg/ml streptomycin, 2.5 μg/ml amphotericin B and 1 mg/ml Geneticin. The cells were passaged with enzyme-free Hank's-based cell dissociation buffer. All cell culture reagents used are from Invitrogen (Carlsbad, USA).

Luminescence measurements were carried out on white 384-well microtitre plates. 2000 cells/well were plated in a volume of 25 μl and cultivated for one day at 30° C. and 5% CO₂ in cell culture medium with coelenterazine (α_{2A} and α_{2B}: 5 μg/ml; α_{1a/c} and α_{2C}: 2.5 μg/ml). Serial dilutions of the test substances (10 μl) were added to the cells. After 5 minutes, noradrenalinee was added to the cells (35 μl; final concentrations: 20 nM (α_{1a/c} and α_{2C}) or 200 nM (α_{2A} and α_{2B})), and the emitted light was measured for 50 seconds using a CCD (charge-coupled device) camera (Hamamatsu Corporation, Shizuoka, Japan) in a light-tight box. The test substances were tested up to a maximum concentration of 10 μM. The IC₅₀ values were calculated from the appropriate dose-response curves. The results for the antagonism against the adrenoreceptor α_{2C} are shown in Table 1:

TABLE 1

| Example No. | IC₅₀ [nM] |
|---|---|
| 1 | 38 |
| 2 | 120 |
| 3 | 268 |
| 4 | 294 |
| 5 | 99 |
| 6 | 91 |
| 7 | 538 |
| 8 | 163 |
| 9 | 44 |
| 10 | 96 |
| 11 | 167 |
| 12 | 87 |
| 13 | 267 |
| 14 | 115 |
| 15 | 98 |
| 16 | 380 |
| 17 | 34 |
| 18 | 129 |
| 19 | 8 |
| 20 | 117 |
| 21 | 49 |
| 22 | 940 |
| 23 | 136 |
| 24 | 17 |
| 25 | 101 |
| 26 | 12 |
| 27 | 162 |
| 28 | 156 |
| 29 | 29 |
| 30 | 166 |
| 31 | 403 |
| 32 | 222 |
| 33 | 497 |
| 34 | 86 |
| 35 | 124 |
| 36 | 479 |
| 37 | 406 |
| 38 | 139 |
| 39 | 244 |
| 40 | 127 |
| 41 | 199 |
| 42 | 26 |
| 43 | 39 |
| 44 | 55 |
| 45 | 82 |
| 46 | 128 |
| 47 | 150 |
| 48 | 199 |
| 49 | 348 |
| 50 | 169 |
| 51 | 180 |
| 52 | 182 |
| 53 | 198 |
| 54 | 199 |
| 55 | 330 |
| 56 | 35 |
| 57 | 18 |
| 58 | 25 |
| 59 | 26 |
| 60 | 45 |
| 61 | 111 |
| 62 | 154 |
| 63 | 128 |
| 64 | 393 |
| 65 | 134 |
| 66 | 15 |
| 67 | 51 |
| 68 | 4 |
| 69 | 25 |
| 70 | 190 |
| 71 | 64 |
| 72 | 116 |
| 73 | 80 |
| 74 | 155 |
| 75 | 36 |
| 76 | 445 |
| 77 | 54 |
| 78 | 24 |
| 79 | 54 |
| 80 | 150 |
| 81 | 57 |
| 82 | 15 |
| 83 | 7 |
| 84 | 3 |
| 85 | 200 |
| 86 | 180 |
| 87 | 16 |
| 88 | 11 |
| 89 | 17 |
| 90 | 28 |
| 91 | 13 |
| 92 | 160 |
| 93 | 135 |
| 94 | 25 |
| 95 | 47 |
| 96 | 10 |
| 97 | 10 |
| 98 | 27 |
| 99 | 84 |
| 100 | 27 |
| 101 | 21 |
| 102 | 67 |
| 103 | 73 |
| 104 | 9 |
| 105 | 10 |
| 106 | 35 |
| 107 | 90 |
| 108 | 112 |
| 109 | 104 |
| 110 | 7 |
| 111 | 10 |
| 112 | 358 |
| 113 | 33 |
| 114 | 72 |
| 115 | 22 |
| 116 | 7 |
| 117 | 5 |
| 118 | 8 |
| 119 | 16 |
| 120 | 10 |
| 121 | 5 |
| 122 | 9 |
| 123 | 7 |
| 124 | 39 |
| 125 | 3 |
| 126 | 12 |
| 127 | 154 |
| 128 | 36 |
| 129 | 16 |

### B-1b) Binding Studies on Human α1- and α2-Adrenergic Receptors

To prepare cell membranes with human α₁- and α₂-adrenergic receptors, CHO cells stably overexpressing α₁- and α₂-adrenergic receptors are lysed and then subjected to differential centrifugation. After lysis in binding buffer (50 mM tris(hydroxymethyl)aminomethane/1 N hydrochloric acid, 5 mM magnesium chloride, pH 7.4) using an Ultra Turrax (Jahnke&Kunkel, Ika-Werk), the homogenate is centrifuged at 1000 g and at 4° C. for 10 min. The resulting sediment is discarded and the supernatant is centrifuged at 20000 g and at 4° C. for 30 min. The supernatant is discarded and the sediment is resuspended in binding buffer and stored at −70° C. until the binding test. For the binding test the radioligands ³H-MK-912 (2.2-3.2 TBq/mmol, PerkinElmer) (0.4 nM for α_{2C}-adrRez and 1 nM for α_{2A}-adrRez), 0.25 nM ³H-prazosin (α_{1AC}-adrRez; 2.6-3.3 TBq/mmol, PerkinElmer), 0.25 nM ³H-rauwolscine (α_{2B}-adrRez, 2.6-3.2 TBq/mmol, PerkinElmer) are incubated for 60 minutes with 5-20 μg cell membranes in binding buffer (total test volume 0.2 ml) in the presence of the test substances at 30° C. in 96-well filter plates (FC/B glass fibre, Multiscreen Millipore). The incubating is terminated by aspiration of the unbound radioactivity and the plates are then washed with binding buffer and subsequently dried at 40° C. for 1 hour. Liquid scintillator (Ultima Gold, PerkinElmer) is then added and the radioactivity that remained on the plates is measured in a liquid scintillation counter (Microbeta, Wallac). Non-specific binding is defined as radioactivity in the presence of 1-10 μM WB-4101 (α_{2C}-adrRez and α_{2A}-adrRez), prazosin (α_{2B}-adrRez and α_{1AC}-adrRez) (all from Sigma) and is generally <25% of the bound total radioactivity. The binding data (IC₅₀ and dissociation constant Kᵢ) are determined using the program GraphPad Prism Version 4.0.

### B-2) In Vivo Assays

### B-2a) Relaxation Measurement on Isolated Rat Tail Arteries

Male Wistar rats (200-250 g) were euthanized with carbon dioxide. The tail artery is prepared and incubated in Krebs-Henseleit buffer at 4° C. for 17 h (composition in mmol/l: NaCl 112, KCl 5.9, CaCl₂ 2.0 MgCl₂ 1.2, NaH₂PO₄ 1.2, NaHCO₃ 25, glucose 11.5). The artery is cut into rings of length 2 mm, transferred to an organ bath filled with 5 ml of Krebs-Henseleit buffer and connected to a wire myograph (DMT, Denmark). The buffer is warmed to 27° C. and sparged with 95% O₂, 5% CO₂. Before each experiment, the responsiveness of the preparation is tested by adding potassium-containing Krebs-Henseleit solution (50 mmol/l KCl). After an equilibration phase of 60 minutes, contraction of the vessel rings is induced with 30 nmol/l UK 14.304. The test substance is then added cumulatively in increasing concentration. Relaxation is shown as a reduction in the contraction induced by UK 14.304.

### B-2b) Haemodynamics CHF Rat

Male old Wistar, ZDF/Crl-Lepr fa/fa, SHR—SP or Sprague Dawley rats (Charles River; 250-300 g) are anaesthetized with 5% isoflurane in an anaesthesis cage, intubated and then ventilated artificially (rate: 60 breaths/min; ratio inspiration to expiration: 50:50; positive end-expiratory pressure: 1 cm H₂O; tidal volume: 10 ml/kg of body weight; FIO₂:0.5; 2% isoflurane). The body temperature is maintained at 37-38° C. by a heating mat. 0.05 mg/kg Temgesic is given s.c. as analgesic. For the haemodynamic measurement, the rats are then tracheotomized and artificially ventilated (frequency: 60 breaths/min; ratio inspiration to expiration: 50:50; positive end-expiratory pressure: 1 cm H₂O; tidal volume: 10 ml/kg of body weight; FIO₂:0.5). Anaesthesia is maintained by inhalative isoflurane anaesthesia. The left-ventricular pressure is determined via the left carotid artery using a Millar microtip catheter (Millar SPR-320 2F). Systolic left-ventricular pressure (sLVP), end-diastolic ventricular pressure (LVEDP), contractility (+dPdt) and relaxation force (−dPdt) are determined as derived parameters. Following the haemodynamic measurements, the heart is removed and the ratio of right to left ventricle including septum is determined. Furthermore, plasma samples are obtained to determine plasma biomarkers and plasma substance concentrations.

### B-2c) Measurement of Blood Flow and Blood Pressure in Rats

Wistar rats (Hsd Cpb:Wu) of a weight of 250-350 g or ZDF rats (ZDF/Crl-Lepr fa/fa) of a weight of 330-520 g were anaesthetized using 2.5% isoflurane in an oxygen/laughing gas mixture (40:60). To determine the blood flow in the carotid artery and the femoral artery, the anaesthetized rat was brought into a supine position, and the left carotid artery and the right femoral artery are then carefully exposed. Blood flow was measured by placing flow probes (Transonic Flowprobe) at the vessels. By introducing a PE50 artery catheter into the left femoral artery, blood pressure and heart rate were determined (Transducer Ref. 5203660: from Braun CH). The substances were administered as a bolus injection or a continuous infusion via a venous catheter in the left femoral vein.

Following the preparation of the animals, there was a 5 min baseline interval. Infusion of the AR alphα2C receptor antagonist was then started. In the steady state (32 min after the start of the experiment), the femoral flow was determined in relation (% difference) to the initial flow.

The compound of Example 8 showed a dose-dependent increase in femoral flow in diabetic ZDF fa/fa animals at doses of 0.1, 0.3 and 1 μg/kg. In the Wistar rat, no increase in femoral flow was observed up to a dose of 1 μg/kg/min. At the same time, no changes in blood pressure and heart rate were measured. Placebo: 10% ethanol/40% PEG400/50% NaCl. The data (means) are shown in Table 2:

TABLE 2

Change in the femoral flow in %

| | ZDF rat (n = 3) | Wistar rat |
|---|---|---|
| Placebo | −7.7 | 0.9 (n = 3) |
| Example 8; 3 μg/kg/min | 12.0 (n = 4) | not measured |
| Example 8; 10 μg/kg/min | 127.9 | not measured |
| Example 8; 30 μg/kg/min | 144.4 | 3.1 (n = 5) |
| Example 8; 10 μg/kg/min | not measured | 14.2 (n = 3) |
| Example 8; 300 μg/kg/min | not measured | 15.3 (n = 5) |

### B-2d) Assay of Perfusion-Enhancing Substances (Haemodynamics)

To reduce perfusion, the right external iliac artery in anaesthetized (for example anaesthesia by inhalating isoflurane, enflurane) rats (for example ZDF/Crl-Lepr fa/fa) is ligated under sterile conditions. Depending on the degree of collateralization of the animals, it is additionally necessary to ligate the femoral artery to reduce perfusion. After the operation or else preventatively, the test animals are treated orally, intragastrically (uptake by stomach tube or through feed or drinking water), intraperitoneally, intravenously, intraarterially, intramuscularly, inhalatively or subcutaneously with the test substances. The test substances are administered enterally or parenterally, once or more than once per day over a period of up to 50 weeks, or administration is continuous via subcutaneously implanted osmotic mini-pumps (for example Alzet pumps). During the experiment, microperfusion and temperature of the lower extremities are documented. Here, under anaesthesia, a temperature-sensitive laser doppler probe (Periflux) is fastened with adhesive to the paws of the rats, allowing the measurement of microperfusion and skin temperature. Depending on the test protocol, samples such as blood (interim diagnostics) and other bodily fluids, urine or organs are removed to carry out further in vitro examinations, or, to document haemodynamics, blood pressure and heart rate are measured via a catheter in the carotid artery. At the end of the experiment, the animals are painlessly sacrificed.

### B-2e) Assay of Perfusion-Enhancing Substances (Microcirculation)

In diabetic (ZDFfa/fa) and healthy rats (Wistar), a laser doppler probe was fastened under anaesthesia conditions (isoflurane anaesthesia) at the sole of the paw for measuring cutaneous microcirculation. The test animals were once treated orally with the test substances. During the experiment, microperfusion and temperature of the lower extremities were documented continuously. Here, a temperature-sensitive laser doppler probe (Periflux, O2C) was fastened with adhesive to the paws of the animals, allowing the measurement of microperfusion and skin temperature. The microcirculation measurement values were measured on both paws 30 min after oral administration of the test substance. From these data, means were calculated and compared to those of placebo-treated animals. What is shown are the minimum effective doses (MED) where the test substances showed a significantly improved microcirculation compared with placebo (vehicle=10% EtOH+30% PEG400+60% water for injection; 1 ml/kg) and the factor by which microcirculation is improved at this dose compared to placebo. Also stated is the MED for the significant increase of skin temperature (ttest).

Microcirculation data for adrenoreceptor α_{2C} receptor antagonist of the compound of Example 8 and for comparative substance ORM12741, an AR α2c receptor antagonist from Orion, are shown in Table 3:

TABLE 3

| | MED [mg/kg] | MED [mg/kg] |
|---|---|---|
| Example No. | microcirculation | skin temperature |
| 8 | 1 (2.3x) | 1 |
| ORM-12741 (Orion) | 0.1 (1.9x) | 0.01 |

### B-2f) Assay of Perfusion-Enhancing Substances (Motoric Function) in the Treadmill Test

To determine the motor function, the running behaviour of mice (for example eNOS knock out mice, wild-type mice C-57 Bl6 or ApoE knock out mice) is examined on treadmills. To get the mice used to using the treadmill voluntarily, 4-5 weeks before the start of the experiment the animals are put singly into cages with the treadmill and trained. 2 weeks before the start of the experiment, the movements of the mice on the treadmill are recorded by a computer-linked photo cell, and various running parameters such as, for example, daily distance run, individual distances covered, but also their temporal distribution over the day are determined. According to their natural running behaviour, the animals are randomized into groups (8-12 animals) (control group, sham group and one or more substance groups). After the customization phase of 2 weeks, to reduce perfusion in the hind legs the femoral arteries on both sides are ligated under anaesthesia and under sterile conditions (for example anaesthesia by inhaling isoflurane). After the operation or else preventatively, the test animals are treated orally, intragastrically (uptake by stomach tube or through feed or drinking water), intraperitoneally, intravenously, intraarterially, intramuscularly, inhalatively or subcutaneously with the test substances. The test substances are administered enterally or parenterally, once or more than once per day over a period of up to 5 weeks, or administration is continuous via subcutaneously implanted osmotic mini-pumps. The running behaviour of the animals is monitored and recorded over a period of several weeks after the operation. At the end of the experiment, the animals are painlessly sacrificed. Depending on the test protocol, samples such as blood and other bodily fluids or organs are removed to carry out further in vitro examinations (S. Vogelsberger Neue Tiermodelle für die Indikation Claudicatio Intermittens [Novel animal models for the indication intermittent claudication](pocket book), publisher: VVB Laufersweiler Verlag (March 2006), ISBN-10: 383595007X, ISBN-13: 978-3835950078).

### B-22) Assay of Perfusion-Enhancing Substances (Measurement of the Occlusion Pressure)

To reduce perfusion, the right external iliac artery in anaesthetized (for example anaesthesia by inhaling isoflurane) rats (for example ZDF rats) is ligated under sterile conditions. Depending on the degree of collateralization of the animals, it is additionally necessary to ligate the femoral artery to reduce perfusion. After the operation or else preventatively, the test animals are treated orally, intragastrically (uptake by stomach tube or through feed or drinking water), intraperitoneally, intravenously, intraarterially, intramuscularly, inhalatively or subcutaneously with the test substances. The test substances are administered enterally or parenterally, once or more than once per day over a period of up to 5 weeks, or administration is continuous via subcutaneously implanted osmotic mini-pumps (for example Alzet pumps). The occlusion pressures of the animals are measured before the operation (subsequent randomization) and once every week over a period of up to 2 months after the operation. Here, under anaesthesia an inflatable cuff is placed around the hind legs of the rats, and a temperature-adjustable laser doppler probe (Periflux) is fastened with adhesive on the paws. The cuffs are inflated until the laser doppler probes no longer measure any blood flow. The pressure in the cuffs is then continuously reduced and the pressure at which blood flow is detected again is determined. Depending on the test protocol, samples such as blood (interim diagnostics) and other bodily fluids or organs are removed for further in vitro examinations. At the end of the experiment, the animals are sacrificed painlessly (S. Vogelsberger Neue Tiermodelle für die Indikation Claudicatio Intermittens [New Animal Models for the Indication Intermittent Claudication](pocket book), publisher: VVB Laufersweiler Verlag (March 2006), ISBN-10: 383595007X, ISBN-13: 978-3835950078.)

### B-2h) Examination of Substances Affecting Wound Healing (Ulcer Model)

To induce a superficial wound, diabetic mice (db/db, i.e. BKS.Cg-m Dock7m+/+Leprdb/J mice) were anaesthetized with isoflurane. A continuous lesion (10 mm×10 mm) is placed on the left side of a skin area where the hairs were removed and which was disinfected. The animals are then randomized to the different treatment groups. In all groups, the wounds are covered with dressings (Systagenix Wound Management, UK). Daily (from day 1 after wound placing) the animals are treated by gavage (200 μl, vehicle=10% EtOH+30% PEG400+60% water for injection) with the substances at the stated dosages. On days 4, 8, 12, 16 and 20, the animals are anaesthetized, the dressings are removed and the wound size is measured using digital photos. The photos are evaluated by an automatic calibrated planimetric process. The results are shown as remaining wound sizes over the course of the experiment. To this end, all individual values are referenced in percent to the individual animal at the day the wound was placed.

### B-2i) Examination of Substances Affecting Kidney Function

In animals suffering from acute or disease-related kidney damage (e.g. STZ rat, ZDF rat, ZDF rat with DOCA implantat, UUO kidney damage model, glomerulonephritis model, diabetes, atherosclerosis), diuresis is carried out at regular intervals before or during continuous treatment with the test substances. The test animals are treated orally, intragastrically (uptake by stomach tube or through feed or drinking water), intraperitoneally, intravenously, intraarterially, intramuscularly, inhalatively or subcutaneously with the test substances. The test substances are administered enterally or parenterally, once or more than once per day, or administration is continuous via subcutaneously implanted osmotic mini-pumps (for example Alzet pumps). Over the entire duration of the test, plasma and urine parameters are determined.

### B-2j) Haemodynamics in Anaesthetized Dogs

Healthy Mongrel® dogs (Marshall BioResources, Marshall Farms Inc; Clyde N.Y.; USA) or Mongrel® dogs suffering from heart failure of both sexes and having a weight of 25-35 kg are used. Anaesthesia is initiated by slow i.v. administration of 25 mg/kg sodium thiopental (Trapanal®) and 0.15 mg/kg alcuronium chloride (Alloferin®) and maintained during the experiment by means of a continuous infusion of 0.04 mg/kg*h fentanyl (Fentanyl®), 0.25 mg/kg*h droperidol (Dihydrobenzperidol®) and 15 μg/kg/h alcuronium chloride (Alloferin®). After intubation, the animals are ventilated by the ventilator at a constant respiratory volume such that an end-tidal CO₂ concentration of about 5% is achieved. Ventilation is performed with room air, enriched with about 30% oxygen (normoxia). To measure the haemodynamic parameters, a liquid-filled catheter is implanted into the femoral artery for measuring blood pressure. A Swan-Ganz® catheter having two lumens is introduced in a flow-directed manner via the jugular vein into the pulmonary artery (distal lumen for measuring the pressure in the pulmonary artery, proximal lumen for measuring the central vein pressure). Using a temperature sensor at the tip of the catheter, the continuous cardiac output (CCO) is determined. Blood flow is measured at various vascular beds such as the coronary artery, the carotid artery or the femoral artery by placing flow probes (Transonic Flowprobe) at the vessels in question. The pressure in the left ventricle is measured after introduction of a microtip catheter (Millar® Instruments) via the carotid artery into the left ventricle, and the dP/dt ratio as a measure of contractility is derived therefrom. Substances are administered i.v. via the femoral vein or intraduodenally as cumulative dose/activity curve (bolus or continuous infusion). The haemodynamic signals are recorded and evaluated by means of pressure transducers/amplifiers and PONEMAH® as data aquisition software.

To induce heart failure, a pacemaker is implanted into the dogs under sterile conditions. After induction of anaesthesia with pentobarbital-Na (15 to 30 mg kg⁻¹ i.v.) followed by intubation and subsequent ventilation (room air; Sulla 808, Dräger®, Germany), anaesthesia is maintained by continuous infusion of pentobarbital (1-5 mg kg⁻¹ h⁻¹) and fentanyl (10-40 μg kg⁻¹ h⁻¹). A pacemaker cable (Setrox S60®, Biotronik, Germany) is implanted via an incision of the left jugular vein and placed in the right ventricle. The cable is connected to the pacemaker (Logos®, Biotronik, Germany), which is positioned in a small subcutaneous pocket between the shoulder blades. Ventricular pacing is started only 7 days after the surgical intervention, to obtain heart failure at a frequency of 220 beats/min over a period of 10-28 days.

### B-2k) Determination of the Antidepressant Effect in the Rat-Forced-Swimming-Test

Rats which are forced to swim in a narrow room from which there is no escape adapt after an initial phase of increased activity by adopting a characteristic rigid posture and only carry out those movements which are absolutely required to keep the head above the water. This immobility can be reduced by a number of clinically active antidepressants (e.g. Cryan J F, Markou A, Lucki I. Assessing antidepressant activity in rodents: recent developments and future needs. Trends Pharmacol. Sci. 2002; 23:238-245). The method used here is based on the protocol of Porsolt et al. (Porsolt R D, Anton G, Blavet N, Jalfre M. Behavioural despair in rats: a new model sensitive to antidepressant treatments. Eur. J. Pharmacol. 1978; 47:379-91; and Porsolt R D, Brossard G, Hautbois C, Roux S. Rodent models of depression: forced swimming and tail suspension behavioral despair tests in rats and mice. Curr. Protoc. Neurosci. 2001; Chapter 8:Unit 8.10A, 1-10) and De Vry et al. (De Vry J, Maurel S, Schreiber R, de Beun R, Jentzsch K R. Comparison of hypericum extracts with imipramine and fluoxetine in animal models of depression and alcoholism. Eur. Neuropsychopharmacology 1999; 9:461-468). In two sessions (training and test) at an interval of 24 h, the rats are forced to swim in a narrow cylinder filled with water from which there is no escape. The training session (duration 15 min) is carried out before the treatment with substance without recording the behaviour in order to familiarize the rats with the 5-minute test session 24 h later. During both sessions, the rats are individually placed into the cylinders filled with water, which are optically separated from one another. After the session, the rats are removed from the water and dried. About 24, 5 and 1 h prior to the test session, the rats are treated with test substance or vehicle solution; the first administration takes place immediately after the training session. 3 substance administrations prior to the test session lead to more stable pharmacological results than a single administration. The test sessions are recorded electronically using a surveillance video camera and, after storage, analysed off-line using a computer. For each animal, the behaviour is analysed by 3-4 independent observers who score the total time of immobility in seconds over the 5-minute test session.

Passive behaviour or immobility is defined as a rat which drifts in the water in an upright position and makes only small movements to keep the head above the water or to maintain its body in a balanced stable position. In contrast, active behaviour is characterized by active swimming movements, e.g. forceful movements of front or hind legs and/or tail, climbing or diving.

For each animal and treatment group, the mean of the duration of immobility determined by the observers is calculated. Differences in the duration of immobility between the groups are examined statistically by ANOVA or a suitable non-parametric test with p<0.05 as significance level.

### B-2l) Radiotelemetric Measurement of Blood Pressure and Heart Rate of Conscious Rats

A commercially available telemetry system from Data Sciences International DSI, USA, was employed for the measurements on conscious rats described below. The system consists of 3 main components: (1) implantable transmitters (Physiotel® telemetry transmitter), (2) receivers (Physiotel® receiver), which are linked via a multiplexer (DSI Data Exchange Matrix) to a (3) data acquisition computer. The telemetry system makes it possible to continuously record blood pressure, heart rate and body motion of conscious animals in their usual habitat.

The studies were conducted on adult female Wistar rats with a body weight of >200 g. After transmitter implantation, the experimental animals were housed singly in type III Makrolon® cages. They had free access to standard feed and water. The day/night rhythm in the test laboratory was set by changing the illumination of the room.

### Transmitter Implantation:

The telemetry transmitters used (PA-C40, DSI) were surgically implanted under aseptic conditions in the experimental animals at least 14 days before the first experimental use.

For the implantation, the fasted animals were anaesthetized with isoflurane (IsoFlo®, Abbott, initiation 5%, maintenance 2%) and shaved and disinfected over a large area of their abdomens. After the abdominal cavity had been opened along the linea alba, the liquid-filled measuring catheter of the system was inserted into the descending aorta in the cranial direction above the bifurcation and fixed with tissue glue (VetBond™, 3M). The transmitter housing was fixed intraperitoneally to the abdominal wall muscle, and the wound is closed layer by layer. Post-operatively, an antibiotic (Ursocyclin® 10%, 60 mg/kg s.c., 0.06 ml/100 g body weight, Serumwerk Bernburg AG, Germany) for infection prophylaxis and an analgesic (Rimadyl®, 4 mg/kg s.c., Pfizer, Germany) were administered.

### Substances and Solutions:

Unless stated otherwise, the substances to be studied were administered orally to a group of animals in each case (n=6). In accordance with an administration volume of 2 ml/kg of body weight, the test substances were dissolved in suitable solvent mixtures. A solvent-treated group of animals (placebo/vehicle=diethylene glycol monoethyl ether, Transcutol®, 2 ml/kg p.o.) was used as control.

### Experimental Outline:

The telemetry measuring system is configured for 24 animals.

Each of the instrumented rats living in the system was assigned a separate receiving antenna (RPC-1 Receiver, DSI). The implanted transmitters were activated externally via an installed magnetic switch and were switched to transmission during the pre-run of the experiment. The signals emitted were detected online by a data acquisition system (Dataquest™ A.R.T. for Windows, DSI) and processed accordingly.

In the standard procedure, the following were measured for 10-second periods in each case: (1) systolic blood pressure (SBP), (2) diastolic blood pressure (DBP), (3) mean arterial pressure (MAP), (4) heart rate (HR) and (5) activity (ACT). These parameters were measured over 24 hours after administration.

The acquisition of measurements was repeated under computer control at 5-minute intervals. The source data obtained as absolute values were corrected in the diagram with the currently measured barometric pressure (Ambient Pressure Reference Monitor, APR-1, DSI).

### Evaluation:

After the end of the experiment, the acquired individual data were sorted using the analysis software (Dataquest™ A.R.T. 4.1 Analysis). The blank value was taken to be the mean of the pre-run (i.e. before substance administration) (4 absolute values) and this was compared to the absolute value of the measurement, giving the deviation in %. The data were smoothed over a presettable period by determination of the means (15 minute mean).

### REFERENCES

K. Witte, K. Hu, J. Swiatek, C. Müssig, G. Ertl and B. Lemmer, Experimental heart failure in rats: effects on cardiovascular circadian rhythms and on myocardial β-adrenergic signaling, Cardiovasc. Res. 47 (2): 203-405, 2000.

### Results:

The results are shown in FIGS. 1 to 4 for the compound of Example 8 in comparison to an adrenoreceptor α_{2C} receptor antagonist from Orion (ORM-12741) which has been tested for the therapy of Alzheimer's disease and Raynaud's syndrome.

At 5 and 15 mg/kg, Example No. 8 showed a slight transient increase of the heart rate, without any effect on blood pressure. In contrast, the comparative substance ORM-12741, an AR α2c receptor antagonist from Orion, showed an additional reduction in blood pressure at 10 mg/kg.

### EXPLANATION OF THE FIGURES

FIG. 1: B-2l) Heart rate in % deviation as a function of the time [h] after substance administration, Example 8

FIG. 2: B-2l) Mean arterial blood pressure in % deviation as a function of the time [h] after substance administration, Example 8

FIG. 3: B-2l) Heart rate in % deviation as a function of the time [h] after substance administration, Comparative example ORM12741

FIG. 4: B-2l) Mean arterial blood pressure in % deviation as a function of the time [h] after substance administration, Comparative Example ORM12741

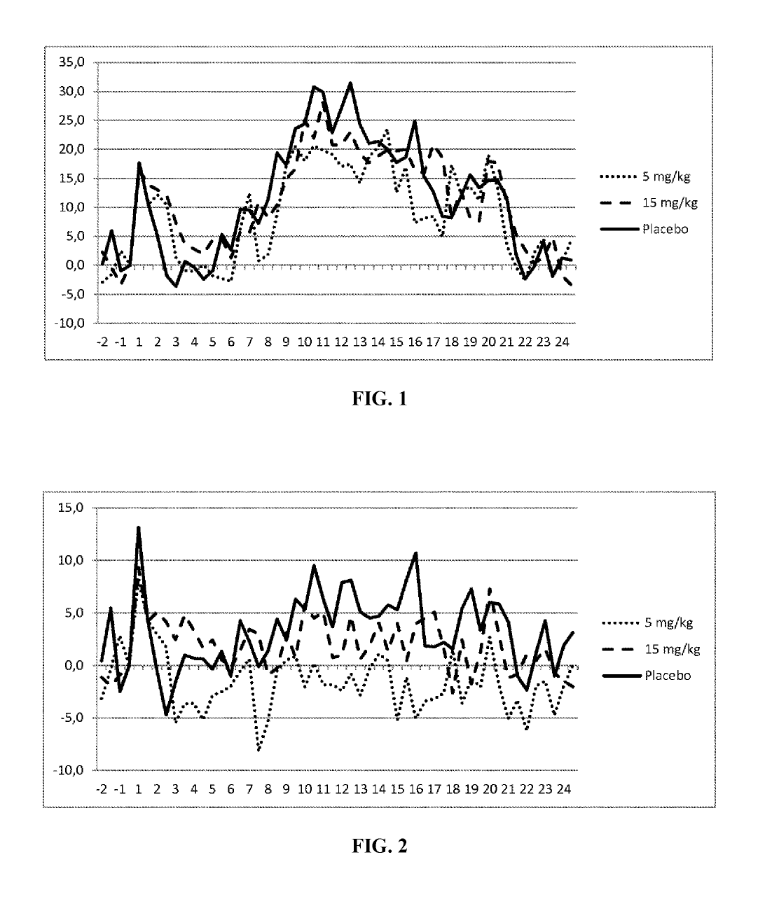

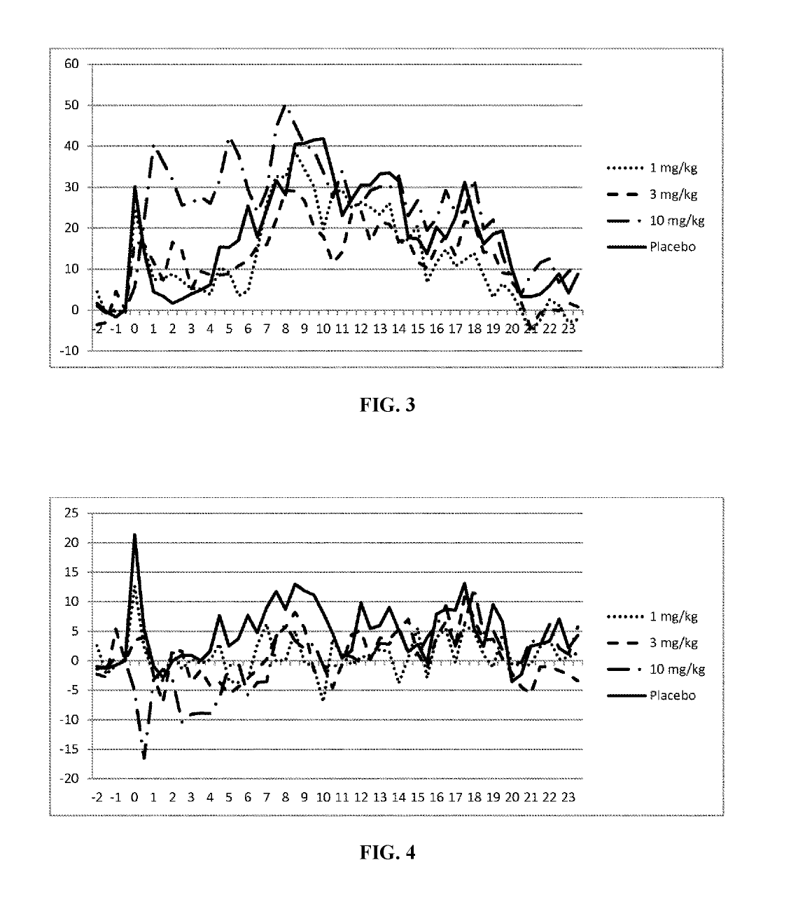

### C) WORKING EXAMPLES OF PHARMACEUTICAL COMPOSITIONS

The substances according to the invention can be converted to pharmaceutical preparations as follows:

### Tablet:

### Composition:

100 mg of the compound of Example 1, 50 mg of lactose (monohydrate), 50 mg of maize starch, 10 mg of polyvinylpyrrolidone (PVP 25) (from BASF, Germany) and 2 mg of magnesium stearate. Tablet weight 212 mg. Diameter 8 mm, radius of curvature 12 mm.

### Production:

The mixture of the compound of Example 1, lactose and starch is granulated with a 5% strength solution (m/m) of the PVP in water. After drying, the granules are mixed with the magnesium stearate for 5 min. This mixture is compressed in a conventional tabletting press (see above for format of the tablet).

### Oral Suspension:

### Composition:

1000 mg of the compound of Example 1, 1000 mg of ethanol (96%), 400 mg of Rhodigel (xanthan gum) (from FMC, USA) and 99 g of water.

10 ml of oral suspension correspond to a single dose of 100 mg of the compound of the invention.

### Production:

The Rhodigel is suspended in ethanol, and the compound of Example 1 is added to the suspension. The water is added while stirring. The mixture is stirred for approx. 6 h until the Rhodigel has finished swelling.

### Intravenously Administrable Solution:

### Composition:

1 mg of the compound of Example 1, 15 g of polyethylene glycol 400 and 250 g of water for injection purposes.

### Production:

The compound of Example 1 is dissolved together with polyethylene glycol 400 by stirring in the water. The solution is sterilized by filtration (pore diameter 0.22 μm) and dispensed under aseptic conditions into heat-sterilized infusion bottles. The latter are closed with infusion stoppers and crimped caps.

1. A compound of the formula (I) 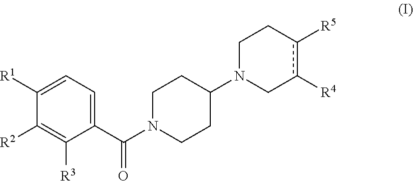
in which [symbol]
represents a single bond or a double bond,
R¹ is selected from the group consisting of C₃-C₆-alkyl, C₁-C₃-alkoxycarbonyl, oxetanyl, 5- or 6-membered heteroaryl, —(CR⁶R⁷)—R⁸ and —CONR⁹R¹⁰,
where oxetanyl may be substituted by 1 or 2 substituents independently of one another selected from the group consisting of 3-hydroxy and 3-C₁-C₄-alkyl,
and
where
R⁶ is selected from the group consisting of hydrogen, methyl and ethyl,
R⁷ is selected from the group consisting of hydrogen, methyl and ethyl,
or
R⁶ and R⁷ together with the carbon atom to which they are attached form a cyclopropyl ring or cyclobutyl ring,
R⁸ is selected from the group consisting of hydroxy, hydroxymethyl, C₁-C₄-alkyl, C₁-C₄-alkoxy, C₁-C₃-alkoxycarbonyl, C₁-C₄-alkylaminocarbonyl, phenoxy, oxetanyl, 5- or 6-membered heteroaryl and —CH₂NR¹³R¹⁴,
where phenoxy and heteroaryl may be substituted by 1 to 3 substituents independently of one another selected from the group consisting of C₁-C₄-alkyl and C1-C₄-alkoxy,
where oxetanyl may be substituted by 1 or 2 substituents independently of one another selected from the group consisting of 3-C₁-C₄-alkyl and 3-OH,
and
where
R¹³ is selected from the group consisting of hydrogen and C₁-C₄-alkyl,
and
R¹⁴ is selected from the group consisting of methyl, methylsulphonyl and formyl,
R⁹ is selected from the group consisting of C₁-C₆-alkyl, C₃-C₆-cycloalkyl and 5- or 6-membered heteroaryl,
where heteroaryl may be substituted by C₁-C₄-alkyl,
where alkyl may be substituted by 1 to 3 substituents independently of one another selected from the group consisting of hydroxy, with the proviso that alkyl is C₂-C₆-alkyl, C₁-C₄-alkoxy, C₁-C₄-haloalkyl, C₃-C₆-cycloalkyl, phenyl, oxetanyl and 5- or 6-membered heteroaryl,
in which this phenyl heteroaryl for its part may be substituted by 1 to 3 substituents independently of one another selected from the group consisting of halogen, trifluoromethyl, difluoromethoxy, trifluoromethoxy and C₁-C₄-alkyl
in which this oxetanyl for its part may be substituted by one or 2 substituents selected from the group consisting of 3-C₁-C₄-alkyl and 3-hydroxy
R¹⁰ is selected from the group consisting of hydrogen and C1-C₄-alkyl,
or
R⁹ and R¹⁰ together with the nitrogen atom to which they are attached form a piperidinyl ring,
where the piperidinyl ring may be substituted by 1 to 3 substituents independently of one another selected from the group consisting of C₁-C₄-alkyl,
R² is selected from the group consisting of hydrogen and halogen,
R³ is selected from the group consisting of hydrogen, halogen, hydroxy and C₁-C₄-alkoxy,
R⁴ is selected from the group consisting of C₁-C₃-alkyl, C₁-C₃-alkoxycarbonyl, C₃-C₆-cycloalkyl, C₃-C₆-cycloalkyl-C₁-C₃-alkoxy, C₃-C₆-cycloalkoxy, trifluoromethoxy-C₁-C₄-alkoxy, 5- or 6-membered heteroaryl and —OCONR¹¹R¹², where alkyl may be substituted by a substituent selected from the group consisting of C₁-C₄-alkoxy, C₃-C₆-cycloalkoxy, trifluoromethoxy and phenoxy,
in which this phenoxy for its part may be substituted by 1 to 3 substituents independently of one another selected from the group consisting of halogen,
and
where heteroaryl may be substituted by 1 to 3 substituents independently of one another selected from the group consisting of C₁-C₄-alkyl and C₃-C₆-cycloalkyl,
in which this alkyl for its part may be substituted by a substituent selected from the group consisting of C₁-C₃-alkoxy and C₃-C₆-cycloalkyl,
R¹¹ represents C1-C₄-alkyl or C₃-C₆-cycloalkyl,
R¹² is selected from the group consisting of hydrogen and C₁-C₄-alkyl,
or
R¹¹ and R¹² together with the nitrogen atom to which they are attached form a pyrrolidinyl ring,
R⁵ represents hydrogen or C₁-C₄-alkyl,
or one of the salts thereof, solvates thereof or solvates of the salts thereof.

2. The compound of the formula (I) according to claim 1 in which [symbol]
represents a single bond,
R¹ represents C₃-C₄-alkyl, C1-C₃-alkoxycarbonyl, oxetanyl, oxazolyl, —(CR⁶R⁷)—R⁸ or —CONR⁹R¹⁰,
where oxetanyl may be substituted by 1 or 2 substituents independently of one another selected from the group consisting of 3-hydroxy and 3-C₁-C₃-alkyl,
and
where
R⁶ is selected from the group consisting of hydrogen, methyl and ethyl,
R⁷ is selected from the group consisting of hydrogen, methyl and ethyl,
or
R⁶ and R⁷ together with the carbon atom to which they are attached form a cyclopropyl ring or cyclobutyl ring,
R⁸ is selected from the group consisting of hydroxy, hydroxymethyl, C₁-C₃-alkyl, C₁-C₃-alkoxy, C₁-C₃-alkoxycarbonyl, C₁-C₃-alkylaminocarbonyl, phenoxy, oxetanyl, pyrazolyl and —CH₂NR¹³R¹⁴,
where phenoxy and pyrazolyl may be substituted by 1 to 3 substituents independently of one another selected from the group consisting of C₁-C₂-alkyl and C1-C₂-alkoxy,
where oxetanyl may be substituted by 1 or 2 substituents independently of one another selected from the group consisting of 3-C₁-C₂-alkyl,
and
where
R¹³ is selected from the group consisting of hydrogen and C1-C₂-alkyl,
and
R¹⁴ is selected from the group consisting of methyl, methylsulphonyl and formyl,
R⁹ is selected from the group consisting of C₁-C₄-alkyl, C₃-C₆-cycloalkyl and oxazolyl,
where alkyl may be substituted by 1 to 3 substituents independently of one another selected from the group consisting of hydroxy, with the proviso that alkyl is C₂-C₄-alkyl, C₁-C₂-alkoxy, C₁-C₂-haloalkyl, C₃-C₄-cycloalkyl, phenyl, oxetanyl, oxazolyl, pyrazolyl and pyridyl,
in which this phenyl or pyridyl for its part may be substituted by 1 to 3 substituents independently of one another selected from the group consisting of halogen, trifluoromethyl, difluoromethoxy, trifluoromethoxy and methyl,
in which this oxetanyl for its part may be substituted by 3-methyl
and
in which this oxazolyl for its part may be substituted by 1 to 3 methyl substituents,
R¹⁰ is selected from the group consisting of hydrogen and C₁-C₃-alkyl,
R² is selected from the group consisting of hydrogen, fluorine and chlorine,
R³ is selected from the group consisting of hydrogen, fluorine, chlorine, hydroxy and C₁-C₂-alkoxy,
R⁴ is selected from the group consisting of C₁-C₂-alkyl, C₁-C₃-alkoxycarbonyl, C₃-C₄-cycloalkyl, C₃-C₄-cycloalkyl-C₁-C₃-alkoxy, C₃-C₄-cycloalkoxy, trifluoromethoxy-C₁-C₂-alkoxy, oxadiazole, triazole and pyrrolidine-1-carboxylate,
where alkyl may be substituted by a substituent selected from the group consisting of C₁-C₄-alkoxy, C₃-C₄-cycloalkoxy, trifluoromethoxy and phenoxy,
in which this phenoxy for its part may be substituted by 1 to 3 substituents independently of one another selected from the group consisting of fluorine and chlorine,
and
where oxadiazole or triazole may be substituted by 1 to 3 substituents independently of one another selected from the group consisting of C₁-C₂-alkyl and C₃-C₄-cycloalkyl,
in which this alkyl for its part may be substituted by a substituent selected from the group consisting of C₁-C₃-alkoxy and C₃-C₄-cycloalkyl,
R⁵ represents hydrogen,
or one of the salts thereof, solvates thereof or solvates of the salts thereof.

3. The compound of the formula (I) according to claim 1 in which [symbol]
represents a single bond,
R¹ represents C₃-C₄-alkyl, oxetanyl, —(CR⁶R⁷)—R⁸ or —CONR⁹R¹⁰,
where oxetanyl may be substituted by a substituent selected from the group consisting of 3-hydroxy and 3-methyl,
and
where
R⁶ is selected from the group consisting of hydrogen, methyl and ethyl,
R⁷ is selected from the group consisting of hydrogen, methyl and ethyl,
or
R⁶ and R⁷ together with the carbon atom to which they are attached form a cyclobutyl ring,
R⁸ is selected from the group consisting of hydroxy, methyl, methoxy, oxetanyl, and —CH₂NR¹³R¹⁴,
where oxetanyl may be substituted by a 3-methyl substituent,
and
where
R¹³ is selected from the group consisting of hydrogen and methyl,
and
R¹⁴ is selected from the group consisting of methyl, methylsulphonyl and formyl,
R⁹ is selected from the group consisting of C₁-C₄-alkyl and oxazolyl,
where alkyl may be substituted by 1 to 3 substituents independently of one another selected from the group consisting of hydroxy, with the proviso that alkyl is C₂-C₆-alkyl, phenyl and pyridyl,
in which this phenyl or pyridyl for its part may be substituted by 1 to 3 substituents independently of one another selected from the group consisting of chlorine, fluorine and trifluoromethyl,
and
where oxazolyl may be substituted by 1 to 3 methyl substituents,
R¹⁰ is selected from the group consisting of hydrogen and methyl,
R² represents hydrogen,
R³ is selected from the group consisting of hydrogen and chlorine,
R⁴ is selected from the group consisting of methyl, ethyl, ethoxycarbonyl, cyclopropyl, C₃-C₄-cycloalkyl-C₁-C₂-alkoxy, oxadiazolyl and triazolyl,
where methyl or ethyl may be substituted by a substituent selected from the group consisting of methoxy, ethoxy, tert-butoxy, C₃-C₄-cycloalkoxy and trifluoromethoxy,
and
where oxadiazolyl or triazolyl may be substituted by 1 to 3 methyl substituents,
in which this methyl for its part may be substituted by C₃-C₄-cycloalkyl,
R⁵ represents hydrogen,
or one of the salts thereof, solvates thereof or solvates of the salts thereof.

4. A method of making the compound of the formula (I) of claim 1 and its starting materials and intermediates, or the salts thereof, the solvates thereof or the solvates of the salts thereof, where
[A] compounds of the formula (II) 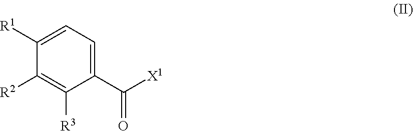
in which
R¹, R² and R³ have the meaning given above, and
X¹ is selected from the group consisting of halogen, preferably bromine or chlorine, and hydroxy,
are reacted with compounds of the formula (III) 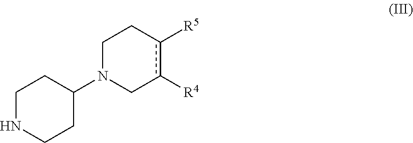
in which [symbol]
, R⁴ and R⁵ have the meaning given above,
in the presence of a dehydrating agent to give compounds of the formula (I)
or
[B] compounds of the formula (II) 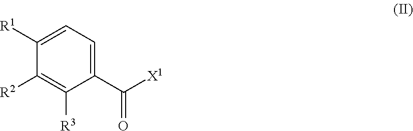
in which
R¹, R² and R³ have the meaning given above, and
X¹ represents hydroxy,
are reacted with 4-piperidinone in the presence of a dehydrating agent to give compounds of the formula (V) 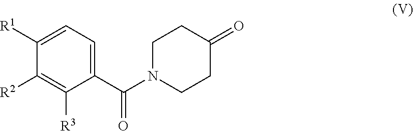
in which
R¹, R² and R³ have the meaning given above,
or
[C] compounds of the formula (V) 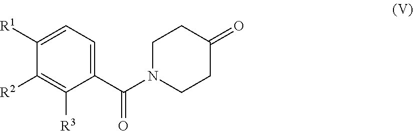
in which
R¹, R² and R³ have the meaning given above,
are reacted with compounds of the formula (VI) 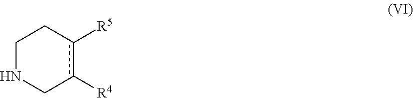
in which [symbol]
, R⁴ and R⁵ have the meaning given above,
in the presence of a reducing agent to give compounds of the formula (I)
or
[D] compounds of the formula (IV) 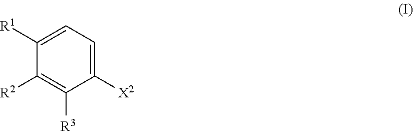
in which
R¹, R² and R³ have the meaning given above, and
X² is selected from the group consisting of halogen, preferably bromine, and trifluoromethanesulphonate,
are reacted with compounds of the formula (III) 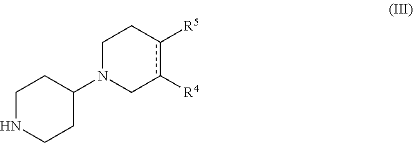
in which [symbol]
, R⁴ and R⁵ have the meaning given above,
in the presence of a carbon monoxide source and a catalyst to give compounds of the formula (I) or
[E] compounds of the formula (VII) 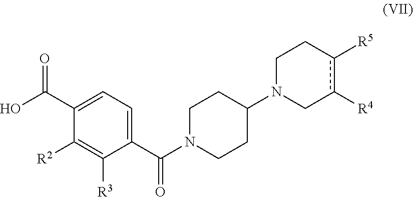
in which [symbol]
, R², R³, R⁴ and R⁵ have the meaning given above,
are reacted with compounds of the formula 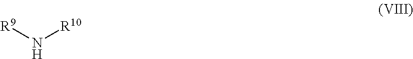
in which
R⁹ and R¹⁰ have the meaning given above,
in the presence of a dehydrating agent to give compounds of the formula 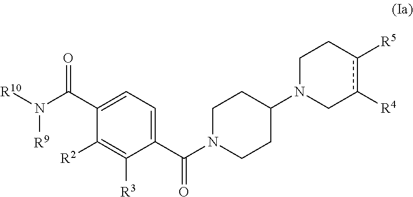
in which [symbol]
, R², R³, R⁴, R⁵, R⁹ and R¹⁰ have the meaning given above,
or
[F] compounds of the formula (VII) 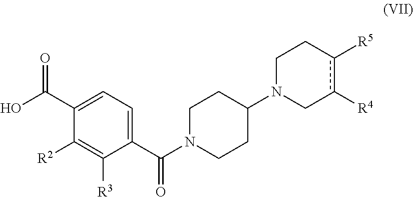
in which [symbol]
, R², R³, R⁴ and R⁵ have the meaning given above,
are, in a first step, reacted with oxalyl chloride or thionyl chloride and, in a second step, with compounds of the formula (VIII) 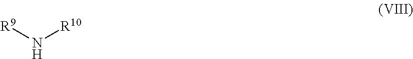
in which
R⁹ and R¹⁰ have the meaning given above,
to give compounds of the formula (Ia)
or
[G] compounds of the formula (IX) 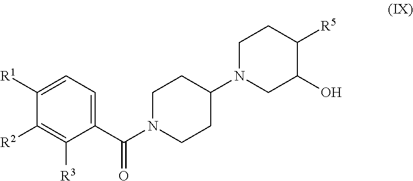
in which
R¹, R², R³ and R⁵ have the meaning given above, are reacted with compounds of the formula (X) 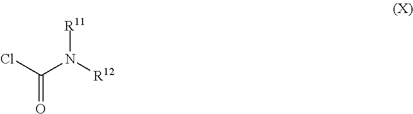
in which
R¹¹ and R¹² have the meaning given above,
to give compounds of the formula (Ib) 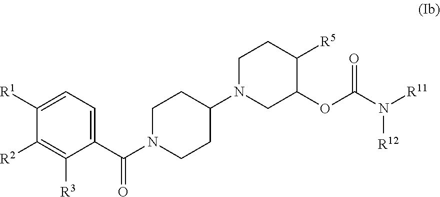
in which
R¹, R², R³, R⁵, R¹¹ and R¹² have the meaning given above,
or
[H] compounds of the formula (IX) 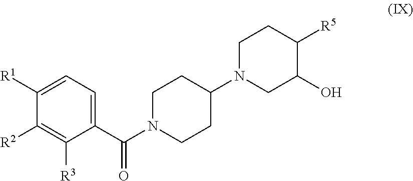
in which
R¹, R², R³ and R⁵ have the meaning given above,
are reacted with compounds of the formula (XI) 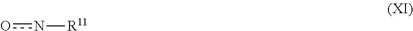
in which
R¹¹ has the meaning given above,
to give compounds of the formula (Ic) 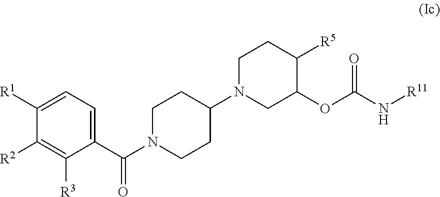
in which
R¹, R², R³, R⁵ and R¹¹ have the meaning given above,
or
[I] compounds of the formula (XII) 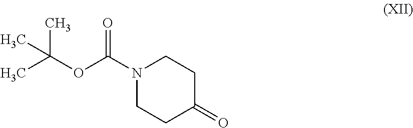
are reacted with compounds of the formula (XIII) 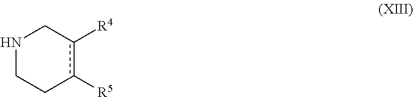
in which R⁴ and R⁵ have the meanings given above,
in the presence of a reducing agent to give compounds of the formula (XIV) 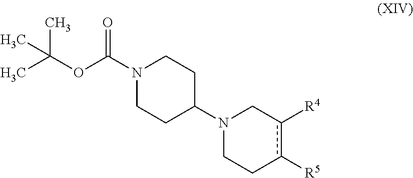
in which R⁴ and R⁵ have the meanings given above,
or
[J] compounds of the formula (XIV) 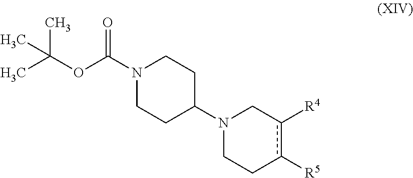
are reacted in the presence of an acid to give compounds of the formula (III) 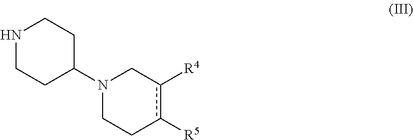
in which R⁴ and R⁵ have the meanings given above.

5. A method of making 3-(cyclopropyloxy)piperidine, where in a first step 3-hydroxypyridine is reacted with cyclopropyl bromide in the presence of an inorganic base in an inert solvent to give 3-(cyclopropyloxy)pyridine hydrochloride and the 3-(cyclopropyloxy)pyridine hydrochloride is reacted in a second step in the presence of hydrogen and a catalyst to give 3-(cyclopropyloxy)piperidine hydrochloride.

6. A method of making 3-[(trifluoromethoxy)methyl]piperidine which carries an amino protective group, where (piperidin-3-yl)methanol, carrying an amino protective group, is reacted in an inert solvent with carbon disulphide and iodomethane in the presence of sodium hydride in a first step to give S-methyl O-(piperidin-3-ylmethyl) carbonodithioate which carries an amino protective group and this is reacted in a second step with hydrogen fluoride/pyridine complex in an inert solvent to give 3-[(trifluoromethoxy)methyl]piperidine which carries an amino protective group.

7. A method of making 3-[(cyclopropyloxy)methyl]piperidine which carries an amino protective group, where in a first reaction step hydroxymethylpiperidine, carrying an amino protective group, is reacted in the presence of a catalyst in an inert solvent with ethyl vinyl ether to give vinyloxymethylpiperidine, which carries an amino protective group, and this is reacted in a second step in an inert solvent with diethylzinc and diiodomethane to give 3-[(cyclopropyloxy)methyl]piperidine, which carries an amino protective group.

8. A method for promoting wound healing of diabetic ulcers on the extremities comprising administering an effective amount of the compound of claim 1.

9. A method for the treatment and/or prophylaxis of primary and secondary forms of diabetic microangiopathies, diabetic wound healing, diabetic ulcers on the extremities, diabetic retinopathy, diabetic nephropathy, diabetic erectile dysfunction, diabetic heart failure, diabetic coronary microvascular heart disorders, peripheral and cardiac vascular disorders, thromboembolic disorders and ischaemias, peripheral circulatory disturbances, Raynaud's phenomenon, CREST syndrome, microcirculatory disturbances, intermittent claudication, and peripheral and autonomous neuropathies comprising administering an effective amount of the compound of claim 1.

10. A method for the treatment and/or prophylaxis of primary and secondary forms of diabetic microangiopathies, diabetic wound healing, diabetic ulcers on the extremities, diabetic retinopathy, diabetic nephropathy, diabetic erectile dysfunction, diabetic heart failure, diabetic coronary microvascular heart disorders, peripheral and cardiac vascular disorders, thromboembolic disorders and ischaemias, peripheral circulatory disturbances, Raynaud's phenomenon, CREST syndrome, microcirculatory disturbances, intermittent claudication, and peripheral and autonomous neuropathies comprising administering an effective amount of the compound of claim 1 with one or more inert non-toxic pharmaceutically suitable auxiliaries.

11. A medicament comprising the compound of as defined in claim 1 in combination with one or more inert non-toxic pharmaceutically suitable auxiliaries.

12. A medicament comprising the compound of claim 1 in combination with one or more further active compounds selected from the group consisting of lipid metabolism-modulating active compounds, antidiabetics, hypotensive agents, agents which lower the sympathetic tone, perfusion-enhancing and/or antithrombotic agents and also antioxidants, aldosterone and mineralocorticoid receptor antagonists, vasopressin receptor antagonists, organic nitrates and NO donors, IP receptor agonists, positive inotropic compounds, calcium sensitizers, ACE inhibitors, cGMP- and cAMP-modulating compounds, natriuretic peptides, NO-independent stimulators of guanylate cyclase, NO-independent activators of guanylate cyclase, inhibitors of human neutrophil elastase, compounds which inhibit the signal transduction cascade, compounds which modulate the energy metabolism of the heart, chemokine receptor antagonists, p38 kinase inhibitors, NPY agonists, orexin agonists, anorectics, PAF-AH inhibitors, antiphlogistics, analgesics, antidepressants and other psychopharmaceuticals.

13. A method for the treatment and/or prophylaxis of primary and secondary forms of diabetic microangiopathies, diabetic wound healing, diabetic ulcers on the extremities for promoting wound healing of diabetic foot ulcers, diabetic retinopathy, diabetic nephropathy, diabetic erectile dysfunction, diabetic heart failure, diabetic coronary microvascular heart disorders, peripheral and cardiac vascular disorders, thromboembolic disorders and ischaemias, peripheral circulatory disturbances, Raynaud's phenomenon, CREST syndrome, microcirculatory disturbances, intermittent claudication, and peripheral and autonomous neuropathies comprising administering an effective amount of the medicament of claim 11.

14. A method for the treatment and/or prophylaxis of primary and secondary forms of diabetic microangiopathies, diabetic wound healing, diabetic ulcers on the extremities, diabetic retinopathy, diabetic nephropathy, diabetic erectile dysfunction, diabetic heart failure, diabetic coronary microvascular heart disorders, peripheral and cardiac vascular disorders, thromboembolic disorders and ischaemias, peripheral circulatory disturbances, Raynaud's phenomenon, CREST syndrome, microcirculatory disturbances, intermittent claudication, and peripheral and autonomous neuropathies, in humans and animals comprising administering an effective amount the compound of claim 1.

15. Adrenoreceptor α2C receptor antagonists for use in a method for the treatment and/or prophylaxis of comorbidities and/or sequelae of diabetes mellitus, diabetic heart disorders, diabetic coronary heart disorders, diabetic coronary microvascular heart disorders, diabetic heart failure, diabetic cardiomyopathy and myocardial infarction, diabetic microangiopathy, diabetic retinopathy, diabetic neuropathy, diabetic nephropathy, diabetic erectile dysfunction, diabetic ulcers on the extremities, diabetic foot ulcers, for promoting diabetic wound healing, and for promoting wound healing of diabetic foot ulcers.

16. A method of promoting wound healing of diabetic ulcers on the extremities comprising administering the medicament of claim 11.

17. A method for the treatment and/or prophylaxis of diabetic disorders or diseases using the compound of claim 1.

18. A method for the treatment and/or prophylaxis of diabetic disorders or diseases by administration of an effective amount of the medicament of claim 11.

19. A medicament comprising the compound of claim 1 in combination with one or more further active compounds selected from the group consisting of rivaroxaban, iloprost, and inhibitors of phosphodiesterases (PDE) 1, 2, 3, 4, and/or 5 comprising administering an effective amount of the compound of claim 1.

20. A method for the treatment and/or prophylaxis of peripheral occlusive disease comprising administering an effective amount of the compound of claim 1.

21. A method for the treatment and/or prophylaxis of peripheral and cardiac vascular disorders, peripheral circulatory disturbances, or intermittent claudication comprising administering an effective amount of the compound of claim 1.
